SEQUENCE LISTING (203 sequences)

SEQ ID NO: 1 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - protein, 157 aa, Homo sapiens [sequence omitted]

SEQ ID NO: 3 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 4 - protein, 158 aa, Homo sapiens
LIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 5 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 6 - protein, 152 aa, Homo sapiens
QIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 7 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 8 - protein, 180 aa, Homo sapiens
LKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 9 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 10 - protein, 426 aa, Homo sapiens
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 11 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 12 - protein, 439 aa, Homo sapiens
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 13 - protein, 283 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 14 - protein, 245 aa, Homo sapiens
LPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 15 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 16 - protein, 310 aa, Homo sapiens
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 17 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 18 - protein, 393 aa, Homo sapiens
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 19 - protein, 300 aa, Homo sapiens
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 20 - protein, 271 aa, Homo sapiens
VAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 21 - protein, 435 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 22 - protein, 405 aa, Homo sapiens
QAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 23 - protein, 95 aa, synthetic construct
TVYVVKSGVNKGKICDTCPPGTYKKRDCDRSLPTVCEPCGDGEYTSMNNSLPECLSCNHCYDPTEIEITPCNATTNTVCSCKEGYTFDSSIQGCI

SEQ ID NO: 24 - protein, 91 aa, synthetic construct
STYRVRSSGLTCSTCPPGTHKERDCSLNTETICKACGEGEYTAHNNSLPKCLACKSCFNATEIETKSCDPTSDTICACREGYSINNLGECN

SEQ ID NO: 25 - protein, 91 aa, synthetic construct
STYRVRSSGLTCSMCPPGTYKERDCSLNTETLCKACGEGEYTAHKNSLPKCLACKSCFNATEIETKSCDPISDTICTCREGYSINNLGECN

SEQ ID NO: 26 - protein, 90 aa, synthetic construct
RSKINSSLICDMCPPGYYKNKDCTSTSTTICLPCGEGEYTAYNNSLTKCIRCKDCYEENEKIFKPCNSTSDTICTCIDGYTKDETTDSCI

SEQ ID NO: 27 - protein, 91 aa, synthetic construct
DTFTDEETGLVCEKCPPGTYREADCTETTKTVCKACGEGTFTDHPNNLPECKSCSTCDEATEVEVQKCTATANTVCACKDGFTVNEKGECV

SEQ ID NO: 28 - protein, 91 aa, synthetic construct
DTYTDEKTGLTCEKCPPGTYREADCGENSKTVCKACGEGTYTDAPNHLPECKKCNTCDAETEVEVKACTATSDTVCACKEGFTVNENGECV

SEQ ID NO: 29 - protein, 91 aa, synthetic construct
STYTDSATGRTCSKCPPGTYRSADCGATTSTVCTACGAGTYTDKDNNLSACLACSTCNAATETEVSACTATANTVCACKAGYTLDANGKCV

SEQ ID NO: 30 - protein, 91 aa, synthetic construct
STYKDEKTGLTCQKCPPGTYRKADCGLFSKTECAACGEGTYTDKDNNLSKCLKCSTCNEETEVEVQKCTATADTVCACKEGFTKNEKGECQ

SEQ ID NO: 31 - protein, 91 aa, synthetic construct
PTYRDEATGRVCQECPPGTYLEAHCGENTSTVCKACGEGTFTDKPNHLPQCLSCSTCEAATEVEVRACTATANTVCACREGFTRNEQGKCN

SEQ ID NO: 32 - protein, 91 aa, synthetic construct
DTFTDEATGLTCAKCPPGTYREADCGLFSKTVCKACGEGTFTAEPNNLPTCKACNTCDAATEVEVQACTATADTVCACKEGFTKNENGECN

SEQ ID NO: 33 - protein, 91 aa, synthetic construct
ATYTDTATGLTCAKCPPGTFRKADCGATTATVCEACGAGTYTAADNHLSACLACTTCDAATETETAACTATANTVCACKAGFTVDANGKCG

SEQ ID NO: 34 - protein, 91 aa, synthetic construct
PTYTDTATGLTCAKCPPGTYRERDCGLFSDTVCKACGEGTYTDTPNHLPKCLSCSTCDAATETEVQACTATANTVCACKDGFTVDSNGKCQ

SEQ ID NO: 35 - protein, 91 aa, synthetic construct
DTYTDTATGLTCAKCPPGTYREADCGLFSETVCKACGAGTYTDKPNHLPACLACGTCDAATEVEVQACTATANTVCACKAGFTKNEKGECV

SEQ ID NO: 36 - protein, 91 aa, synthetic construct
PTYTDTATGLTCEQCPPGTYRRADCGATTATVCAACGAGTYTSAPNNLPTCLACPTCDAATEVEVQPCVATANTVCACRAGFTVDAQGRCQ

SEQ ID NO: 37 - protein, 91 aa, synthetic construct
ATYTDTATGLTCTKCPPGTHRKADCGLLTSTVCEACGEGTYTSEDNNLSSCLSCSTCNEATETEVQACTATSDTVCECKEGFTVDANGKCG

SEQ ID NO: 38 - protein, 91 aa, synthetic construct
ATYKDEKTGLTCEKCPPGTYREADCTETTKTVCKACGEGTYTDKDNNLPKCLECNTCDEKTEVEVQKCTATSNTVCACKEGFTKNEKGECK

SEQ ID NO: 39 - protein, 91 aa, synthetic construct
KTYKDPETGLTCEKCPPGTYLEANCTPTSPTVCKPCGEGTYTEEENALTACKPCSSCDPATEEVVQPCTPTSDTVCSCKPGYSFNSNGLCV

SEQ ID NO: 40 - protein, 91 aa, synthetic construct
ETYTDSSTGLTCAKCPPGYYLSQTCTETSPTVCKACGSGTYTTTPNALTSCKACSTCDSSTEVTTKSCTPTSDTVCSCKSGYSKNSSGKCV

SEQ ID NO: 41 - protein, 91 aa, synthetic construct
QTYTDPATGLTCDLCPPGTYLEAACTATSPTVCKPCPSGTYTTAPNALTSCRKCSTCNPATEVTTQPCTPTSDTVCSCKSGYTLDSNGQCV

SEQ ID NO: 42 - protein, 91 aa, synthetic construct
KTYVDPTTGLTCDMCPPGTYLKKTCSLNSRTVCKPCGKGTYTSTYNNLKKCKKCSTCDPRTEVVSQPCTQTSDTVCECRPGYSRDSNGRCV

SEQ ID NO: 43 - protein, 91 aa, synthetic construct
ATYTDPETGLTCEKCPPGTYLEAPCTETTPTVCKPCGEGEYTTVPNALRKCRECTTCNPATEEVSADCTPTSNTVCTCKPGYVKDANGKCV

SEQ ID NO: 44 - protein, 91 aa, synthetic construct
QTYVDPATGLTCTLCPPGTYLKKPCTATSPTVCKPCGSGTYTSKPNALTKCQECTTCDPATERVVRPCTPTHNTVCECKPGYKRNSKGQCV

SEQ ID NO: 45 - protein, 91 aa, synthetic construct
ATYTDPATGLVCELCPPGSYLAAPCTATTPTVCKPCPEGYYTTEPNALPRCLPCTTCNPETEVVIEPCTPTSDTVCECKPGYSLNENGECV

SEQ ID NO: 46 - protein, 91 aa, synthetic construct
DTYTDPTTGLTCKKCPPGTYLKKNCTATSPTVCKKCGPGQYTTKYNNLKKCNKCSTCDPTTEEVSQACTPTHDTVCKCKPGYSLNSKGRCV

SEQ ID NO: 47 - protein, 91 aa, synthetic construct
STYTNPSTGLTCSMCPPGTYLERDCSSSSGTVCKPCGPGTYTTAYNNLKSCKKCTKCDPKTEKVVKECTPTSNTVCECKPGFKFDSNGKCR

SEQ ID NO: 48 - protein, 91 aa, synthetic construct
KTFVDDKTGLTCEQCPPGTYLESTCSETSPTVCKPCGPGEYTTEYNNLTKCKKCSTCDPATEVVVSACTPTSDTVCACKPGFSFNEEGKCV

SEQ ID NO: 49 - protein, 91 aa, synthetic construct
STYVNPTTGLTCSKCPPGTYLKASCTATSKTVCTPCGSGTYTTTANNLSSCKQCSTCDPATETVSQPCTPTSDTVCSCKSGYTFDSNGKCV

SEQ ID NO: 50 - protein, 91 aa, synthetic construct
ETYKDPSTGLLCEKCPPGTYLSSPCTTTSGTVCKPCGSGTYTTQPNALSSCSSCSTCDSATQTVSKACTSTSDTVCSCKSGYSLDSNGNCV

SEQ ID NO: 51 - protein, 91 aa, synthetic construct
ETYTDPATGRVCEKCPPGTYVAAPCTATSPTVCRPCGPGTYTSAPNALTSCLACTSCDPATEAVSQPCTPTANTVCVCKPGFSFNANGQCV

SEQ ID NO: 52 - protein, 91 aa, synthetic construct
ETYTNASTGLTCNKCPPGTYLAKDCTSTSDTVCQDCGPGTYTTSPNAKSSCNACSTCDPATEEVITPCTPTSDTVCACKPGFTWNQNGQCV

SEQ ID NO: 53 - protein, 91 aa, synthetic construct
ETYTDPATGLTCQKCPPGTYLKAPCTSTSPTVCTPCGSGYYTSSSNNLTSCSKCNTCDPSTESVSSACTPTSNTVCSCKSGYTLDSNGNCV

SEQ ID NO: 54 - protein, 112 aa, synthetic construct
MIITLLALLLVVSTESSTVYVVKSGVNKGKICDTCPPGTYKKRDCDRSLPTVCEPCGDGEYTSMNNSLPECLSCNHCYDPTEIEITPCNATTNTVCSCKEGYTFDSSIQGCI

SEQ ID NO: 55 - protein, 116 aa, synthetic construct
MGKNSLVLTIGVLMTLITLRSSFSASTYRVRSSGLTCSTCPPGTHKERDCSLNTETICKACGEGEYTAHNNSLPKCLACKSCFNATEIETKSCDPTSDTICACREGYSINNLGECN

SEQ ID NO: 56 - protein, 116 aa, synthetic construct
MGKNSLVLMIGVLMTLITLRSSFSASTYRVRSSGLTCSMCPPGTYKERDCSLNTETLCKACGEGEYTAHKNSLPKCLACKSCFNATEIETKSCDPISDTICTCREGYSINNLGECN

SEQ ID NO: 57 - protein, 117 aa, synthetic construct
MHYKNKMSVNILIITVLIGISFQASTYRSKINSSLICDMCPPGYYKNKDCTSTSTTICLPCGEGEYTAYNNSLTKCIRCKDCYEENEKIFKPCNSTSDTICTCIDGYTKDETTDSCI

SEQ ID NO: 58 - protein, 116 aa, synthetic construct
MGKNSLVLTIGVLMTLITLRSSFSADTFTDEETGLVCEKCPPGTYREADCTETTKTVCKACGEGTFTDHPNNLPECKSCSTCDEATEVEVQKCTATANTVCACKDGFTVNEKGECV

SEQ ID NO: 59 - protein, 116 aa, synthetic construct
MGKNSLVLTIGVLMTLITLRSSFSADTYTDEKTGLTCEKCPPGTYREADCGENSKTVCKACGEGTYTDAPNHLPECKKCNTCDAETEVEVKACTATSDTVCACKEGFTVNENGECV

SEQ ID NO: 60 - protein, 116 aa, synthetic construct
MGKNSLVLTIGVLMTLITLRSSFSASTYTDSATGRTCSKCPPGTYRSADCGATTSTVCTACGAGTYTDKDNNLSACLACSTCNAATETEVSACTATANTVCACKAGYTLDANGKCV

SEQ ID NO: 61 - protein, 116 aa, synthetic construct
MGKNSLVLTIGVLMTLITLRSSFSASTYKDEKTGLTCQKCPPGTYRKADCGLFSKTECAACGEGTYTDKDNNLSKCLKCSTCNEETEVEVQKCTATADTVCACKEGFTKNEKGECQ

SEQ ID NO: 62 - protein, 116 aa, synthetic construct
MGKNSLVLTIGVLMTLITLRSSFSAPTYRDEATGRVCQECPPGTYLEAHCGENTSTVCKACGEGTFTDKPNHLPQCLSCSTCEAATEVEVRACTATANTVCACREGFTRNEQGKCN

SEQ ID NO: 63 - protein, 116 aa, synthetic construct
MGKNSLVLTIGVLMTLITLRSSFSADTFTDEATGLTCAKCPPGTYREADCGLFSKTVCKACGEGTFTAEPNNLPTCKACNTCDAATEVEVQACTATADTVCACKEGFTKNENGECN

SEQ ID NO: 64 - protein, 116 aa, synthetic construct
MGKNSLVLTIGVLMTLITLRSSFSAATYTDTATGLTCAKCPPGTFRKADCGATTATVCEACGAGTYTAADNHLSACLACTTCDAATETETAACTATANTVCACKAGFTVDANGKCG

SEQ ID NO: 65 - protein, 116 aa, synthetic construct
MGKNSLVLTIGVLMTLITLRSSFSAPTYTDTATGLTCAKCPPGTYRERDCGLFSDTVCKACGEGTYTDTPNHLPKCLSCSTCDAATETEVQACTATANTVCACKDGFTVDSNGKCQ

SEQ ID NO: 66 - protein, 116 aa, synthetic construct
MGKNSLVLTIGVLMTLITLRSSFSADTYTDTATGLTCAKCPPGTYREADCGLFSETVCKACGAGTYTDKPNHLPACLACGTCDAATEVEVQACTATANTVCACKAGFTKNEKGECV

SEQ ID NO: 67 - protein, 116 aa, synthetic construct
MGKNSLVLTIGVLMTLITLRSSFSAPTYTDTATGLTCEQCPPGTYRRADCGATTATVCAACGAGTYTSAPNNLPTCLACPTCDAATEVEVQPCVATANTVCACRAGFTVDAQGRCQ

SEQ ID NO: 68 - protein, 116 aa, synthetic construct
MGKNSLVLTIGVLMTLITLRSSFSAATYTDTATGLTCTKCPPGTHRKADCGLLTSTVCEACGEGTYTSEDNNLSSCLSCSTCNEATETEVQACTATSDTVCECKEGFTVDANGKCG

SEQ ID NO: 69 - protein, 116 aa, synthetic construct
MGKNSLVLTIGVLMTLITLRSSFSAATYKDEKTGLTCEKCPPGTYREADCTETTKTVCKACGEGTYTDKDNNLPKCLECNTCDEKTEVEVQKCTATSNTVCACKEGFTKNEKGECK

SEQ ID NO: 70 - protein, 116 aa, synthetic construct
MGKNSLVLTIGVLMTLITLRSSFSAKTYKDPETGLTCEKCPPGTYLEANCTPTSPTVCKPCGEGTYTEEENALTACKPCSSCDPATEEVVQPCTPTSDTVCSCKPGYSFNSNGLCV

SEQ ID NO: 71 - protein, 116 aa, synthetic construct
MGKNSLVLTIGVLMTLITLRSSFSAETYTDSSTGLTCAKCPPGYYLSQTCTETSPTVCKACGSGTYTTTPNALTSCKACSTCDSSTEVTTKSCTPTSDTVCSCKSGYSKNSSGKCV

SEQ ID NO: 72 - protein, 116 aa, synthetic construct
MGKNSLVLTIGVLMTLITLRSSFSAQTYTDPATGLTCDLCPPGTYLEAACTATSPTVCKPCPSGTYTTAPNALTSCRKCSTCNPATEVTTQPCTPTSDTVCSCKSGYTLDSNGQCV

SEQ ID NO: 73 - protein, 116 aa, synthetic construct
MGKNSLVLTIGVLMTLITLRSSFSAKTYVDPTTGLTCDMCPPGTYLKKTCSLNSRTVCKPCGKGTYTSTYNNLKKCKKCSTCDPRTEVVSQPCTQTSDTVCECRPGYSRDSNGRCV

SEQ ID NO: 74 - protein, 116 aa, synthetic construct
MGKNSLVLTIGVLMTLITLRSSFSAATYTDPETGLTCEKCPPGTYLEAPCTETTPTVCKPCGEGEYTTVPNALRKCRECTTCNPATEEVSADCTPTSNTVCTCKPGYVKDANGKCV

SEQ ID NO: 75 - protein, 116 aa, synthetic construct
MGKNSLVLTIGVLMTLITLRSSFSAQTYVDPATGLTCTLCPPGTYLKKPCTATSPTVCKPCGSGTYTSKPNALTKCQECTTCDPATERVVRPCTPTHNTVCECKPGYKRNSKGQCV

SEQ ID NO: 76 - protein, 116 aa, synthetic construct
MGKNSLVLTIGVLMTLITLRSSFSAATYTDPATGLVCELCPPGSYLAAPCTATTPTVCKPCPEGYYTTEPNALPRCLPCTTCNPETEVVIEPCTPTSDTVCECKPGYSLNENGECV

SEQ ID NO: 77 - protein, 116 aa, synthetic construct
MGKNSLVLTIGVLMTLITLRSSFSADTYTDPTTGLTCKKCPPGTYLKKNCTATSPTVCKKCGPGQYTTKYNNLKKCNKCSTCDPTTEEVSQACTPTHDTVCKCKPGYSLNSKGRCV

SEQ ID NO: 78 - protein, 116 aa, synthetic construct
MGKNSLVLTIGVLMTLITLRSSFSASTYTNPSTGLTCSMCPPGTYLERDCSSSSGTVCKPCGPGTYTTAYNNLKSCKKCTKCDPKTEKVVKECTPTSNTVCECKPGFKFDSNGKCR

SEQ ID NO: 79 - protein, 116 aa, synthetic construct
MGKNSLVLTIGVLMTLITLRSSFSAKTFVDDKTGLTCEQCPPGTYLESTCSETSPTVCKPCGPGEYTTEYNNLTKCKKCSTCDPATEVVVSACTPTSDTVCACKPGFSFNEEGKCV

SEQ ID NO: 80 - protein, 116 aa, synthetic construct
MGKNSLVLTIGVLMTLITLRSSFSASTYVNPTTGLTCSKCPPGTYLKASCTATSKTVCTPCGSGTYTTTANNLSSCKQCSTCDPATETVSQPCTPTSDTVCSCKSGYTFDSNGKCV

SEQ ID NO: 81 - protein, 116 aa, synthetic construct
MGKNSLVLTIGVLMTLITLRSSFSAETYKDPSTGLLCEKCPPGTYLSSPCTTTSGTVCKPCGSGTYTTQPNALSSCSSCSTCDSATQTVSKACTSTSDTVCSCKSGYSLDSNGNCV

SEQ ID NO: 82 - protein, 116 aa, synthetic construct
MGKNSLVLTIGVLMTLITLRSSFSAETYTDPATGRVCEKCPPGTYVAAPCTATSPTVCRPCGPGTYTSAPNALTSCLACTSCDPATEAVSQPCTPTANTVCVCKPGFSFNANGQCV

SEQ ID NO: 83 - protein, 116 aa, synthetic construct
MGKNSLVLTIGVLMTLITLRSSFSAETYTNASTGLTCNKCPPGTYLAKDCTSTSDTVCQDCGPGTYTTSPNAKSSCNACSTCDPATEEVITPCTPTSDTVCACKPGFTWNQNGQCV

SEQ ID NO: 84 - protein, 116 aa, synthetic construct
MGKNSLVLTIGVLMTLITLRSSFSAETYTDPATGLTCQKCPPGTYLKAPCTSTSPTVCTPCGSGYYTSSSNNLTSCSKCNTCDPSTESVSSACTPTSNTVCSCKSGYTLDSNGNCV

SEQ ID NO: 85 - protein, 20 aa, Homo sapiens
MYRMQLLSCIALSLALVTNS

SEQ ID NO: 86 - protein, 25 aa, Homo sapiens
MGVLLTQRTLLSLVLALLFPSMASM

SEQ ID NO: 87 - protein, 18 aa, Homo sapiens
MASLWLLSCFSLVGAAFG

SEQ ID NO: 88 - protein, 15 aa, Homo sapiens
MNLLLILTFVAAAVA

SEQ ID NO: 89 - protein, 24 aa, Homo sapiens
MALWMRLLPLLALLALWGPDPAAA

SEQ ID NO: 90 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 91 - protein, 12 aa, Homo sapiens
EPKSCDKTHTCP

SEQ ID NO: 92 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 93 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 106 aa, synthetic construct [sequence omitted]

SEQ ID NO: 95 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 219 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 330 aa, synthetic construct [sequence omitted]

SEQ ID NO: 102 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 104 - protein, 12 aa, Homo sapiens
ESKYGPPCPSCP

SEQ ID NO: 105 - protein, 13 aa, synthetic construct
AESKYGPPCPSCP

SEQ ID NO: 106 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 107 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 108 - protein, 106 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 109 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 216 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 111 - protein, 223 aa, synthetic construct
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 112 - protein, 222 aa, synthetic construct
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 113 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 114 - protein, 228 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 115 - protein, 230 aa, synthetic construct
AESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 229 aa, synthetic construct
AESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 117 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 119 - protein, 107 aa, Homo sapiens [sequence omitted]

SEQ ID NO: 120 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 121 - protein, 97 aa, Mus sp.
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKI

SEQ ID NO: 122 - protein, 13 aa, Mus sp.
VPRDCGCKPCICT

SEQ ID NO: 123 - protein, 107 aa, Mus sp.
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTK

SEQ ID NO: 124 - protein, 107 aa, synthetic construct
GRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 125 - protein, 106 aa, synthetic construct
GRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 126 - protein, 214 aa, synthetic construct
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 127 - protein, 213 aa, synthetic construct
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 128 - protein, 227 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 129 - protein, 227 aa, synthetic construct; identical to SEQ ID NO: 128

SEQ ID NO: 130 - protein, 16 aa, Mus sp.
EPRGPTIKPCPPCKCP

SEQ ID NO: 131 - protein, 134 aa, Mus sp.
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVT

SEQ ID NO: 132 - protein, 83 aa, synthetic construct
LTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 133 - protein, 82 aa, synthetic construct
LTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 134 - protein, 217 aa, synthetic construct
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 135 - protein, 216 aa, synthetic construct
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 136 - protein, 233 aa, synthetic construct
EPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 137 - protein, 232 aa, synthetic construct
EPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 138 - protein, 330 aa, synthetic construct
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIEPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 139 - protein, 329 aa, synthetic construct
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIEPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 140 - protein, 107 aa, Mus sp.
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 141 - protein, 105 aa, Mus sp.
QPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 142 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 144 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 146 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 148 - protein, 217 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 149 - protein, 216 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 150 - protein, 223 aa, synthetic construct
PCPSCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 151 - protein, 222 aa, synthetic construct
PCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 152 - protein, 229 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 153 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 154 - protein, 230 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 155 - protein, 229 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 156 - protein, 217 aa, synthetic construct
APNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 157 - protein, 216 aa, synthetic construct
APNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 158 - protein, 233 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 159 - protein, 232 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 160 - protein, 217 aa, synthetic construct
APNAAGGPSVFIFAAKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 161 - protein, 216 aa, synthetic construct
APNAAGGPSVFIFAAKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 162 - protein, 233 aa, synthetic construct
EPRGPTIKPCAACKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 163 - protein, 232 aa, synthetic construct
EPRGPTIKPCAACKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 164 - protein, 8 aa, synthetic construct
GGGGGGGS

SEQ ID NO: 165 - protein, 16 aa, synthetic construct
GGGGGGGSGGGGGGGS

SEQ ID NO: 166 - protein, 24 aa, synthetic construct
GGGGGGGSGGGGGGGSGGGGGGGS

SEQ ID NO: 167 - protein, 5 aa, synthetic construct
GGGGS

SEQ ID NO: 168 - protein, 10 aa, synthetic construct
GGGGSGGGGS

SEQ ID NO: 169 - protein, 15 aa, synthetic construct
GGGGSGGGGSGGGGS

SEQ ID NO: 170 - protein, 4 aa, synthetic construct
GGGS

SEQ ID NO: 171 - protein, 8 aa, synthetic construct
GGGSGGGS

SEQ ID NO: 172 - protein, 12 aa, synthetic construct
GGGSGGGSGGGS

SEQ ID NO: 173 - protein, 18 aa, synthetic construct
GGGGGGGSGGGGSGGGGS

SEQ ID NO: 174 - protein, 362 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSTVYVVKSGVNKGKICDTCPPGTYKKRDCDRSLPTVCEPCGDGEYTSMNNSLPECLSCNHCYDPTEIEITPCNATTNTVCSCKEGYTFDSSIQGCI

SEQ ID NO: 175 - protein, 342 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSTVYVVKSGVNKGKICDTCPPGTYKKRDCDRSLPTVCEPCGDGEYTSMNNSLPECLSCNHCYDPTEIEITPCNATTNTVCSCKEGYTFDSSIQGCI

SEQ ID NO: 176 - protein, 341 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSTVYVVKSGVNKGKICDTCPPGTYKKRDCDRSLPTVCEPCGDGEYTSMNNSLPECLSCNHCYDPTEIEITPCNATTNTVCSCKEGYTFDSSIQGCI

SEQ ID NO: 177 - protein, 358 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 178 - protein, 338 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 179 - protein, 337 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 180 - protein, 358 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSMCPPGTYKERDCSLNTETLCKACGEGEYTAHKNSLPKCLACKSCFNATEIETKSCDPISDTICTCREGYSINNLGECN

SEQ ID NO: 181 - protein, 338 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSMCPPGTYKERDCSLNTETLCKACGEGEYTAHKNSLPKCLACKSCFNATEIETKSCDPISDTICTCREGYSINNLGECN

SEQ ID NO: 182 - protein, 337 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSMCPPGTYKERDCSLNTETLCKACGEGEYTAHKNSLPKCLACKSCFNATEIETKSCDPISDTICTCREGYSINNLGECN

SEQ ID NO: 183 - protein, 357 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRSKINSSLICDMCPPGYYKNKDCTSTSTTICLPCGEGEYTAYNNSLTKCIRCKDCYEENEKIFKPCNSTSDTICTCIDGYTKDETTDSCI

SEQ ID NO: 184 - protein, 337 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRSKINSSLICDMCPPGYYKNKDCTSTSTTICLPCGEGEYTAYNNSLTKCIRCKDCYEENEKIFKPCNSTSDTICTCIDGYTKDETTDSCI

SEQ ID NO: 185 - protein, 336 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRSKINSSLICDMCPPGYYKNKDCTSTSTTICLPCGEGEYTAYNNSLTKCIRCKDCYEENEKIFKPCNSTSDTICTCIDGYTKDETTDSCI

SEQ ID NO: 186 - protein, 235 aa, Homo sapiens
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 187 - protein, 95 aa, synthetic construct
MSTYNTKTSPSLKCDMCPPGYYKHEDCTSNTKTVCSPCGEGEYTAYNNSLTKCLRCSDCYGENEITTKQCTNTSNTVCECMDGYTKDETIDACIK

SEQ ID NO: 188 - protein, 100 aa, synthetic construct
ASTYTSKIDASLICDMCPPGSYKYKDCTYDSKTVCLPCGDGEYTSYNNSLAKCLRCDDCYDENEVTAKPCDSTSNTICKCMDGYTKDNTISACVKTSHIT

SEQ ID NO: 189 - protein, 117 aa, synthetic construct
MSNNLCQIITICIITMVCISYQMSTYNTKTSPSLKCDMCPPGYYKHEDCTSNTKTVCSPCGEGEYTAYNNSLTKCLRCSDCYGENEITTKQCTNTSNTVCECMDGYTKDETIDACIK

SEQ ID NO: 190 - protein, 123 aa, synthetic construct
MTNYLYISANIFVIILLAEITFQASTYTSKIDASLICDMCPPGSYKYKDCTYDSKTVCLPCGDGEYTSYNNSLAKCLRCDDCYDENEVTAKPCDSTSNTICKCMDGYTKDNTISACVKTSHIT

SEQ ID NO: 191 - protein, 362 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMSTYNTKTSPSLKCDMCPPGYYKHEDCTSNTKTVCSPCGEGEYTAYNNSLTKCLRCSDCYGENEITTKQCTNTSNTVCECMDGYTKDETIDACIK

SEQ ID NO: 192 - protein, 342 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMSTYNTKTSPSLKCDMCPPGYYKHEDCTSNTKTVCSPCGEGEYTAYNNSLTKCLRCSDCYGENEITTKQCTNTSNTVCECMDGYTKDETIDACIK

SEQ ID NO: 193 - protein, 341 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMSTYNTKTSPSLKCDMCPPGYYKHEDCTSNTKTVCSPCGEGEYTAYNNSLTKCLRCSDCYGENEITTKQCTNTSNTVCECMDGYTKDETIDACIK

SEQ ID NO: 194 - protein, 367 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSASTYTSKIDASLICDMCPPGSYKYKDCTYDSKTVCLPCGDGEYTSYNNSLAKCLRCDDCYDENEVTAKPCDSTSNTICKCMDGYTKDNTISACVKTSHIT

SEQ ID NO: 195 - protein, 347 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSASTYTSKIDASLICDMCPPGSYKYKDCTYDSKTVCLPCGDGEYTSYNNSLAKCLRCDDCYDENEVTAKPCDSTSNTICKCMDGYTKDNTISACVKTSHIT

SEQ ID NO: 196 - protein, 346 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSASTYTSKIDASLICDMCPPGSYKYKDCTYDSKTVCLPCGDGEYTSYNNSLAKCLRCDDCYDENEVTAKPCDSTSNTICKCMDGYTKDNTISACVKTSHIT

SEQ ID NO: 197 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 199 - protein, 258 aa, synthetic construct
KVPTWTDPVTGLTCDRCPAGTHVVKQCTATTPTECGACPANHYTEFWNYLNRCLYCGVRCTEQQVEKSTCSATHNRVCECKPGYHIYADDFCLRHSTCPPGQGLLVAGTPTSNTRCGPCQAGYFSAEDSTEACKPHTPCKDTERSVPGTATQDTFCTSCQARKCDITAPPAPDKAVCDDAFIDFVERYPLNAKKAKNLMRKLRKMGKGKTIRESFQAIMAKRNDQPLACEVLSTLNDVDSDMGCRIKTFFLGWNEDEC

SEQ ID NO: 200 - protein, 276 aa, synthetic construct
MFRGTLMVLACVAACFSEKVPTWTDPVTGLTCDRCPAGTHVVKQCTATTPTECGACPANHYTEFWNYLNRCLYCGVRCTEQQVEKSTCSATHNRVCECKPGYHIYADDFCLRHSTCPPGQGLLVAGTPTSNTRCGPCQAGYFSAEDSTEACKPHTPCKDTERSVPGTATQDTFCTSCQARKCDITAPPAPDKAVCDDAFIDFVERYPLNAKKAKNLMRKLRKMGKGKTIRESFQAIMAKRNDQPLACEVLSTLNDVDSDMGCRIKTFFLGWNEDEC

SEQ ID NO: 201 - protein, 525 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSKVPTWTDPVTGLTCDRCPAGTHVVKQCTATTPTECGACPANHYTEFWNYLNRCLYCGVRCTEQQVEKSTCSATHNRVCECKPGYHIYADDFCLRHSTCPPGQGLLVAGTPTSNTRCGPCQAGYFSAEDSTEACKPHTPCKDTERSVPGTATQDTFCTSCQARKCDITAPPAPDKAVCDDAFIDFVERYPLNAKKAKNLMRKLRKMGKGKTIRESFQAIMAKRNDQPLACEVLSTLNDVDSDMGCRIKTFFLGWNEDEC

SEQ ID NO: 202 - protein, 505 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSKVPTWTDPVTGLTCDRCPAGTHVVKQCTATTPTECGACPANHYTEFWNYLNRCLYCGVRCTEQQVEKSTCSATHNRVCECKPGYHIYADDFCLRHSTCPPGQGLLVAGTPTSNTRCGPCQAGYFSAEDSTEACKPHTPCKDTERSVPGTATQDTFCTSCQARKCDITAPPAPDKAVCDDAFIDFVERYPLNAKKAKNLMRKLRKMGKGKTIRESFQAIMAKRNDQPLACEVLSTLNDVDSDMGCRIKTFFLGWNEDEC

SEQ ID NO: 203 - protein, 504 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSKVPTWTDPVTGLTCDRCPAGTHVVKQCTATTPTECGACPANHYTEFWNYLNRCLYCGVRCTEQQVEKSTCSATHNRVCECKPGYHIYADDFCLRHSTCPPGQGLLVAGTPTSNTRCGPCQAGYFSAEDSTEACKPHTPCKDTERSVPGTATQDTFCTSCQARKCDITAPPAPDKAVCDDAFIDFVERYPLNAKKAKNLMRKLRKMGKGKTIRESFQAIMAKRNDQPLACEVLSTLNDVDSDMGCRIKTFFLGWNEDEC

(19) United States
(12) Patent Application Publication
(10) Pub. No.: US 2025/0375499 A1
(43) Pub. Date: Dec. 11, 2025

(54) IMMUNORECEPTOR INHIBITORY PROTEINS AND RELATED METHODS

(71) Applicant: Flagship Pioneering Innovations VII, LLC, Cambridge, MA (US)

(72) Inventors: Hozefa Saifuddin BANDUKWALA, Southborough, MA (US); Williams Ernesto MIRANDA DELGADO, Calgary (CA); Samuel Clement HASSAN, Somerville, MA (US); Angela Wynne NORTON, Reading, MA (US); Sofia Marques Tuna Ribeiro BRITES BOSS, Boston, MA (US); Ellen Lovisa Larsdotter AFZELIUS, Belmont, MA (US)

(21) Appl. No.: 19/036,498

(22) Filed: Jan. 24, 2025

Related U.S. Application Data
(60) Provisional application No. 63/625,702, filed on Jan. 26, 2024.
(60) Provisional application No. 63/625,755, filed on Jan. 26, 2024.
(60) Provisional application No. 63/653,350, filed on May 30, 2024.
(60) Provisional application No. 63/653,360, filed on May 30, 2024.
(60) Provisional application No. 63/671,469, filed on Jul. 15, 2024.
(60) Provisional application No. 63/671,523, filed on Jul. 15, 2024.
(60) Provisional application No. 63/684,950, filed on Aug. 20, 2024.
(60) Provisional application No. 63/715,911, filed on Nov. 4, 2024.
(60) Provisional application No. 63/715,916, filed on Nov. 4, 2024.

(51) Int. Cl. A61K 38/00; A61K 31/7088; A61K 39/00; A61K 39/12; A61K 51/08; A61P 29/00; C07K 14/00; C07K 16/00; C12N 7/00; C12N 15/62; C12N 15/63; G01N 33/68

(52) U.S. Cl. CPC A61K 38/00; A61K 31/7088; A61K 39/12; A61K 51/08; A61P 29/00; C07K 14/00; C07K 16/00; C12N 7/00; C12N 15/62; C12N 15/63; G01N 33/68; A61K 2039/53; A61K 2039/55516; C07K 2319/00; G01N 2800/26

(57) ABSTRACT

Provided herein are, inter alia, immunoreceptor inhibitory proteins and compositions (e.g., pharmaceutical compositions) comprising the same; as well as methods of making the immunoreceptor inhibitory proteins and compositions. The immunoreceptor inhibitory proteins provided herein are useful in e.g., pharmaceutical compositions and methods of use, including e.g., in the treatments of pro-inflammatory (e.g., autoimmune) diseases.

### RELATED APPLICATIONS

This application claims priority to U.S. Ser. No. 63/625,702, filed Jan. 26, 2024, U.S. Ser. No. 63/625,755, filed Jan. 26, 2024, U.S. Ser. No. 63/653,350, filed May 30, 2024, U.S. Ser. No. 63/653,360, filed May 30, 2024, U.S. Ser. No. 63/671,469, filed Jul. 15, 2024, U.S. Ser. No. 63/671,523, filed Jul. 15, 2024, U.S. Ser. No. 63/684,950, filed Aug. 20, 2024, U.S. Ser. No. 63/715,911, filed Nov. 4, 2024, and U.S. Ser. No. 63/715,916, filed Nov. 4, 2024, the entire contents of each of which is incorporated herein by reference.

### SEQUENCE LISTING

The instant application contains a Sequence Listing which has been submitted electronically in XML format and is hereby incorporated by reference in its entirety. Said XML copy, created on Jan. 22, 2024, is named 62801_39US01_SL.xml and is 211,580 bytes in size.

### 1. FIELD

This disclosure relates to immunoreceptor inhibitory proteins that bind to TL1A (e.g., human TL1A (hTL1A)) and nucleic acid molecules encoding the same. The disclosure further relates to methods of making and utilizing the same.

### 2. BACKGROUND

The TNF superfamily (TNFSF) comprises 19 ligands and 29 receptors that regulate multiple cellular functions, including e.g., immune responses, cell proliferation, cell survival, cell differentiation, and programmed cell death. Exemplary TNFSF ligands and exemplary cognate receptors, include, e.g., TNFα and TNFR1/TNFR2; FasL and Fas; LIGHT and LIGHTR and LTβR; and TL1A and DR3. A subset of TNFSF ligands are known to interact with more than one TNFSF receptor (e.g., TNFα is known to interact with both TNFR1 and TNFR2). The intracellular domains and signaling properties of the various TNFSF receptors are known to vary. For example, a subset of TNFSF receptors comprise a death domain; others comprise one or more TRAF interacting motif (TIM); while other subsets of TNFSF receptors do not contain functional intracellular signaling domains or motifs.

### 3. SUMMARY

Provided herein are, inter alia, immunoreceptor inhibitory proteins and nucleic acid molecules encoding the same; fusions and conjugates comprising the immunoreceptor inhibitory proteins; methods of manufacturing; pharmaceutical compositions; and methods of use including e.g., methods of inhibiting or reducing (e.g., preventing) binding of TL1A to DR3 and/or DcR3, inhibiting signaling of DR3 and/or DcR3 (including, e.g., signaling mediated through the binding of TL1A to DR3 and/or DcR3), and modulating (e.g., suppressing) an immune response, as well as diagnostics.

Accordingly, in one aspect provided herein are proteins (e.g., immunoreceptor inhibitory proteins (IIPs)) comprising an amino acid sequence at least about 85%, 86%, 87%, 88%, 89%, 90%, 91%, 92%, 93%, 94%, 95%, 96%, 97%, 98%, 99%, or 100% identical to the amino acid sequence of any protein set forth in Table 2 or set forth in any one of SEQ ID NOS: 23-84, 187-190, or 199-200.

In some embodiments, the protein comprises an amino acid sequence at least about 90%, 91%, 92%, 93%, 94%, 95%, 96%, 97%, 98%, 99%, or 100% identical to the amino acid sequence of any protein set forth in Table 2 or set forth in any one of SEQ ID NOS: 23-84, 187-190, or 199-200. In some embodiments, the protein comprises an amino acid sequence at least about 95%, 96%, 97%, 98%, 99%, or 100% identical to the amino acid sequence of any protein set forth in Table 2 or set forth in any one of SEQ ID NOS: 23-84, 187-190, or 199-200.

In some embodiments, the protein comprises an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 24, 55, 187, 188, 189, or 190. In some embodiments, the protein comprises an amino acid sequence at least about 90%, 91%, 92%, 93%, 94%, 95%, 96%, 97%, 98%, 99%, or 100% identical to the amino acid sequence set forth in any one of SEQ ID NOS: 24, 55, 187, 188, 189, or 190. In some embodiments, the protein comprises an amino acid sequence at least about 95%, 96%, 97%, 98%, 99%, or 100% identical to the amino acid sequence set forth in any one of SEQ ID NOS: 24, 55, 187, 188, 189, or 190. In some embodiments, the protein comprises the amino acid sequence set forth in any one of SEQ ID NOS: 24, 55, 187, 188, 189, or 190.

In some embodiments, the protein exhibits anti-inflammatory properties (e.g., upon administration to a subject).

In specific embodiments, the protein specifically binds human TL1A (hTL1A).

In some embodiments, the protein inhibits or reduces (e.g., prevents) binding of hTL1A to human DcR3 (hDcR3). In some embodiments, the protein inhibits or reduces (e.g., prevents) binding of hTL1A to human DR3 (hDR3).

In some embodiments, the protein specifically binds to trimeric forms of TL1A (e.g., hTL1A) and monomeric forms of TL1A (e.g., hTL1A). In some embodiments, the protein preferentially specifically binds to trimeric forms of TL1A (e.g., hTL1A) relative to monomeric forms of TL1A (e.g., hTL1A). In some embodiments, the protein specifically binds to trimeric forms of TL1A (e.g., hTL1A) with higher affinity relative to monomeric forms of TL1A (e.g., hTL1A) (e.g., as measured by SPR (e.g., as described in Example 10)).

In some embodiments, the binding affinity of the protein to trimeric TL1A (e.g., hTL1A) as measured by dissociation equilibrium constant (KD-trimer) is higher than the binding affinity of the protein to monomeric TL1A as measured by dissociation equilibrium constant (KD-monomer) (e.g., as measured by SPR (e.g., as described in Example 10)).

In some embodiments, the binding affinity of the protein to trimeric TL1A (e.g., hTL1A) as measured by dissociation equilibrium constant (KD-trimer) is at least 1, 2, 3, 4, 5, 6, 7, 8, 9, or 10-fold higher than the binding affinity of the protein to monomeric TL1A as measured by dissociation equilibrium constant (KD-monomer) (e.g., as measured by SPR (e.g., as described in Example 10)).

In some embodiments, the KD value of the protein binding to trimeric TL1A (e.g., hTL1A) (KD-trimer) is lower than the KD value of the protein binding to monomeric TL1A (e.g., hTL1A) (KD-monomer) (e.g., as measured by SPR (e.g., as described in Example 10)).

In some embodiments, the KD value of the protein binding to trimeric TL1A (e.g., hTL1A) (KD-trimer) is at least 1, 2, 3, 4, 5, 6, 7, 8, 9, or 10-fold lower than the KD value of the protein binding to monomeric TL1A (KD-monomer) (e.g., as measured by SPR (e.g., as described in Example 10)).

In some embodiments, the binding affinity of the protein to trimeric TL1A (e.g., hTL1A) as measured by dissociation equilibrium constant (KD-trimer) is less than 20 pM, 15 pM, or 10 pM (e.g., as measured by SPR (e.g., as described in Example 10)).

In some embodiments, the binding affinity of the protein to monomeric TL1A (e.g., hTL1A) as measured by dissociation equilibrium constant (KD-monomer) is more than 60 pM, 70 pM, or 80 pM (e.g., as measured by SPR (e.g., as described in Example 10)).

In some embodiments, the protein comprises a homologous or heterologous signal peptide (e.g., operably connected to the N-terminus of the protein).

In some embodiments, the protein is operably connected to a heterologous moiety (e.g., described herein).

In some embodiments, the heterologous moiety is a protein, peptide, small molecule, nucleic acid molecule (e.g., DNA, RNA, DNA/RNA hybrid molecule), lipid, or synthetic polymer. In some embodiments, the heterologous moiety is a protein. In some embodiments, the heterologous moiety is a half-life extension moiety.

In specific embodiments, the protein is isolated. In specific embodiments, the protein is recombinant. In specific embodiments, the protein is recombinant and isolated.

In one aspect, provided herein are conjugates comprising a protein described herein (e.g., an IIP described herein) operably connected to a heterologous moiety (e.g., a heterologous moiety described herein).

In one aspect, provided herein are radioligands comprising a protein described herein (e.g., an IIP described herein) operably connected to a radionuclide.

In one aspect, provided herein are fusion proteins comprising a protein described herein (e.g., an IIP described herein) operably connected to a heterologous protein (e.g., a heterologous protein described herein).

In specific embodiments, the heterologous protein comprises an antibody. In specific embodiments, the antibody specifically binds a cytokine. In specific embodiments, the cytokine is an interleukin. In specific embodiments, the interleukin is interleukin 23 (IL-23) (e.g., human IL-23).

In specific embodiments, the heterologous protein comprises a half-life extension protein.

In specific embodiments, the heterologous protein comprises an immunoglobulin (Ig) (e.g., a human Ig (hIg)) Fc region. In specific embodiments, the Ig (e.g., hIg) Fc region comprises at least a portion of a hinge region, a CH2 region, and a CH3 region. In specific embodiments, the Ig (e.g., hIg) Fc region comprises a hinge region, a CH2 region, and a CH3 region. In specific embodiments, the Ig is a hIg. In specific embodiments, the hIg is a human IgG (hIgG). In specific embodiments, the hIgG is hIgG1 or hIgG4.

In specific embodiments, the Ig (e.g., hIg) Fc region comprises one or more amino acid substitutions relative to a reference Ig (e.g., hIg) Fc region that reduces or abolishes one or more of the following effector functions relative to the reference Ig (e.g., hIg) Fc region: antibody dependent cell mediated cytotoxicity (ADCC), complement dependent cytotoxicity (CDC), and/or affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))). In specific embodiments, the Ig (e.g., hIg) Fc region does not substantially mediate ADCC, does not substantially mediate CDC, and/or does not bind to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In specific embodiments, the Ig is an hIgG4 and the amino acid sequence of the Fc region comprises an amino acid substitution at amino acid position S228, an amino acid substitution at amino acid position F234, and/or an amino acid substitution at amino acid position L235, numbering according to the EU index of Kabat. In specific embodiments, the Ig is hIgG4 and the amino acid sequence of the Fc region comprises a proline at amino acid position S228, an alanine at amino acid position F234, and/or an alanine at amino acid position L235, numbering according to EU index of Kabat.

In specific embodiments, the Ig is hIgG1 and the amino acid sequence of the Fc region comprises an amino acid substitution at amino acid position L234, and/or an amino acid substitution at amino acid position L235, numbering according to the EU index of Kabat. In specific embodiments, the Ig is hIgG1 and the amino acid sequence of the Fc region comprises an alanine at amino acid position L234 and/or an alanine at amino acid position L235, numbering according to the EU index of Kabat. In specific embodiments, the Ig is hIgG1 and the amino acid sequence of the Fc region comprises an alanine at amino acid position L234, an alanine at amino acid position L235, and/or a glycine at position P329 numbering according to the EU index of Kabat. In specific embodiments, the Ig (e.g., hIg) Fc region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any protein set forth in any one of Tables 4-7 or set forth in any one of SEQ ID NOS: 90-163 or 197-198.

In specific embodiments, the Ig (e.g., hIg) Fc region comprises an amino acid sequence comprising a set of amino acid variations set forth in any one of Tables 12-14.

In some embodiments, the protein described herein is directly operably connected to the heterologous protein through a peptide bond. In some embodiments, the protein described herein is indirectly operably connected to the heterologous protein through a peptide linker. In some embodiments, the amino acid sequence of the peptide linker comprises or consists of glycine or glycine and serine amino acid residues. In some embodiments, the amino acid of the peptide linker comprises or consists of (a) the amino acid sequence set forth in any one of SEQ ID NOS: 164-173; or (b) the amino acid sequence set forth in any one of SEQ ID NOS: 164-173 comprising or consisting of 1, 2, or 3 amino acid substitutions.

In some embodiments, the fusion protein comprises from N- to C-terminus: the protein described herein and the heterologous protein. In some embodiments, the fusion protein comprises from N- to C-terminus: the protein described herein, a peptide linker, and the heterologous protein. In some embodiments, the fusion protein comprises from N- to C-terminus: a signal peptide, the protein described herein, a peptide linker, and the heterologous protein. In some embodiments, the fusion protein comprises from N- to C-terminus: the heterologous protein and the protein described herein. In some embodiments, the fusion protein comprises from N- to C-terminus: the heterologous protein, a peptide linker, and the protein described herein. In some embodiments, the fusion protein comprises from N- to C-terminus: a signal peptide, the heterologous protein, a peptide linker, and the protein described herein.

In some embodiments, the amino acid sequence of the fusion protein is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 174-185 or 191-196 or set forth in Table 9. In some embodiments, the amino acid sequence of the fusion protein is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 175, 178, 181, or 184. In some embodiments, the amino acid sequence of the fusion protein is at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 177-179, 194-196, or 201-203. In some embodiments, the amino acid sequence of the fusion protein is at least about 90%, 91%, 92%, 93%, 94%, 95%, 96%, 97%, 98%, 99%, or 100% identical to the amino acid sequence set forth in any one of SEQ ID NOS: 177-179, 194-196, or 201-203. In some embodiments, the amino acid sequence of the fusion protein is at least about 95%, 96%, 97%, 98%, 99%, or 100% identical to the amino acid sequence set forth in any one of SEQ ID NOS: 177-179, 194-196, or 201-203. In some embodiments, the amino acid sequence of the fusion protein comprises the amino acid sequence set forth in any one of SEQ ID NOS: 177-179, 194-196, or 201-203.

In one aspect, provided herein are fusion proteins comprising a first protein and a second protein, wherein the first protein comprises a first Ig (e.g., hIg) Fc region operably connected to a first protein described herein; and wherein the second protein comprises a second Ig (e.g., hIg) Fc region operably connected to a second protein described herein.

In specific embodiments, the first Fc region and the second Fc region associate to form a dimer.

In some embodiments, the first protein comprises an amino acid sequence at least about 90%, 91%, 92%, 93%, 94%, 95%, 96%, 97%, 98%, 99%, or 100% identical to the amino acid sequence of the second protein.

In some embodiments, the first Ig (e.g., hIg, mIg) Fc region comprises at least a portion of a hinge region, a CH2 region, and a CH3 region; and the second Ig (e.g., hIg) Fc region comprises at least a portion of a hinge region, a CH2 region, and a CH3 region. In some embodiments, the first Ig (e.g., hIg) Fc region comprises a hinge region, a CH2 region, and a CH3 region; and the second Ig (e.g., hIg) Fc region comprises a hinge region, a CH2 region, and a CH3 region.

In some embodiments, the Ig of the first Ig Fc region is a hIg and the Ig of the second Ig Fc region is a hIg. In some embodiments, the hIg of the first hIg Fc region is a hIgG and the hIg of the second hIg Fc region is a hIgG. In some embodiments, the hIgG of the first hIg Fc region is hIgG4 and the hIgG of the first hIg Fc region is hIgG4. In some embodiments, the hIgG of the first hIg Fc region is hIgG1 and the hIgG of the first hIg Fc region is hIgG1.

In some embodiments, the first Ig (e.g., hIg) Fc region and the second Ig (e.g., hIg) Fc region each comprises one or more amino acid substitutions relative to a reference Ig (e.g., hIg) Fc region that reduces or abolishes one or more of the following effector functions relative to the reference Ig (e.g., hIg) Fc region: ADCC, CDC, and/or binding affinity to one or more Fe receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))). In some embodiments, the fusion protein does not substantially mediate ADCC, does not substantially mediate CDC, and/or does not bind to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In some embodiments, the Ig of the first Ig Fc region and the second Ig Fc region is hIgG4 and the amino acid sequence of the first Fc region and the second Fc region each comprise an amino acid substitution at amino acid position S228, an amino acid substitution at amino acid position F234, and/or an amino acid substitution at amino acid position E235, numbering according to the EU index of Kabat. In some embodiments, the Ig of the first Ig Fc region and the second Ig Fc region is hIgG4 and the amino acid sequence of the first Fc region and the second Fc region each comprise a proline at amino acid position S228, an alanine at amino acid position F234, and/or an alanine at amino acid position E235, numbering according to EU index of Kabat.

In some embodiments, the Ig of the first Ig Fc region and the second Ig Fc region is hIgG1 and the amino acid sequence of the first Fc region and the second Fc region each comprise an amino acid substitution at amino acid position L234, and/or an amino acid substitution at amino acid position L235, numbering according to the EU index of Kabat. In some embodiments, the Ig of the first Ig Fc region and the second Ig Fc region is hIgG1 and the amino acid sequence of the first Fc region and the second Fc region each comprise an alanine at amino acid position L234 and/or an alanine at amino acid position L235, numbering according to the EU index of Kabat. In some embodiments, the Ig of the first Ig Fc region and the second Ig Fc region is hIgG1 and the amino acid sequence of the first Fc region and the second Fc region each comprise a proline (or alanine) at amino acid position L234 and/or a proline (or alanine) at amino acid position L235, numbering according to the EU index of Kabat. In some embodiments, the Ig of the first Ig Fc region and the second Ig Fc region is hIgG1 and the amino acid sequence of the first Fc region and the second Fc region each comprise a proline (or alanine) at amino acid position L234 a proline (or alanine) at amino acid position L235, and/or a glycine at amino acid position P329, numbering according to the EU index of Kabat.

In some embodiments, the first Ig (e.g., hIg) Fc region comprises an amino acid sequence at least about 90%, 91%, 92%, 93%, 94%, 95%, 96%, 97%, 98%, 99%, or 100% identical to the amino acid sequence of the second Ig (e.g., hIg) Fc region.

In some embodiments, the first protein comprises from N- to C-terminus: the first Ig (e.g., hIg) Fc region and the first protein described herein; and the second protein comprises from N- to C-terminus: the second Ig (e.g., hIg) Fc region and the second protein described herein.

In some embodiments, the first protein comprises from N- to C-terminus: the first Ig (e.g., hIg) Fc region, a first peptide linker, and the first protein described herein; and the second protein comprises from N- to C-terminus: the second Ig (e.g., hIg) Fc region, a second peptide linker, and the second protein described herein.

In some embodiments, the first protein comprises from N- to C-terminus: the first protein described herein and the first Ig (e.g., hIg) Fc region; and the second protein comprises from N- to C-terminus: the second protein described herein and the second Ig (e.g., hIg) Fc region.

In some embodiments, the first protein comprises from N- to C-terminus: the first protein described herein, a first peptide linker, and the first Ig (e.g., hIg) Fc region; and the second protein comprises from N- to C-terminus: the second protein described herein, a second peptide linker, and the second Ig (e.g., hIg) Fc region.

In some embodiments, the amino acid sequence of the first peptide linker and the second peptide linker each comprises or consists of glycine or glycine and serine amino acid residues. In some embodiments, the amino acid of the first peptide linker and the second peptide linker each comprises or consists of (a) the amino acid sequence set forth in any one of SEQ ID NOS: 164-173; or (b) the amino acid sequence set forth in any one of SEQ ID NOS: 164-173 comprising or consisting of 1, 2, or 3 amino acid substitutions.

In some embodiments, the amino acid sequence of the first protein is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 174-185 or 191-196 or set forth in Table 9; and the amino acid sequence of the first protein is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 174-185 or 191-196 or set forth in Table 9. In some embodiments, the amino acid sequence of the first protein is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 175, 178, 181, 184, 192, or 195; and the amino acid sequence of the first protein is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 175, 178, 181, 184, 192, or 195.

In one aspect, provided herein are immunogenic peptides or proteins comprising at least an immunogenic fragment of a protein described herein (e.g., an IIP described herein).

In some embodiments, the immunogenic peptide or protein does not specifically bind hTL1A or binds one or more of hTL1A with lower affinity relative to a reference protein (e.g., a protein described herein (e.g., an IIP described herein)).

In some embodiments, the immunogenic peptide or protein comprises a full-length protein described herein. In some embodiments, the immunogenic peptide or protein comprises an immunogenic fragment of a protein of a protein described herein (e.g., an IIP described herein).

In some embodiments, the immunogenic peptide or protein comprises at least about 10, 15, 20, 30, 40, 50, 60, 70, 80, 90, 100, 110, 120, or 130 amino acids. In some embodiments, the immunogenic peptide or protein comprises from about 10, 15, 20, 30, 40, 50, 60, 70, 80, 90, 100, 110, 120, or 130 amino acids.

In some embodiments, the amino acid sequence of the immunogenic peptide or protein comprises one or more (e.g., 1, 2, 3, 4, 5, 6, 7, 8, 9, 10, or more) amino acid variations (e.g., substitutions, additions, deletions) relative to a reference protein described herein.

In some embodiments, the immunogenic peptide or protein comprises an amino acid sequence that is at least about 80%, 85%, 90%, 91%, 92%, 93%, 94%, 95%, 96%, 97%, 98%, 99% or 100% identical to a contiguous stretch of at least about 10, 15, 20, 30, 40, 50, 60, 70, 80, 90, 100, 110, 120, or 130 amino acids set forth in any one of SEQ ID NOS: 23-84, 187-190, or 199-200. In some embodiments, the immunogenic peptide or protein comprises an amino acid sequence that, other than the one or more amino acid variation (e.g., substitution, addition, deletion), is at least about 80%, 85%, 90%, 91%, 92%, 93%, 94%, 95%, 96%, 97%, 98%, 99% or 100% identical to the amino acid sequence set forth in any one of SEQ ID NOS: 23-84, 187-190, or 199-200.

In some embodiments, the immunogenic peptide or protein is formulated with an adjuvant.

In one aspect, provided herein are isolated antibodies that specifically binds to a protein described herein (e.g., an IIP described herein).

In one aspect, provided herein are nucleic acid molecules encoding a protein described herein (e.g., an IIP described herein), a conjugate described herein, a radioligand described herein, a fusion protein described herein, an immunogenic peptide or protein described herein, or an antibody described herein. In some embodiments, the nucleic acid molecule is an RNA (e.g., mRNA, circular RNA) molecule or a DNA molecule.

In one aspect, provided herein are mRNA encoding a protein described herein (e.g., an IIP described herein), a conjugate described herein, a radioligand described herein, a fusion protein described herein, an immunogenic peptide or protein described herein, or an antibody described herein.

In some embodiments, the nucleic acid molecule or the mRNA molecule comprises a heterologous 5′-untranslated region (UTR), 3′-UTR, or both a 5′-UTR and 3′-UTR. In some embodiments, the nucleic acid molecule or the mRNA molecule comprises a poly(A) sequence. In some embodiments, the nucleic acid molecule or the mRNA molecule comprises a 5′ cap structure. In some embodiments, the nucleic acid molecule or the mRNA molecule comprises at least one variant nucleotide. In some embodiments, the sequence of the nucleic acid molecule or mRNA molecule, respectively, is codon optimized.

In one aspect, provided herein are vectors (e.g., expression vectors) comprising a nucleic acid molecule described herein or an mRNA molecule described herein.

In some embodiments, the vector is a viral vector or a non-viral vector (e.g., a plasmid).

In one aspect, provided herein are viral particles conjugated to a protein described herein (e.g., an IIP described herein), a conjugate described herein, a radioligand described herein, or a fusion protein described herein.

In one aspect, provided herein are cells (e.g., host cell) (or populations of cells (e.g., populations of host cells)) comprising a protein described herein (e.g., an IIP described herein), a conjugate described herein, a radioligand described herein, a fusion protein described herein, an immunogenic peptide or protein described herein, an antibody described herein, a nucleic acid molecule described herein, an mRNA molecule described herein, a vector described herein, a viral particle described herein, a carrier described herein, a lipid nanoparticle described herein, a vaccine composition described herein, or a pharmaceutical composition described herein.

In one aspect, provided herein are cells (e.g., a therapeutic cell) (e.g., a CAR cell) (or a population of therapeutic cells) expressing and/or genetically encoding a protein described herein (e.g., an IIP described herein), a conjugate described herein, a radioligand described herein, or a fusion protein described herein (e.g., on the surface of the cell).

In one aspect, provided herein are carriers a protein described herein (e.g., an IIP described herein), a conjugate described herein, a radioligand described herein, a fusion protein described herein, an immunogenic peptide or protein described herein, an antibody described herein, a nucleic acid molecule described herein, an mRNA molecule described herein, a vector described herein, a viral particle described herein, a cell described herein, a lipid nanoparticle described herein, a vaccine composition described herein, or a pharmaceutical composition described herein.

In one aspect, provided herein are carriers conjugated to a protein described herein (e.g., an IIP described herein), a conjugate described herein, a radioligand described herein, or a fusion protein described herein.

In some embodiments, the carrier is a lipid nanoparticle, liposome, lipoplex, or nanoliposome.

In one aspect, provided herein are lipid nanoparticles comprising a protein described herein (e.g., an IIP described herein), a conjugate described herein, a radioligand described herein, a fusion protein described herein, an immunogenic peptide or protein described herein, an antibody described herein, a nucleic acid molecule described herein, an mRNA molecule described herein, a vector described herein, a viral particle described herein, a cell described herein, a lipid nanoparticle described herein, a vaccine composition described herein, or a pharmaceutical composition described herein.

In one aspect, provided herein are lipid nanoparticles conjugated to a protein described herein (e.g., an IIP described herein), a conjugate described herein, a radioligand described herein, or a fusion protein described herein.

In one aspect, provided herein are vaccine compositions comprising an immunogenic peptide or protein described herein (or a nucleic acid molecule encoding the same (or a vector encoding the nucleic acid molecule) or a carrier comprising any of the foregoing).

In one aspect, provided herein are pharmaceutical compositions comprising a protein described herein (e.g., an IIP described herein), a conjugate described herein, a radioligand described herein, a fusion protein described herein, an immunogenic peptide or protein described herein, an antibody described herein, a nucleic acid molecule described herein, an mRNA molecule described herein, a vector described herein, a viral particle described herein, a cell (or population of cells) described herein, a carrier described herein, a lipid nanoparticle described herein, or a vaccine composition described herein; and a pharmaceutically acceptable excipient.

In one aspect, provided herein are kits comprising a protein described herein (e.g., an IIP described herein), a conjugate described herein, a radioligand described herein, a fusion protein described herein, an immunogenic peptide or protein described herein, an antibody described herein, a nucleic acid molecule described herein, an mRNA molecule described herein, a vector described herein, a viral particle described herein, a cell (or population of cells) described herein, a carrier described herein, a lipid nanoparticle described herein, a vaccine composition described herein, or a pharmaceutical composition described herein; and optionally instructions for using any one or more of the foregoing.

In one aspect, provided herein are methods of delivering a protein, fusion protein, conjugate, radioligand, nucleic acid molecule, mRNA molecule, expression vector, viral particle cell, carrier, lipid nanoparticle, immunogenic peptide or protein, an antibody, a vaccine composition, or pharmaceutical composition to a subject, the method comprising administering to the subject a protein described herein (e.g., an IIP described herein), a conjugate described herein, a radioligand described herein, a fusion protein described herein, an immunogenic peptide or protein described herein, an antibody described herein, a nucleic acid molecule described herein, an mRNA molecule described herein, a vector described herein, a viral particle described herein, a cell (or population of cells) described herein, a carrier described herein, a lipid nanoparticle described herein, a vaccine composition described herein, or a pharmaceutical composition described herein, to thereby deliver the protein, fusion protein, conjugate, radioligand, immunogenic peptide or protein, antibody, mRNA molecule, vector, viral particle, cell, carrier, lipid nanoparticle, vaccine composition, or pharmaceutical composition to a subject.

In one aspect, provided herein are methods of inhibiting or reducing (e.g., preventing) binding of one or more TNFSF ligand to one or more of the TNFSF ligand's cognate TNFSF receptors in a subject in need thereof, the method comprising administering to the subject a protein described herein (e.g., an IIP described herein), a conjugate described herein, a radioligand described herein, a fusion protein described herein, an immunogenic peptide or protein described herein, an antibody described herein, a nucleic acid molecule described herein, an mRNA molecule described herein, a vector described herein, a viral particle described herein, a cell (or population of cells) described herein, a carrier described herein, a lipid nanoparticle described herein, a vaccine composition described herein, or a pharmaceutical composition described herein, to thereby inhibit or reduce (e.g., prevent) binding of one or more TNFSF ligand to one or more of the TNFSF ligand's cognate TNFSF receptors in the subject.

In some embodiments, the method comprises inhibiting or reducing (e.g., preventing) binding of any one or more (e.g., 1, 2, 3, 4, 5, 6, 7, 8, and/or 9) of the following: (a) binding of TLA1 to DR3, (b) binding of TNFα to TNFR2, (c) binding of TNFα to TNFR1, (d) binding of LIGHT to HVEM, (e) binding of LIGHT to LTβR, and/or (f) binding of FASL to FAS.

In one aspect, provided herein are methods of inhibiting or reducing (e.g., preventing) signaling mediated by the binding of one or more TNFSF ligand to one or more of the TNFSF ligand's cognate TNFSF receptors in a subject in need thereof, the method comprising administering to the subject a protein described herein (e.g., an IIP described herein), a conjugate described herein, a radioligand described herein, a fusion protein described herein, an immunogenic peptide or protein described herein, an antibody described herein, a nucleic acid molecule described herein, an mRNA molecule described herein, a vector described herein, a viral particle described herein, a cell (or population of cells) described herein, a carrier described herein, a lipid nanoparticle described herein, a vaccine composition described herein, or a pharmaceutical composition described herein, to thereby inhibit or reduce (e.g., prevent) signaling mediated by the binding of one or more TNFSF ligand to one or more of the TNFSF ligand's cognate TNFSF receptors in the subject.

In some embodiments, the method comprises inhibiting or reducing (e.g., preventing) signaling mediated by binding of any one or more (e.g., 1, 2, 3, 4, 5, 6, 7, 8, and/or 9) of the following: (a) binding of TLA1 to DR3, (b) binding of TNFα to TNFR2, (c) binding of TNFα to TNFR1, (d) binding of LIGHT to HVEM, (e) binding of LIGHT to LTβR, and/or (f) binding of FASL to FAS.

In one aspect, provided herein are methods of inhibiting or reducing (e.g., preventing) binding of TL1A to DR3 in a subject in need thereof, the method comprising administering to the subject a protein described herein (e.g., an IIP described herein), a conjugate described herein, a radioligand described herein, a fusion protein described herein, an immunogenic peptide or protein described herein, an antibody described herein, a nucleic acid molecule described herein, an mRNA molecule described herein, a vector described herein, a viral particle described herein, a cell (or population of cells) described herein, a carrier described herein, a lipid nanoparticle described herein, a vaccine composition described herein, or a pharmaceutical composition described herein, to thereby inhibit or reduce (e.g., prevent) binding of TL1A to DR3 in the subject.

In one aspect, provided herein are methods of inhibiting or reducing (e.g., preventing) signaling mediated by the binding of TL1A to DR3 in a subject in need thereof, the method comprising administering to the subject a protein described herein (e.g., an IIP described herein), a conjugate described herein, a radioligand described herein, a fusion protein described herein, an immunogenic peptide or protein described herein, an antibody described herein, a nucleic acid molecule described herein, an mRNA molecule described herein, a vector described herein, a viral particle described herein, a cell (or population of cells) described herein, a carrier described herein, a lipid nanoparticle described herein, a vaccine composition described herein, or a pharmaceutical composition described herein, to thereby inhibit or reduce (e.g., prevent) signaling mediated by the binding of TL1A to DR3 in the subject.

In one aspect, provided herein are methods of inhibiting or reducing (e.g., preventing) binding of TL1A to DcR3 in a subject in need thereof, the method comprising administering to the subject a protein described herein (e.g., an IIP described herein), a conjugate described herein, a radioligand described herein, a fusion protein described herein, an immunogenic peptide or protein described herein, an antibody described herein, a nucleic acid molecule described herein, an mRNA molecule described herein, a vector described herein, a viral particle described herein, a cell (or population of cells) described herein, a carrier described herein, a lipid nanoparticle described herein, a vaccine composition described herein, or a pharmaceutical composition described herein, to thereby inhibit or reduce (e.g., prevent) binding of TL1A to DcR3 in the subject.

In one aspect, provided herein are methods of inhibiting or reducing (e.g., preventing) signaling mediated by the binding of TL1A to DcR3 in a subject in need thereof, the method comprising administering to the subject a protein described herein (e.g., an IIP described herein), a conjugate described herein, a radioligand described herein, a fusion protein described herein, an immunogenic peptide or protein described herein, an antibody described herein, a nucleic acid molecule described herein, an mRNA molecule described herein, a vector described herein, a viral particle described herein, a cell (or population of cells) described herein, a carrier described herein, a lipid nanoparticle described herein, a vaccine composition described herein, or a pharmaceutical composition described herein, to thereby inhibit or reduce (e.g., prevent) signaling mediated by the binding of TL1A to DcR3 in the subject.

In some embodiments, the protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) signaling mediated by binding of any one or more (e.g., 1, 2, 3, 4, and/or 5) of the following: (a) binding of TNFα to TNFR2, (b) binding of TNFα to TNFR1, (c) binding of LIGHT to HVEM, (d) binding of LIGHT to LTβR, and/or (e) binding of FASL to FAS.

In one aspect, provided herein are methods of suppressing or preventing a pro-inflammatory immune response in a subject in need thereof, the method comprising administering to the subject a protein described herein (e.g., an IIP described herein), a conjugate described herein, a radioligand described herein, a fusion protein described herein, an immunogenic peptide or protein described herein, an antibody described herein, a nucleic acid molecule described herein, an mRNA molecule described herein, a vector described herein, a viral particle described herein, a cell (or population of cells) described herein, a carrier described herein, a lipid nanoparticle described herein, a vaccine composition described herein, or a pharmaceutical composition described herein, to thereby suppress or prevent a pro-inflammatory immune response in the subject.

In one aspect, provided herein are methods of preventing, treating, or ameliorating a disease in a subject in need thereof, the method comprising administering to the subject a protein described herein (e.g., an IIP described herein), a conjugate described herein, a radioligand described herein, a fusion protein described herein, an immunogenic peptide or protein described herein, an antibody described herein, a nucleic acid molecule described herein, an mRNA molecule described herein, a vector described herein, a viral particle described herein, a cell (or population of cells) described herein, a carrier described herein, a lipid nanoparticle described herein, a vaccine composition described herein, or a pharmaceutical composition described herein, to thereby prevent, treat, or ameliorate the disease in the subject.

In some embodiments, the disease is a pro-inflammatory disease, an autoimmune disease, or a fibrotic disease. In some embodiments, the disease is a proinflammatory (e.g., autoimmune disease). In some embodiments, the disease is inflammatory bowel disease, Crohn's disease, ulcerative colitis, rheumatoid arthritis, psoriatic arthritis, ankylosing spondylitis, asthma, systemic lupus erythematosus, or fibrosis.

In one aspect, provided herein are methods of inducing or enhancing an immune response in a subject in need thereof, the method comprising administering to the subject (i) an immunogenic peptide or protein described herein (or a conjugate or a fusion protein thereof); (ii) a nucleic acid molecule encoding (i); (iii) a vector comprising (ii); (iv) a carrier comprising (i), (ii), or (iii); a vaccine composition comprising (i), (ii), (iii), or (iv); or a pharmaceutical composition comprising (i), (ii), (iii), (iv), or (v), to thereby induce or enhance an immune response in the subject. In some embodiments, the method is an in vitro method. In some embodiments, the sample is a blood, cell, tissue, or saliva, or nasal swab. In some embodiments, an antibody described herein is utilized to determine the presence or absence of the protein described herein (or the fragment or variant thereof).

In one aspect, provided herein are methods of vaccinating a subject in need thereof (e.g., against a viral infection), the method comprising administering to the subject (i) an immunogenic peptide or protein described herein (or a conjugate or a fusion protein thereof); (ii) a nucleic acid molecule encoding (i); (iii) a vector comprising (ii); (iv) a carrier comprising (i), (ii), or (iii); a vaccine composition comprising (i), (ii), (iii), or (iv); or a pharmaceutical composition comprising (i), (ii), (iii), (iv), or (v), to thereby vaccinate the subject in need thereof (e.g., against a virus). In some embodiments, the method is an in vitro method. In some embodiments, the sample is a blood, cell, tissue, or saliva, or nasal swab. In some embodiments, an antibody described herein is utilized to determine the presence or absence of the protein described herein (or the fragment or variant thereof).

In one aspect, provided herein are methods of determining the presence of a virus in a subject, the method comprising (a) obtaining the sample from a subject or providing a sample that has been obtained from a subject, and (b) determining the presence or absence of a protein described herein (e.g., an IIP described herein) (or a fragment or variant thereof) or a nucleic acid molecule encoding a protein described herein (e.g., an IIP described herein) (or the fragment or variant thereof) in the sample. In some embodiments, the method is an in vitro method. In some embodiments, the sample is a blood, cell, tissue, or saliva, or nasal swab. In some embodiments, an antibody described herein is utilized to determine the presence or absence of the protein described herein (or the fragment or variant thereof).

In one aspect, provided herein are methods of diagnosing a viral infection in a subject, the method comprising (a) obtaining a sample from a subject or providing a sample that has been obtained from a subject, (b) determining the presence or absence of a protein described herein (e.g., an IIP described herein) (or a fragment or variant thereof) or a nucleic acid molecule encoding a protein described herein (e.g., an IIP described herein) (or a fragment or variant thereof), and (c) diagnosing the subject as having the viral infection if a protein described herein (e.g., an IIP described herein) (or a fragment or variant thereof) or a nucleic acid molecule encoding a protein described herein (e.g., an IIP described herein) (or the fragment or variant thereof) is determined to be present in the sample in step (b). In some embodiments, the method is an in vitro method. In some embodiments, the sample is a blood, cell, tissue, or saliva, or nasal swab. In some embodiments, an antibody described herein is utilized to determine the presence or absence of the protein described herein (or the fragment or variant thereof).

In one aspect, provided herein are methods of treating a viral infection in a subject, the method comprising (a) receiving testing results that determined the presence of a protein described herein (e.g., an IIP described herein) (or a fragment or variant thereof) or a nucleic acid molecule encoding a protein described herein (e.g., an IIP described herein) (or the fragment or variant thereof) in a sample from the subject, (b) diagnosing the subject as having the viral infection, and (c) administering a therapeutic agent to treat the viral infection.

In some embodiments, the sample is a blood, cell, tissue, or saliva, or nasal swab. In some embodiments, an antibody described herein is utilized to determine the presence or absence of the protein described herein (or the fragment or variant thereof).

In specific preferred embodiments of any of the foregoing methods, the subject is a human.

### 4. BRIEF DESCRIPTION OF THE FIGURES

FIG. 1 is a line graph showing the binding affinity of the indicated protein (IIP-1, DcR3-hFc, TNFR2 ECD-Fc, or hFc) (Y axis) at the indicated dose (X axis) to hTL1A.

FIG. 2 is a line graph showing the binding affinity of the indicated protein (IIP-2, DcR3-hFc, TNFR2 ECD-Fc, or hFc) (Y axis) at the indicated dose (X axis) to hTL1A.

FIG. 3 is a line graph showing the binding affinity of the indicated protein (IIP-3, DcR3-hFc, TNFR2 ECD-Fc, or hFc) (Y axis) at the indicated dose (X axis) to hTL1A.

FIG. 4 is a line graph showing the binding affinity of the indicated protein (IIP-4, DcR3-hFc, TNFR2 ECD-Fc, or hFc) (Y axis) at the indicated dose (X axis) to hTL1A.

FIG. 5 is a line graph showing the binding affinity of the indicated protein (IIP-1, DcR3-hFc, TNFR2 ECD-Fc, or hFc) (Y axis) at the indicated dose (X axis) to hTNFα.

FIG. 6 is a line graph showing the binding affinity of the indicated protein (IIP-2, DcR3-hFc, TNFR2 ECD-Fc, or hFc) (Y axis) at the indicated dose (X axis) to hTNFα.

FIG. 7 is a line graph showing the binding affinity of the indicated protein (IIP-3, DcR3-hFc, TNFR2 ECD-Fc, or hFc) (Y axis) at the indicated dose (X axis) to hTNFα.

FIG. 8 is a line graph showing the binding affinity of the indicated protein (IIP-4, DcR3-hFc, TNFR2 ECD-Fc, or hFc) (Y axis) at the indicated dose (X axis) to hTNFα.

FIG. 9 is a line graph showing the binding affinity of the indicated protein (IIP-1, DcR3-hFc, TNFR2 ECD-Fc, or hFc) (Y axis) at the indicated dose (X axis) to hLIGHT.

FIG. 10 is a line graph showing the binding affinity of the indicated protein (IIP-2, DcR3-hFc, TNFR2 ECD-Fc, or hFc) (Y axis) at the indicated dose (X axis) to hLIGHT.

FIG. 11 is a line graph showing the binding affinity of the indicated protein (IIP-3, DcR3-hFc, TNFR2 ECD-Fc, or hFc) (Y axis) at the indicated dose (X axis) to hLIGHT.

FIG. 12 is a line graph showing the binding affinity of the indicated protein (IIP-4, DcR3-hFc, TNFR2 ECD-Fc, or hFc) (Y axis) at the indicated dose (X axis) to hLIGHT.

FIG. 13 is a line graph showing the binding affinity of the indicated protein (IIP-1, DcR3-hFc, TNFR2 ECD-Fc, or hFc) (Y axis) at the indicated dose (X axis) to hFASL.

FIG. 14 is a line graph showing the binding affinity of the indicated protein (IIP-2, DcR3-hFc, TNFR2 ECD-Fc, or hFc) (Y axis) at the indicated dose (X axis) to hFASL.

FIG. 15 is a line graph showing the binding affinity of the indicated protein (IIP-3, DcR3-hFc, TNFR2 ECD-Fc, or hFc) (Y axis) at the indicated dose (X axis) to hFASL.

FIG. 16 is a line graph showing the binding affinity of the indicated protein (IIP-4, DcR3-hFc, TNFR2 ECD-Fc, or hFc) (Y axis) at the indicated dose (X axis) to hFASL.

FIG. 17 is a line graph showing the binding affinity of the indicated protein (IIP-34, DcR3-hFc, TNFR2 ECD-Fc, or hFc) (Y axis) at the indicated dose (X axis) to hTL1A.

FIG. 18 is a line graph showing the binding affinity of the indicated protein (IIP-34, DcR3, TNFR2 ECD-Fc, or hFc) (Y axis) at the indicated dose (X axis) to hTNFα.

FIG. 19 is a line graph showing the binding affinity of the indicated protein (IIP-34, DcR3, TNFR2 ECD-Fc, or hFc) (Y axis) at the indicated dose (X axis) to hLIGHT.

FIG. 20 is a line graph showing the binding affinity of the indicated protein (IIP-34, DcR3, TNFR2 ECD-Fc, or hFc) (Y axis) at the indicated dose (X axis) to hFASL.

FIG. 21 is a line graph showing the relative binding of the indicated agent hIgG4-Fc (control), IIFP-2, IIFP-32, and IIFP-33, to plate bound human TL1A.

FIG. 22 is a line graph showing the relative binding of the indicated agent hIgG4-Fc (control), IIFP-2, IIFP-32, and IIFP-33, to membrane bound human TL1A.

FIG. 23 is a line graph showing NFκB activation in TL1A responsive luciferase reporter Jurkat cells treated with the indicated agent: hIgG4-Fc (control), IIFP-2, IIFP-32, and IIFP-33.

FIG. 24 is a line graph showing the inhibition of human TL1A binding to DR3 mediated by the indicated agent: hIgG4-Fc (control), IIFP-2, IIFP-32, or IIFP-33.

FIG. 25 is a line graph showing the inhibition of human TL1A binding to DcR3 mediated by the indicated agent: hIgG4-Fc (control), IIFP-2, IIFP-32, or IIFP-33.

FIG. 26 is a line graph showing the percent of inhibition of TL1A induced apoptosis in TF1 cells by the indicated gent: hIgG4-Fc (control), IIFP-2, IIFP-32, or IIFP-33.

FIG. 27 is a bar graph showing the concentration of IFNγ (pg/mL) in the serum of mice treated with the indicated agent: PBS (control), human IgG4 Fc (control), anti-murineTL1A (mTL1A) antibody (5G4.6) (20 mg/kg, 10 mg/kg, or 5 mg/kg), or IIFP-2 (20 mg/kg, 10 mg/kg, or 5 mg/kg).

FIG. 28A is a line graph showing the clinical disease score (as described in Example 4) at the indicated study day and indicated treatment group (IFP-2, hIgG4 Fc control, no disease control) in the CAIA mouse model of rheumatoid arthritis. FIG. 28B is a bar graph showing the clinical disease score area under the curve (as described in Example 4) at the indicated study day and indicated treatment group (IFP-2, hIgG4 Fc control) in the CAIA mouse model of rheumatoid arthritis.

FIG. 29A is a bar graph showing the level of IL-1β expression (pg IL-1β/mg of total protein) in the inflamed joints of CAIA mice at study day 14 in the indicated treatment group (IFP-2 or hIgG4 Fc control). FIG. 29B is a bar graph showing the level of IL-6 expression (pg IL-6/mg of total protein) in the inflamed joints of CAIA mice at study day 14 in the indicated treatment group (IFP-2 or hIgG4 Fc control). FIG. 29C is a bar graph showing the level of KC/GRO expression (pg KC/GRO/mg of total protein) in the inflamed joints of CAIA mice at study day 14 in the indicated treatment group (IFP-2 or hIgG4 Fc control). FIG. 29D is a bar graph showing the level of TNF expression (pg TNF/mg of total protein) in the inflamed joints of CAIA mice at study day 14 in the indicated treatment group (IFP-2 or hIgG4 Fc control).

FIG. 30A is a bar graph showing the level of IL-1β expression (pg IL-1β/mg of total protein) in the scrum joints of CAIA mice at study day 14 in the indicated treatment group (IFP-2 or hIgG4 Fc control). FIG. 30B is a bar graph showing the level of IL-6 expression (pg IL-6/mg of total protein) in the serum joints of CAIA mice at study day 14 in the indicated treatment group (IFP-2 or hIgG4 Fc control). FIG. 30C is a bar graph showing the level of KC/GRO expression (pg KC/GRO/mg of total protein) in the serum joints of CAIA mice at study day 14 in the indicated treatment group (IFP-2 or hIgG4 Fc control). FIG. 30D is a bar graph showing the level of TNF expression (pg TNF/mg of total protein) in the serum joints of CAIA mice at study day 14 in the indicated treatment group (IFP-2 or hIgG4 Fc control).

### 5. DETAILED DESCRIPTION

The inventors have, inter alia, identified and developed immunoreceptor inhibitory proteins that specifically bind to TNFSF ligand TL1A. Accordingly, the novel immunoreceptor inhibitory proteins disclosed herein may be useful for various methods, including, e.g., selectively inhibiting or reducing (e.g., preventing) binding of TL1A to DR3 and/or DcR3, inhibiting signaling of DR3 and/or DcR3 (including, e.g., signaling mediated through the binding of DR3 to TL1A), and modulating (e.g., suppressing) an immune response, as well as in diagnostic assays. As such, the current disclosure provides, inter alia, novel immunoreceptor inhibitory proteins, nucleic acid molecules encoding, the methods for utilizing the same.

TABLE OF CONTENTS

| | |
|---|---|
| 5.1 | Definitions |
| 5.2 | Immunoreceptor Inhibitory Proteins |
| 5.3 | Exemplary Properties of Immunoreceptor Inhibitory Proteins |
| 5.4 | Immunoreceptor Inhibitory Protein Fusions & Conjugates |
| 5.4.1 | Radioligands |
| 5.4.2 | Chimeric Antigen Receptors |
| 5.4.3 | Signal Peptides |
| 5.4.4 | Half-Life Extension Moieties |
| 5.4.5 | Ig Fusion Proteins |
| 5.4.5.1 | Antibody Fusion Proteins |
| 5.4.5.2 | Ig Fusion Proteins |
| 5.4.5.3 | Half-Life Extension |
| 5.4.5.4 | Ig Effector Function |
| 5.4.5.4(i) | Reduced Effector Function |
| 5.4.5.4(ii) | Enhanced Effector Function |
| 5.4.6 | Linkers |
| 5.4.7 | Orientation |
| 5.4.8 | Multimeric Fusion Proteins |
| 5.4.9 | Exemplary Ig Fusion Proteins |
| 5.5 | Immunogenic Peptides & Proteins |
| 5.5.1 | Fragments of Immunoreceptor Inhibitory Proteins |
| 5.5.2 | Variants of Immunoreceptor Inhibitory Proteins |
| 5.5.3 | Peptide and Protein-Based Vaccines |
| 5.5.4 | Nucleic Acid-Based Vaccines |
| 5.5.4.1 | DNA Molecules |
| 5.5.4.2 | RNA Molecules |
| 5.6 | Methods of Making Proteins |
| 5.7 | Nucleic Acid Molecules |
| 5.7.1 | DNA Molecules |
| 5.7.2 | RNA Molecules |
| 5.8 | Vectors |
| 5.8.1 | Non-Viral Vectors |
| 5.8.2 | Viral Vectors |
| 5.9 | Cells |
| 5.10 | Antibodies |
| 5.11 | Carriers |
| 5.11.1 | Carriers of IIPs |
| 5.11.2 | Carriers Conjugated to IIPs |
| 5.11.3 | Lipid Based Carriers/Lipid Nanoformulations |
| 5.11.3.1 | Cationic Lipids (Positively Charged) and Ionizable Lipids |
| 5.11.3.2 | Non-Cationic Lipids (e.g., Phospholipids) |
| 5.11.3.3 | Structural Lipids |
| 5.11.3.4 | Polymers and Polyethylene Glycol (PEG) - Lipids |
| 5.11.3.5 | Percentages of Lipid Nanoformulation Components |
| 5.12 | Adjuvants |
| 5.13 | Pharmaceutical Compositions |
| 5.14 | Methods of Use |
| 5.14.1 | Methods of Delivery |
| 5.14.2 | Methods of Inhibiting or Reducing Binding of a TNFSF |
| | Ligand to One or More Cognate TNFSF Receptor |
| 5.14.3 | Methods of Inhibiting or Reducing Signaling Mediated by |
| | TNFSF Ligand binding to One or More Cognate TNFSF |
| | Receptor |
| 5.14.4 | Methods of Inhibiting or Reducing Binding of TL1A to DR3 |
| 5.14.5 | Methods of Inhibiting of Reducing Signaling Mediated by |
| | TL1A Binding to DR3 |
| 5.14.6 | Methods of Inhibiting or Reducing Binding of TL1A to DcR3 |
| 5.14.7 | Methods of Inhibiting or Reducing Signaling Mediated by |
| | TL1A Binding to DcR3 |
| 5.14.8 | Methods of Suppressing or Preventing a Pro-Inflammatory |
| | Immune Response |
| 5.14.9 | Methods of Preventing, Treating, or Ameliorating a Disease in |
| | a Subject in Need Thereof |
| 5.14.10 | Methods of Inducing or Enhancing an Immune Response |
| 5.14.11 | Methods of Vaccinating a Subject |
| 5.14.12 | Diagnostic Methods |
| 5.15 | Kits |

### 5.1 Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

Use of the singular herein includes the plural unless specifically stated otherwise. For example, as used herein, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The term “about” refers to a value or composition that is within an acceptable error range for the particular value or composition as determined by one of ordinary skill in the art, which will depend in part on how the value or composition is measured or determined, i.e., the limitations of the measurement system. When particular values or compositions are provided herein, unless otherwise stated, the meaning of “about” should be assumed to be within an acceptable error range for that particular value or composition.

Where proteins and/or polypeptides are described herein, it is understood that nucleic acid molecules (e.g., RNA (e.g., mRNA) or DNA molecules) encoding the protein are also provided herein.

Where proteins, peptides, nucleic acid molecules, vectors, carriers, etc. are described herein, it is understood that isolated forms of the proteins, peptides, nucleic acid molecules, vectors, carriers, etc. are also provided herein.

Where proteins, peptides, nucleic acid molecules, etc. are described herein, it is understood that recombinant forms of the proteins, peptides, nucleic acid molecules, etc. are also provided herein.

Where polypeptides or sets of polypeptides are described herein, it is understood that proteins comprising the polypeptides or sets of polypeptides folded into their three-dimensional structure (i.e., tertiary or quaternary structure) are also provided herein and vice versa.

As used herein, the term “adjuvant” refers to a substance that causes stimulation of the immune system of a subject when administered to the subject.

As used herein, the term “administering” refers to the physical introduction of an agent, e.g., a therapeutic agent (or a precursor of the therapeutic agent that is metabolized or altered within the body of the subject to produce the therapeutic agent in vivo) or vaccine to a subject, using any of the various methods and delivery systems known to those skilled in the art. Administering can also be performed, for example, once, a plurality of times, and/or over one or more extended periods. The term administering includes both self-administration by the subject themselves and administration to the subject by another.

As used herein, the term “affinity” refers to the strength of the binding of one protein (e.g., a Ligand) to another protein (e.g., a Receptor). The affinity of a protein is measured by the dissociation constant Kd, defined as [Ligand]×[Receptor]/[Ligand-Receptor] where [Ligand-Receptor] is the molar concentration of the Ligand-Receptor complex, [Ligand] is the molar concentration of the unbound Ligand and [Receptor] is the molar concentration of the unbound Receptor. The affinity constant Ka is defined by 1/Kd. Standard methods of measuring affinity are known to the person of ordinary skill in the art and described herein, see, e.g., § 5.3.

As used herein, the term “agent” is used generically to describe any macro or micro molecule. Exemplary agents include, but are not limited proteins, peptides, nucleic acid molecules (e.g., DNA molecules, RNA molecules), vectors, carriers, carbohydrates, lipids, synthetic polymers, etc.

As used herein, the term “antibody” or “antibodies” is used in the broadest sense and encompasses various immunoglobulin (Ig) (e.g., human Ig (hIg), murine Ig (mIg)) structures, including, but not limited to monoclonal antibodies, polyclonal antibodies, multispecific (e.g., bispecific, trispecific) antibodies, and antibody fragments so long as they exhibit the desired antigen-binding activity (i.e., antigen binding fragments or variants). The term antibody thus includes, for example, full-length antibodies; antigen-binding fragments of full-length antibodies; molecules comprising antibody CDRs, VH regions, and/or VL regions; and antibody-like scaffolds (e.g., fibronectins). Examples of antibodies include, without limitation, monoclonal antibodies, polyclonal antibodies, monospecific antibodies, multispecific antibodies, human antibodies, humanized antibodies, chimeric antibodies, camelized antibodies, intrabodies, affybodies, diabodies, tribodies, heteroconjugate antibodies, antibody-drug conjugates, single domain antibodies (e.g., VHH, (VHH)₂), single chain antibodies, single-chain Fvs (scFv; (scFv)₂), Fab fragments (e.g., Fab, single chain Fab (scFab), F(ab′)₂ fragments, disulfide-linked Fvs (sdFv), Fc fusions (e.g., Fab-Fc, scFv-Fc, VHH-Fc, (scFv)₂-Fc, (VHH) 2-Fc), and antigen-binding fragments of any of the above, and conjugates or fusion proteins comprising any of the above. Antibodies can be of Ig isotype (e.g., IgG, IgE, IgM, IgD, or IgA), any class (e.g., IgG₁, IgG₂, IgG₃, IgG₄, IgA₁ or IgA₂), or any subclass (e.g., IgG₂ₐ or IgG_{2b}) of Ig). In certain embodiments, antibodies described herein are IgG antibodies, or a class (e.g., human IgG₁ or IgG₄) or subclass thereof. In certain embodiments, antibodies described herein are mIgG antibodies, or a class (e.g., mIgG1 or mIgG2a) or subclass thereof. In some embodiments, the antibody is a human, humanized, or chimeric IgG₁ or IgG₄ monoclonal antibody. In some embodiments, the term antibodies refers to a monoclonal or polyclonal antibody population. Antibodies described herein can be produced by any standard methods known in the art, e.g., recombinant production in host cells, see, e.g., § 5.6; or synthetic production.

As used herein, the term “antibody mimetic” refers to non-Ig based antigen binding domain. Various antibody-like scaffolds are known in the art. For example, 10th type III domain of fibronectin (e.g., AdNectins®) and designed ankyrin repeat proteins (e.g., DARPins®) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13:695-701 (2008), the full contents of each of which is incorporated by reference herein for all purposes. Exemplary antibody-like scaffolds include, but are not limited to, lipocalins (see, e.g., U.S. Pat. No. 7,250,297) (e.g., Anticalin®), protein A-derived molecules such as z-domains of protein a (see, e.g., U.S. Pat. No. 5,831,012) (e.g., Affibody®), A domains of membrane receptors stabilized by disulfide bonds and Ca2+ (see, e.g., U.S. Pat. No. 7,803,907) (e.g., Avimer/Maxibody®), a serum transferrin (see, e.g., US2004023334) (e.g., Transbody®); a designed ankyrin repeat protein (see, e.g., U.S. Pat. No. 7,417,130) (e.g., DARPin®), a fibronectin (see, e.g., U.S. Pat. No. 6,818,418) (e.g., AdNectin®), a C-type lectin domain (see, e.g., US2004132094) (e.g., Tetranectin®); a human gamma-crystallin or ubiquitin (see, e.g., U.S. Pat. No. 7,838,629) (e.g., Affilin®); a kunitz type domain of human protease inhibitors (see, e.g., US2004209243), C-Type Lectins (see, e.g., US2004132094) (e.g., Tetranectins®), cysteine knots or knottins (see, e.g., U.S. Pat. No. 7,186,524) (e.g., Microbodies®), nucleic acid aptamers (see, e.g., U.S. Pat. No. 5,475,096), thioredoxin A scaffold (see, e.g., U.S. Pat. No. 6,004,746) (peptide aptamers), and 10th type III domain of fibronectin (see, e.g., U.S. Pat. No. 6,818,418) (e.g., AdNectins®), and cystine-dense peptides (see, e.g., WO2023023031). Additional exemplary antibody-like scaffolds are known in the art and for example described in Storz U. Intellectual property protection: strategies for antibody inventions. MAbs. 2011; 3 (3): 310-317. doi:10.4161/mabs.3.3.15530. The entire contents of each of the foregoing references is incorporated herein by reference for all purposes. Antibody like scaffolds include e.g., naturally occurring antigen binders, variant (e.g., functional variants) of naturally occurring antigen binders, fragments (e.g., functional fragments) of naturally occurring antigen binders, and synthetic antigen binders (i.e., not naturally occurring antigen binders).

The terms “CH1” and “CH1 region” are used interchangeably herein and refer to the first constant region of an immunoglobulin heavy chain. The amino acid sequence of an exemplary reference hIgG1 CH1 region is set forth in SEQ ID NO: 90; and the amino acid sequence of an exemplary reference hIgG4 CH1 region is set forth in SEQ ID NO: 103.

The terms “CH2” and “CH2 region” are used interchangeably herein and refer to the second constant region of an immunoglobulin heavy chain. The amino acid sequence of an exemplary reference hIgG1 CH2 region is set forth in SEQ ID NO: 92; and the amino acid sequence of an exemplary reference hIgG4 CH2 region is set forth in SEQ ID NO: 106.

The terms “CH3” and “CH3 region” are used interchangeably herein and refer to the third constant region of an immunoglobulin heavy chain. The amino acid sequence of an exemplary reference hIgG1 CH3 region is set forth in SEQ ID NO: 93; and the amino acid sequence of an exemplary reference hIgG4 CH3 region is set forth in SEQ ID NO: 107.

As used herein, the term “chimeric antigen receptor” or “CAR” refers to a recombinant polypeptide construct comprising at least an extracellular antigen-binding domain (e.g., comprising a immunoreceptor inhibitory protein described herein), a transmembrane domain, and an intracellular signaling domain comprising one or more functional signaling domains derived from a stimulatory molecule. In some embodiments, the domains in the CAR polypeptide construct are in the same polypeptide chain. In some embodiments, the domains in the CAR polypeptide construct are not contiguous with each other, for example, are in different polypeptide chains.

As used herein, the term “circular RNA” refers to a translatable RNA molecule that forms a circular structure through covalent or non-covalent bonds. In some embodiments, the circular RNA is covalently closed.

As used herein, the term “conjugation” refers to chemical conjugation of a protein with a moiety (e.g., small molecule, polypeptide, nucleic acid molecule, carbohydrate, lipid, synthetic polymer (e.g., polymers of polyethylene glycol (PEG)), etc.). The moiety can be directly connected to the protein or indirectly connected through a linker, e.g., as described herein. Chemical conjugation methods are well known in the art, as are commercially available conjugation reagents and kits, with detailed instructions for their use readily available from the commercial suppliers.

As used herein, the term “derived from,” with reference to a nucleic acid molecule refers to a nucleic acid molecule that has at least 70% sequence identity to a reference nucleic acid molecule (e.g., a naturally occurring nucleic acid molecule) or a fragment thereof. The term “derived from,” with reference to a protein refers to a protein that comprises an amino acid sequence that has at least 70% sequence identity to the amino acid sequence of a reference protein (e.g., a naturally occurring protein). The term “derived from” as used herein does not denote any specific process or method for obtaining the nucleic acid molecule, polypeptide, or protein. For example, the nucleic acid molecule, polypeptide, or protein can be recombinantly produced or chemically synthesized.

As used herein the term “DcR3” or “Tumor Necrosis Factor Receptor Superfamily Member 6B” refers to the receptor of the TNFSF that binds, e.g., FASL, LIGHT. The amino acid sequence of an exemplary reference immature human DR3 (hDR3) protein is set forth in SEQ ID NO: 19 and the amino acid sequence of an exemplary reference mature hDR3 protein is set forth in SEQ ID NO: 20.

As used herein, the term “diagnosing” or “diagnosis” refers to a determination of the presence, absence, severity, or course of treatment of a disease (e.g., an infection, e.g., a viral infection). The term “diagnosing” encompasses an initial determination as well as subsequent determinations (e.g., monitoring) after the initial determination.

As used herein, the term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

As used herein the term “DR3” or “Tumor Necrosis Factor Receptor Superfamily Member 25” refers to the receptor of the TNFSF that binds, e.g., TL1A. The amino acid sequence of an exemplary reference immature human DR3 (hDR3) protein is set forth in SEQ ID NO: 17 and the amino acid sequence of an exemplary reference mature hDR3 protein is set forth in SEQ ID NO: 18.

The term “effector function” when used in reference to an antibody refers to those biological activities attributable to the Fc region of an antibody, which therefore vary with the antibody isotype. Antibody effector functions include, but are not limited to, antibody-dependent cell-mediated cytotoxicity (ADCC), antibody-dependent cellular phagocytosis (ADCP), complement dependent cytotoxicity (CDC), Fc receptor binding (e.g., FcγRI, FcγRIIa, FcγRIIc, FcγRIIIa, and/or FcγRIIIb (e.g., FcγRI, FcγIIa, and/or FcγIIIa)), and C1q binding.

As used herein the term “FAS” or “Tumor Necrosis Factor Receptor Superfamily Member 6” refers to the receptor of the TNFSF that binds, e.g., FASL. The amino acid sequence of an exemplary reference immature human FAS (hFAS) protein is set forth in SEQ ID NO: 15 and the amino acid sequence of an exemplary reference mature hFAS protein is set forth in SEQ ID NO: 16.

As used herein the term “FASL” or “FAS Ligand” or “Tumor Necrosis Factor Ligand Superfamily Member 6” refers to the immunomodulatory cytokine of the TNFSF. The amino acid sequence of an exemplary reference membrane human FASL (hFASL) protein is set forth in SEQ ID NO: 5 and the amino acid sequence of an exemplary reference soluble hFASL protein is set forth in SEQ ID NO: 6.

As used herein, the term “Fc region” refers to the C-terminal region of an Ig heavy chain that comprises from N- to C-terminus at least a CH2 region operably connected to a CH3 region. In some embodiments, the Fc region comprises an Ig hinge region or at least a portion of an Ig hinge region operably connected to the N-terminus of the CH2 region. In some embodiments, the Fc region is engineered relative to a reference Fc region, see, e.g., § 5.4.5.4. Additional examples of proteins with engineered Fc regions can be found in Saunders 2019 (K. O. Saunders, “Conceptual Approaches to Modulating Antibody Effector Functions and Circulation Half-Life,” 2019, Frontiers in Immunology, V. 10, Art. 1296, pp. 1-20, the entire contents of which is incorporated by reference herein for all purposes).

The term “functional variant” as used herein in reference to a protein refers to a protein that comprises at least one but no more than 15%, not more than 12%, no more than 10%, no more than 8% amino acid variation (e.g., substitution, deletion, addition) compared to the amino acid sequence of a reference protein, wherein the protein retains at least one particular function of the reference protein. Not all functions of the reference protein (e.g., wild type) need be retained by the functional variant of the protein. In some instances, one or more functions are selectively reduced or eliminated. In some embodiments, the reference protein is a wild type protein. For example, a functional variant of a TNF superfamily ligand binding protein can refer to a TNF superfamily ligand binding protein comprising one or more amino acid substitution as compared to a reference TNF superfamily ligand binding protein (e.g., a wild type protein) that retains the ability to specifically bind the TNF superfamily ligand.

The term “functional fragment” as used herein in reference to a protein refers to a fragment of a reference protein that retains at least one particular function. Not all functions of the reference protein need be retained by a functional fragment of the protein. In some instances, one or more functions are selectively reduced or eliminated. In some embodiments, the reference protein is a wild type protein. For example, a functional fragment of a TNF superfamily ligand binding protein can refer to a fragment of a TNF superfamily ligand binding protein that retains the ability to specifically bind the TNF superfamily ligand.

As used herein, the term “fuse” and grammatical equivalents thereof refer to the operable connection of at least a first polypeptide to a second polypeptide, wherein the first and second polypeptides are not naturally found operably connected together. For example, the first and second polypeptides are derived from different proteins. The term fuse encompasses both a direct connection of the at least two polypeptides through a peptide bond, and the indirect connection through a linker (e.g., a peptide linker).

As used herein, the term “fusion protein” and grammatical equivalents thereof refers to a protein that comprises at least one polypeptide operably connected to another polypeptide, wherein the first and second polypeptides are not naturally found operably connected together. For example, the first and second polypeptides of the fusion protein are each derived from different proteins. The at least two polypeptides of the fusion protein can be directly operably connected through a peptide bond; or can be indirectly operably connected through a linker (e.g., a peptide linker). Therefore, for example, the term fusion polypeptide encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B).

As used herein, the term “half-life extension moiety” refers to a moiety (e.g., small molecule, polypeptide, nucleic acid molecule, carbohydrate, lipid, synthetic polymer (e.g., polymers of PEG), etc.) that when conjugated or otherwise operably connected (e.g., fused) to a protein (the subject protein), increases the half-life of the subject protein in vivo when administered to a subject (e.g., a human subject). The pharmacokinetic properties of the protein can be evaluated utilizing in vivo models known in the art.

As used herein, the term “half-life extension polypeptide” or “half-life extension protein” refers to a protein that when operably connected to another protein (the subject protein), increases the half-life of the subject protein in vivo when administered to a subject (e.g., a human subject). The pharmacokinetic properties of the protein can be evaluated utilizing in vivo models known in the art.

As used herein, the term “heterologous”, when used to describe a first element in reference to a second element means that the first element and second element do not exist in nature disposed as described. For example, a polypeptide comprising a “heterologous moiety” means a polypeptide that is joined to a moiety (e.g., small molecule, polypeptide, nucleic acid molecule, carbohydrate, lipid, synthetic polymer (e.g., polymers of PEG), etc.) that is not joined to the polypeptide in nature. In one embodiment, the heterologous moiety is not derived from a protein comprising or consisting of the amino acid sequence set forth in any one of SEQ ID NOS: 23-84, 187-190, or 199-200. For example, a non-limiting example of a heterologous moiety is a heterologous polypeptide (as defined herein). In one embodiment, the heterologous polypeptide is a polypeptide derived from a protein other than a protein comprising or consisting of the amino acid sequence set forth in any one of SEQ ID NOS: 23-84, 187-190, or 199-200. For example, a non-limiting example of a heterologous polypeptide, as described herein, is a human Ig Fc region.

As used, herein the term “heterologous signal peptide” refers to a signal peptide that is not operably connected to a subject protein in nature. For example, in reference to a polypeptide comprising a signal peptide from human IL-2 operably connected to human IL-12, the human IL-2 signal peptide would constitute a heterologous signal peptide. The terms “signal peptide” and “signal sequence” are used interchangeably herein.

The terms “hinge” or “hinge region” are used interchangeably herein and refer to the hinge region of an immunoglobulin heavy chain. The amino acid sequence of an exemplary reference hIgG1 hinge region is set forth in SEQ ID NO: 91; and the amino acid sequence of an exemplary reference hIgG4 hinge region is set forth in SEQ ID NO: 104.

As used herein, the term “homologous signal peptide” refers to a signal peptide that is operably connected to a subject protein in nature. For example, in reference to a polypeptide comprising a signal peptide from human IL-2 operably connected to human IL-2, the human IL-2 signal peptide would constitute a homologous signal peptide.

As used herein, the term “immunogen” refers to a substance that is capable of inducing an immune response (e.g., an adaptive immune response) in a subject (e.g., a human subject). An immunogen may have one or more isoforms, sequence variants, or splice variants that have equivalent biological and immunological activity, and are thus also considered for the purposes of this disclosure to be immunogenic equivalents of the immunogen.

As used herein, the term “immunogenic peptide or protein” refers to a peptide or protein that comprises an immunogen.

As used herein, the term “immunoreceptor inhibitory protein” refers to a protein (e.g., a protein described herein) that inhibits (e.g., partially, fully) a function of one or more immune receptor (e.g., one or more of TNFSF receptor). Exemplary functions include binding to a cognate ligand (e.g., one or more cognate TNFSF ligand), signaling (e.g., signaling induced by binding to a cognate ligand), etc.

As used herein, the term “in combination with” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed. In some embodiments, administration of two or more agents or treatments in combination is such that the reduction in a symptom, or other parameter related to the condition is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, and intramuscular routes. The therapeutic agents can be administered by the same route or by different routes.

As used herein, the term “isolated” with reference to a polypeptide, protein, or nucleic acid molecule refers to a polypeptide, protein, or nucleic acid molecule that is substantially free of other cellular components with which it is associated in the natural state.

As used herein the term “LIGHT” or “Tumor Necrosis Factor Ligand Superfamily Member 14” refers to the immunomodulatory cytokine of the TNFSF. The amino acid sequence of an exemplary reference membrane human LIGHT (hLIGHT) protein is set forth in SEQ ID NO: 3 and the amino acid sequence of an exemplary reference soluble hLIGHT protein is set forth in SEQ ID NO: 4.

As used herein the term “LIGHTR” or “Tumor Necrosis Factor Receptor Superfamily Member 14” refers to the receptor of the TNFSF that binds, e.g., LIGHT. The amino acid sequence of an exemplary reference immature human LIGHTR (hLIGHTR) protein is set forth in SEQ ID NO: 13 and the amino acid sequence of an exemplary reference mature hLIGHTR protein is set forth in SEQ ID NO: 14.

As used herein the term “LTβR” or “Tumor Necrosis Factor Receptor Superfamily Member 3” refers to the receptor of the TNFSF that binds, e.g., LIGHT. The amino acid sequence of an exemplary reference immature human LTβR (hLTβR) protein is set forth in SEQ ID NO: 21 and the amino acid sequence of an exemplary reference mature hLTβR protein is set forth in SEQ ID NO: 22.

As used herein, the term “moiety” is used generically to describe any macro or micro molecule that can be operably connected to a protein described herein. Exemplary moieties include, but are not limited small molecules, polypeptides, nucleic acid molecules (e.g., DNA, RNA), carbohydrates, lipids, synthetic polymers (e.g., polymers of PEG).

As used herein, the term “modified nucleotide,” “nucleotide modification,” or use of the term “modification” and the like in reference to a nucleotide or nucleic acid sequence refers to a nucleotide comprising a chemical modification, e.g., a modified sugar moiety, a modified nucleobase, and/or a modified internucleoside linkage, or any combination thereof. Exemplary modifications are provided herein, see, e.g., § 5.5.4.2. In certain embodiments of the instant disclosure, inclusion of a deoxynucleotide-which is acknowledged as a naturally occurring form of nucleotide-if present within an RNA molecule is considered to constitute a modified nucleotide.

As used herein, the term “obtaining a sample” refers to the acquisition of a sample. The term includes the direct acquisition from a subject and the indirect acquisition through one or more third parties wherein one of the third parties directly acquired the sample from the subject.

As used herein, the term “operably connected” refers to the linkage of two moieties in a functional relationship. For example, a polypeptide is operably connected to another polypeptide when they are linked (either directly or indirectly via a peptide linker) in frame such that both polypeptides are functional (e.g., a fusion protein described herein). Or for example, a transcription regulatory nucleic acid molecule e.g., a promoter, enhancer, or other expression control element is operably linked to a nucleic acid molecule that encodes a protein if it affects the transcription of the nucleic acid molecule that encodes the protein. The term “operably connected” can also refer to the conjugation of a moiety to e.g., a nucleic acid molecule or polypeptide (e.g., the conjugation of a PEG polymer to a protein).

The determination of “percent identity” between two sequences (e.g., peptide or protein (amino acid sequences) or polynucleotide (nucleic acid sequences)) can be accomplished using a mathematical algorithm. A specific, non-limiting example of a mathematical algorithm utilized for the comparison of two sequences is the algorithm of Karlin S & Altschul S F (1990) PNAS 87:2264-2268, modified as in Karlin S & Altschul S F (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance intended for use in contact with the tissues of human beings and/or non-human animals, and without excessive toxicity, irritation, allergic response, or other problem or complication, commensurate with a reasonable therapeutic benefit/risk ratio.

As used herein, the term “plurality” means 2 or more (e.g., 3 or more, 4 or more, 5 or more, 6 or more, 7 or more, 9 or more, or 10 or more).

As used herein, the term “poly(A) sequence,” refers to a sequence of adenosine nucleotides, typically located at the 3′-end of a coding linear RNA, of up to about 1000 adenosine nucleotides. In some embodiments, the poly(A) sequence is essentially homopolymeric, e.g., a poly(A) sequence of e.g., 100 adenosine nucleotides having essentially the length of 100 nucleotides. In other embodiments, the poly(A) sequence may be interrupted by at least one nucleotide different from an adenosine nucleotide, e.g., a poly(A) sequence of e.g., 100 adenosine nucleotides may have a length of more than 100 nucleotides (comprising 100 adenosine nucleotides and in addition said at least one nucleotide—or a stretch of nucleotides—different from an adenosine nucleotide). It has to be understood that “poly(A) sequence” as defined herein typically relates to mRNA—however in the context of the invention, the term likewise relates to corresponding sequences in a DNA molecule (e.g., a “poly(T) sequence”).

The terms “polynucleotide” and “nucleic acid molecule” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA molecules encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the terms “protein” and “polypeptide” refers to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” and “protein” are used interchangeably herein. In some embodiments, the protein is folded into its three-dimensional structure. Where linear polypeptides are contemplated herein (i.e., primary structure (amino acid sequence)), it should be understood that proteins folded into their three-dimensional structure are also provided herein. Where proteins are contemplated herein (i.e., folded into their three-dimensional structure) polypeptides in their primary structure (i.e., the amino acid sequence) are also provided herein.

A “prophylactic” treatment is a treatment administered to a subject who does not exhibit signs of a disease or exhibits only early signs for the purpose of decreasing the risk of developing pathology.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester linkage found between the nucleotides of an unmodified nucleic acid molecule.

As used herein, the term “sample” encompass a variety of biological specimens obtained from a subject. Exemplary sample types include, e.g., blood and other liquid samples of biological origin (including, but not limited to, whole-blood, peripheral blood mononuclear cells (PBMCs), serum, plasma, urine, saliva, amniotic fluid, stool, synovial fluid, etc.), nasopharyngeal swabs, solid tissue samples such as biopsies (or cells derived therefrom and the progeny thereof), tissue cultures (or cells derived therefrom and the progeny thereof), and cell cultures (or cells derived therefrom and the progeny thereof). The term also includes samples that have been manipulated in any way after their procurement from a subject, such as by centrifugation, filtration, washing, precipitation, dialysis, chromatography, lysis, treatment with reagents, enriched for certain cell populations, refrigeration, freezing, staining, etc.

As used herein, the term “translatable RNA” refers to any RNA that encodes at least one polypeptide and can be translated to produce the encoded protein in vitro, in vivo, in situ or ex vivo. A translatable RNA may be an mRNA or a circular RNA encoding a polypeptide.

The term “(scFv)₂” as used herein refers to an antibody that comprises a first and a second scFv operably connected (e.g., via a peptide linker). The first and second scFv can specifically bind the same or different antigens. In some embodiments, the first and second scFv are operably connected by a peptide linker.

The term “scFv-Fc” as used herein refers to an antibody that comprises a scFv operably linked (e.g., via a peptide linker) to an Fc domain or subunit of an Fc domain. In some embodiments, a scFv is operably connected to only a first Fc domain of a first and a second Fc domain pair. In some embodiments, a first scFv is operably connected to a first Fc domain and a second scFv is operably connected to a second Fc domain of a first and second Fc domain pair.

The term “(scFv)₂-Fc” as used herein refers to a (scFv)₂ operably linked (e.g., via a peptide linker) to an Fc domain or a subunit of an Fc domain. In some embodiments, a (scFv)₂ is operably connected to only a first Fc domain of a first and a second Fc domain pair. In some embodiments, a first (scFv)₂ is operably connected to a first Fc domain and a second (scFv)₂ is operably connected to a second Fc domain of a first and second Fc domain pair.

As used herein, the term “single domain antibody” or “sdAb” refers to an antibody having a single monomeric variable antibody domain. A sdAb is able to specifically bind to a specific antigen. A VHH (as defined herein) is an example of a sdAb.

As used herein, the term “signal peptide” or “signal sequence” refers to a sequence (e.g., an amino acid sequence) that can direct the transport or localization of a protein to a certain organelle, cell compartment, or extracellular export. The term encompasses both the signal sequence peptide and the nucleic acid sequence encoding the signal peptide. Thus, references to a signal peptide in the context of a nucleic acid refers to the nucleic acid sequence encoding the signal peptide.

As used herein, the term “specifically binds” refers to preferential interaction, i.e., significantly higher binding affinity, between a first protein (e.g., a ligand) and a second protein (e.g., the ligand's cognate receptor) relative to other amino acid sequences. Herein, when a first protein is said to “specifically bind” to a second protein, it is understood that the first protein specifically binds to an epitope of the second protein. The term “epitope” refers to the portion of the second protein that the first protein specifically recognizes. The term specifically binds includes molecules that are cross reactive with the same epitope of a different species. For example, an antibody that specifically binds human TL1A may be cross reactive with TL1A of another species (e.g., cynomolgus, murine, etc.), and still be considered herein to specifically bind human TL1A. A protein can specifically bind more than one different protein.

As used herein, the term “subject” includes any animal, such as a human or other animal. In some embodiments, the subject is a vertebrate animal (e.g., mammal, bird, fish, reptile, or amphibian). In some embodiments, the subject is a human. In some embodiments, the method subject is a non-human mammal. In some embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

As used herein, the term “therapeutically effective amount” of a therapeutic agent refers to any amount of the therapeutic agent that, when used alone or in combination with another therapeutic agent, improves a disease condition, e.g., protects a subject against the onset of a disease (or infection); improves a symptom of disease or infection, e.g., decreases severity of disease or infection symptoms, decreases frequency or duration of disease or infection symptoms, increases disease or infection symptom-free periods; prevents or reduces impairment or disability due to the disease or infection; or promotes disease (or infection) regression. The ability of a therapeutic agent to improve a disease condition can be evaluated using a variety of methods known to the skilled practitioner, such as in human subjects during clinical trials, in animal model systems predictive of efficacy in humans, or by assaying the activity of the agent in in vitro assays.

As used herein, the terms “treat,” treating,” “treatment,” and the like refer to reducing or ameliorating a disease or infection and/or symptom(s) associated therewith or obtaining a desired pharmacologic and/or physiologic effect. It will be appreciated that, although not precluded, treating a disease or infection does not require that the disease or infection, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease or infection. In some embodiments, the effect is preventative, i.e., the effect protects or prevents an occurrence or reoccurrence of a disease or infection. To this end, the presently disclosed methods comprise administering a therapeutically effective amount of a composition as described herein.

As used herein the term “TL1A” or “Tumor Necrosis Factor Ligand Superfamily Member 15” refers to the immunomodulatory cytokine of the TNFSF. The amino acid sequence of an exemplary reference membrane human TL1A (hTL1A) protein is set forth in SEQ ID NO: 7 and the amino acid sequence of an exemplary reference soluble hTL1A protein is set forth in SEQ ID NO: 8.

As used herein the term “TNFα” or “Tumor Necrosis Factor α” or “Tumor Necrosis Factor Ligand Superfamily Member 2” refers to the multifunctional immunomodulatory cytokine of the TNFSF. The amino acid sequence of an exemplary reference membrane human TNFα (hTNFα) protein is set forth in SEQ ID NO: 1 and the amino acid sequence of an exemplary reference soluble hTNFα protein is set forth in SEQ ID NO: 2.

As used herein the term “TNFR1” or “Tumor Necrosis Factor Receptor Superfamily Member 1A” refers to the receptor of the TNFSF that binds, e.g., TNFα. TNFR1 can be expressed both as a transmembrane protein and as a secreted protein through proteolytic processing. The amino acid sequence of an exemplary reference immature human TNFR1 (hTNFR1) protein is set forth in SEQ ID NO: 9 and the amino acid sequence of an exemplary reference mature hTNFR1 protein is set forth in SEQ ID NO: 10.

As used herein the term “TNFR2” or “Tumor Necrosis Factor Receptor Superfamily Member 1B” refers to the receptor of the TNFSF that binds, e.g., TNFα. The amino acid sequence of an exemplary reference immature human TNFR2 (hTNFR2) protein is set forth in SEQ ID NO: 11 and the amino acid sequence of an exemplary reference mature hTNFR2 protein is set forth in SEQ ID NO: 12.

As used herein, the term “variant” or “variation” with reference to a nucleic acid molecule, refers to a nucleic acid molecule that comprises at least one substitution, alteration, inversion, addition, or deletion of nucleotide compared to a reference nucleic acid molecule. As used herein, the term “variant” or “variation” with reference to a protein refers to a protein that comprises at least one substitution, alteration, inversion, addition, or deletion of an amino acid residue compared to a reference protein.

As used herein, the term “variant Ig Fc fusion protein” refers to a fusion protein comprising an immunoreceptor inhibitory protein described herein and an Ig Fe region, wherein the Ig Fc region comprises one or more variation (e.g., one or more amino acid substitution, deletion, or addition)) that decreases or abolishes one or more Fc effector function, relative to a reference Ig Fc fusion protein that does not comprise the one or more variation.

The terms “VH” and “VH domain” are used interchangeably to refer to the heavy chain variable region of an antibody.

The term “VHH” as used herein refers to a type of single domain antibody (sdAb) that has a single monomeric heavy chain variable antibody domain (VH). Such antibodies can be found in or produced from camelid mammals (e.g., camels, llamas) which are naturally devoid of light chains or synthetically produced.

The terms “VL” and “VL domain” are used interchangeably to refer to the light chain variable region of an antibody.

As used herein, the term “5′-untranslated region” or “5′-UTR” refers to a part of a nucleic acid molecule located 5′ (i.e., “upstream”) of a coding sequence and which is not translated into protein. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g., by enzymatic or post-transcriptional addition of a 5′-cap structure.

As used herein the term “3′-untranslated region” or “3′-UTR” refers to a part of a nucleic acid molecule located 3′ (i.e., downstream) of a coding sequence and which is not translated into protein. A 3′-UTR may located between a coding sequence and an (optional) terminal poly(A) sequence of a nucleic acid sequence. A 3′-UTR may comprise elements for controlling gene expression, also called regulatory elements. Such regulatory elements may be, e.g., ribosomal binding sites, miRNA binding sites etc.

### 5.2 Immunoreceptor Inhibitory Proteins

The present disclosure provides, inter alia, immunoreceptor inhibitory proteins (IIPs) (and functional fragments and variants thereof) that specifically bind to TL1A (e.g., hTL1A). In specific embodiments, the IIPSs described herein specifically bind one or more TNF superfamily (TNFSF) ligands (e.g., a subset of human TNFSF ligands) (e.g., TL1A). The TNF superfamily of ligands and receptors regulates multiple cellular functions, including e.g., immune responses, cell proliferation, cell survival, cell differentiation, and programmed cell death. The TNF superfamily comprises 19 ligands and 29 receptors. Exemplary TNF superfamily ligands and their cognate receptors, include, e.g., TNFα and TNFR1/TNFR2; FasL and Fas; LIGHT and LIGHTR and LTβR; and TL1A and DR3/DcR3. A subset of TNF superfamily ligands are known to interact with more than one TNF superfamily receptor (e.g., TNFα is known to interact with both TNFR1 and TNFR2).

The amino acid sequence of exemplary reference human TNF superfamily ligands (e.g., human TNFα (hTNFα), human LIGHT (hLIGHT), human FasL (hFASL), and human TL1A (hTL1A)) and receptors (e.g., human TNFR1 (hTNFR1), human TNFR2 (hTNFR2), human LIGHTR (hLIGHTR), human FAS (hFAS), human DR3 (hDR3), and human DcR3 (hDcR3)) are set forth in Table 1 below. More specifically, the amino acid sequence of the membrane and soluble form of hTNFα is set forth in SEQ ID NOS: 1 and 2, respectively. The amino acid sequence of the membrane and soluble form of hLIGHT is set forth in SEQ ID NOS: 3 and 4, respectively. The amino acid sequence of the membrane and soluble form of hFASL is set forth in SEQ ID NOS: 5 and 6, respectively. The amino acid sequence of the membrane and soluble form of hTL1A is set forth in SEQ ID NOS: 7 and 8, respectively. The amino acid sequence of the immature and mature form of hTNFR1 is set forth in SEQ ID NOS: 9 and 10, respectively. The amino acid sequence of the immature and mature form of hTNFR2 is set forth in SEQ ID NOS: 11 and 12, respectively. The amino acid sequence of the immature and mature form of hLIGHTR is set forth in SEQ ID NOS: 13 and 14, respectively. The amino acid sequence of the immature and mature form of hFAS is set forth in SEQ ID NOS: 15 and 16, respectively. The amino acid sequence of the immature and mature form of hDR3 is set forth in SEQ ID NOS: 17 and 18, respectively. The amino acid sequence of the immature and mature form of hDcR3 is set forth in SEQ ID NOS: 19 and 20, respectively. The amino acid sequence of the immature and mature form of hLTβR is set forth in SEQ ID NOS: 21 and 22, respectively. See Table 1, herein.

TABLE 1
The Amino Acid Sequence of human TNF Superfamily Reference Ligands and
Receptors.

| | | |
|---|---|---|
| | | SEQ |
| Description | Amino Acid Sequence | ID NO |

Exemplary Ligands

| | | |
|---|---|---|
| hTNFα Membrane | MSTESMIRDVELAEEALPKKTGGPQGSRRCLFLSLFSFLIVAGATT | 1 |
| Form | LFCLLHFGVIGPQREEFPRDLSLISPLAQAVRSSSRTPSDKPVAHV | |
| Uniprot ID: P01375 | VANPQAEGQLQWLNRRANALLANGVELRDNQLVVPSEGLYLIYSQV | |
| | LFKGQGCPSTHVLLTHTISRIAVSYQTKVNLLSAIKSPCQRETPEG | |
| | AEAKPWYEPIYLGGVFQLEKGDRLSAEINRPDYLDFAESGQVYFGI | |
| | IAL | |
| hTNFα Soluble | VRSSSRTPSDKPVAHVVANPQAEGQLQWLNRRANALLANGVELRDN | 2 |
| Form | QLVVPSEGLYLIYSQVLFKGQGCPSTHVLLTHTISRIAVSYQTKVN | |
| Uniprot ID: P01375 | LLSAIKSPCQRETPEGAEAKPWYEPIYLGGVFQLEKGDRLSAEINR | |
| | PDYLDFAESGQVYFGIIAL | |
| hLIGHT Membrane | MEESVVRPSVFVVDGQTDIPFTRLGRSHRRQSCSVARVGLGLLLLL | 3 |
| Form | MGAGLAVQGWELLQLHWRLGEMVTRLPDGPAGSWEQLIQERRSHEV | |
| Uniprot ID: 043557 | NPAAHLTGANSSLTGSGGPLLWETQLGLAFLRGLSYHDGALVVTKA | |
| | GYYYIYSKVQLGGVGCPLGLASTITHGLYKRTPRYPEELELLVSQQ | |
| | SPCGRATSSSRVWWDSSFLGGVVHLEAGEKVVVRVLDERLVRLRDG | |
| | TRSYFGAFMV | |
| hLIGHT Soluble | LIQERRSHEVNPAAHLTGANSSLTGSGGPLLWETQLGLAFLRGLSY | 4 |
| Form | HDGALVVTKAGYYYIYSKVQLGGVGCPLGLASTITHGLYKRTPRYP | |
| Uniprot ID: 043557 | EELELLVSQQSPCGRATSSSRVWWDSSFLGGVVHLEAGEKVVVRVL | |
| | DERLVRLRDGTRSYFGAFMV | |
| hFASL Membrane | MQQPFNYPYPQIYWVDSSASSPWAPPGTVLPCPTSVPRRPGQRRPP | 5 |
| Form | PPPPPPPLPPPPPPPPLPPLPLPPLKKRGNHSTGLCLLVMFFMVLV | |
| Uniprot ID: P48023 | ALVGLGLGMFQLFHLQKELAELRESTSQMHTASSLEKQIGHPSPPP | |
| | EKKELRKVAHLTGKSNSRSMPLEWEDTYGIVLLSGVKYKKGGLVIN | |
| | ETGLYFVYSKVYFRGQSCNNLPLSHKVYMRNSKYPQDLVMMEGKMM | |
| | SYCTTGQMWARSSYLGAVENLTSADHLYVNVSELSLVNFEESQTFF | |
| | GLYKL | |
| hFASL Soluble | QIGHPSPPPEKKELRKVAHLTGKSNSRSMPLEWEDTYGIVLLSGVK | 6 |
| Form | YKKGGLVINETGLYFVYSKVYFRGQSCNNLPLSHKVYMRNSKYPQD | |
| Uniprot ID: P48023 | LVMMEGKMMSYCTTGQMWARSSYLGAVENLTSADHLYVNVSELSLV | |
| | NFEESQTFFGLYKL | |
| hTL1A Membrane | MAEDLGLSFGETASVEMLPEHGSCRPKARSSSARWALTCCLVLLPF | 7 |
| Form | LAGLTTYLLVSQLRAQGEACVQFQALKGQEFAPSHQQVYAPLRADG | |
| Uniprot ID: 095150 | DKPRAHLTVVRQTPTQHFKNQFPALHWEHELGLAFTKNRMNYTNKF | |
| | LLIPESGDYFIYSQVTFRGMTSECSEIRQAGRPNKPDSITVVITKV | |
| | TDSYPEPTQLLMGTKSVCEVGSNWFQPIYLGAMFSLQEGDKLMVNV | |
| | SDISLVDYTKEDKTFFGAFLL | |
| hTL1A Soluble Form | LKGQEFAPSHQQVYAPLRADGDKPRAHLTVVRQTPTQHFKNQFPAL | 8 |
| Uniprot ID: 095150 | HWEHELGLAFTKNRMNYTNKFLLIPESGDYFIYSQVTFRGMTSECS | |
| | EIRQAGRPNKPDSITVVITKVTDSYPEPTQLLMGTKSVCEVGSNWF | |
| | QPIYLGAMFSLQEGDKLMVNVSDISLVDYTKEDKTFFGAFLL | |

Exemplary Receptors

| | | |
|---|---|---|
| hTNFR1 | MGLSTVPDLLLPLVLLELLVGIYPSGVIGLVPHLGDREKRDSVCPQ | 9 |
| Immature Form | GKYIHPQNNSICCTKCHKGTYLYNDCPGPGQDTDCRECESGSFTAS | |
| Uniprot ID: P19438 | ENHLRHCLSCSKCRKEMGQVEISSCTVDRDTVCGCRKNQYRHYWSE | |
| | NLFQCFNCSLCLNGTVHLSCQEKQNTVCTCHAGFFLRENECVSCSN | |
| | CKKSLECTKLCLPQIENVKGTEDSGTTVLLPLVIFFGLCLLSLLFI | |
| | GLMYRYQRWKSKLYSIVCGKSTPEKEGELEGTTTKPLAPNPSFSPT | |
| | PGFTPTLGFSPVPSSTFTSSSTYTPGDCPNFAAPRREVAPPYQGAD | |
| | PILATALASDPIPNPLQKWEDSAHKPQSLDTDDPATLYAVVENVPP | |
| | LRWKEFVRRLGLSDHEIDRLELQNGRCLREAQYSMLATWRRRTPRR | |
| | EATLELLGRVLRDMDLLGCLEDIEEALCGPAALPPAPSLLR | |
| hTNFR1 | LVPHLGDREKRDSVCPQGKYIHPQNNSICCTKCHKGTYLYNDCPGP | 10 |
| Mature Form | GQDTDCRECESGSFTASENHLRHCLSCSKCRKEMGQVEISSCTVDR | |
| Uniprot ID: P19438 | DTVCGCRKNQYRHYWSENLFQCFNCSLCLNGTVHLSCQEKQNTVCT | |
| | CHAGFFLRENECVSCSNCKKSLECTKLCLPQIENVKGTEDSGTTVL | |
| | LPLVIFFGLCLLSLLFIGLMYRYQRWKSKLYSIVCGKSTPEKEGEL | |
| | EGTTTKPLAPNPSFSPTPGFTPTLGFSPVPSSTFTSSSTYTPGDCP | |
| | NFAAPRREVAPPYQGADPILATALASDPIPNPLQKWEDSAHKPQSL | |
| | DTDDPATLYAVVENVPPLRWKEFVRRLGLSDHEIDRLELQNGRCLR | |
| | EAQYSMLATWRRRTPRREATLELLGRVLRDMDLLGCLEDIEEALCG | |
| | PAALPPAPSLLR | |
| hTNFR2 | MAPVAVWAALAVGLELWAAAHALPAQVAFTPYAPEPGSTCRLREYY | 11 |
| Immature Form | DQTAQMCCSKCSPGQHAKVFCTKTSDTVCDSCEDSTYTQLWNWVPE | |
| Uniprot ID: P20333 | CLSCGSRCSSDQVETQACTREQNRICTCRPGWYCALSKQEGCRLCA | |
| | PLRKCRPGFGVARPGTETSDVVCKPCAPGTFSNTTSSTDICRPHQI | |
| | CNVVAIPGNASMDAVCTSTSPTRSMAPGAVHLPQPVSTRSQHTQPT | |
| | PEPSTAPSTSFLLPMGPSPPAEGSTGDFALPVGLIVGVTALGLLII | |
| | GVVNCVIMTQVKKKPLCLQREAKVPHLPADKARGTQGPEQQHLLIT | |
| | APSSSSSSLESSASALDRRAPTRNQPQAPGVEASGAGEARASTGSS | |
| | DSSPGGHGTQVNVTCIVNVCSSSDHSSQCSSQASSTMGDTDSSPSE | |
| | SPKDEQVPFSKEECAFRSQLETPETLLGSTEEKPLPLGVPDAGMKP | |
| | S | |
| hTNFR2 | LPAQVAFTPYAPEPGSTCRLREYYDQTAQMCCSKCSPGQHAKVFCT | 12 |
| Mature Form | KTSDTVCDSCEDSTYTQLWNWVPECLSCGSRCSSDQVETQACTREQ | |
| Uniprot ID: P20333 | NRICTCRPGWYCALSKQEGCRLCAPLRKCRPGFGVARPGTETSDVV | |
| | CKPCAPGTFSNTTSSTDICRPHQICNVVAIPGNASMDAVCTSTSPT | |
| | RSMAPGAVHLPQPVSTRSQHTQPTPEPSTAPSTSFLLPMGPSPPAE | |
| | GSTGDFALPVGLIVGVTALGLLIIGVVNCVIMTQVKKKPLCLQREA | |
| | KVPHLPADKARGTQGPEQQHLLITAPSSSSSSLESSASALDRRAPT | |
| | RNQPQAPGVEASGAGEARASTGSSDSSPGGHGTQVNVTCIVNVCSS | |
| | SDHSSQCSSQASSTMGDTDSSPSESPKDEQVPFSKEECAFRSQLET | |
| | PETLLGSTEEKPLPLGVPDAGMKPS | |
| hLIGHTR | MEPPGDWGPPPWRSTPKTDVLRLVLYLTFLGAPCYAPALPSCKEDE | 13 |
| Immature Form | YPVGSECCPKCSPGYRVKEACGELTGTVCEPCPPGTYIAHLNGLSK | |
| Uniprot ID: Q92956 | CLQCQMCDPAMGLRASRNCSRTENAVCGCSPGHFCIVQDGDHCAAC | |
| | RAYATSSPGQRVQKGGTESQDTLCQNCPPGTFSPNGTLEECQHQTK | |
| | CSWLVTKAGAGTSSSHWVWWFLSGSLVIVIVCSTVGLIICVKRRKP | |
| | RGDVVKVIVSVQRKRQEAEGEATVIEALQAPPDVTTVAVEETIPSF | |
| | TGRSPNH | |
| hLIGHTR | LPSCKEDEYPVGSECCPKCSPGYRVKEACGELTGTVCEPCPPGTYI | 14 |
| Mature Form | AHLNGLSKCLQCQMCDPAMGLRASRNCSRTENAVCGCSPGHFCIVQ | |
| Uniprot ID: Q92956 | DGDHCAACRAYATSSPGQRVQKGGTESQDTLCQNCPPGTFSPNGTL | |
| | EECQHQTKCSWLVTKAGAGTSSSHWVWWFLSGSLVIVIVCSTVGLI | |
| | ICVKRRKPRGDVVKVIVSVQRKRQEAEGEATVIEALQAPPDVTTVA | |
| | VEETIPSFTGRSPNH | |
| hFAS | MLGIWTLLPLVLTSVARLSSKSVNAQVTDINSKGLELRKTVTTVET | 15 |
| Immature Form | QNLEGLHHDGQFCHKPCPPGERKARDCTVNGDEPDCVPCQEGKEYT | |
| Uniprot ID: P25445 | DKAHFSSKCRRCRLCDEGHGLEVEINCTRTQNTKCRCKPNFFCNST | |
| | VCEHCDPCTKCEHGIIKECTLTSNTKCKEEGSRSNLGWLCLLLLPI | |
| | PLIVWVKRKEVQKTCRKHRKENQGSHESPTLNPETVAINLSDVDLS | |
| | KYITTIAGVMTLSQVKGFVRKNGVNEAKIDEIKNDNVQDTAEQKVQ | |
| | LLRNWHQLHGKKEAYDTLIKDLKKANLCTLAEKIQTIILKDITSDS | |
| | ENSNFRNEIQSLV | |
| hFAS | QVTDINSKGLELRKTVTTVETQNLEGLHHDGQFCHKPCPPGERKAR | 16 |
| Mature Form | DCTVNGDEPDCVPCQEGKEYTDKAHFSSKCRRCRLCDEGHGLEVEI | |
| Uniprot ID: P25445 | NCTRTQNTKCRCKPNFFCNSTVCEHCDPCTKCEHGIIKECTLTSNT | |
| | KCKEEGSRSNLGWLCLLLLPIPLIVWVKRKEVQKTCRKHRKENQGS | |
| | HESPTLNPETVAINLSDVDLSKYITTIAGVMTLSQVKGFVRKNGVN | |
| | EAKIDEIKNDNVQDTAEQKVQLLRNWHQLHGKKEAYDTLIKDLKKA | |
| | NLCTLAEKIQTIILKDITSDSENSNFRNEIQSLV | |
| hDR3 | MEQRPRGCAAVAAALLLVLLGARAQGGTRSPRCDCAGDFHKKIGLF | 17 |
| Immature Form | CCRGCPAGHYLKAPCTEPCGNSTCLVCPQDTFLAWENHHNSECARC | |
| Uniprot ID: Q93038 | QACDEQASQVALENCSAVADTRCGCKPGWFVECQVSQCVSSSPFYC | |
| | QPCLDCGALHRHTRLLCSRRDTDCGTCLPGFYEHGDGCVSCPTSTL | |
| | GSCPERCAAVCGWRQMFWVQVLLAGLVVPLLLGATLTYTYRHCWPH | |
| | KPLVTADEAGMEALTPPPATHLSPLDSAHTLLAPPDSSEKICTVQL | |
| | VGNSWTPGYPETQEALCPQVTWSWDQLPSRALGPAAAPTLSPESPA | |
| | GSPAMMLQPGPQLYDVMDAVPARRWKEFVRTLGLREAEIEAVEVEI | |
| | GRFRDQQYEMLKRWRQQQPAGLGAVYAALERMGLDGCVEDLRSRLQ | |
| | RGP | |
| hDR3 | QGGTRSPRCDCAGDFHKKIGLFCCRGCPAGHYLKAPCTEPCGNSTC | 18 |
| Mature Form | LVCPQDTFLAWENHHNSECARCQACDEQASQVALENCSAVADTRCG | |
| Uniprot ID: Q93038 | CKPGWFVECQVSQCVSSSPFYCQPCLDCGALHRHTRLLCSRRDTDC | |
| | GTCLPGFYEHGDGCVSCPTSTLGSCPERCAAVCGWRQMFWVQVLLA | |
| | GLVVPLLLGATLTYTYRHCWPHKPLVTADEAGMEALTPPPATHLSP | |
| | LDSAHTLLAPPDSSEKICTVQLVGNSWTPGYPETQEALCPQVTWSW | |
| | DQLPSRALGPAAAPTLSPESPAGSPAMMLQPGPQLYDVMDAVPARR | |
| | WKEFVRTLGLREAEIEAVEVEIGRFRDQQYEMLKRWRQQQPAGLGA | |
| | VYAALERMGLDGCVEDLRSRLQRGP | |
| hDcR3 | MRALEGPGLSLLCLVLALPALLPVPAVRGVAETPTYPWRDAETGER | 19 |
| Immature Form | LVCAQCPPGTFVQRPCRRDSPTTCGPCPPRHYTQFWNYLERCRYCN | |
| Uniprot ID: 095407 | VLCGEREEEARACHATHNRACRCRTGFFAHAGFCLEHASCPPGAGV | |
| | IAPGTPSQNTQCQPCPPGTFSASSSSSEQCQPHRNCTALGLALNVP | |
| | GSSSHDTLCTSCTGFPLSTRVPGAEECERAVIDFVAFQDISIKRLQ | |
| | RLLQALEAPEGWGPTPRAGRAALQLKLRRRLTELLGAQDGALLVRL | |
| | LQALRVARMPGLERSVRERFLPVH | |
| hDcR3 | VAETPTYPWRDAETGERLVCAQCPPGTFVQRPCRRDSPTTCGPCPP | 20 |
| Mature Form | RHYTQFWNYLERCRYCNVLCGEREEEARACHATHNRACRCRTGFFA | |
| Uniprot ID: 095407 | HAGFCLEHASCPPGAGVIAPGTPSQNTQCQPCPPGTFSASSSSSEQ | |
| LTBR | CQPHRNCTALGLALNVPGSSSHDTLCTSCTGFPLSTRVPGAEECER | |
| | AVIDFVAFQDISIKRLQRLLQALEAPEGWGPTPRAGRAALQLKLRR | |
| | RLTELLGAQDGALLVRLLQALRVARMPGLERSVRERFLPVH | |
| hLTβR | MLLPWATSAPGLAWGPLVLGLFGLLAASQPQAVPPYASENQTCRDQ | 21 |
| Immature Form | EKEYYEPQHRICCSRCPPGTYVSAKCSRIRDTVCATCAENSYNEHW | |
| Uniprot ID: P36941 | NYLTICQLCRPCDPVMGLEEIAPCTSKRKTQCRCQPGMFCAAWALE | |
| | CTHCELLSDCPPGTEAELKDEVGKGNNHCVPCKAGHFQNTSSPSAR | |
| | CQPHTRCENQGLVEAAPGTAQSDTTCKNPLEPLPPEMSGTMLMLAV | |
| | LLPLAFFLLLATVFSCIWKSHPSLCRKLGSLLKRRPQGEGPNPVAG | |
| | SWEPPKAHPYFPDLVQPLLPISGDVSPVSTGLPAAPVLEAGVPQQQ | |
| | SPLDLTREPQLEPGEQSQVAHGINGIHVTGGSMTITGNIYIYNGPV | |
| | LGGPPGPGDLPATPEPPYPIPEEGDPGPPGLSTPHQEDGKAWHLAE | |
| | TEHCGATPSNRGPRNQFITHD | |
| hLTβR | QAVPPYASENQTCRDQEKEYYEPQHRICCSRCPPGTYVSAKCSRIR | 22 |
| Mature Form | DTVCATCAENSYNEHWNYLTICQLCRPCDPVMGLEEIAPCTSKRKT | |
| Uniprot ID: P36941 | QCRCQPGMFCAAWALECTHCELLSDCPPGTEAELKDEVGKGNNHCV | |
| | PCKAGHFQNTSSPSARCQPHTRCENQGLVEAAPGTAQSDTTCKNPL | |
| | EPLPPEMSGTMLMLAVLLPLAFFLLLATVESCIWKSHPSLCRKLGS | |
| | LLKRRPQGEGPNPVAGSWEPPKAHPYFPDLVQPLLPISGDVSPVST | |
| | GLPAAPVLEAGVPQQQSPLDLTREPQLEPGEQSQVAHGINGIHVTG | |
| | GSMTITGNIYIYNGPVLGGPPGPGDLPATPEPPYPIPEEGDPGPPG | |
| | LSTPHQEDGKAWHLAETEHCGATPSNRGPRNQFITHD | |

The present disclosure provides, inter alia, immunoreceptor inhibitory proteins (and functional fragments and variants thereof) that specifically bind TL1A. The amino acid sequence of the immunoreceptor inhibitory proteins provided herein is set forth in Table 2. The amino acid sequence of the mature form of the immunoreceptor inhibitory proteins and polypeptides (i.e., lacking the native signal peptide) is set forth in SEQ ID NOS: 23-53, 187-188, or 199. The amino acid sequence of the immature form of the immunoreceptor inhibitory proteins (i.e., containing the native signal peptide) is set forth in SEQ ID NOS: 54-84, 189-190, or 200.

The signal peptides have been computationally predicted using standard methods (see, e.g., Teufel, F., Almagro Armenteros, J. J., Johansen, A. R. et al. SignalP 6.0 predicts all five types of signal peptides using protein language models. Nat Biotechnol (2022). https://doi.org/10.1038/s41587-021-01156-3, the entire contents of which is incorporated by reference herein for all purposes). A person of ordinary skill in the art would know how to experimentally identify and/or validate a computationally predicted signal peptide using standard methods known in the art, e.g., expression of the immunoreceptor inhibitory protein from a host cell and sequencing of the intracellular form and the extracellular form of the expressed protein (see, e.g., Zhang Z, Henzel W J. Signal peptide prediction based on analysis of experimentally verified cleavage sites. Protein Sci. 2004; 13 (10): 2819-2824. doi: 10.1110/ps.04682504, the entire contents of which is incorporated by reference herein for all purposes).

TABLE 2
The Amino Acid Sequence of Immunoreceptor Inhibitory Proteins.

| | | SEQ |
|---|---|---|
| Description | Amino Acid Sequence | ID NO |
| Immunoreceptor | TVYVVKSGVNKGKICDTCPPGTYKKRDCDRSLPTVCEPCGDGEYTS | 23 |
| Inhibitory Protein | MNNSLPECLSCNHCYDPTEIEITPCNATTNTVCSCKEGYTFDSSIQ | |
| (IIP)-1 | GCI | |
| without native signal | | |
| peptide | | |
| IIP-2 | STYRVRSSGLTCSTCPPGTHKERDCSLNTETICKACGEGEYTAHNN | 24 |
| without native signal | SLPKCLACKSCFNATEIETKSCDPTSDTICACREGYSINNLGECN | |
| peptide | | |
| IIP-3 | STYRVRSSGLTCSMCPPGTYKERDCSLNTETLCKACGEGEYTAHKN | 25 |
| without native signal | SLPKCLACKSCENATEIETKSCDPISDTICTCREGYSINNLGECN | |
| peptide | | |
| IIP-4 | RSKINSSLICDMCPPGYYKNKDCTSTSTTICLPCGEGEYTAYNNSL | 26 |
| without native signal | TKCIRCKDCYEENEKIFKPCNSTSDTICTCIDGYTKDETTDSCI | |
| peptide | | |
| IIP-5 | DTFTDEETGLVCEKCPPGTYREADCTETTKTVCKACGEGTFTDHPN | 27 |
| without native signal | NLPECKSCSTCDEATEVEVQKCTATANTVCACKDGFTVNEKGECV | |
| peptide | | |
| IIP-6 | DTYTDEKTGLTCEKCPPGTYREADCGENSKTVCKACGEGTYTDAPN | 28 |
| without native signal | HLPECKKCNTCDAETEVEVKACTATSDTVCACKEGFTVNENGECV | |
| peptide | | |
| IIP-7 | STYTDSATGRTCSKCPPGTYRSADCGATTSTVCTACGAGTYTDKDN | 29 |
| without native signal | NLSACLACSTCNAATETEVSACTATANTVCACKAGYTLDANGKCV | |
| peptide | | |
| IIP-8 | STYKDEKTGLTCQKCPPGTYRKADCGLFSKTECAACGEGTYTDKDN | 30 |
| without native signal | NLSKCLKCSTCNEETEVEVQKCTATADTVCACKEGFTKNEKGECQ | |
| peptide | | |
| IIP-9 | PTYRDEATGRVCQECPPGTYLEAHCGENTSTVCKACGEGTFTDKPN | 31 |
| without native signal | HLPQCLSCSTCEAATEVEVRACTATANTVCACREGFTRNEQGKCN | |
| peptide | | |
| HIP-10 | DTFTDEATGLTCAKCPPGTYREADCGLFSKTVCKACGEGTFTAEPN | 32 |
| without native signal | NLPTCKACNTCDAATEVEVQACTATADTVCACKEGETKNENGECN | |
| peptide | | |
| IIP-11 | ATYTDTATGLTCAKCPPGTFRKADCGATTATVCEACGAGTYTAADN | 33 |
| without native signal | HLSACLACTTCDAATETETAACTATANTVCACKAGFTVDANGKCG | |
| peptide | | |
| HIP-12 | PTYTDTATGLTCAKCPPGTYRERDCGLFSDTVCKACGEGTYTDTPN | 34 |
| without native signal | HLPKCLSCSTCDAATETEVQACTATANTVCACKDGFTVDSNGKCQ | |
| peptide | | |
| HIP-13 | DTYTDTATGLTCAKCPPGTYREADCGLFSETVCKACGAGTYTDKPN | 35 |
| without native signal | HLPACLACGTCDAATEVEVQACTATANTVCACKAGFTKNEKGECV | |
| peptide | | |
| IIP-14 | PTYTDTATGLTCEQCPPGTYRRADCGATTATVCAACGAGTYTSAPN | 36 |
| without native signal | NLPTCLACPTCDAATEVEVQPCVATANTVCACRAGFTVDAQGRCQ | |
| peptide | | |
| IIP-15 | ATYTDTATGLTCTKCPPGTHRKADCGLLTSTVCEACGEGTYTSEDN | 37 |
| without native signal | NLSSCLSCSTCNEATETEVQACTATSDTVCECKEGFTVDANGKCG | |
| peptide | | |
| IIP-16 | ATYKDEKTGLTCEKCPPGTYREADCTETTKTVCKACGEGTYTDKDN | 38 |
| without native signal | NLPKCLECNTCDEKTEVEVQKCTATSNTVCACKEGFTKNEKGECK | |
| peptide | | |
| HIP-17 | KTYKDPETGLTCEKCPPGTYLEANCTPTSPTVCKPCGEGTYTEEEN | 39 |
| without native signal | ALTACKPCSSCDPATEEVVQPCTPTSDTVCSCKPGYSFNSNGLCV | |
| peptide | | |
| HIP-18 | ETYTDSSTGLTCAKCPPGYYLSQTCTETSPTVCKACGSGTYTTTPN | 40 |
| without native signal | ALTSCKACSTCDSSTEVTTKSCTPTSDTVCSCKSGYSKNSSGKCV | |
| peptide | | |
| IIP-19 | QTYTDPATGLTCDLCPPGTYLEAACTATSPTVCKPCPSGTYTTAPN | 41 |
| without native signal | ALTSCRKCSTCNPATEVTTQPCTPTSDTVCSCKSGYTLDSNGQCV | |
| peptide | | |
| IIP-20 | KTYVDPTTGLTCDMCPPGTYLKKTCSLNSRTVCKPCGKGTYTSTYN | 42 |
| without native signal | NLKKCKKCSTCDPRTEVVSQPCTQTSDTVCECRPGYSRDSNGRCV | |
| peptide | | |
| IIP-21 | ATYTDPETGLTCEKCPPGTYLEAPCTETTPTVCKPCGEGEYTTVPN | 43 |
| without native signal | ALRKCRECTTCNPATEEVSADCTPTSNTVCTCKPGYVKDANGKCV | |
| peptide | | |
| HIP-22 | QTYVDPATGLTCTLCPPGTYLKKPCTATSPTVCKPCGSGTYTSKPN | 44 |
| without native signal | ALTKCQECTTCDPATERVVRPCTPTHNTVCECKPGYKRNSKGQCV | |
| peptide | | |
| HIP-23 | ATYTDPATGLVCELCPPGSYLAAPCTATTPTVCKPCPEGYYTTEPN | 45 |
| without native signal | ALPRCLPCTTCNPETEVVIEPCTPTSDTVCECKPGYSLNENGECV | |
| peptide | | |
| IIP-24 | DTYTDPTTGLTCKKCPPGTYLKKNCTATSPTVCKKCGPGQYTTKYN | 46 |
| without native signal | NLKKCNKCSTCDPTTEEVSQACTPTHDTVCKCKPGYSLNSKGRCV | |
| peptide | | |
| IIP-25 | STYTNPSTGLTCSMCPPGTYLERDCSSSSGTVCKPCGPGTYTTAYN | 47 |
| without native signal | NLKSCKKCTKCDPKTEKVVKECTPTSNTVCECKPGFKFDSNGKCR | |
| peptide | | |
| IIP-26 | KTFVDDKTGLTCEQCPPGTYLESTCSETSPTVCKPCGPGEYTTEYN | 48 |
| without native signal | NLTKCKKCSTCDPATEVVVSACTPTSDTVCACKPGFSFNEEGKCV | |
| peptide | | |
| IIP-27 | STYVNPTTGLTCSKCPPGTYLKASCTATSKTVCTPCGSGTYTTTAN | 49 |
| without native signal | NLSSCKQCSTCDPATETVSQPCTPTSDTVCSCKSGYTFDSNGKCV | |
| peptide | | |
| IIP-28 | ETYKDPSTGLLCEKCPPGTYLSSPCTTTSGTVCKPCGSGTYTTQPN | 50 |
| without native signal | ALSSCSSCSTCDSATQTVSKACTSTSDTVCSCKSGYSLDSNGNCV | |
| peptide | | |
| IIP-29 | ETYTDPATGRVCEKCPPGTYVAAPCTATSPTVCRPCGPGTYTSAPN | 51 |
| without native signal | ALTSCLACTSCDPATEAVSQPCTPTANTVCVCKPGFSFNANGQCV | |
| peptide | | |
| IIP-30 | ETYTNASTGLTCNKCPPGTYLAKDCTSTSDTVCQDCGPGTYTTSPN | 52 |
| without native signal | AKSSCNACSTCDPATEEVITPCTPTSDTVCACKPGFTWNQNGQCV | |
| peptide | | |
| IIP-31 | ETYTDPATGLTCQKCPPGTYLKAPCTSTSPTVCTPCGSGYYTSSSN | 53 |
| without native signal | NLTSCSKCNTCDPSTESVSSACTPTSNTVCSCKSGYTLDSNGNCV | |
| peptide | | |
| IIP-32 | MSTYNTKTSPSLKCDMCPPGYYKHEDCTSNTKTVCSPCGEGEYTAY | 187 |
| without native signal | NNSLTKCLRCSDCYGENEITTKQCTNTSNTVCECMDGYTKDETIDA | |
| peptide | CIK | |
| IIP-33 | ASTYTSKIDASLICDMCPPGSYKYKDCTYDSKTVCLPCGDGEYTSY | 188 |
| without native signal | NNSLAKCLRCDDCYDENEVTAKPCDSTSNTICKCMDGYTKDNTISA | |
| peptide | CVKTSHIT | |
| IIP-34 | KVPTWTDPVTGLTCDRCPAGTHVVKQCTATTPTECGACPANHYTEF | 199 |
| without native signal | WNYLNRCLYCGVRCTEQQVEKSTCSATHNRVCECKPGYHIYADDFC | |
| peptide | LRHSTCPPGQGLLVAGTPTSNTRCGPCQAGYFSAEDSTEACKPHTP | |
| | CKDTERSVPGTATQDTFCTSCQARKCDITAPPAPDKAVCDDAFIDF | |
| | VERYPLNAKKAKNLMRKLRKMGKGKTIRESFQAIMAKRNDQPLACE | |
| | VLSTLNDVDSDMGCRIKTFFLGWNEDEC | |
| IIP-1 | MIITLLALLLVVSTESSTVYVVKSGVNKGKICDTCPPGTYKKRDCD | 54 |
| with native signal | RSLPTVCEPCGDGEYTSMNNSLPECLSCNHCYDPTEIEITPCNATT | |
| peptide | NTVCSCKEGYTFDSSIQGCI | |
| IIP-2 | MGKNSLVLTIGVLMTLITLRSSFSASTYRVRSSGLTCSTCPPGTHK | 55 |
| with native signal | ERDCSLNTETICKACGEGEYTAHNNSLPKCLACKSCENATEIETKS | |
| peptide | CDPTSDTICACREGYSINNLGECN | |
| IIP-3 | MGKNSLVLMIGVLMTLITLRSSFSASTYRVRSSGLTCSMCPPGTYK | 56 |
| with native signal | ERDCSLNTETLCKACGEGEYTAHKNSLPKCLACKSCENATEIETKS | |
| peptide | CDPISDTICTCREGYSINNLGECN | |
| IIP-4 | MHYKNKMSVNILIITVLIGISFQASTYRSKINSSLICDMCPPGYYK | 57 |
| with native signal | NKDCTSTSTTICLPCGEGEYTAYNNSLTKCIRCKDCYEENEKIFKP | |
| peptide | CNSTSDTICTCIDGYTKDETTDSCI | |
| IIP-5 | MGKNSLVLTIGVLMTLITLRSSFSADTFTDEETGLVCEKCPPGTYR | 58 |
| with native signal | EADCTETTKTVCKACGEGTFTDHPNNLPECKSCSTCDEATEVEVQK | |
| peptide | CTATANTVCACKDGFTVNEKGECV | |
| IIP-6 | MGKNSLVLTIGVLMTLITLRSSFSADTYTDEKTGLTCEKCPPGTYR | 59 |
| with native signal | EADCGENSKTVCKACGEGTYTDAPNHLPECKKCNTCDAETEVEVKA | |
| peptide | CTATSDTVCACKEGFTVNENGECV | |
| IIP-7 | MGKNSLVLTIGVLMTLITLRSSFSASTYTDSATGRTCSKCPPGTYR | 60 |
| with native signal | SADCGATTSTVCTACGAGTYTDKDNNLSACLACSTCNAATETEVSA | |
| peptide | CTATANTVCACKAGYTLDANGKCV | |
| IIP-8 | MGKNSLVLTIGVLMTLITLRSSFSASTYKDEKTGLTCQKCPPGTYR | 61 |
| with native signal | KADCGLFSKTECAACGEGTYTDKDNNLSKCLKCSTCNEETEVEVQK | |
| peptide | CTATADTVCACKEGFTKNEKGECQ | |
| IIP-9 | MGKNSLVLTIGVLMTLITLRSSFSAPTYRDEATGRVCQECPPGTYL | 62 |
| with native signal | EAHCGENTSTVCKACGEGTFTDKPNHLPQCLSCSTCEAATEVEVRA | |
| peptide | CTATANTVCACREGFTRNEQGKCN | |
| IIP-10 | MGKNSLVLTIGVLMTLITLRSSFSADTFTDEATGLTCAKCPPGTYR | 63 |
| with native signal | EADCGLFSKTVCKACGEGTFTAEPNNLPTCKACNTCDAATEVEVQA | |
| peptide | CTATADTVCACKEGFTKNENGECN | |
| IIP-11 | MGKNSLVLTIGVLMTLITLRSSFSAATYTDTATGLTCAKCPPGTER | 64 |
| with native signal | KADCGATTATVCEACGAGTYTAADNHLSACLACTTCDAATETETAA | |
| peptide | CTATANTVCACKAGFTVDANGKCG | |
| HIP-12 | MGKNSLVLTIGVLMTLITLRSSFSAPTYTDTATGLTCAKCPPGTYR | 65 |
| with native signal | ERDCGLFSDTVCKACGEGTYTDTPNHLPKCLSCSTCDAATETEVQA | |
| peptide | CTATANTVCACKDGFTVDSNGKCQ | |
| IIP-13 | MGKNSLVLTIGVLMTLITLRSSFSADTYTDTATGLTCAKCPPGTYR | 66 |
| with native signal | EADCGLFSETVCKACGAGTYTDKPNHLPACLACGTCDAATEVEVQA | |
| peptide | CTATANTVCACKAGFTKNEKGECV | |
| IIP-14 | MGKNSLVLTIGVLMTLITLRSSFSAPTYTDTATGLTCEQCPPGTYR | 67 |
| with native signal | RADCGATTATVCAACGAGTYTSAPNNLPTCLACPTCDAATEVEVQP | |
| peptide | CVATANTVCACRAGFTVDAQGRCQ | |
| IIP-15 | MGKNSLVLTIGVLMTLITLRSSFSAATYTDTATGLTCTKCPPGTHR | 68 |
| with native signal | KADCGLLTSTVCEACGEGTYTSEDNNLSSCLSCSTCNEATETEVQA | |
| peptide | CTATSDTVCECKEGFTVDANGKCG | |
| IIP-16 | MGKNSLVLTIGVLMTLITLRSSFSAATYKDEKTGLTCEKCPPGTYR | 69 |
| with native signal | EADCTETTKTVCKACGEGTYTDKDNNLPKCLECNTCDEKTEVEVQK | |
| peptide | CTATSNTVCACKEGFTKNEKGECK | |
| IIP-17 | MGKNSLVLTIGVLMTLITLRSSFSAKTYKDPETGLICEKCPPGTYL | 70 |
| with native signal | EANCTPTSPTVCKPCGEGTYTEEENALTACKPCSSCDPATEEVVQP | |
| peptide | CTPTSDTVCSCKPGYSFNSNGLCV | |
| IIP-18 | MGKNSLVLTIGVLMTLITLRSSFSAETYTDSSTGLTCAKCPPGYYL | 71 |
| with native signal | SQTCTETSPTVCKACGSGTYTTTPNALTSCKACSTCDSSTEVTTKS | |
| peptide | CTPTSDTVCSCKSGYSKNSSGKCV | |
| IIP-19 | MGKNSLVLTIGVLMTLITLRSSFSAQTYTDPATGLTCDLCPPGTYL | 72 |
| with native signal | EAACTATSPTVCKPCPSGTYTTAPNALTSCRKCSTCNPATEVTTQP | |
| peptide | CTPTSDTVCSCKSGYTLDSNGQCV | |
| IIP-20 | MGKNSLVLTIGVLMTLITLRSSFSAKTYVDPTTGLTCDMCPPGTYL | 73 |
| with native signal | KKTCSLNSRTVCKPCGKGTYTSTYNNLKKCKKCSTCDPRTEVVSQP | |
| peptide | CTQTSDTVCECRPGYSRDSNGRCV | |
| IIP-21 | MGKNSLVLTIGVLMTLITLRSSFSAATYTDPETGLTCEKCPPGTYL | 74 |
| with native signal | EAPCTETTPTVCKPCGEGEYTTVPNALRKCRECTTCNPATEEVSAD | |
| peptide | CTPTSNTVCTCKPGYVKDANGKCV | |
| IIP-22 | MGKNSLVLTIGVLMTLITLRSSFSAQTYVDPATGLTCTLCPPGTYL | 75 |
| with native signal | KKPCTATSPTVCKPCGSGTYTSKPNALTKCQECTTCDPATERVVRP | |
| peptide | CTPTHNTVCECKPGYKRNSKGQCV | |
| IIP-23 | MGKNSLVLTIGVLMTLITLRSSFSAATYTDPATGLVCELCPPGSYL | 76 |
| with native signal | AAPCTATTPTVCKPCPEGYYTTEPNALPRCLPCTTCNPETEVVIEP | |
| peptide | CTPTSDTVCECKPGYSLNENGECV | |
| IIP-24 | MGKNSLVLTIGVLMTLITLRSSFSADTYTDPTTGLTCKKCPPGTYL | 77 |
| with native signal | KKNCTATSPTVCKKCGPGQYTTKYNNLKKCNKCSTCDPTTEEVSQA | |
| peptide | CTPTHDTVCKCKPGYSLNSKGRCV | |
| HIP-25 | MGKNSLVLTIGVLMTLITLRSSFSASTYTNPSTGLTCSMCPPGTYL | 78 |
| with native signal | ERDCSSSSGTVCKPCGPGTYTTAYNNLKSCKKCTKCDPKTEKVVKE | |
| peptide | CTPTSNTVCECKPGFKFDSNGKCR | |
| IIP-26 | MGKNSLVLTIGVLMTLITLRSSFSAKTFVDDKTGLTCEQCPPGTYL | 79 |
| with native signal | ESTCSETSPTVCKPCGPGEYTTEYNNLTKCKKCSTCDPATEVVVSA | |
| peptide | CTPTSDTVCACKPGFSFNEEGKCV | |
| IIP-27 | MGKNSLVLTIGVLMTLITLRSSFSASTYVNPTTGLTCSKCPPGTYL | 80 |
| with native signal | KASCTATSKTVCTPCGSGTYTTTANNLSSCKQCSTCDPATETVSQP | |
| peptide | CTPTSDTVCSCKSGYTFDSNGKCV | |
| IIP-28 | MGKNSLVLTIGVLMTLITLRSSFSAETYKDPSTGLLCEKCPPGTYL | 81 |
| with native signal | SSPCTTTSGTVCKPCGSGTYTTQPNALSSCSSCSTCDSATQTVSKA | |
| peptide | CTSTSDTVCSCKSGYSLDSNGNCV | |
| IIP-29 | MGKNSLVLTIGVLMTLITLRSSFSAETYTDPATGRVCEKCPPGTYV | 82 |
| with native signal | AAPCTATSPTVCRPCGPGTYTSAPNALTSCLACTSCDPATEAVSQP | |
| peptide | CTPTANTVCVCKPGFSFNANGQCV | |
| IIP-30 | MGKNSLVLTIGVLMTLITLRSSFSAETYTNASTGLTCNKCPPGTYL | 83 |
| with native signal | AKDCTSTSDTVCQDCGPGTYTTSPNAKSSCNACSTCDPATEEVITP | |
| peptide | CTPTSDTVCACKPGFTWNQNGQCV | |
| IIP-31 | MGKNSLVLTIGVLMTLITLRSSFSAETYTDPATGLTCQKCPPGTYL | 84 |
| with native signal | KAPCTSTSPTVCTPCGSGYYTSSSNNLTSCSKCNTCDPSTESVSSA | |
| peptide | CTPTSNTVCSCKSGYTLDSNGNCV | |
| IIP-32 | MSNNLCQIITICIITMVCISYQMSTYNTKTSPSLKCDMCPPGYYKH | 189 |
| with native signal | EDCTSNTKTVCSPCGEGEYTAYNNSLIKCLRCSDCYGENEITTKQC | |
| peptide | TNTSNTVCECMDGYTKDETIDACIK | |
| IIP-33 | MTNYLYISANIFVIILLAEITFQASTYTSKIDASLICDMCPPGSYK | 190 |
| with native signal | YKDCTYDSKTVCLPCGDGEYTSYNNSLAKCLRCDDCYDENEVTAKP | |
| peptide | CDSTSNTICKCMDGYTKDNTISACVKTSHIT | |
| IIP-34 | MERGTLMVLACVAACFSEKVPTWTDPVTGLTCDRCPAGTHVVKQCT | 200 |
| with native signal | ATTPTECGACPANHYTEFWNYLNRCLYCGVRCTEQQVEKSTCSATH | |
| peptide | NRVCECKPGYHIYADDFCLRHSTCPPGQGLLVAGTPTSNTRCGPCQ | |
| | AGYFSAEDSTEACKPHTPCKDTERSVPGTATQDTFCTSCQARKCDI | |
| | TAPPAPDKAVCDDAFIDFVERYPLNAKKAKNLMRKLRKMGKGKTIR | |
| | ESFQAIMAKRNDQPLACEVLSTLNDVDSDMGCRIKTFFLGWNEDEC | |

As such, the individual immunoreceptor inhibitory proteins (or functional fragments, functional variants, or functional fragment/variants thereof) are provided in Table 2.

In some embodiments, the amino acid sequence of the immunoreceptor inhibitory protein (or a functional fragment, functional variant, or functional fragment/variant thereof) comprises an amino acid sequence at least about 85%, 86%, 87%, 88%, 89%, 90%, 91%, 92%, 93%, 94%, 95%, 96%, 97%, 98%, 99%, or 100% identical to the amino acid sequence of a protein set forth in Table 2. In some embodiments, the amino acid sequence of the immunoreceptor inhibitory protein (or a functional fragment, functional variant, or functional fragment/variant thereof) comprises an amino acid sequence at least about 85% identical to the amino acid sequence of a protein set forth in Table 2. In some embodiments, the amino acid sequence of the immunoreceptor inhibitory protein (or a functional fragment, functional variant, or functional fragment/variant thereof) comprises an amino acid sequence at least about 90% identical to the amino acid sequence of a protein set forth in Table 2. In some embodiments, the amino acid sequence of the immunoreceptor inhibitory protein (or a functional fragment, functional variant, or functional fragment/variant thereof) comprises an amino acid sequence at least about 95% identical to the amino acid sequence of a protein set forth in Table 2. In some embodiments, the amino acid sequence of the immunoreceptor inhibitory protein (or a functional fragment, functional variant, or functional fragment/variant thereof) comprises an amino acid sequence at least about 99% identical to the amino acid sequence of a protein set forth in Table 2.

In some embodiments, the amino acid sequence of the immunoreceptor inhibitory protein (or a functional fragment, functional variant, or functional fragment/variant thereof) comprises an amino acid sequence about 85%, 86%, 87%, 88%, 89%, 90%, 91%, 92%, 93%, 94%, 95%, 96%, 97%, 98%, 99%, or 100% identical to the amino acid sequence of a protein set forth in Table 2. In some embodiments, the amino acid sequence of the immunoreceptor inhibitory protein (or a functional fragment, functional variant, or functional fragment/variant thereof) comprises an amino acid sequence about 85% identical to the amino acid sequence of a protein set forth in Table 2. In some embodiments, the amino acid sequence of the immunoreceptor inhibitory protein (or a functional fragment, functional variant, or functional fragment/variant thereof) comprises an amino acid sequence about 90% identical to the amino acid sequence of a protein set forth in Table 2. In some embodiments, the amino acid sequence of the immunoreceptor inhibitory protein (or a functional fragment, functional variant, or functional fragment/variant thereof) comprises an amino acid sequence about 95% identical to the amino acid sequence of a protein set forth in Table 2. In some embodiments, the amino acid sequence of the immunoreceptor inhibitory protein (or a functional fragment, functional variant, or functional fragment/variant thereof) comprises an amino acid sequence about 99% identical to the amino acid sequence of a protein set forth in Table 2. In some embodiments, the amino acid sequence of the immunoreceptor inhibitory protein (or a functional fragment, functional variant, or functional fragment/variant thereof) comprises an amino acid sequence about 100% identical to the amino acid sequence of a protein set forth in Table 2.

In some embodiments, the amino acid sequence of the immunoreceptor inhibitory protein (or a functional fragment, functional variant, or functional fragment/variant thereof) consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of a protein set forth in Table 2. In some embodiments, the amino acid sequence of the immunoreceptor inhibitory protein consists of an amino acid sequence at least about 85% identical to the amino acid sequence of a protein set forth in Table 2. In some embodiments, the amino acid sequence of the immunoreceptor inhibitory protein (or a functional fragment, functional variant, or functional fragment/variant thereof) consists of an amino acid sequence at least about 90% identical to the amino acid sequence of a protein set forth in Table 2. In some embodiments, the amino acid sequence of the immunoreceptor inhibitory protein (or a functional fragment, functional variant, or functional fragment/variant thereof) consists of an amino acid sequence at least about 95% identical to the amino acid sequence of a protein set forth in Table 2. In some embodiments, the amino acid sequence of the immunoreceptor inhibitory protein (or a functional fragment, functional variant, or functional fragment/variant thereof) consists of an amino acid sequence at least about 99% identical to the amino acid sequence of a protein set forth in Table 2.

In some embodiments, the amino acid sequence of the immunoreceptor inhibitory protein (or a functional fragment, functional variant, or functional fragment/variant thereof) consists of an amino acid sequence about 85%, 86%, 87%, 88%, 89%, 90%, 91%, 92%, 93%, 94%, 95%, 96%, 97%, 98%, 99%, or 100% identical to the amino acid sequence of a protein set forth in Table 2. In some embodiments, the amino acid sequence of the immunoreceptor inhibitory protein (or a functional fragment, functional variant, or functional fragment/variant thereof) consists of an amino acid sequence about 85% identical to the amino acid sequence of a protein set forth in Table 2. In some embodiments, the amino acid sequence of the immunoreceptor inhibitory protein (or a functional fragment, functional variant, or functional fragment/variant thereof) consists of an amino acid sequence about 90% identical to the amino acid sequence of a protein set forth in Table 2. In some embodiments, the amino acid sequence of the immunoreceptor inhibitory protein (or a functional fragment, functional variant, or functional fragment/variant thereof) consists of an amino acid sequence about 95% identical to the amino acid sequence of a protein set forth in Table 2. In some embodiments, the amino acid sequence of the immunoreceptor inhibitory protein (or a functional fragment, functional variant, or functional fragment/variant thereof) consists of an amino acid sequence about 99% identical to the amino acid sequence of a protein set forth in Table 2. In some embodiments, the amino acid sequence of the immunoreceptor inhibitory protein (or a functional fragment, functional variant, or functional fragment/variant thereof) consists of an amino acid sequence about 100% identical to the amino acid sequence of a protein set forth in Table 2.

In some embodiments, the amino acid sequence of the immunoreceptor inhibitory protein (or a functional fragment, functional variant, or functional fragment/variant thereof) comprises the amino acid sequence of a protein set forth in Table 2, and further comprises 1 or more but less than 15% (less than 12%, less than 10%, less than 8%), amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence of a protein set forth in Table 2, and further comprises at least about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence of a protein set forth in Table 2, and further consists of at least about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence of a protein set forth in Table 2, and further comprises about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence of a protein set forth in Table 2, and further consists of about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence of a protein set forth in Table 2, and further comprises or no more than about 1, 2, 3, 4, 5, 6, 7, 8, 9, or 10 amino acid variations (e.g., substitutions, additions, deletions, etc.).

In some embodiments, the amino acid sequence of the immunoreceptor inhibitory protein (or a functional fragment, functional variant, or functional fragment/variant thereof) consists of the amino acid sequence of a protein set forth in Table 2, and further comprises 1 or more but less than 15% (less than 12%, less than 10%, less than 8%), amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence of a protein set forth in Table 2, and further comprises at least about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence of a protein set forth in Table 2, and further consists of at least about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence of a protein set forth in Table 2, and further comprises about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence of a protein set forth in Table 2, and further consists of about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence of a protein set forth in Table 2, and further comprises or no more than about 1, 2, 3, 4, 5, 6, 7, 8, 9, or 10 amino acid variations (e.g., substitutions, additions, deletions, etc.).

In some embodiments, the amino acid sequence of the immunoreceptor inhibitory protein (or a functional fragment, functional variant, or functional fragment/variant thereof) comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 23-84, 187-190, or 199-200. For example, the amino acid sequence of the immunoreceptor inhibitory protein (or a functional fragment, functional variant, or functional fragment/variant thereof) may comprise an amino acid sequence at least 85% identical to the amino acid sequence set forth in any one of SEQ ID NOS: 23-84, 187-190, or 199-200. The amino acid sequence of the immunoreceptor inhibitory protein (or a functional fragment, functional variant, or functional fragment/variant thereof) may comprise an amino acid sequence at least 90% identical to the amino acid sequence set forth in any one of SEQ ID NOS: 23-84, 187-190, or 199-200. The amino acid sequence of the immunoreceptor inhibitory protein (or a functional fragment, functional variant, or functional fragment/variant thereof) may comprise an amino acid sequence at least 95% identical to the amino acid sequence set forth in any one of SEQ ID NOS: 23-84, 187-190, or 199-200. In some embodiments, the amino acid sequence of the immunoreceptor inhibitory protein (or a functional fragment, functional variant, or functional fragment/variant thereof) may comprise an amino acid sequence 100% identical to the amino acid sequence set forth in any one of SEQ ID NOS: 23-84, 187-190, or 199-200.

In some embodiments, the amino acid sequence of the immunoreceptor inhibitory protein (or a functional fragment, functional variant, or functional fragment/variant thereof) consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 23-84, 187-190, or 199-200. For example, the amino acid sequence of the immunoreceptor inhibitory protein (or a functional fragment, functional variant, or functional fragment/variant thereof) may consist of an amino acid sequence at least 85% identical to the amino acid sequence set forth in any one of SEQ ID NOS: 23-84, 187-190, or 199-200. The amino acid sequence of the immunoreceptor inhibitory protein (or a functional fragment, functional variant, or functional fragment/variant thereof) may consist of an amino acid sequence at least 90% identical to the amino acid sequence set forth in any one of SEQ ID NOS: 23-84, 187-190, or 199-200. The amino acid sequence of the immunoreceptor inhibitory protein (or a functional fragment, functional variant, or functional fragment/variant thereof) may consist of an amino acid sequence at least 95% identical to the amino acid sequence set forth in any one of SEQ ID NOS: 23-84, 187-190, or 199-200. In some embodiments, the amino acid sequence of the immunoreceptor inhibitory protein (or a functional fragment, functional variant, or functional fragment/variant thereof) may consist of an amino acid sequence 100% identical to the amino acid sequence set forth in any one of SEQ ID NOS: 23-84, 187-190, or 199-200.

In embodiments, the amino acid sequence of the immunoreceptor inhibitory protein (or a functional fragment, functional variant, or functional fragment/variant thereof) comprises the amino acid sequence set forth in any one of SEQ ID NOS: 23-84, 187-190, or 199-200, and further comprises 1 or more but less than 15% (less than 12%, less than 10%, less than 8%), amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence set forth in any one of SEQ ID NOS: 23-84, 187-190, or 199-200, and further comprises at least about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence set forth in any one of SEQ ID NOS: 23-84, 187-190, or 199-200, and further comprises about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence set forth in any one of SEQ ID NOS: 23-84, 187-190, or 199-200, and further consists of about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence set forth in any one of SEQ ID NOS: 23-84, 187-190, or 199-200, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunoreceptor inhibitory protein (or a functional fragment, functional variant, or functional fragment/variant thereof) consists of the amino acid sequence set forth in any one of SEQ ID NOS: 23-84, 187-190, or 199-200, and further comprises 1 or more but less than 15% (less than 12%, less than 10%, less than 8%), amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence set forth in any one of SEQ ID NOS: 23-84, 187-190, or 199-200, and further comprises at least about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence set forth in any one of SEQ ID NOS: 23-84, 187-190, or 199-200, and further comprises about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence set forth in any one of SEQ ID NOS: 23-84, 187-190, or 199-200, and further consists of about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence set forth in any one of SEQ ID NOS: 23-84, 187-190, or 199-200, and further comprises or consists of no more than about 1, 2, 3, 4, 5, 6, 7, 8, 9, or 10 amino acid variations (e.g., substitutions, additions, deletions, etc.).

In some embodiments, the immunoreceptor inhibitory protein (or a functional fragment, functional variant, or functional fragment/variant thereof) comprises a homologous signal peptide operably connected to the immunoreceptor inhibitory protein. In some embodiments, the immunoreceptor inhibitory protein (or a functional fragment, functional variant, or functional fragment/variant thereof) comprises a homologous signal peptide operably connected to the N-terminus of the immunoreceptor inhibitory protein. In some embodiments, the amino acid sequence of the immunoreceptor inhibitory protein (or a functional fragment, functional variant, or functional fragment/variant thereof) comprises the amino acid sequence set forth in any one of SEQ ID NOS: 23-84, 187-190, or 199-200 and comprises a homologous signal peptide operably connected to the N-terminus of the immunoreceptor inhibitory protein. In some embodiments, the amino acid sequence of the immunoreceptor inhibitory protein (or a functional fragment, functional variant, or functional fragment/variant thereof) consists of the amino acid sequence set forth in any one of SEQ ID NOS: 23-84, 187-190, or 199-200 and comprises a homologous signal peptide operably connected to the N-terminus of the immunoreceptor inhibitory protein.

In some embodiments, the amino acid sequence of the immunoreceptor inhibitory protein (or a functional fragment, functional variant, or functional fragment/variant thereof) comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 23-53, 187-188, or 199. For example, the amino acid sequence of the immunoreceptor inhibitory protein (or a functional fragment, functional variant, or functional fragment/variant thereof) may comprise an amino acid sequence at least 85% identical to the amino acid sequence set forth in any one of SEQ ID NOS: 23-53, 187-188, or 199. The amino acid sequence of the immunoreceptor inhibitory protein (or a functional fragment, functional variant, or functional fragment/variant thereof) may comprise an amino acid sequence at least 90% identical to the amino acid sequence set forth in any one of SEQ ID NOS: 23-53, 187-188, or 199. The amino acid sequence of the immunoreceptor inhibitory protein (or a functional fragment, functional variant, or functional fragment/variant thereof) may comprise an amino acid sequence at least 95% identical to the amino acid sequence set forth in any one of SEQ ID NOS: 23-53, 187-188, or 199. In some embodiments, the amino acid sequence of the immunoreceptor inhibitory protein (or a functional fragment, functional variant, or functional fragment/variant thereof) may comprise an amino acid sequence 100% identical to the amino acid sequence set forth in any one of SEQ ID NOS: 23-53, 187-188, or 199.

In some embodiments, the amino acid sequence of the immunoreceptor inhibitory protein (or a functional fragment, functional variant, or functional fragment/variant thereof) consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 23-53, 187-188, or 199. For example, the amino acid sequence of the immunoreceptor inhibitory protein (or a functional fragment, functional variant, or functional fragment/variant thereof) may consist of an amino acid sequence at least 85% identical to the amino acid sequence set forth in any one of SEQ ID NOS: 23-53, 187-188, or 199. The amino acid sequence of the immunoreceptor inhibitory protein (or a functional fragment, functional variant, or functional fragment/variant thereof) may consist of an amino acid sequence at least 90% identical to the amino acid sequence set forth in any one of SEQ ID NOS: 23-53, 187-188, or 199. The amino acid sequence of the immunoreceptor inhibitory protein (or a functional fragment, functional variant, or functional fragment/variant thereof) may consist of an amino acid sequence at least 95% identical to the amino acid sequence set forth in any one of SEQ ID NOS: 23-53, 187-188, or 199. In some embodiments, the amino acid sequence of the immunoreceptor inhibitory protein (or a functional fragment, functional variant, or functional fragment/variant thereof) may consist of an amino acid sequence 100% identical to the amino acid sequence set forth in any one of SEQ ID NOS: 23-53, 187-188, or 199.

In embodiments, the amino acid sequence of the immunoreceptor inhibitory protein (or a functional fragment, functional variant, or functional fragment/variant thereof) comprises the amino acid sequence set forth in any one of SEQ ID NOS: 23-53, 187-188, or 199, and further comprises 1 or more but less than 15% (less than 12%, less than 10%, less than 8%), amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence set forth in any one of SEQ ID NOS: 23-53, 187-188, or 199, and further comprises at least about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence set forth in any one of SEQ ID NOS: 23-53, 187-188, or 199, and further comprises about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence set forth in any one of SEQ ID NOS: 23-53, 187-188, or 199, and further consists of about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence set forth in any one of SEQ ID NOS: 23-53, 187-188, or 199, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunoreceptor inhibitory protein (or a functional fragment, functional variant, or functional fragment/variant thereof) consists of the amino acid sequence set forth in any one of SEQ ID NOS: 23-53, 187-188, or 199, and further comprises 1 or more but less than 15% (less than 12%, less than 10%, less than 8%), amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence set forth in any one of SEQ ID NOS: 23-53, 187-188, or 199, and further comprises at least about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence set forth in any one of SEQ ID NOS: 23-53, 187-188, or 199, and further comprises about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence set forth in any one of SEQ ID NOS: 23-53, 187-188, or 199, and further consists of about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence set forth in any one of SEQ ID NOS: 23-53, 187-188, or 199, and further comprises or consists of no more than about 1, 2, 3, 4, 5, 6, 7, 8, 9, or 10 amino acid variations (e.g., substitutions, additions, deletions, etc.).

In some embodiments, the immunoreceptor inhibitory protein (or a functional fragment, functional variant, or functional fragment/variant thereof) comprises a homologous signal peptide operably connected to the immunoreceptor inhibitory protein. In some embodiments, the immunoreceptor inhibitory protein (or a functional fragment, functional variant, or functional fragment/variant thereof) comprises a homologous signal peptide operably connected to the N-terminus of the immunoreceptor inhibitory protein. In some embodiments, the amino acid sequence of the immunoreceptor inhibitory protein (or a functional fragment, functional variant, or functional fragment/variant thereof) comprises the amino acid sequence set forth in any one of SEQ ID NOS: 23-53, 187-188, or 199 and comprises a homologous signal peptide operably connected to the N-terminus of the immunoreceptor inhibitory protein. In some embodiments, the amino acid sequence of the immunoreceptor inhibitory protein (or a functional fragment, functional variant, or functional fragment/variant thereof) consists of the amino acid sequence set forth in any one of SEQ ID NOS: 23-53, 187-188, or 199 and comprises a homologous signal peptide operably connected to the N-terminus of the immunoreceptor inhibitory protein.

In some embodiments, the amino acid sequence of the immunoreceptor inhibitory protein (or a functional fragment, functional variant, or functional fragment/variant thereof) comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 54-84, 189-190, or 200. For example, the amino acid sequence of the immunoreceptor inhibitory protein (or a functional fragment, functional variant, or functional fragment/variant thereof) may comprise an amino acid sequence at least 85% identical to the amino acid sequence set forth in any one of SEQ ID NOS: 54-84, 189-190, or 200. The amino acid sequence of the immunoreceptor inhibitory protein (or a functional fragment, functional variant, or functional fragment/variant thereof) may comprise an amino acid sequence at least 90% identical to the amino acid sequence set forth in any one of SEQ ID NOS: 54-84, 189-190, or 200. The amino acid sequence of the immunoreceptor inhibitory protein (or a functional fragment, functional variant, or functional fragment/variant thereof) may comprise an amino acid sequence at least 95% identical to the amino acid sequence set forth in any one of SEQ ID NOS: 54-84, 189-190, or 200. In some embodiments, the amino acid sequence of the immunoreceptor inhibitory protein (or a functional fragment, functional variant, or functional fragment/variant thereof) may comprise an amino acid sequence 100% identical to the amino acid sequence set forth in any one of SEQ ID NOS: 54-84, 189-190, or 200.

In some embodiments, the amino acid sequence of the immunoreceptor inhibitory protein (or a functional fragment, functional variant, or functional fragment/variant thereof) consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 54-84, 189-190, or 200. For example, the amino acid sequence of the immunoreceptor inhibitory protein (or a functional fragment, functional variant, or functional fragment/variant thereof) may consist of an amino acid sequence at least 85% identical to the amino acid sequence set forth in any one of SEQ ID NOS: 54-84, 189-190, or 200. The amino acid sequence of the immunoreceptor inhibitory protein (or a functional fragment, functional variant, or functional fragment/variant thereof) may consist of an amino acid sequence at least 90% identical to the amino acid sequence set forth in any one of SEQ ID NOS: 54-84, 189-190, or 200. The amino acid sequence of the immunoreceptor inhibitory protein (or a functional fragment, functional variant, or functional fragment/variant thereof) may consist of an amino acid sequence at least 95% identical to the amino acid sequence set forth in any one of SEQ ID NOS: 54-84, 189-190, or 200. In some embodiments, the amino acid sequence of the immunoreceptor inhibitory protein (or a functional fragment, functional variant, or functional fragment/variant thereof) may consist of an amino acid sequence 100% identical to the amino acid sequence set forth in any one of SEQ ID NOS: 54-84, 189-190, or 200.

In embodiments, the amino acid sequence of the immunoreceptor inhibitory protein (or a functional fragment, functional variant, or functional fragment/variant thereof) comprises the amino acid sequence set forth in any one of SEQ ID NOS: 54-84, 189-190, or 200, and further comprises 1 or more but less than 15% (less than 12%, less than 10%, less than 8%), amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence set forth in any one of SEQ ID NOS: 54-84, 189-190, or 200, and further comprises at least about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence set forth in any one of SEQ ID NOS: 54-84, 189-190, or 200, and further comprises about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence set forth in any one of SEQ ID NOS: 54-84, 189-190, or 200, and further consists of about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) comprises the amino acid sequence set forth in any one of SEQ ID NOS: 54-84, 189-190, or 200, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunoreceptor inhibitory protein (or a functional fragment, functional variant, or functional fragment/variant thereof) consists of the amino acid sequence set forth in any one of SEQ ID NOS: 54-84, 189-190, or 200, and further comprises 1 or more but less than 15% (less than 12%, less than 10%, less than 8%), amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence set forth in any one of SEQ ID NOS: 54-84, 189-190, or 200, and further comprises at least about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence set forth in any one of SEQ ID NOS: 54-84, 189-190, or 200, and further comprises about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence set forth in any one of SEQ ID NOS: 54-84, 189-190, or 200, and further consists of about 1, 2, 3, 4, 5, 6, 7, 8, 9, or 10 amino acid variations (e.g., substitutions, additions, deletions, etc.). In some embodiments, the amino acid sequence of the immunoreceptor inhibitory protein (or a functional fragment, functional variant, or functional fragment/variant thereof) consists of the amino acid sequence set forth in any one of SEQ ID NOS: 54-84, 189-190, or 200, and further comprises or consists of no more than about 1, 2, 3, 4, 5, 6, 7, 8, 9, or 10 amino acid variations (e.g., substitutions, additions, deletions, etc.).

In some embodiments, the immunoreceptor inhibitory protein (or a functional fragment, functional variant, or functional fragment/variant thereof) comprises a homologous signal peptide operably connected to the immunoreceptor inhibitory protein. In some embodiments, the immunoreceptor inhibitory protein (or a functional fragment, functional variant, or functional fragment/variant thereof) comprises a homologous signal peptide operably connected to the N-terminus of the immunoreceptor inhibitory protein. In some embodiments, the amino acid sequence of the immunoreceptor inhibitory protein (or a functional fragment, functional variant, or functional fragment/variant thereof) comprises the amino acid sequence set forth in any one of SEQ ID NOS: 54-84, 189-190, or 200 and comprises a homologous signal peptide operably connected to the N-terminus of the immunoreceptor inhibitory protein. In some embodiments, the amino acid sequence of the immunoreceptor inhibitory protein (or a functional fragment, functional variant, or functional fragment/variant thereof) consists of the amino acid sequence set forth in any one of SEQ ID NOS: 54-84, 189-190, or 200 and comprises a homologous signal peptide operably connected to the N-terminus of the immunoreceptor inhibitory protein.

In specific embodiments, the amino acid sequence of the immunoreceptor inhibitory protein (or a functional fragment, functional variant, or functional fragment/variant thereof) comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 24. For example, the amino acid sequence of the immunoreceptor inhibitory protein (or a functional fragment, functional variant, or functional fragment/variant thereof) may comprise an amino acid sequence at least 85% identical to the amino acid sequence set forth in SEQ ID NO: 24. The amino acid sequence of the immunoreceptor inhibitory protein (or a functional fragment, functional variant, or functional fragment/variant thereof) may comprise an amino acid sequence at least 90% identical to the amino acid sequence set forth in any one of in SEQ ID NO: 24. The amino acid sequence of the immunoreceptor inhibitory protein (or a functional fragment, functional variant, or functional fragment/variant thereof) may comprise an amino acid sequence at least 95% identical to the amino acid sequence set forth in SEQ ID NO: 24. In specific embodiments, the amino acid sequence of the immunoreceptor inhibitory protein (or a functional fragment, functional variant, or functional fragment/variant thereof) may comprise an amino acid sequence 100% identical to the amino acid sequence set forth in SEQ ID NO: 24.

In specific embodiments, the amino acid sequence of the immunoreceptor inhibitory protein (or a functional fragment, functional variant, or functional fragment/variant thereof) consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 24. For example, the amino acid sequence of the immunoreceptor inhibitory protein (or a functional fragment, functional variant, or functional fragment/variant thereof) may consist of an amino acid sequence at least 85% identical to the amino acid sequence set forth in SEQ ID NO: 24. The amino acid sequence of the immunoreceptor inhibitory protein (or a functional fragment, functional variant, or functional fragment/variant thereof) may consist of an amino acid sequence at least 90% identical to the amino acid sequence set forth in SEQ ID NO: 24. The amino acid sequence of the immunoreceptor inhibitory protein (or a functional fragment, functional variant, or functional fragment/variant thereof) may consist of an amino acid sequence at least 95% identical to the amino acid sequence set forth in SEQ ID NO: 24. In specific embodiments, the amino acid sequence of the immunoreceptor inhibitory protein (or a functional fragment, functional variant, or functional fragment/variant thereof) may consist of an amino acid sequence 100% identical to the amino acid sequence set forth in SEQ ID NO: 24.

In embodiments, the amino acid sequence of the immunoreceptor inhibitory protein (or a functional fragment, functional variant, or functional fragment/variant thereof) comprises the amino acid sequence set forth in SEQ ID NO: 24, and further comprises 1 or more but less than 15% (less than 12%, less than 10%, less than 8%), amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 24, and further comprises at least about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 24, and further comprises about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 24, and further consists of about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 24, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunoreceptor inhibitory protein (or a functional fragment, functional variant, or functional fragment/variant thereof) consists of the amino acid sequence set forth in SEQ ID NO: 24, and further comprises 1 or more but less than 15% (less than 12%, less than 10%, less than 8%), amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 24, and further comprises at least about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 24, and further comprises about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 24, and further consists of about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 24, and further comprises or consists of no more than about 1, 2, 3, 4, 5, 6, 7, 8, 9, or 10 amino acid variations (e.g., substitutions, additions, deletions, etc.).

In specific embodiments, the immunoreceptor inhibitory protein (or a functional fragment, functional variant, or functional fragment/variant thereof) comprises a homologous signal peptide operably connected to the immunoreceptor inhibitory protein. In specific embodiments, the immunoreceptor inhibitory protein (or a functional fragment, functional variant, or functional fragment/variant thereof) comprises a homologous signal peptide operably connected to the N-terminus of the immunoreceptor inhibitory protein. In specific embodiments, the amino acid sequence of the immunoreceptor inhibitory protein (or a functional fragment, functional variant, or functional fragment/variant thereof) comprises the amino acid sequence set forth in SEQ ID NO: 24 and comprises a homologous signal peptide operably connected to the N-terminus of the immunoreceptor inhibitory protein. In specific embodiments, the amino acid sequence of the immunoreceptor inhibitory protein (or a functional fragment, functional variant, or functional fragment/variant thereof) consists of the amino acid sequence set forth in SEQ ID NO: 24 and comprises a homologous signal peptide operably connected to the N-terminus of the immunoreceptor inhibitory protein.

In specific embodiments, the amino acid sequence of the immunoreceptor inhibitory protein (or a functional fragment, functional variant, or functional fragment/variant thereof) comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 55. For example, the amino acid sequence of the immunoreceptor inhibitory protein (or a functional fragment, functional variant, or functional fragment/variant thereof) may comprise an amino acid sequence at least 85% identical to the amino acid sequence set forth in SEQ ID NO: 55. The amino acid sequence of the immunoreceptor inhibitory protein (or a functional fragment, functional variant, or functional fragment/variant thereof) may comprise an amino acid sequence at least 90% identical to the amino acid sequence set forth in any one of in SEQ ID NO: 55. The amino acid sequence of the immunoreceptor inhibitory protein (or a functional fragment, functional variant, or functional fragment/variant thereof) may comprise an amino acid sequence at least 95% identical to the amino acid sequence set forth in SEQ ID NO: 55. In specific embodiments, the amino acid sequence of the immunoreceptor inhibitory protein (or a functional fragment, functional variant, or functional fragment/variant thereof) may comprise an amino acid sequence 100% identical to the amino acid sequence set forth in SEQ ID NO: 55.

In specific embodiments, the amino acid sequence of the immunoreceptor inhibitory protein (or a functional fragment, functional variant, or functional fragment/variant thereof) consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 55. For example, the amino acid sequence of the immunoreceptor inhibitory protein (or a functional fragment, functional variant, or functional fragment/variant thereof) may consist of an amino acid sequence at least 85% identical to the amino acid sequence set forth in SEQ ID NO: 55. The amino acid sequence of the immunoreceptor inhibitory protein (or a functional fragment, functional variant, or functional fragment/variant thereof) may consist of an amino acid sequence at least 90% identical to the amino acid sequence set forth in SEQ ID NO: 55. The amino acid sequence of the immunoreceptor inhibitory protein (or a functional fragment, functional variant, or functional fragment/variant thereof) may consist of an amino acid sequence at least 95% identical to the amino acid sequence set forth in SEQ ID NO: 55. In specific embodiments, the amino acid sequence of the immunoreceptor inhibitory protein (or a functional fragment, functional variant, or functional fragment/variant thereof) may consist of an amino acid sequence 100% identical to the amino acid sequence set forth in SEQ ID NO: 55.

In embodiments, the amino acid sequence of the immunoreceptor inhibitory protein (or a functional fragment, functional variant, or functional fragment/variant thereof) comprises the amino acid sequence set forth in SEQ ID NO: 55, and further comprises 1 or more but less than 15% (less than 12%, less than 10%, less than 8%), amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 55, and further comprises at least about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 55, and further comprises about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 55, and further consists of about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 55, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunoreceptor inhibitory protein (or a functional fragment, functional variant, or functional fragment/variant thereof) consists of the amino acid sequence set forth in SEQ ID NO: 55, and further comprises 1 or more but less than 15% (less than 12%, less than 10%, less than 8%), amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 55, and further comprises at least about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 55, and further comprises about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 55, and further consists of about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 55, and further comprises or consists of no more than about 1, 2, 3, 4, 5, 6, 7, 8, 9, or 10 amino acid variations (e.g., substitutions, additions, deletions, etc.).

In specific embodiments, the immunoreceptor inhibitory protein (or a functional fragment, functional variant, or functional fragment/variant thereof) comprises a homologous signal peptide operably connected to the immunoreceptor inhibitory protein. In specific embodiments, the immunoreceptor inhibitory protein (or a functional fragment, functional variant, or functional fragment/variant thereof) comprises a homologous signal peptide operably connected to the N-terminus of the immunoreceptor inhibitory protein. In specific embodiments, the amino acid sequence of the immunoreceptor inhibitory protein (or a functional fragment, functional variant, or functional fragment/variant thereof) comprises the amino acid sequence set forth in SEQ ID NO: 55 and comprises a homologous signal peptide operably connected to the N-terminus of the immunoreceptor inhibitory protein. In specific embodiments, the amino acid sequence of the immunoreceptor inhibitory protein (or a functional fragment, functional variant, or functional fragment/variant thereof) consists of the amino acid sequence set forth in SEQ ID NO: 55 and comprises a homologous signal peptide operably connected to the N-terminus of the immunoreceptor inhibitory protein.

In specific embodiments, the amino acid sequence of the immunoreceptor inhibitory protein (or a functional fragment, functional variant, or functional fragment/variant thereof) comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 188. For example, the amino acid sequence of the immunoreceptor inhibitory protein (or a functional fragment, functional variant, or functional fragment/variant thereof) may comprise an amino acid sequence at least 85% identical to the amino acid sequence set forth in SEQ ID NO: 188. The amino acid sequence of the immunoreceptor inhibitory protein (or a functional fragment, functional variant, or functional fragment/variant thereof) may comprise an amino acid sequence at least 90% identical to the amino acid sequence set forth in any one of in SEQ ID NO: 188. The amino acid sequence of the immunoreceptor inhibitory protein (or a functional fragment, functional variant, or functional fragment/variant thereof) may comprise an amino acid sequence at least 95% identical to the amino acid sequence set forth in SEQ ID NO: 188. In specific embodiments, the amino acid sequence of the immunoreceptor inhibitory protein (or a functional fragment, functional variant, or functional fragment/variant thereof) may comprise an amino acid sequence 100% identical to the amino acid sequence set forth in SEQ ID NO: 188.

In specific embodiments, the amino acid sequence of the immunoreceptor inhibitory protein (or a functional fragment, functional variant, or functional fragment/variant thereof) consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 188. For example, the amino acid sequence of the immunoreceptor inhibitory protein (or a functional fragment, functional variant, or functional fragment/variant thereof) may consist of an amino acid sequence at least 85% identical to the amino acid sequence set forth in SEQ ID NO: 188. The amino acid sequence of the immunoreceptor inhibitory protein (or a functional fragment, functional variant, or functional fragment/variant thereof) may consist of an amino acid sequence at least 90% identical to the amino acid sequence set forth in SEQ ID NO: 188. The amino acid sequence of the immunoreceptor inhibitory protein (or a functional fragment, functional variant, or functional fragment/variant thereof) may consist of an amino acid sequence at least 95% identical to the amino acid sequence set forth in SEQ ID NO: 188. In specific embodiments, the amino acid sequence of the immunoreceptor inhibitory protein (or a functional fragment, functional variant, or functional fragment/variant thereof) may consist of an amino acid sequence 100% identical to the amino acid sequence set forth in SEQ ID NO: 188.

In embodiments, the amino acid sequence of the immunoreceptor inhibitory protein (or a functional fragment, functional variant, or functional fragment/variant thereof) comprises the amino acid sequence set forth in SEQ ID NO: 188, and further comprises 1 or more but less than 15% (less than 12%, less than 10%, less than 8%), amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 188, and further comprises at least about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 188, and further comprises about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 188, and further consists of about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 188, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunoreceptor inhibitory protein (or a functional fragment, functional variant, or functional fragment/variant thereof) consists of the amino acid sequence set forth in SEQ ID NO: 188, and further comprises 1 or more but less than 15% (less than 12%, less than 10%, less than 8%), amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 188, and further comprises at least about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 188, and further comprises about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 188, and further consists of about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 188, and further comprises or consists of no more than about 1, 2, 3, 4, 5, 6, 7, 8, 9, or 10 amino acid variations (e.g., substitutions, additions, deletions, etc.).

In specific embodiments, the immunoreceptor inhibitory protein (or a functional fragment, functional variant, or functional fragment/variant thereof) comprises a homologous signal peptide operably connected to the immunoreceptor inhibitory protein. In specific embodiments, the immunoreceptor inhibitory protein (or a functional fragment, functional variant, or functional fragment/variant thereof) comprises a homologous signal peptide operably connected to the N-terminus of the immunoreceptor inhibitory protein. In specific embodiments, the amino acid sequence of the immunoreceptor inhibitory protein (or a functional fragment, functional variant, or functional fragment/variant thereof) comprises the amino acid sequence set forth in SEQ ID NO: 188 and comprises a homologous signal peptide operably connected to the N-terminus of the immunoreceptor inhibitory protein. In specific embodiments, the amino acid sequence of the immunoreceptor inhibitory protein (or a functional fragment, functional variant, or functional fragment/variant thereof) consists of the amino acid sequence set forth in SEQ ID NO: 188 and comprises a homologous signal peptide operably connected to the N-terminus of the immunoreceptor inhibitory protein.

In specific embodiments, the amino acid sequence of the immunoreceptor inhibitory protein (or a functional fragment, functional variant, or functional fragment/variant thereof) comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 190. For example, the amino acid sequence of the immunoreceptor inhibitory protein (or a functional fragment, functional variant, or functional fragment/variant thereof) may comprise an amino acid sequence at least 85% identical to the amino acid sequence set forth in SEQ ID NO: 190. The amino acid sequence of the immunoreceptor inhibitory protein (or a functional fragment, functional variant, or functional fragment/variant thereof) may comprise an amino acid sequence at least 90% identical to the amino acid sequence set forth in any one of in SEQ ID NO: 190. The amino acid sequence of the immunoreceptor inhibitory protein (or a functional fragment, functional variant, or functional fragment/variant thereof) may comprise an amino acid sequence at least 95% identical to the amino acid sequence set forth in SEQ ID NO: 190. In specific embodiments, the amino acid sequence of the immunoreceptor inhibitory protein (or a functional fragment, functional variant, or functional fragment/variant thereof) may comprise an amino acid sequence 100% identical to the amino acid sequence set forth in SEQ ID NO: 190.

In specific embodiments, the amino acid sequence of the immunoreceptor inhibitory protein (or a functional fragment, functional variant, or functional fragment/variant thereof) consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 190. For example, the amino acid sequence of the immunoreceptor inhibitory protein (or a functional fragment, functional variant, or functional fragment/variant thereof) may consist of an amino acid sequence at least 85% identical to the amino acid sequence set forth in SEQ ID NO: 190. The amino acid sequence of the immunoreceptor inhibitory protein (or a functional fragment, functional variant, or functional fragment/variant thereof) may consist of an amino acid sequence at least 90% identical to the amino acid sequence set forth in SEQ ID NO: 190. The amino acid sequence of the immunoreceptor inhibitory protein (or a functional fragment, functional variant, or functional fragment/variant thereof) may consist of an amino acid sequence at least 95% identical to the amino acid sequence set forth in SEQ ID NO: 190. In specific embodiments, the amino acid sequence of the immunoreceptor inhibitory protein (or a functional fragment, functional variant, or functional fragment/variant thereof) may consist of an amino acid sequence 100% identical to the amino acid sequence set forth in SEQ ID NO: 190.

In embodiments, the amino acid sequence of the immunoreceptor inhibitory protein (or a functional fragment, functional variant, or functional fragment/variant thereof) comprises the amino acid sequence set forth in SEQ ID NO: 190, and further comprises 1 or more but less than 15% (less than 12%, less than 10%, less than 8%), amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 190, and further comprises at least about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 190, and further comprises about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 190, and further consists of about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 190, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunoreceptor inhibitory protein (or a functional fragment, functional variant, or functional fragment/variant thereof) consists of the amino acid sequence set forth in SEQ ID NO: 190, and further comprises 1 or more but less than 15% (less than 12%, less than 10%, less than 8%), amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 190, and further comprises at least about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 190, and further comprises about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 190, and further consists of about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 190, and further comprises or consists of no more than about 1, 2, 3, 4, 5, 6, 7, 8, 9, or 10 amino acid variations (e.g., substitutions, additions, deletions, etc.).

In specific embodiments, the immunoreceptor inhibitory protein (or a functional fragment, functional variant, or functional fragment/variant thereof) comprises a homologous signal peptide operably connected to the immunoreceptor inhibitory protein. In specific embodiments, the immunoreceptor inhibitory protein (or a functional fragment, functional variant, or functional fragment/variant thereof) comprises a homologous signal peptide operably connected to the N-terminus of the immunoreceptor inhibitory protein. In specific embodiments, the amino acid sequence of the immunoreceptor inhibitory protein (or a functional fragment, functional variant, or functional fragment/variant thereof) comprises the amino acid sequence set forth in SEQ ID NO: 190 and comprises a homologous signal peptide operably connected to the N-terminus of the immunoreceptor inhibitory protein. In specific embodiments, the amino acid sequence of the immunoreceptor inhibitory protein (or a functional fragment, functional variant, or functional fragment/variant thereof) consists of the amino acid sequence set forth in SEQ ID NO: 190 and comprises a homologous signal peptide operably connected to the N-terminus of the immunoreceptor inhibitory protein.

In specific embodiments, the amino acid sequence of the immunoreceptor inhibitory protein (or a functional fragment, functional variant, or functional fragment/variant thereof) comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 187. For example, the amino acid sequence of the immunoreceptor inhibitory protein (or a functional fragment, functional variant, or functional fragment/variant thereof) may comprise an amino acid sequence at least 85% identical to the amino acid sequence set forth in SEQ ID NO: 187. The amino acid sequence of the immunoreceptor inhibitory protein (or a functional fragment, functional variant, or functional fragment/variant thereof) may comprise an amino acid sequence at least 90% identical to the amino acid sequence set forth in any one of in SEQ ID NO: 187. The amino acid sequence of the immunoreceptor inhibitory protein (or a functional fragment, functional variant, or functional fragment/variant thereof) may comprise an amino acid sequence at least 95% identical to the amino acid sequence set forth in SEQ ID NO: 187. In specific embodiments, the amino acid sequence of the immunoreceptor inhibitory protein (or a functional fragment, functional variant, or functional fragment/variant thereof) may comprise an amino acid sequence 100% identical to the amino acid sequence set forth in SEQ ID NO: 187.

In specific embodiments, the amino acid sequence of the immunoreceptor inhibitory protein (or a functional fragment, functional variant, or functional fragment/variant thereof) consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 187. For example, the amino acid sequence of the immunoreceptor inhibitory protein (or a functional fragment, functional variant, or functional fragment/variant thereof) may consist of an amino acid sequence at least 85% identical to the amino acid sequence set forth in SEQ ID NO: 187. The amino acid sequence of the immunoreceptor inhibitory protein (or a functional fragment, functional variant, or functional fragment/variant thereof) may consist of an amino acid sequence at least 90% identical to the amino acid sequence set forth in SEQ ID NO: 187. The amino acid sequence of the immunoreceptor inhibitory protein (or a functional fragment, functional variant, or functional fragment/variant thereof) may consist of an amino acid sequence at least 95% identical to the amino acid sequence set forth in SEQ ID NO: 187. In specific embodiments, the amino acid sequence of the immunoreceptor inhibitory protein (or a functional fragment, functional variant, or functional fragment/variant thereof) may consist of an amino acid sequence 100% identical to the amino acid sequence set forth in SEQ ID NO: 187.

In embodiments, the amino acid sequence of the immunoreceptor inhibitory protein (or a functional fragment, functional variant, or functional fragment/variant thereof) comprises the amino acid sequence set forth in SEQ ID NO: 187, and further comprises 1 or more but less than 15% (less than 12%, less than 10%, less than 8%), amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 187, and further comprises at least about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 187, and further comprises about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 187, and further consists of about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 187, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunoreceptor inhibitory protein (or a functional fragment, functional variant, or functional fragment/variant thereof) consists of the amino acid sequence set forth in SEQ ID NO: 187, and further comprises 1 or more but less than 15% (less than 12%, less than 10%, less than 8%), amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 187, and further comprises at least about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 187, and further comprises about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 187, and further consists of about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 187, and further comprises or consists of no more than about 1, 2, 3, 4, 5, 6, 7, 8, 9, or 10 amino acid variations (e.g., substitutions, additions, deletions, etc.).

In specific embodiments, the immunoreceptor inhibitory protein (or a functional fragment, functional variant, or functional fragment/variant thereof) comprises a homologous signal peptide operably connected to the immunoreceptor inhibitory protein. In specific embodiments, the immunoreceptor inhibitory protein (or a functional fragment, functional variant, or functional fragment/variant thereof) comprises a homologous signal peptide operably connected to the N-terminus of the immunoreceptor inhibitory protein. In specific embodiments, the amino acid sequence of the immunoreceptor inhibitory protein (or a functional fragment, functional variant, or functional fragment/variant thereof) comprises the amino acid sequence set forth in SEQ ID NO: 187 and comprises a homologous signal peptide operably connected to the N-terminus of the immunoreceptor inhibitory protein. In specific embodiments, the amino acid sequence of the immunoreceptor inhibitory protein (or a functional fragment, functional variant, or functional fragment/variant thereof) consists of the amino acid sequence set forth in SEQ ID NO: 187 and comprises a homologous signal peptide operably connected to the N-terminus of the immunoreceptor inhibitory protein.

In specific embodiments, the amino acid sequence of the immunoreceptor inhibitory protein (or a functional fragment, functional variant, or functional fragment/variant thereof) comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 189. For example, the amino acid sequence of the immunoreceptor inhibitory protein (or a functional fragment, functional variant, or functional fragment/variant thereof) may comprise an amino acid sequence at least 85% identical to the amino acid sequence set forth in SEQ ID NO: 189. The amino acid sequence of the immunoreceptor inhibitory protein (or a functional fragment, functional variant, or functional fragment/variant thereof) may comprise an amino acid sequence at least 90% identical to the amino acid sequence set forth in any one of in SEQ ID NO: 189. The amino acid sequence of the immunoreceptor inhibitory protein (or a functional fragment, functional variant, or functional fragment/variant thereof) may comprise an amino acid sequence at least 95% identical to the amino acid sequence set forth in SEQ ID NO: 189. In specific embodiments, the amino acid sequence of the immunoreceptor inhibitory protein (or a functional fragment, functional variant, or functional fragment/variant thereof) may comprise an amino acid sequence 100% identical to the amino acid sequence set forth in SEQ ID NO: 189.

In specific embodiments, the amino acid sequence of the immunoreceptor inhibitory protein (or a functional fragment, functional variant, or functional fragment/variant thereof) consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 189. For example, the amino acid sequence of the immunoreceptor inhibitory protein (or a functional fragment, functional variant, or functional fragment/variant thereof) may consist of an amino acid sequence at least 85% identical to the amino acid sequence set forth in SEQ ID NO: 189. The amino acid sequence of the immunoreceptor inhibitory protein (or a functional fragment, functional variant, or functional fragment/variant thereof) may consist of an amino acid sequence at least 90% identical to the amino acid sequence set forth in SEQ ID NO: 189. The amino acid sequence of the immunoreceptor inhibitory protein (or a functional fragment, functional variant, or functional fragment/variant thereof) may consist of an amino acid sequence at least 95% identical to the amino acid sequence set forth in SEQ ID NO: 189. In specific embodiments, the amino acid sequence of the immunoreceptor inhibitory protein (or a functional fragment, functional variant, or functional fragment/variant thereof) may consist of an amino acid sequence 100% identical to the amino acid sequence set forth in SEQ ID NO: 189.

In embodiments, the amino acid sequence of the immunoreceptor inhibitory protein (or a functional fragment, functional variant, or functional fragment/variant thereof) comprises the amino acid sequence set forth in SEQ ID NO: 189, and further comprises 1 or more but less than 15% (less than 12%, less than 10%, less than 8%), amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 189, and further comprises at least about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 189, and further comprises about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 189, and further consists of about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) comprises the amino acid sequence set forth in SEQ ID NO: 189, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunoreceptor inhibitory protein (or a functional fragment, functional variant, or functional fragment/variant thereof) consists of the amino acid sequence set forth in SEQ ID NO: 189, and further comprises 1 or more but less than 15% (less than 12%, less than 10%, less than 8%), amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 189, and further comprises at least about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 189, and further comprises about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 189, and further consists of about 1, 2, 3, 4, 5, 6, 7, 8, 9, or 10 amino acid variations (e.g., substitutions, additions, deletions, etc.). In specific embodiments, the amino acid sequence of the immunoreceptor inhibitory protein (or a functional fragment, functional variant, or functional fragment/variant thereof) consists of the amino acid sequence set forth in SEQ ID NO: 189, and further comprises or consists of no more than about 1, 2, 3, 4, 5, 6, 7, 8, 9, or 10 amino acid variations (e.g., substitutions, additions, deletions, etc.).

In specific embodiments, the immunoreceptor inhibitory protein (or a functional fragment, functional variant, or functional fragment/variant thereof) comprises a homologous signal peptide operably connected to the immunoreceptor inhibitory protein. In specific embodiments, the immunoreceptor inhibitory protein (or a functional fragment, functional variant, or functional fragment/variant thereof) comprises a homologous signal peptide operably connected to the N-terminus of the immunoreceptor inhibitory protein. In specific embodiments, the amino acid sequence of the immunoreceptor inhibitory protein (or a functional fragment, functional variant, or functional fragment/variant thereof) comprises the amino acid sequence set forth in SEQ ID NO: 189 and comprises a homologous signal peptide operably connected to the N-terminus of the immunoreceptor inhibitory protein. In specific embodiments, the amino acid sequence of the immunoreceptor inhibitory protein (or a functional fragment, functional variant, or functional fragment/variant thereof) consists of the amino acid sequence set forth in SEQ ID NO: 189 and comprises a homologous signal peptide operably connected to the N-terminus of the immunoreceptor inhibitory protein.

In some embodiments, the amino acid sequence of the immunoreceptor inhibitory protein (or a functional fragment, functional variant, or functional fragment/variant thereof) no more than 120, 115, 110, 100, 95, 90, 80, 70, 60, or 50 amino acids in length. In some embodiments, the amino acid sequence of the immunoreceptor inhibitory protein (or a functional fragment, functional variant, or functional fragment/variant thereof) is less than 120, 115, 110, 100, 95, 90, 80, 70, 60, or 50 amino acids in length. In some embodiments, the amino acid sequence of the immunoreceptor inhibitory protein (or a functional fragment, functional variant, or functional fragment/variant thereof) is from about 50-120, 50-110, 50-100, 50-90, 50-60, 60-120, 60-110, 60-100, 60-90, 60-80, 60-70, 70-120, 70-110, 70-100, 70-90, 70-80, 80-120, 80-110, 80-100, 80-90, 90-120, 90-110, 90-100, 100-120, or 100-110.

### 5.3 Exemplary Properties of Immunoreceptor Inhibitory Proteins

In specific embodiments, the immunoreceptor inhibitory proteins described herein are immunosuppressive (e.g., when administered to a subject). In specific embodiments, the immunoreceptor inhibitory proteins described herein are anti-inflammatory (e.g., when administered to a subject). In specific embodiments, the immunoreceptor inhibitory proteins described herein suppress a pro-inflammatory response (e.g., when administered to a subject). In specific embodiments, the immunoreceptor inhibitory proteins described herein suppress a pro-inflammatory immune response (e.g., when administered to a subject). In specific embodiments, the immunoreceptor inhibitory protein can act as a decoy receptor for a TNFSF ligand described herein.

In some embodiments, the immunoreceptor inhibitory proteins described herein binds one or more of TNFSF ligands. In some embodiments, the immunoreceptor inhibitory proteins described herein binds a subset of TNFSF ligands. In some embodiments, the immunoreceptor inhibitory proteins described herein binds a plurality of TNFSF ligands. In some embodiments, the immunoreceptor inhibitory protein binds TL1A. In some embodiments, the immunoreceptor inhibitory protein binds hTL1A. In preferred embodiments, the immunoreceptor inhibitory protein specifically binds TL1A. In preferred embodiments, the immunoreceptor inhibitory protein specifically binds hTL1A.

In some embodiments, the immunoreceptor inhibitory protein inhibits reduces (e.g., prevents) binding of TL1A to DR3. In some embodiments, the immunoreceptor inhibitory protein specifically binds to TL1A and inhibits reduces (e.g., prevents) binding of TL1A to DR3. In some embodiments, the immunoreceptor inhibitory protein inhibits reduces (e.g., prevents) binding of hTL1A to hDR3. In some embodiments, the immunoreceptor inhibitory protein specifically binds to hTL1A and inhibits reduces (e.g., prevents) binding of hTL1A to hDR3. In some embodiments, the immunoreceptor inhibitory protein inhibits reduces (e.g., prevents) DR3 signaling. In some embodiments, the immunoreceptor inhibitory protein inhibits reduces (e.g., prevents) DR3 signaling mediated by DR3 binding to TL1A. In some embodiments, the immunoreceptor inhibitory protein inhibits reduces (e.g., prevents) NFκB activation. In some embodiments, the immunoreceptor inhibitory protein inhibits reduces (e.g., prevents) NFκB activation mediating through DR3. In some embodiments, the immunoreceptor inhibitory protein inhibits reduces (e.g., prevents) NFκB activation mediated through DR3 binding to TL1A.

In some embodiments, the immunoreceptor inhibitory protein inhibits reduces (e.g., prevents) binding of TL1A to DcR3. In some embodiments, the immunoreceptor inhibitory protein specifically binds to TL1A and inhibits reduces (e.g., prevents) binding of TL1A to DcR3. In some embodiments, the immunoreceptor inhibitory protein inhibits reduces (e.g., prevents) binding of hTL1A to hDcR3. In some embodiments, the immunoreceptor inhibitory protein specifically binds to hTL1A and inhibits reduces (e.g., prevents) binding of hTL1A to hDcR3. In some embodiments, the immunoreceptor inhibitory protein inhibits reduces (e.g., prevents) DcR3 signaling. In some embodiments, the immunoreceptor inhibitory protein inhibits reduces (e.g., prevents) DcR3 signaling mediated by DcR3 binding to TL1A.

In some embodiments, the immunoreceptor inhibitory protein does not specifically binds one or more of TNFα, LIGHT, and/or FASL. In some embodiments, the immunoreceptor inhibitory protein does not specifically bind 2 or 3 of TNFα, LIGHT, and/or FASL. In some embodiments, the immunoreceptor inhibitory protein does not specifically bind TNFα, LIGHT, and/or FASL. In some embodiments, the immunoreceptor inhibitory protein does not specifically bind TNFα, LIGHT, and/or FASL. In some embodiments, the immunoreceptor inhibitory protein does not specifically bind a plurality of TNFα, LIGHT, and/or FASL.

In some embodiments, the immunoreceptor inhibitory protein does not specifically binds one or more of hTNFα, hLIGHT, and/or hFASL. In some embodiments, the immunoreceptor inhibitory protein does not specifically bind 2 or 3 of hTNFα, hLIGHT, and/or hFASL. In some embodiments, the immunoreceptor inhibitory protein does not specifically bind hTNFα, hLIGHT, and/or hFASL. In some embodiments, the immunoreceptor inhibitory protein does not specifically bind hTNFα, hLIGHT, and/or hFASL. In some embodiments, the immunoreceptor inhibitory protein does not specifically bind a plurality of hTNFα, hLIGHT, and/or hFASL.

In some embodiments, the immunoreceptor inhibitory protein does not inhibit or reduce (e.g., prevent) binding of one or more TNFSF ligand (e.g., TNFα, LIGHT, and/or FASL) to one or more of the TNFSF ligand's (e.g., TNFα, LIGHT, and/or FASL) cognate receptor.

In some embodiments, the immunoreceptor inhibitory protein exhibits one or more (e.g., 1, 2, 3, 4, and/or 5) of (a) does not inhibit or reduce (e.g., prevent) binding of TNFα to TNFR1, (b) does not inhibit or reduce (e.g., prevent) binding of TNFα to TNFR2, (c) does not inhibit or reduce (e.g., prevent) binding of LIGHT to LIGHTR, (d) does not inhibit or reduce (e.g., prevent) binding of LIGHT to LTβR, and/or (e) does not inhibit or reduce (e.g., prevent) binding of FASL to FAS. In some embodiments, the immunoreceptor inhibitory protein does not inhibit or reduce (e.g., prevent) binding of TNFα to TNFR1. In some embodiments, the immunoreceptor inhibitory protein does not inhibit or reduce (e.g., prevent) binding of TNFα to TNFR2. In some embodiments, the immunoreceptor inhibitory protein does not inhibit or reduce (e.g., prevent) binding of LIGHT to LIGHTR. In some embodiments, the immunoreceptor inhibitory protein does not inhibit or reduce (e.g., prevent) binding of LIGHT to LTβR. In some embodiments, the immunoreceptor inhibitory protein does not inhibit or reduce (e.g., prevent) binding of FASL to FAS. In some embodiments, the immunoreceptor inhibitory protein (a) does not inhibit or reduce (e.g., prevent) binding of TNFα to TNFR1, (b) does not inhibit or reduce (e.g., prevent) binding of TNFα to TNFR2, (c) does not inhibit or reduce (e.g., prevent) binding of LIGHT to LIGHTR, (d) does not inhibit or reduce (e.g., prevent) binding of LIGHT to LTβR, and (e) does not inhibit or reduce (e.g., prevent) binding of FASL to FAS.

In some embodiments, the immunoreceptor inhibitory protein exhibits one or more (e.g., 1, 2, 3, 4, and/or 5) of (a) does not specifically bind to TNFα and does not inhibit or reduce (e.g., prevent) binding of TNFα to TNFR1, (b) does not specifically bind to TNFα and does not inhibit or reduce (e.g., prevent) binding of TNFα to TNFR2, (c) does not specifically bind to LIGHT and does not inhibit or reduce (e.g., prevent) binding of LIGHT to LIGHTR, (d) does not specifically bind to LIGHT and does not inhibit or reduce (e.g., prevent) binding of LIGHT to LTβR, and/or (e) does not specifically bind to FASL and does not inhibit or reduce (e.g., prevent) binding of FASL to FAS. In some embodiments, the immunoreceptor inhibitory protein does not specifically bind to TNFα and does not inhibit or reduce (e.g., prevent) binding of TNFα to TNFR1. In some embodiments, the immunoreceptor inhibitory protein does not specifically bind to TNFα and does not inhibit or reduce (e.g., prevent) binding of TNFα to TNFR2. In some embodiments, the immunoreceptor inhibitory protein does not specifically bind to LIGHT and does not inhibit or reduce (e.g., prevent) binding of LIGHT to LIGHTR. In some embodiments, the immunoreceptor inhibitory protein does not specifically bind to LIGHT and does not inhibit or reduce (e.g., prevent) binding of LIGHT to LTβR. In some embodiments, the immunoreceptor inhibitory protein does not specifically bind to FASL and does not inhibit or reduce (e.g., prevent) binding of FASL to FAS. In some embodiments, the immunoreceptor inhibitory protein (a) docs not specifically bind to TNFα and does not inhibit or reduce (e.g., prevent) binding of TNFα to TNFR1, (b) does not specifically bind to TNFα and does not inhibit or reduce (e.g., prevent) binding of TNFα to TNFR2, (c) does not specifically bind to LIGHT and does not inhibit or reduce (e.g., prevent) binding of LIGHT to LIGHTR, (d) does not specifically bind to LIGHT and does not inhibit or reduce (e.g., prevent) binding of LIGHT to LTβR, and (e) does not specifically bind to FASL and does not inhibit or reduce (e.g., prevent) binding of FASL to FAS.

In some embodiments, the immunoreceptor inhibitory protein exhibits one or more (e.g., 1, 2, 3, 4, and/or 5) of (a) does not inhibit or reduce (e.g., prevent) binding of hTNFα to hTNFR1, (b) does not inhibit or reduce (e.g., prevent) binding of hTNFα to hTNFR2, (c) does not inhibit or reduce (e.g., prevent) binding of hLIGHT to hLIGHTR, (d) does not inhibit or reduce (e.g., prevent) binding of hLIGHT to hLTβR, and/or (e) does not inhibit or reduce (e.g., prevent) binding of hFASL to hFAS. In some embodiments, the immunoreceptor inhibitory protein docs not inhibit or reduce (e.g., prevent) binding of hTNFα to hTNFR1. In some embodiments, the immunoreceptor inhibitory protein does not inhibit or reduce (e.g., prevent) binding of hTNFα to hTNFR2. In some embodiments, the immunoreceptor inhibitory protein does not inhibit or reduce (e.g., prevent) binding of hLIGHT to hLIGHTR. In some embodiments, the immunoreceptor inhibitory protein does not inhibit or reduce (e.g., prevent) binding of hLIGHT to hLTβR. In some embodiments, the immunoreceptor inhibitory protein does not inhibit or reduce (e.g., prevent) binding of FASL to FAS. In some embodiments, the immunoreceptor inhibitory protein (a) does not inhibit or reduce (e.g., prevent) binding of hTNFα to hTNFR1, (b) does not inhibit or reduce (e.g., prevent) binding of hTNFα to hTNFR2, (c) does not inhibit or reduce (e.g., prevent) binding of hLIGHT to hLIGHTR, (d) does not inhibit or reduce (e.g., prevent) binding of hLIGHT to hLTβR, and (e) does not inhibit or reduce (e.g., prevent) binding of hFASL to hFAS.

In some embodiments, the immunoreceptor inhibitory protein exhibits one or more (e.g., 1, 2, 3, 4, and/or 5) of (a) does not specifically bind to hTNFα and does not inhibit or reduce (e.g., prevent) binding of hTNFα to hTNFR1, (b) does not specifically bind to hTNFα and docs not inhibit or reduce (e.g., prevent) binding of hTNFα to hTNFR2, (c) does not specifically bind to hLIGHT and does not inhibit or reduce (e.g., prevent) binding of hLIGHT to hLIGHTR, (d) does not specifically bind to hLIGHT and does not inhibit or reduce (e.g., prevent) binding of hLIGHT to hLTβR, and/or (e) does not specifically bind to hFASL and does not inhibit or reduce (e.g., prevent) binding of hFASL to hFAS. In some embodiments, the immunoreceptor inhibitory protein does not specifically bind to hTNFα and does not inhibit or reduce (e.g., prevent) binding of hTNFα to hTNFR1. In some embodiments, the immunoreceptor inhibitory protein does not specifically bind to hTNFα and does not inhibit or reduce (e.g., prevent) binding of hTNFα to hTNFR2. In some embodiments, the immunoreceptor inhibitory protein does not specifically bind to hLIGHT and does not inhibit or reduce (e.g., prevent) binding of hLIGHT to hLIGHTR. In some embodiments, the immunoreceptor inhibitory protein does not specifically bind to hLIGHT and does not inhibit or reduce (e.g., prevent) binding of hLIGHT to hLTβR. In some embodiments, the immunoreceptor inhibitory protein does not specifically bind to hFASL and does not inhibit or reduce (e.g., prevent) binding of hFASL to hFAS. In some embodiments, the immunoreceptor inhibitory protein (a) does not specifically bind to hTNFα and does not inhibit or reduce (e.g., prevent) binding of hTNFα to hTNFR1, (b) does not specifically bind to hTNFα and does not inhibit or reduce (e.g., prevent) binding of hTNFα to hTNFR2, (c) does not specifically bind to hLIGHT and does not inhibit or reduce (e.g., prevent) binding of hLIGHT to hLIGHTR, (d) does not specifically bind to hLIGHT and does not inhibit or reduce (e.g., prevent) binding of hLIGHT to hLTβR, and (e) does not specifically bind to hFASL and does not inhibit or reduce (e.g., prevent) binding of hFASL to hFAS.

In some embodiments, the immunoreceptor inhibitory protein does not specifically bind heparan sulfate proteoglycans. In some embodiments, the immunoreceptor inhibitory protein does not comprise a heparan sulfate proteoglycan binding domain.

In some embodiments, the immunoreceptor inhibitory proteins described herein bind a subset (e.g., one or more) of TNFSF ligands. In some embodiments, the immunoreceptor inhibitory protein specifically binds one or more of TNFα, LIGHT, FASL, and/or TL1A. In some embodiments, the immunoreceptor inhibitory protein specifically binds 2, 3, or 4 of TNFα, LIGHT, FASL, and/or TL1A. In some embodiments, the immunoreceptor inhibitory protein specifically binds TNFα, LIGHT, FASL, and/or TL1A. In some embodiments, the immunoreceptor inhibitory protein specifically binds TNFα, LIGHT, FASL, and TL1A. In some embodiments, the immunoreceptor inhibitory protein specifically binds a plurality of TNFα, LIGHT, FASL, and/or TL1A.

In some embodiments, the immunoreceptor inhibitory protein specifically binds one or more of hTNFα, hLIGHT, hFASL, and/or hTL1A. In some embodiments, the immunoreceptor inhibitory protein specifically binds 2, 3, or 4 of hTNFα, hLIGHT, hFASL, and/or hTL1A. In some embodiments, the immunoreceptor inhibitory protein specifically binds hTNFα, hLIGHT, hFASL, and/or hTL1A. In some embodiments, the immunoreceptor inhibitory protein specifically binds hTNFα, hLIGHT, hFASL, and hTL1A. In some embodiments, the immunoreceptor inhibitory protein specifically binds a plurality of hTNFα, hLIGHT, hFASL, and/or hTL1A.

In some embodiments, the immunoreceptor inhibitory protein inhibits or reduces (e.g., prevents) binding of one or more TNFSF ligand (e.g., TNFα, LIGHT, FASL, and/or TL1A) to one or more of the TNFSF ligand's (e.g., TNFα, LIGHT, FASL, and/or TL1A) cognate receptor.

In some embodiments, the immunoreceptor inhibitory protein exhibits one or more (e.g., 1, 2, 3, 4, 5, and/or 6) of (a) inhibits or reduces (e.g., prevents) binding of TNFα to TNFR1, (b) inhibits or reduces (e.g., prevents) binding of TNFα to TNFR2, (c) inhibits or reduces (e.g., prevents) binding of LIGHT to LIGHTR, (d) inhibits or reduces (e.g., prevents) binding of LIGHT to LTβR, (c) inhibits or reduces (e.g., prevents) binding of FASL to FAS, and/or (f) inhibits or reduces (e.g., prevents) binding of TL1A to DR3. In some embodiments, the immunoreceptor inhibitory protein inhibits or reduces (e.g., prevents) binding of TNFα to TNFR1. In some embodiments, the immunoreceptor inhibitory protein inhibits or reduces (e.g., prevents) binding of TNFα to TNFR2. In some embodiments, the immunoreceptor inhibitory protein inhibits or reduces (e.g., prevents) binding of LIGHT to LIGHTR. In some embodiments, the immunoreceptor inhibitory protein inhibits or reduces (e.g., prevents) binding of LIGHT to LTβR. In some embodiments, the immunoreceptor inhibitory protein inhibits or reduces (e.g., prevents) binding of FASL to FAS. In some embodiments, the immunoreceptor inhibitory protein inhibits or reduces (e.g., prevents) binding of TL1A to DR3. In some embodiments, the immunoreceptor inhibitory protein (a) inhibits or reduces (e.g., prevents) binding of TNFα to TNFR1, (b) inhibits or reduces (e.g., prevents) binding of TNFα to TNFR2, (c) inhibits or reduces (e.g., prevents) binding of LIGHT to LIGHTR, (d) inhibits or reduces (e.g., prevents) binding of LIGHT to LTβR, (c) inhibits or reduces (e.g., prevents) binding of FASL to FAS, and/or (f) inhibits or reduces (e.g., prevents) binding of TL1A to DR3.

In some embodiments, the immunoreceptor inhibitory protein exhibits one or more (e.g., 1, 2, 3, 4, 5, and/or 6) of (a) specifically binds to TNFα and inhibits or reduces (e.g., prevents) binding of TNFα to TNFR1, (b) specifically binds to TNFα and inhibits or reduces (e.g., prevents) binding of TNFα to TNFR2, (c) specifically binds to LIGHT and inhibits or reduces (e.g., prevents) binding of LIGHT to LIGHTR, (d) specifically binds to LIGHT and inhibits or reduces (e.g., prevents) binding of LIGHT to LTβR, (e) specifically binds to FASL and inhibits or reduces (e.g., prevents) binding of FASL to FAS, and/or (f) specifically binds to TL1A and inhibits or reduces (e.g., prevents) binding of TL1A to DR3. In some embodiments, the immunoreceptor inhibitory protein specifically binds to TNFα and inhibits or reduces (e.g., prevents) binding of TNFα to TNFR1. In some embodiments, the immunoreceptor inhibitory protein specifically binds to TNFα and inhibits or reduces (e.g., prevents) binding of TNFα to TNFR2. In some embodiments, the immunoreceptor inhibitory protein specifically binds to LIGHT and inhibits or reduces (e.g., prevents) binding of LIGHT to LIGHTR. In some embodiments, the immunoreceptor inhibitory protein specifically binds to LIGHT and inhibits or reduces (e.g., prevents) binding of LIGHT to LTβR. In some embodiments, the immunoreceptor inhibitory protein specifically binds to FASL and inhibits or reduces (e.g., prevents) binding of FASL to FAS. In some embodiments, the immunoreceptor inhibitory protein specifically binds to TL1A and inhibits or reduces (e.g., prevents) binding of TL1A to DR3. In some embodiments, the immunoreceptor inhibitory protein (a) specifically binds to TNFα and inhibits or reduces (e.g., prevents) binding of TNFα to TNFR1, (b) specifically binds to TNFα and inhibits or reduces (e.g., prevents) binding of TNFα to TNFR2, (c) specifically binds to LIGHT and inhibits or reduces (e.g., prevents) binding of LIGHT to LIGHTR, (d) specifically binds to LIGHT and inhibits or reduces (e.g., prevents) binding of LIGHT to LTβR, (c) specifically binds to FASL and inhibits or reduces (e.g., prevents) binding of FASL to FAS, and (f) specifically binds to TL1A and inhibits or reduces (e.g., prevents) binding of TL1A to DR3.

In some embodiments, the immunoreceptor inhibitory protein exhibits one or more (e.g., 1, 2, 3, 4, 5, and/or 6) of (a) inhibits or reduces (e.g., prevents) binding of hTNFα to hTNFR1, (b) inhibits or reduces (e.g., prevents) binding of hTNFα to hTNFR2, (c) inhibits or reduces (e.g., prevents) binding of hLIGHT to hLIGHTR, (d) inhibits or reduces (e.g., prevents) binding of hLIGHT to hLTβR, (c) inhibits or reduces (e.g., prevents) binding of hFASL to hFAS, and/or (f) inhibits or reduces (e.g., prevents) binding of hTL1A to hDR3. In some embodiments, the immunoreceptor inhibitory protein inhibits or reduces (e.g., prevents) binding of hTNFα to hTNFR1. In some embodiments, the immunoreceptor inhibitory protein inhibits or reduces (e.g., prevents) binding of hTNFα to hTNFR2. In some embodiments, the immunoreceptor inhibitory protein inhibits or reduces (e.g., prevents) binding of hLIGHT to hLIGHTR. In some embodiments, the immunoreceptor inhibitory protein inhibits or reduces (e.g., prevents) binding of hLIGHT to hLTβR. In some embodiments, the immunoreceptor inhibitory protein inhibits or reduces (e.g., prevents) binding of FASL to FAS. In some embodiments, the immunoreceptor inhibitory protein inhibits or reduces (e.g., prevents) binding of hTL1A to hDR3. In some embodiments, the immunoreceptor inhibitory protein (a) inhibits or reduces (e.g., prevents) binding of hTNFα to hTNFR1, (b) inhibits or reduces (e.g., prevents) binding of hTNFα to hTNFR2, (c) inhibits or reduces (e.g., prevents) binding of hLIGHT to hLIGHTR, (d) inhibits or reduces (e.g., prevents) binding of hLIGHT to hLTβR, (c) inhibits or reduces (e.g., prevents) binding of hFASL to hFAS, and (f) inhibits or reduces (e.g., prevents) binding of hTL1A to hDR3.

In some embodiments, the immunoreceptor inhibitory protein exhibits one or more (e.g., 1, 2, 3, 4, 5, and/or 6) of (a) specifically binds to hTNFα and inhibits or reduces (e.g., prevents) binding of hTNFα to hTNFR1, (b) specifically binds to hTNFα and inhibits or reduces (e.g., prevents) binding of hTNFα to hTNFR2, (c) specifically binds to hLIGHT and inhibits or reduces (e.g., prevents) binding of hLIGHT to hLIGHTR, (d) specifically binds to hLIGHT and inhibits or reduces (e.g., prevents) binding of hLIGHT to hLTβR, (c) specifically binds to hFASL and inhibits or reduces (e.g., prevents) binding of hFASL to hFAS, and/or (f) specifically binds to hTL1A and inhibits or reduces (e.g., prevents) binding of hTL1A to hDR3. In some embodiments, the immunoreceptor inhibitory protein specifically binds to hTNFα and inhibits or reduces (e.g., prevents) binding of hTNFα to hTNFR1. In some embodiments, the immunoreceptor inhibitory protein specifically binds to hTNFα and inhibits or reduces (e.g., prevents) binding of hTNFα to hTNFR2. In some embodiments, the immunoreceptor inhibitory protein specifically binds to hLIGHT and inhibits or reduces (e.g., prevents) binding of hLIGHT to hLIGHTR. In some embodiments, the immunoreceptor inhibitory protein specifically binds to hLIGHT and inhibits or reduces (e.g., prevents) binding of hLIGHT to hLTβR. In some embodiments, the immunoreceptor inhibitory protein specifically binds to hFASL and inhibits or reduces (e.g., prevents) binding of hFASL to hFAS. In some embodiments, the immunoreceptor inhibitory protein specifically binds to hTL1A and inhibits or reduces (e.g., prevents) binding of hTL1A to hDR3. In some embodiments, the immunoreceptor inhibitory protein (a) specifically binds to hTNFα and inhibits or reduces (e.g., prevents) binding of hTNFα to hTNFR1, (b) specifically binds to hTNFα and inhibits or reduces (e.g., prevents) binding of hTNFα to hTNFR2, (c) specifically binds to hLIGHT and inhibits or reduces (e.g., prevents) binding of hLIGHT to hLIGHTR, (d) specifically binds to hLIGHT and inhibits or reduces (e.g., prevents) binding of hLIGHT to hLTβR, (e) specifically binds to hFASL and inhibits or reduces (e.g., prevents) binding of hFASL to hFAS, and (f) specifically binds to hTL1A and inhibits or reduces (e.g., prevents) binding of hTL1A to hDR3.

For the sake of clarity, and as further described herein, where an immunoreceptor inhibitory protein described herein specifically binds more than one TNFSF ligand (e.g., specifically binds TNFα, LIGHT, FASL, and TL1A), the binding affinity of the immunoreceptor inhibitory protein for each TNFSF ligand can vary.

In some embodiments, the immunoreceptor inhibitory protein specifically binds heparan sulfate proteoglycans. In some embodiments, the immunoreceptor inhibitory protein comprises a heparan sulfate proteoglycan binding domain.

In some embodiments, the protein specifically binds to trimeric forms of TL1A (e.g., hTL1A) and monomeric forms of TL1A (e.g., hTL1A). In some embodiments, the protein preferentially specifically binds to trimeric forms of TL1A (e.g., hTL1A) relative to monomeric forms of TL1A (e.g., hTL1A). In some embodiments, the protein specifically binds to trimeric forms of TL1A (e.g., hTL1A) with higher affinity relative to monomeric forms of TL1A (e.g., hTL1A). In some embodiments, the protein specifically binds to trimeric forms of TL1A (e.g., hTL1A) with higher affinity relative to monomeric forms of TL1A (e.g., hTL1A), as measured by surface plasmon resonance (SPR) (e.g., as described in Example 10).

In some embodiments, the binding affinity of the protein to trimeric TL1A (e.g., hTL1A) is higher than the binding affinity of the protein to monomeric TL1A (e.g., hTL1A). In some embodiments, the binding affinity of the protein to trimeric TL1A (e.g., hTL1A) is at least 1, 2, 3, 4, 5, 6, 7, 8, 9, or 10-fold higher than the binding affinity of the protein to monomeric TL1A (e.g., hTL1A).

In some embodiments, the binding affinity of the protein to trimeric TL1A (e.g., hTL1A) is higher than the binding affinity of the protein to monomeric TL1A (e.g., hTL1A), as measured by SPR (e.g., as described in Example 10). In some embodiments, the binding affinity of the protein to trimeric TL1A (e.g., hTL1A) is at least 1, 2, 3, 4, 5, 6, 7, 8, 9, or 10-fold higher than the binding affinity of the protein to monomeric TL1A (e.g., hTL1A), as measured by SPR (e.g., as described in Example 10).

In some embodiments, the binding affinity of the protein to trimeric TL1A (e.g., hTL1A) as measured by dissociation equilibrium constant (KD-trimer) is higher than the binding affinity of the protein to monomeric TL1A (e.g., hTL1A) as measured by dissociation equilibrium constant (KD-monomer). In some embodiments, the binding affinity of the protein to trimeric TL1A (e.g., hTL1A) as measured by dissociation equilibrium constant (KD-trimer) is at least 1, 2, 3, 4, 5, 6, 7, 8, 9, or 10-fold higher than the binding affinity of the protein to monomeric TL1A (e.g., hTL1A) as measured by dissociation equilibrium constant (KD-monomer).

In some embodiments, the binding affinity of the protein to trimeric TL1A (e.g., hTL1A) as measured by dissociation equilibrium constant (KD-trimer) is higher than the binding affinity of the protein to monomeric TL1A (e.g., hTL1A) as measured by dissociation equilibrium constant (KD-monomer), as measured by SPR (e.g., as described in Example 10). In some embodiments, the binding affinity of the protein to trimeric TL1A (e.g., hTL1A) as measured by dissociation equilibrium constant (KD-trimer) is at least 1, 2, 3, 4, 5, 6, 7, 8, 9, or 10-fold higher than the binding affinity of the protein to monomeric TL1A (e.g., hTL1A) as measured by dissociation equilibrium constant (KD-monomer), as measured by SPR (e.g., as described in Example 10).

In some embodiments, the KD value of the protein binding to trimeric TL1A (e.g., hTL1A) (KD-trimer) is lower than the KD value of the protein binding to monomeric TL1A (e.g., hTL1A) (KD-monomer). In some embodiments, the KD value of the protein binding to trimeric TL1A (e.g., hTL1A) (KD-trimer) is at least 1, 2, 3, 4, 5, 6, 7, 8, 9, or 10-fold lower than the KD value of the protein binding to monomeric TL1A (e.g., hTL1A) (KD-monomer).

In some embodiments, the KD value of the protein binding to trimeric TL1A (e.g., hTL1A) (KD-trimer) is lower than the KD value of the protein binding to monomeric TL1A (e.g., hTL1A) (KD-monomer), as measured by SPR (e.g., as described in Example 10). In some embodiments, the KD value of the protein binding to trimeric TL1A (e.g., hTL1A) (KD-trimer) is at least 1, 2, 3, 4, 5, 6, 7, 8, 9, or 10-fold lower than the KD value of the protein binding to monomeric TL1A (e.g., hTL1A) (KD-monomer), as measured by SPR (e.g., as described in Example 10).

In some embodiments, the binding affinity of the protein to trimeric TL1A (e.g., hTL1A) as measured by dissociation equilibrium constant (KD-trimer) is less than 20 pM, 15 pM, or 10 pM. In some embodiments, the binding affinity of the protein to monomeric TL1A (e.g., hTL1A) as measured by dissociation equilibrium constant (KD-monomer) is more than 60 pM, 70 pM, or 80 pM.

In some embodiments, the binding affinity of the protein to trimeric TL1A (e.g., hTL1A) as measured by dissociation equilibrium constant (KD-trimer) is less than 20 pM, 15 pM, or 10 pM, as measured by SPR (e.g., as described in Example 10). In some embodiments, the binding affinity of the protein to monomeric TL1A (e.g., hTL1A) as measured by dissociation equilibrium constant (KD-monomer) is more than 60 pM, 70 pM, or 80 pM, as measured by SPR (e.g., as described in Example 10).

In some embodiments, the binding affinity of the protein to trimeric TL1A (e.g., hTL1A) as measured by dissociation equilibrium constant (KD-trimer) is from about 1-20 pM, 1-19 pM, 1-18 pM, 1-17 pM, 1-16 pM, 1-15 pM, 1-14 pM, 1-13 pM, 1-12 pM, 1-11 pM, 1-10 pM, 1-9 pM, 1-8 pM, 1-7 pM, 1-6 pM, 1-5 pM, 1-4 pM, 1-3 pM, 1-2 pM. In some embodiments, the binding affinity of the protein to trimeric TL1A (e.g., hTL1A) as measured by dissociation equilibrium constant (KD-trimer) is from about 5-15 pM, 5-13 pM, 5-12 pM, 5-11 pM, 5-10 pM. In some embodiments, the binding affinity of the protein to trimeric TL1A (e.g., hTL1A) as measured by dissociation equilibrium constant (KD-trimer) is from about 5-15 pM.

In some embodiments, the binding affinity of the protein to trimeric TL1A (e.g., hTL1A) as measured by dissociation equilibrium constant (KD-trimer) is from about 1-20 pM, 1-19 pM, 1-18 pM, 1-17 pM, 1-16 pM, 1-15 pM, 1-14 pM, 1-13 pM, 1-12 pM, 1-11 pM, 1-10 pM, 1-9 pM, 1-8 pM, 1-7 pM, 1-6 pM, 1-5 pM, 1-4 pM, 1-3 pM, 1-2 pM, as measured by SPR (e.g., as described in Example 10). In some embodiments, the binding affinity of the protein to trimeric TL1A (e.g., hTL1A) as measured by dissociation equilibrium constant (KD-trimer) is from about 5-15 pM, 5-13 pM, 5-12 pM, 5-11 pM, 5-10 pM, as measured by SPR (e.g., as described in Example 10). In some embodiments, the binding affinity of the protein to trimeric TL1A (e.g., hTL1A) as measured by dissociation equilibrium constant (KD-trimer) is from about 5-15 pM, as measured by SPR (e.g., as described in Example 10).

In some embodiments, the binding affinity of the protein to monomeric TL1A (e.g., hTL1A) as measured by dissociation equilibrium constant (KD-monomer) is from about 60-100 pM, 60-95 pM, 60-90 pM, 60-85 pM, 60-80 pM, 60-75 pM, 60-70 pM, or 60-65 pM. In some embodiments, the binding affinity of the protein to monomeric TL1A (e.g., hTL1A) as measured by dissociation equilibrium constant (KD-monomer) is from about 65-85 pM, 65-80 pM, 70-85 pM, or 70-80 pM.

In some embodiments, the binding affinity of the protein to monomeric TL1A (e.g., hTL1A) as measured by dissociation equilibrium constant (KD-monomer) is from about 60-100 pM, 60-95 pM, 60-90 pM, 60-85 pM, 60-80 pM, 60-75 pM, 60-70 pM, or 60-65 pM, as measured by SPR (e.g., as described in Example 10). In some embodiments, the binding affinity of the protein to monomeric TL1A (e.g., hTL1A) as measured by dissociation equilibrium constant (KD-monomer) is from about 65-85 pM, 65-80 pM, 70-85 pM, or 70-80 pM, as measured by SPR (e.g., as described in Example 10).

In some embodiments, the association rate (kₐ) of the protein to trimeric TL1A (e.g., hTL1A) (kₐ-trimer) is at least 1, 2, 3, 4, 5, 6, 7, 8, 9, or 10-fold higher than the association rate (kₐ) of the protein to monomeric TL1A (e.g., hTL1A) as (kₐ-monomer).

In some embodiments, the association rate (kₐ) of the protein to trimeric TL1A (e.g., hTL1A) (kₐ-trimer) is at least 1, 2, 3, 4, 5, 6, 7, 8, 9, or 10-fold higher than the association rate (kₐ) of the protein to monomeric TL1A (e.g., hTL1A) as (kₐ-monomer), as measured by SPR (e.g., as described in Example 10).

In some embodiments, the disassociation rate (k_{d}) of the protein to trimeric TL1A (e.g., hTL1A) (k_{d}-trimer) is at least 0.1, 0.2, 0.3, 0.4, 0.5, 1, 2, 3, 4, 5, 6, 7, 8, 9, or 10-fold lower than the disassociation rate (k_{d}) of the protein to monomeric TL1A (e.g., hTL1A) as (k_{d}-monomer).

In some embodiments, the disassociation rate (k_{d}) of the protein to trimeric TL1A (e.g., hTL1A) (k_{d}-trimer) is at least 0.1, 0.2, 0.3, 0.4, 0.5, 1, 2, 3, 4, 5, 6, 7, 8, 9, or 10-fold lower than the disassociation rate (k_{d}) of the protein to monomeric TL1A (e.g., hTL1A) as (k_{d}-monomer), as measured by SPR (e.g., as described in Example 10).

Binding affinity can be measured by standard assays known in the art. For example, binding affinity can be measured by surface plasmon resonance (SPR) (e.g., BIAcore®-based assay), a common method known in the art (see, e.g., Wilson, Science 295:2103, 2002; Wolff et al., Cancer Res. 55:2560, 1993; and U.S. Pat. Nos. 5,283,173, 5,468,614, the full contents of each of which are incorporated by reference herein for all purposes). SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules (e.g., proteins). The dissociation constant for the complex can be determined by monitoring changes in the refractive index with respect to time as buffer is passed over the chip.

Other suitable assays for measuring the binding of one protein to another (e.g., binding of a protein described herein to a TNFSF ligand) include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, analytical ultracentrifugation, spectroscopy, flow cytometry, sequencing and other methods for detection of binding of proteins.

### 5.4 Immunoreceptor Inhibitory Protein Fusions & Conjugates

In some embodiments, the immunoreceptor inhibitory protein (e.g., described herein) is operably connected to a heterologous moiety (e.g., a heterologous polypeptide) forming a fusion or conjugate protein, respectively. As such, further provided herein are, inter alia, fusion proteins comprising an immunoreceptor inhibitory protein (e.g., described herein) and one or more heterologous proteins (or a functional fragment, functional variant, or domain thereof). Further provided herein are, inter alia, conjugates comprising an immunoreceptor inhibitory protein (e.g., described herein) (or a nucleic acid molecule encoding an immunoreceptor inhibitory protein (e.g., described herein) and one or more heterologous moieties.

Heterologous moieties can include a variety of molecules, that can be operably connected to the an immunoreceptor inhibitory protein (e.g., described herein). Such operable connection allows for the function of the immunoreceptor inhibitory protein (e.g., described herein) to be maintained when connected to the heterologous moiety.

Heterologous moieties include, but are not limited to, proteins, peptides, small molecules, nucleic acid molecules (e.g., DNA, RNA, DNA/RNA hybrid molecules), carbohydrates, lipids, synthetic polymers (e.g., polymers of PEG), and any combination thereof. In some embodiments, the heterologous moiety is a detectable moiety (e.g., a protein, e.g., a fluorescent protein). In some embodiments, the heterologous moiety is an imaging agent. In some embodiments, the heterologous moiety comprises a radioligand. In some embodiments, the heterologous moiety is a diagnostic agent. In some embodiments, the heterologous moiety is a non-effector moiety, e.g., a protein sequence that acts as a “handle” or linker but has otherwise no independent biological effect. In some embodiments, the heterologous moiety is a therapeutic agent.

In some embodiments, the heterologous moiety (e.g., protein) comprises an antibody, an antibody mimetic, or one or more Ig constant region (e.g., an Fc region). In some embodiments, the heterologous moiety comprises one or more Ig constant region (e.g., Fc region). In some embodiments, the heterologous moiety comprises an Fc region. The heterologous moiety can be any one or more of (any combination of) the foregoing.

### 5.4.1 Radioligands

In some embodiments, the heterologous moiety comprises a radioisotope. As such, provided herein are radioligands comprising an immunoreceptor inhibitory protein (e.g., described herein) operably connected (e.g., through a linker) to one more radioisotope. In some embodiments, the radioisotope acts as a therapeutic agent. In some embodiments, the radioisotope acts as an imaging agent. In some embodiments, the immunoreceptor inhibitory protein (e.g., described herein) acts as a targeting moiety for the radioisotope. In some embodiments, the radioisotope and the immunoreceptor inhibitory protein (e.g., described herein) are operably connected through a linker.

Radioisotopes are known in the art. See, e.g., Sgouros, G., Bodei, L., McDevitt, M. R. et al. Radiopharmaceutical therapy in cancer: clinical advances and challenges. Nat Rev Drug Discov 19, 589-608 (2020). https://doi.org/10.1038/s41573-020-0073-9; and Zhang, Longjiang et al. “Delivery of therapeutic radioisotopes using nanoparticle platforms: potential benefit in systemic radiation therapy.” Nanotechnology, science and applications vol. 3 159-70. 3 Dec. 2010, doi: 10.2147/NSA.S7462; the entire contents of each of which are incorporated herein by reference for all purposes.

Exemplary radioisotopes include, but are not limited to, Lutetium-177, Radium-223, Iodine-131, Iodine-125, Fluorine-18, Ir-192, Xenon-133, Yttrium-90, Carbon-11, Idium-111, Strontium-89, Copper-67, Copper-64, Rhenium-186, Actinium-225, Astatine-211, Bismuth-213, Bismuth-212, Samarium-153, Holmium-166, Thorium-227, and Lead-212.

Radioisotopes releases radiation as they break down and become more stable. As such, any radioisotope will be applicable to the radioligands described herein, independent of the specific species.

Methods of operably connecting proteins to radionuclides (e.g., through one or more linkers) are known in the art. See, e.g., Gupta, Suprit et al. “Antibody labeling with radioiodine and radiometals.” Methods in molecular biology (Clifton, N.J.) vol. 1141 (2014): 147-57. doi: 10.1007/978-1-4939-0363-4_9; Marion Chomet, State of the Art in Radiolabeling of Antibodies with Common and Uncommon Radiometals for Preclinical and Clinical Immuno-PET, Bioconjugate Chem. 2021, 32, 7, 1315-1330; Martina Steiner, Dario Neri; Antibody-Radionuclide Conjugates for Cancer Therapy: Historical Considerations and New Trends. Clin Cancer Res 15 Oct. 2011; 17 (20): 6406-6416. https://doi.org/10.1158/1078-0432.CCR-11-0483; the entire contents of each of which are incorporated herein by reference for all purposes.

### 5.4.2 Chimeric Antigen Receptors

In some embodiments, an immunoreceptor inhibitory protein described herein is part of a chimeric antigen receptor (CAR). In some embodiments, an immunoreceptor inhibitory protein described herein is the extracellular antigen-binding domain of a CAR. Standard CAR domains are known in art, including, e.g., transmembrane domains and intracellular signaling domains. See, e.g., WO2024056809, WO2023240064A1, and WO2023205148A1, WO2023133092A1, the entire contents of each of which is incorporated herein by reference for all purposes. The general structure and function of CARs is known the art (see, e.g., references cited above); and as such any CAR will be applicable to the CARs described herein, independent of the specific species.

Exemplary transmembrane domains include, e.g., the alpha, beta or zeta chain of T-cell receptor, CD28, CD3 epsilon, CD45, CD4, CD5, CD8 (for example, CD8 alpha, CD8 beta), CD9, CD 16, CD22, CD33, CD37, CD64, CD80, CD86, CD134, CD137, CD154. In some embodiments, a transmembrane domain may include at least the transmembrane region(s) of a costimulatory molecule, for example, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CDlla/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDlld, ITGAE, CD103, ITGAL, CDlla, LFA-1, ITGAM, CDllb, ITGAX, CDllc, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD 150, IPO-3), BLAME (SLAMF8), SELPLG (CD 162), LTBR, LAT, GADS, SLP-76, PAG/Cbp, CD 19a, and a ligand that specifically binds with CD83. In some instances, the transmembrane domain can be attached to the extracellular region of the CAR, for example, the antigen-binding domain of the CAR, via a hinge, for example, a hinge from a human protein. For example, in some embodiments, the hinge can be a human Ig (immunoglobulin) hinge, for example, an IgG4 hinge, or a CD8a hinge.

Exemplary intracellular signaling domains include, e.g., the cytoplasmic sequences of the T cell receptor (TCR) and co-receptors that act in concert to initiate signal transduction following antigen receptor engagement, as well as any derivative or variant of these sequences and any recombinant sequence that has the same functional capability. In some embodiments, the intracellular signaling domain comprises a primary signaling domain and one or more costimulatory signaling domain. Exemplary primary signaling domains, include, e.g., intracellular signaling domains of TCR zeta, FcR gamma, FcR beta, CD3 gamma, CD3 delta, CD3 epsilon, CD5, CD22, CD79a, CD79b, CD278 (also known as “ICOS”), FccRI, DAP10, DAP12, CD32, and CD66d. Exemplary of proteins with costimulatory domains suitable for use in CAR described herein include, e.g., MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD1 la/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD lid, ITGAE, CD 103, ITGAL, CDlla, LFA-1, ITGAM, CDllb, ITGAX, CDllc, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRT AM, Ly9 (CD229), CD 160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, CD 19a, and a ligand that specifically binds with CD83, and the like.

### 5.4.3 Signal Peptides

In some embodiments, the heterologous polypeptide is a heterologous signal peptide. Heterologous signal peptides are known in the art. In some embodiments, the immunoreceptor inhibitory protein comprises a heterologous signal peptide operably connected to the immunoreceptor inhibitory protein. In some embodiments, the immunoreceptor inhibitory protein comprises a heterologous signal peptide operably connected to the N-terminus of the immunoreceptor inhibitory protein. In some embodiments, the amino acid sequence of the immunoreceptor inhibitory protein comprises the amino acid sequence set forth in any one of SEQ ID NOS: 23-84, 187-190, or 199-200 and comprises a heterologous signal peptide operably connected to the N-terminus of the immunoreceptor inhibitory protein. In some embodiments, the amino acid sequence of the immunoreceptor inhibitory protein consists of the amino acid sequence set forth in any one of SEQ ID NOS: 23-84, 187-190, or 199-200 and comprises a heterologous signal peptide operably connected to the N-terminus of the immunoreceptor inhibitory protein. In some embodiments, the amino acid sequence of the immunoreceptor inhibitory protein comprises the amino acid sequence set forth in any one of SEQ ID NOS: 23-53, 187-188, or 199 and comprises a heterologous signal peptide operably connected to the N-terminus of the immunoreceptor inhibitory protein. In some embodiments, the amino acid sequence of the immunoreceptor inhibitory protein consists of the amino acid sequence set forth in any one of SEQ ID NOS: 23-53, 187-188, or 199 and comprises a heterologous signal peptide operably connected to the N-terminus of the immunoreceptor inhibitory protein.

Commonly used heterologous signal peptides are known in the art, for example, the native signal peptide of human interleukin 2 (hIL-2), human oncostatin M (hOSM), human chymotrypsinogen (hCTRB1), human trypsinogen 2 (hTRY2), and human insulin (hINS). A person of ordinary skill can determine the appropriate signal peptide using standard methodology known in the art. The amino acid sequence of exemplary signal peptides is provided in Table 3.

TABLE 3
The Amino Acid Sequence of Exemplary
Signal Peptides.

| | | SEQ |
|---|---|---|
| | | ID |
| Description | Amino Acid Sequence | NO |
| hIL-2 | MYRMQLLSCIALSLALVINS | 85 |
| hOSM | MGVLLTQRTLLSLVLALLFP | 86 |
| | SMASM | |
| hCTRB1 | MASLWLLSCFSLVGAAFG | 87 |
| hTRY2 | MNLLLILTFVAAAVA | 88 |
| hINS | MALWMRLLPLLALLALWGP | 89 |
| | DPAAA | |

In some embodiments, the amino acid sequence of the signal peptide comprises the amino acid sequence of any one of the signal peptides set forth in Table 3. In some embodiments, the amino acid sequence of the signal peptide comprises the amino acid sequence of any one of the signal peptides set forth in Table 3, and further comprises 1 or more but less than 15% (less than 12%, less than 10%, less than 8%), amino acid variations (e.g., amino acid substitutions, deletions, or additions). In some embodiments, the amino acid sequence of the signal peptide comprises the amino acid sequence of any one of the signal peptides set forth in Table 3, comprising 1, 2, or 3 amino acid variations (e.g., substitutions, deletions, additions). In some embodiments, the amino acid sequence of the signal peptide comprises the amino acid sequence of any one of the signal peptides set forth in Table 3, and further comprises 1 or more but less than 15% (less than 12%, less than 10%, less than 8%), amino acid substitutions. In some embodiments, the amino acid sequence of the signal peptide comprises the amino acid sequence of any one of the signal peptides set forth in Table 3, comprising 1, 2, or 3 amino acid substitutions.

In some embodiments, the amino acid sequence of the signal peptide consists of the amino acid sequence of any one of the signal peptides set forth in Table 3. In some embodiments, the amino acid sequence of the signal peptide consists of the amino acid sequence of any one of the signal peptides set forth in Table 3, and further consists of 1 or more but less than 15% (less than 12%, less than 10%, less than 8%), amino acid variations (e.g., amino acid substitutions, deletions, or additions). In some embodiments, the amino acid sequence of the signal peptide consists of the amino acid sequence of any one of the signal peptides set forth in Table 3, comprising 1, 2, or 3 amino acid variations (e.g., substitutions, deletions, additions). In some embodiments, the amino acid sequence of the signal peptide consists of the amino acid sequence of any one of the signal peptides set forth in Table 3, and further consists of 1 or more but less than 15% (less than 12%, less than 10%, less than 8%), amino acid substitutions. In some embodiments, the amino acid sequence of the signal peptide consists of the amino acid sequence of any one of the signal peptides set forth in Table 3, comprising 1, 2, or 3 amino acid substitutions.

In some embodiments, the amino acid sequence of the signal peptide comprises the amino acid sequence set forth in any one of SEQ ID NOS: 85-89. In some embodiments, the amino acid sequence of the signal peptide comprises the amino acid sequence set forth in any one of SEQ ID NOS: 85-89, and further comprises 1 or more but less than 15% (less than 12%, less than 10%, less than 8%), amino acid variations (e.g., amino acid substitutions, deletions, or additions). In some embodiments, the amino acid sequence of the signal peptide comprises the amino acid sequence set forth in any one of SEQ ID NOS: 85-89, comprising 1, 2, or 3 amino acid variations (e.g., substitutions, deletions, additions). In some embodiments, the amino acid sequence of the signal peptide comprises the amino acid sequence set forth in any one of SEQ ID NOS: 85-89, and further comprises 1 or more but less than 15% (less than 12%, less than 10%, less than 8%), amino acid substitutions. In some embodiments, the amino acid sequence of the signal peptide comprises the amino acid sequence set forth in any one of SEQ ID NOS: 85-89, comprising 1, 2, or 3 amino acid substitutions.

In some embodiments, the amino acid sequence of the signal peptide consists of the amino acid sequence set forth in any one of SEQ ID NOS: 85-89. In some embodiments, the amino acid sequence of the signal peptide consists of the amino acid sequence set forth in any one of SEQ ID NOS: 85-89, and further consists of 1 or more but less than 15% (less than 12%, less than 10%, less than 8%), amino acid variations (e.g., amino acid substitutions, deletions, or additions). In some embodiments, the amino acid sequence of the signal peptide consists of the amino acid sequence set forth in any one of SEQ ID NOS: 85-89, comprising 1, 2, or 3 amino acid variations (e.g., substitutions, deletions, additions). In some embodiments, the amino acid sequence of the signal peptide consists of the amino acid sequence set forth in any one of SEQ ID NOS: 85-89, and further consists of 1 or more but less than 15% (less than 12%, less than 10%, less than 8%), amino acid substitutions. In some embodiments, the amino acid sequence of the signal peptide consists of the amino acid sequence set forth in any one of SEQ ID NOS: 85-89, comprising 1, 2, or 3 amino acid substitutions.

### 5.4.4 Half-Life Extension Moieties

In some embodiments, the heterologous moiety (e.g., protein) is a half-life extension moiety (e.g., protein).

As described herein, half-life extension moieties increase the half-life of the subject protein in vivo when administered to a subject (e.g., a human subject) (e.g., relative to the subject protein absent the half-life extension moiety). As such, any half-life extension moiety (e.g., described herein, otherwise known in the art, etc.) will be applicable in the same way to the conjugate and fusion proteins described herein, independent of the specific species of half-life extension moiety utilized. Various moieties sharing said half-life extension property are known in the art and described herein. See, e.g., Ko S, Jo M, Jung S T. Recent Achievements and Challenges in Prolonging the Serum Half-Lives of Therapeutic IgG Antibodies Through Fc Engineering. BioDrugs. 2021; 35 (2): 147-157. doi: 10.1007/s40259-021-00471-0 (hereinafter “Ko 2021”); Bech, E. M., Pedersen, S. L., & Jensen, K. J. (2018). Chemical Strategies for Half-Life Extension of Biopharmaceuticals: Lipidation and Its Alternatives. ACS medicinal chemistry letters, 9 (7), 577-580. https://doi.org/10.1021/acsmedchemlett.8b00226 (hereinafter “Bech 2018”); Mester S, Evers M, Meyer S, et al. Extended plasma half-life of albumin-binding domain fused human IgA upon pH-dependent albumin engagement of human FcRn in vitro and in vivo. MAbs. 2021; 13 (1): 1893888. doi: 10.1080/19420862.2021.1893888 (hereinafter “Mester 2021”); Kontermann R E. Strategies for extended serum half-life of protein therapeutics. Curr Opin Biotechnol. 2011; 22 (6): 868-876. doi: 10.1016/j.copbio.2011.06.012 (hereinafter “Kontermann 2011”); Strohl W. R. (2015). Fusion Proteins for Half-Life Extension of Biologics as a Strategy to Make Biobetters. BioDrugs: clinical immunotherapeutics, biopharmaceuticals and gene therapy, 29 (4), 215-239. https://doi.org/10.1007/s40259-015-0133-6; Zaman R, Islam R A, Ibnat N, et al. Current strategies in extending half-lives of therapeutic proteins. J Control Release. 2019; 301:176-189. doi: 10.1016/j.jconrel.2019.02.016; Chen C, Constantinou A, Chester K A, et al. Glycoengineering approach to half-life extension of recombinant biotherapeutics. Bioconjug Chem. 2012; 23 (8): 1524-1533. doi: 10.1021/bc200624a; Gupta, Vijayalaxmi et al. “Protein PEGylation for cancer therapy: bench to bedside.” Journal of cell communication and signaling vol. 13, 3 (2019): 319-330. doi: 10.1007/s12079-018-0492-0; Martin Schlapschy, et al, PASylation: a biological alternative to PEGylation for extending the plasma half-life of pharmaceutically active proteins, Protein Engineering, Design and Selection, Volume 26, Issue 8, August 2013, Pages 489-501, https://doi.org/10.1093/protein/gzt023; Strohl, William R. “Fusion Proteins for Half-Life Extension of Biologics as a Strategy to Make Biobetters.” BioDrugs: clinical immunotherapeutics, biopharmaceuticals and gene therapy vol. 29, 4 (2015): 215-39. doi: 10.1007/s40259-015-0133-6; the entire contents of each of which are incorporated by reference herein for all purposes.

Exemplary half-life extension moieties include, but are not limited to, an immunoglobulin (e.g., human Ig (hIg), murine Ig (mIg)), a fragment of an Ig (e.g., hIg, mIg), an Ig (e.g., hIg, mIg) constant region, a fragment of an Ig (e.g., hIg, mIg) constant region, an Ig (e.g., hIg, mIg) Fc region, human transferrin, a human transferrin binding moiety (e.g., small molecule, lipid, protein, peptide, etc.), human serum albumin (HSA), a fragment of HSA, an HSA binding moiety (e.g., small molecule, lipid, protein, peptide, etc.) (e.g., an antibody, a Streptococcal protein G (see, e.g., Mester 2021)), polyethylene glycol (PEG) (and polymers thereof) (e.g., pegylation), lipids, small molecules, carbohydrates (e.g., glycosylation, polysialic acid (polysialylation), hydroxyethyl starch (HES) (HESylation), heparosan (HEPylation)), acetylated peptides that bind HSA (see, e.g., Zorzi, A., Middendorp, S., Wilbs, J. et al. Acylated heptapeptide binds albumin with high affinity and application as tag furnishes long-acting peptides. Nat Commun 8, 16092 (2017). https://doi.org/10.1038/ncomms16092 (hereinafter “Zorzi 2017”), the entire contents of which are incorporated herein by reference for all purposes).

In specific embodiments, the half-life extension moiety is HSA (or a functional fragment thereof). In specific embodiments, the half-life extension moiety is HSA binding moiety (e.g., small molecule, lipid, protein, peptide, etc.) (e.g., an acetylated peptide (see, e.g.,)) (e.g., an antibody, a Streptococcal protein G (see, e.g., Mester 2021)). In specific embodiments, the half-life extension moiety is HSA binding antibody or antibody mimetic. In specific embodiments, the half-life extension moiety is polyethylene glycol (PEG) (and polymers thereof) (e.g., pegylation). In specific embodiments, the half-life extension moiety is an acetylated peptide that specifically bind HAS (see, e.g., Zorzi 2017).

In some embodiments, the heterologous polypeptide is a half-life extension polypeptide. Exemplary half-life extension polypeptides include, but are not limited to, an Ig, a fragment of an Ig, one or more Ig heavy chain constant region, a fragment of an Ig constant region, an Ig Fc region, a hIg, a fragment of a hIg, one or more hIg heavy chain constant region, a fragment of a hIg constant region, a hIg Fc region, a mIg, a fragment of a mIg, one or more mIg heavy chain constant region, a fragment of a mIg constant region, a mIg Fc region, human transferrin, a fragment of human transferrin, a human transferrin binding protein (e.g., an antibody) HSA, and HSA binding proteins (e.g., an antibody, a Streptococcal protein G).

In some embodiments, the half-life extension polypeptide comprises an Ig Fc region (e.g., hIg Fc region). See, e.g., §§ 5.4.5.2, 5.4.5.3, and 5.4.5.4. In specific embodiments, the half-life extension polypeptide comprises an Ig Fc region described herein. In specific embodiments, the half-life extension polypeptide comprises an Ig Fc region described in any one of §§ 5.4.5.2, 5.4.5.3, or 5.4.5.4. Ig Fc regions of various isotypes, classes, and subclasses (e.g., IgG (e.g., IgG1, IgG2, IgG3, and IgG4)) (and variants (e.g., engineered variants (e.g., described herein))) share a common general property known in the art of enhancing half-life of proteins to which they are fused. As such, any Ig Fc region (e.g., described herein, known in the art, etc.) will work independently in the fusion proteins described herein regardless of the specific isotype, class, subclass, etc of the Ig Fc.

In some embodiments, the Ig (e.g., hIg, mIg) Fc region of a fusion protein described herein comprises one or more amino acid variation (e.g., relative to a reference Ig (e.g., hIg, mIg) Fc region (e.g., a wild-type Ig (e.g., hIg, mIg) Fc region)) that enhances serum half-life of the fusion protein (e.g., relative to a reference Ig (e.g., hIg, mIg) Fc region (e.g., a wild-type Ig (e.g., hIg, mIg) Fc region)). See, e.g., § 5.4.5.3. In some embodiments, the Ig (e.g., hIg, mIg) Fc region of a fusion protein described herein comprises one or more amino acid variation (e.g., relative to a reference Ig (e.g., hIg, mIg) Fc region (e.g., a wild-type Ig (e.g., hIg, mIg) Fc region)) described in § 5.4.5.3.

In some embodiments, half-life extension is mediated through one or more of lipidation, glycosylation, polysialylation, HESylation, HEPylation, and/or pegylation. In some embodiments, half-life extension is mediated through one or more of lipidation, HESylation, HEPylation, and/or pegylation. In some embodiments, half-life extension is mediated through glycosylation. In some embodiments, half-life extension is mediated through polysialylation.

In some embodiments, the half-life extension moiety comprises one or more lipids. See, e.g., Bech 2018. In some embodiments, the half-life extension moiety comprises one or more post translational modifications (e.g., glycosylation, polysialylation, etc.).

In some embodiments, the half-life extension moiety (e.g., protein) is altered (e.g., compared to a reference half-life extension moiety (e.g., protein)) to further enhance half-life of the fusion protein or conjugate. Various alterations to known half-life extension moieties (e.g., proteins) are known in the art. See, e.g., Ko 2021, Bech 2018, Mester 2021, and Kontermann 2011. Modifications include, e.g., amino acid variations (e.g., substitutions, additions, deletions) and post translational modifications (e.g., altered lipidation, glycosylation, polysialylation, HESylation, HEPylation, pegylation, etc.).

The immunoreceptor inhibitory protein described herein fused or conjugated to a half-life extending moiety or a half-life extending moiety can be evaluated for their pharmacokinetic properties utilizing standard in vivo methods known in the art. See, e.g., Avery, Lindsay B et al. “Utility of a human FcRn transgenic mouse model in drug discovery for early assessment and prediction of human pharmacokinetics of monoclonal antibodies.” mAbs vol. 8, 6 (2016): 1064-78. doi: 10.1080/19420862.2016.1193660; Conner, Christopher M et al. “A precisely humanized FCRN transgenic mouse for preclinical pharmacokinetics studies.” Biochemical pharmacology vol. 210 (2023): 115470. doi: 10.1016/j.bcp.2023.115470; and Kathryn Ball et al., PK and Biodistribution of Therapeutic Proteins, Drug Metabolism and Disposition Jun. 1, 2022, 50 (6) 858-866; DOI: https://doi.org/10.1124/dmd.121.000463 (hereinafter “Ball 2022”), the entire contents of each of which are incorporated herein by reference for all purposes.

### 5.4.5 Ig Fusion Proteins

### 5.4.5.1 Antibody Fusion Proteins

In some embodiments, the heterologous protein comprises an antibody. In some embodiments, the antibody can act to further target the immunoreceptor inhibitory protein e.g., to a specified cell or tissue type expressing a specific protein (e.g., cell surface protein). As such, in some embodiments, the antibody is a targeting moiety. Exemplary antibodies include, full-length antibodies, scFvs, Fabs, single domain antibodies (e.g., VHHs), scFv-Fcs, Fab-Fcs, and single domain antibody-Fcs (e.g., VHH-Fcs). In some embodiments, the antibody comprises a full-length antibody. In some embodiments, the antibody comprises a scFv. In some embodiments, the antibody comprises a Fab. In some embodiments, the antibody comprises a single domain antibody. In some embodiments, the antibody comprises a VHH. In some embodiments, the antibody comprises an Fc region.

In specific embodiments, the heterologous protein comprises an antibody that specifically binds a cytokine (e.g., an interleukin). In specific embodiments, the heterologous protein comprises an antibody that specifically binds an interleukin (e.g., a human interleukin). In specific embodiments, the heterologous protein comprises an antibody that specifically binds interleukin 23 (IL-23). In specific embodiments, the heterologous protein comprises an antibody that specifically binds human IL-23.

In some embodiments, the heterologous protein comprises an antibody mimetic. In some embodiments, the antibody mimetic can act to further target the immunoreceptor inhibitory protein e.g., to a specified cell or tissue type expressing a specific protein (e.g., cell surface protein). As such, in some embodiments, the antibody mimetic is a targeting moiety.

In specific embodiments, the heterologous protein comprises an antibody mimetic that specifically binds a cytokine (e.g., an interleukin). In specific embodiments, the heterologous protein comprises an antibody mimetic that specifically binds an interleukin (e.g., a human interleukin). In specific embodiments, the heterologous protein comprises an antibody mimetic that specifically binds interleukin 23 (IL-23). In specific embodiments, the heterologous protein comprises an antibody mimetic that specifically binds human IL-23.

### 5.4.5.2 Ig Fusion Proteins

In some embodiments, the heterologous protein comprises one or more Ig heavy chain constant regions (e.g., a CH2 region, a CH3 region, a hinge region, an Fc region (e.g., in some embodiments, preferably an Fc region) (or any combination of the foregoing). In some embodiments, the Ig is an IgG. In some embodiments, the IgG is IgG1, IgG2, IgG3, or IgG4 (e.g., in some embodiments preferably an IgG1 or IgG4).

In some embodiments, the heterologous protein comprises an IgG CH2 region and an IgG CH3 region. In some embodiments, the heterologous protein comprises a partial IgG hinge region, IgG CH2 region, and IgG CH3 region. In some embodiments, the heterologous protein comprises an IgG hinge region, IgG CH2 region, and IgG CH3 region. In some embodiments, the heterologous protein comprises an IgG1 CH2 region and an IgG1 CH3 region. In some embodiments, the heterologous protein comprises a partial IgG1 hinge region, IgG1 CH2 region, and IgG1 CH3 region. In some embodiments, the heterologous protein comprises an IgG1 hinge region, IgG1 CH2 region, and IgG1 CH3 region. In some embodiments, the heterologous protein comprises an IgG4 CH2 region and an IgG4 CH3 region. In some embodiments, the heterologous protein comprises a partial IgG4 hinge region, IgG4 CH2 region, and IgG4 CH3 region. In some embodiments, the heterologous protein comprises an IgG4 hinge region, IgG4 CH2 region, and IgG4 CH3 region.

In some embodiments, the heterologous protein consists of an IgG CH2 region and an IgG CH3 region. In some embodiments, the heterologous protein consists of a partial IgG hinge region, IgG CH2 region, and IgG CH3 region. In some embodiments, the heterologous protein consists of an IgG hinge region, IgG CH2 region, and IgG CH3 region. In some embodiments, the heterologous protein consists of an IgG1 CH2 region and an IgG1 CH3 region. In some embodiments, the heterologous protein consists of a partial IgG1 hinge region, IgG1 CH2 region, and IgG1 CH3 region. In some embodiments, the heterologous protein consists of an IgG1 hinge region, IgG1 CH2 region, and IgG1 CH3 region. In some embodiments, the heterologous protein consists of an IgG4 CH2 region and an IgG4 CH3 region. In some embodiments, the heterologous protein consists of a partial IgG4 hinge region, IgG4 CH2 region, and IgG4 CH3 region. In some embodiments, the heterologous protein consists of an IgG4 hinge region, IgG4 CH2 region, and IgG4 CH3 region.

In some embodiments, the heterologous protein comprises an Ig Fc region. In some embodiments, the Ig Fc region comprises at least a portion of a hinge region, a CH2 region, and a CH3 region. In some embodiments, the Ig Fc region comprises a hinge region, a CH2 region, and a CH3 region. In some embodiments, the Ig Fc region comprises at least a portion of an IgG hinge region, an IgG CH2 region, and an IgG CH3 region. In some embodiments, the Ig Fc region comprises an IgG hinge region, an IgG CH2 region, and an IgG CH3 region. In some embodiments, the Ig Fc region comprises at least a portion of an IgG1 hinge region, an IgG1 CH2 region, and an IgG1 CH3 region. In some embodiments, the Ig Fc region comprises an IgG1 hinge region, an IgG1 CH2 region, and an IgG1 CH3 region. In some embodiments, the Ig Fc region comprises at least a portion of an IgG4 hinge region, an IgG4 CH2 region, and an IgG4 CH3 region. In some embodiments, the Ig Fc region comprises an IgG4 hinge region, an IgG4 CH2 region, and an IgG4 CH3 region.

In some embodiments, the heterologous protein consists of an Ig Fc region. In some embodiments, the Ig Fc region consists of at least a portion of a hinge region, a CH2 region, and a CH3 region. In some embodiments, the Ig Fc region consists of a hinge region, a CH2 region, and a CH3 region. In some embodiments, the Ig Fc region consists of at least a portion of an IgG hinge region, an IgG CH2 region, and an IgG CH3 region. In some embodiments, the Ig Fc region consists of an IgG hinge region, an IgG CH2 region, and an IgG CH3 region. In some embodiments, the Ig Fc region consists of at least a portion of an IgG1 hinge region, an IgG1 CH2 region, and an IgG1 CH3 region. In some embodiments, the Ig Fc region consists of an IgG1 hinge region, an IgG1 CH2 region, and an IgG1 CH3 region. In some embodiments, the Ig Fc region consists of at least a portion of an IgG4 hinge region, an IgG4 CH2 region, and an IgG4 CH3 region. In some embodiments, the Ig Fc region consists of an IgG4 hinge region, an IgG4 CH2 region, and an IgG4 CH3 region.

In some embodiments, the heterologous protein comprises one or more hIg heavy chain constant regions (e.g., a CH2 region, a CH3 region, a hinge region, an Fc region). In some embodiments, the hIg is a human IgG (hIgG). In some embodiments, the hIgG is hIgG1, IgG2, IgG3, or IgG4. In some embodiments, the hIgG is IgG1 or IgG4. In some embodiments, the hIgG is hIgG1. In some embodiments, the hIgG is hIgG4.

In some embodiments, the heterologous protein comprises a hIgG CH2 region and a hIgG CH3 region. In some embodiments, the heterologous protein comprises a partial hIgG hinge region, hIgG CH2 region, and hIgG CH3 region. In some embodiments, the heterologous protein comprises a hIgG hinge region, hIgG CH2 region, and hIgG CH3 region. In some embodiments, the heterologous protein comprises a hIgG1 CH2 region and a hIgG1 CH3 region. In some embodiments, the heterologous protein comprises a partial hIgG1 hinge region, hIgG1 CH2 region, and hIgG1 CH3 region. In some embodiments, the heterologous protein comprises a hIgG1 hinge region, hIgG1 CH2 region, and hIgG1 CH3 region. In some embodiments, the heterologous protein comprises a hIgG4 CH2 region and a hIgG4 CH3 region. In some embodiments, the heterologous protein comprises a partial hIgG4 hinge region, hIgG4 CH2 region, and hIgG4 CH3 region. In some embodiments, the heterologous protein comprises a hIgG4 hinge region, hIgG4 CH2 region, and hIgG4 CH3 region.

In some embodiments, the heterologous protein consists of a hIgG CH2 region and a hIgG CH3 region. In some embodiments, the heterologous protein consists of a partial hIgG hinge region, hIgG CH2 region, and hIgG CH3 region. In some embodiments, the heterologous protein consists of a hIgG hinge region, hIgG CH2 region, and hIgG CH3 region. In some embodiments, the heterologous protein consists of a hIgG1 CH2 region and a hIgG1 CH3 region. In some embodiments, the heterologous protein consists of a partial hIgG1 hinge region, hIgG1 CH2 region, and hIgG1 CH3 region. In some embodiments, the heterologous protein consists of a hIgG1 hinge region, hIgG1 CH2 region, and hIgG1 CH3 region. In some embodiments, the heterologous protein consists of a hIgG4 CH2 region and a hIgG4 CH3 region. In some embodiments, the heterologous protein consists of a partial hIgG4 hinge region, hIgG4 CH2 region, and hIgG4 CH3 region. In some embodiments, the heterologous protein consists of a hIgG4 hinge region, hIgG4 CH2 region, and hIgG4 CH3 region.

In some embodiments, the heterologous protein comprises a hIg Fc region. In some embodiments, the hIg Fc region comprises at least a portion of a hinge region, a CH2 region, and a CH3 region. In some embodiments, the hIg Fc region comprises a hinge region, a CH2 region, and a CH3 region. In some embodiments, the hIg Fc region comprises at least a portion of a hIgG hinge region, a hIgG CH2 region, and a hIgG CH3 region. In some embodiments, the hIg Fc region comprises a hIgG hinge region, a hIgG CH2 region, and a hIgG CH3 region. In some embodiments, the hIg Fc region comprises at least a portion of a hIgG1 hinge region, a hIgG1 CH2 region, and a hIgG1 CH3 region. In some embodiments, the hIg Fc region comprises a hIgG1 hinge region, a hIgG1 CH2 region, and a hIgG1 CH3 region. In some embodiments, the hIg Fc region comprises at least a portion of a hIgG4 hinge region, a hIgG4 CH2 region, and a hIgG4 CH3 region. In some embodiments, the hIg Fc region comprises a hIgG4 hinge region, a hIgG4 CH2 region, and a hIgG4 CH3 region.

In some embodiments, the heterologous protein consists of a hIg Fc region. In some embodiments, the hIg Fc region consists of at least a portion of a hinge region, a CH2 region, and a CH3 region. In some embodiments, the hIg Fc region consists of a hinge region, a CH2 region, and a CH3 region. In some embodiments, the hIg Fc region consists of at least a portion of a hIgG hinge region, a hIgG CH2 region, and a hIgG CH3 region. In some embodiments, the hIg Fc region consists of a hIgG hinge region, a hIgG CH2 region, and a hIgG CH3 region. In some embodiments, the hIg Fc region consists of at least a portion of a hIgG1 hinge region, a hIgG1 CH2 region, and a hIgG1 CH3 region. In some embodiments, the hIg Fc region consists of a hIgG1 hinge region, a hIgG1 CH2 region, and a hIgG1 CH3 region. In some embodiments, the hIg Fc region consists of at least a portion of a hIgG4 hinge region, a hIgG4 CH2 region, and a hIgG4 CH3 region. In some embodiments, the hIg Fc region consists of a hIgG4 hinge region, a hIgG4 CH2 region, and a hIgG4 CH3 region.

The amino acid sequence of exemplary reference hIgG1 and hIgG4 heavy chain constant regions and hIg light chain constant regions, which can be incorporated in one or more of the embodiments described herein (e.g., fusion proteins and polypeptide), is provided in Table 4.

TABLE 4
The Amino Acid Sequence of Exemplary hlg heavy chain constant region
components and hlg light chain constant regions.

| | | SEQ |
|---|---|---|
| Description | Amino Acid Sequence | ID NO |
| hIgG1 CH1 Region | ASTKGPSVFPLAPSSKSTSGGTAALGCLVKDYFPEPVTVSWNS | 90 |
| | GALTSGVHTFPAVLQSSGLYSLSSVVTVPSSSLGTQTYICNVN | |
| | HKPSNTKVDKKV | |
| hIgG1 Hinge Region | EPKSCDKTHTCP | 91 |
| hIgG1 CH2 Region | PCPAPELLGGPSVFLFPPKPKDTLMISRTPEVTCVVVDVSHED | 92 |
| | PEVKFNWYVDGVEVHNAKTKPREEQYNSTYRVVSVLTVLHQDW | |
| | LNGKEYKCKVSNKALPAPIEKTISKAK | |
| hIgG1 CH3 Region | GQPREPQVYTLPPSRDELTKNQVSLTCLVKGFYPSDIAVEWES | 93 |
| With C-terminal Lysine | NGQPENNYKTTPPVLDSDGSFFLYSKLTVDKSRWQQGNVFSCS | |
| | VMHEALHNHYTQKSLSLSPGK | |
| hIgG1 CH3 Region | GQPREPQVYTLPPSRDELTKNQVSLTCLVKGFYPSDIAVEWES | 94 |
| Without C-terminal | NGQPENNYKTTPPVLDSDGSFFLYSKLTVDKSRWQQGNVFSCS | |
| Lysine | VMHEALHNHYTQKSLSLSPG | |
| hIgG1 CH2 Region + | PCPAPELLGGPSVFLFPPKPKDTLMISRTPEVTCVVVDVSHED | 95 |
| CH3 Region | PEVKFNWYVDGVEVHNAKTKPREEQYNSTYRVVSVLTVLHQDW | |
| With C-terminal Lysine | LNGKEYKCKVSNKALPAPIEKTISKAKGQPREPQVYTLPPSRD | |
| | ELTKNQVSLTCLVKGFYPSDIAVEWESNGQPENNYKTTPPVLD | |
| | SDGSFFLYSKLTVDKSRWQQGNVFSCSVMHEALHNHYTQKSLS | |
| | LSPGK | |
| hIgG1 CH2 Region + | PCPAPELLGGPSVFLFPPKPKDTLMISRTPEVTCVVVDVSHED | 96 |
| CH3 Region | PEVKFNWYVDGVEVHNAKTKPREEQYNSTYRVVSVLTVLHQDW | |
| Without C-terminal | LNGKEYKCKVSNKALPAPIEKTISKAKGQPREPQVYTLPPSRD | |
| Lysine | ELTKNQVSLTCLVKGFYPSDIAVEWESNGQPENNYKTTPPVLD | |
| | SDGSFFLYSKLTVDKSRWQQGNVFSCSVMHEALHNHYTQKSLS | |
| | LSPG | |
| hIgG1 Partial Hinge | TCPPCPAPELLGGPSVFLFPPKPKDTLMISRTPEVTCVVVDVS | 97 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| With C-terminal Lysine | SRDELTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPGK | |
| hIgG1 Partial Hinge | TCPPCPAPELLGGPSVFLFPPKPKDTLMISRTPEVTCVVVDVS | 98 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Without C-terminal | SRDELTKNQVSLTCLVKGFYPSDIAVEWESNGQPENNYKTTPP | |
| Lysine | VLDSDGSFFLYSKLTVDKSRWQQGNVFSCSVMHEALHNHYTQK | |
| | SLSLSPG | |
| hIgG1 Partial Hinge | DKTHTCPPCPAPELLGGPSVFLFPPKPKDTLMISRTPEVTCVV | 197 |
| Region + CH2 Region + | VDVSHEDPEVKFNWYVDGVEVHNAKTKPREEQYNSTYRVVSVL | |
| CH3 Region | TVLHQDWLNGKEYKCKVSNKALPAPIEKTISKAKGQPREPQVY | |
| With C-terminal Lysine | TLPPSRDELTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPGK | |
| hIgG1 Partial Hinge | DKTHTCPPCPAPELLGGPSVFLFPPKPKDTLMISRTPEVTCVV | 198 |
| Region + CH2 Region + | VDVSHEDPEVKFNWYVDGVEVHNAKTKPREEQYNSTYRVVSVL | |
| CH3 Region | TVLHQDWLNGKEYKCKVSNKALPAPIEKTISKAKGQPREPQVY | |
| Without C-terminal | TLPPSRDELTKNQVSLTCLVKGFYPSDIAVEWESNGQPENNYK | |
| Lysine | TTPPVLDSDGSFFLYSKLTVDKSRWQQGNVFSCSVMHEALHNH | |
| | YTQKSLSLSPG | |
| hIgG1 Hinge Region + | EPKSCDKTHTCPPCPAPELLGGPSVFLFPPKPKDTLMISRTPE | 99 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| With C-terminal Lysine | EPQVYTLPPSRDELTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPGK | |
| hIgG1 Hinge Region + | EPKSCDKTHTCPPCPAPELLGGPSVFLFPPKPKDTLMISRTPE | 100 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Without C-terminal | EPQVYTLPPSRDELTKNQVSLTCLVKGFYPSDIAVEWESNGQP | |
| Lysine | ENNYKTTPPVLDSDGSFFLYSKLTVDKSRWQQGNVFSCSVMHE | |
| | ALHNHYTQKSLSLSPG | |
| hIgG1 CH1+ Hinge | ASTKGPSVFPLAPSSKSTSGGTAALGCLVKDYFPEPVTVSWNS | 101 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| With C-terminal Lysine | PKDTLMISRTPEVTCVVVDVSHEDPEVKFNWYVDGVEVHNAKT | |
| | KPREEQYNSTYRVVSVLTVLHQDWLNGKEYKCKVSNKALPAPI | |
| | EKTISKAKGQPREPQVYTLPPSRDELTKNQVSLTCLVKGFYPS | |
| | DIAVEWESNGQPENNYKTTPPVLDSDGSFFLYSKLTVDKSRWQ | |
| | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 102 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Without C-terminal | PKDTLMISRTPEVTCVVVDVSHEDPEVKFNWYVDGVEVHNAKT | |
| Lysine | KPREEQYNSTYRVVSVLTVLHQDWLNGKEYKCKVSNKALPAPI | |
| | EKTISKAKGQPREPQVYTLPPSRDELTKNQVSLTCLVKGFYPS | |
| | DIAVEWESNGQPENNYKTTPPVLDSDGSFFLYSKLTVDKSRWQ | |
| | QGNVFSCSVMHEALHNHYTQKSLSLSPG | |
| hIgG4 CH1 Region | ASTKGPSVFPLAPCSRSTSESTAALGCLVKDYFPEPVTVSWNS | 103 |
| | GALTSGVHTFPAVLQSSGLYSLSSVVTVPSSSLGTKTYTCNVD | |
| | HKPSNTKVDKRV | |
| hIgG4 Hinge Region | ESKYGPPCPSCP | 104 |
| hIgG4 Hinge Region | AESKYGPPCPSCP | 105 |
| (Variant) | | |
| hIgG4 CH2 Region | APEFLGGPSVFLFPPKPKDTLMISRTPEVTCVVVDVSQEDPEV | 106 |
| | QFNWYVDGVEVHNAKTKPREEQFNSTYRVVSVLTVLHQDWLNG | |
| | KEYKCKVSNKGLPSSIEKTISKAK | |
| hIgG4 CH3 Region | GQPREPQVYTLPPSQEEMTKNQVSLTCLVKGFYPSDIAVEWES | 107 |
| With C-terminal Lysine | NGQPENNYKTTPPVLDSDGSFFLYSRLTVDKSRWQEGNVESCS | |
| | VMHEALHNHYTQKSLSLSLGK | |
| hIgG4 CH3 Region | GQPREPQVYTLPPSQEEMTKNQVSLTCLVKGFYPSDIAVEWES | 108 |
| Without C-terminal | NGQPENNYKTTPPVLDSDGSFFLYSRLTVDKSRWQEGNVFSCS | |
| Lysine | VMHEALHNHYTQKSLSLSLG | |
| hIgG4 CH2 Region + | APEFLGGPSVFLFPPKPKDTLMISRTPEVTCVVVDVSQEDPEV | 109 |
| CH3 Region | QFNWYVDGVEVHNAKTKPREEQFNSTYRVVSVLTVLHQDWLNG | |
| With C-terminal Lysine | KEYKCKVSNKGLPSSIEKTISKAKGQPREPQVYTLPPSQEEMT | |
| | KNQVSLTCLVKGFYPSDIAVEWESNGQPENNYKTTPPVLDSDG | |
| | SFFLYSRLTVDKSRWQEGNVFSCSVMHEALHNHYTQKSLSLSL | |
| | GK | |
| hIgG4 CH2 Region + | APEFLGGPSVFLFPPKPKDTLMISRTPEVTCVVVDVSQEDPEV | 110 |
| CH3 Region | QFNWYVDGVEVHNAKTKPREEQFNSTYRVVSVLTVLHQDWLNG | |
| Without C-terminal | KEYKCKVSNKGLPSSIEKTISKAKGQPREPQVYTLPPSQEEMT | |
| Lysine | KNQVSLTCLVKGFYPSDIAVEWESNGQPENNYKTTPPVLDSDG | |
| | SFFLYSRLTVDKSRWQEGNVFSCSVMHEALHNHYTQKSLSLSL | |
| | G | |
| hIgG4 Partial Hinge | PCPSCPAPEFLGGPSVFLFPPKPKDTLMISRTPEVTCVVVDVS | 111 |
| Region + CH2 Region + | QEDPEVQFNWYVDGVEVHNAKTKPREEQFNSTYRVVSVLTVLH | |
| CH3 Region | QDWLNGKEYKCKVSNKGLPSSIEKTISKAKGQPREPQVYTLPP | |
| With C-terminal Lysine | SQEEMTKNQVSLTCLVKGFYPSDIAVEWESNGQPENNYKTTPP | |
| | VLDSDGSFFLYSRLTVDKSRWQEGNVFSCSVMHEALHNHYTQK | |
| | SLSLSLGK | |
| hIgG4 Partial Hinge | PCPSCPAPEFLGGPSVFLFPPKPKDTLMISRTPEVTCVVVDVS | 112 |
| Region + CH2 Region + | QEDPEVQFNWYVDGVEVHNAKTKPREEQFNSTYRVVSVLTVLH | |
| CH3 Region | QDWLNGKEYKCKVSNKGLPSSIEKTISKAKGQPREPQVYTLPP | |
| Without C-terminal | SQEEMTKNQVSLTCLVKGFYPSDIAVEWESNGQPENNYKTTPP | |
| Lysine | VLDSDGSFFLYSRLTVDKSRWQEGNVFSCSVMHEALHNHYTQK | |
| | SLSLSLG | |
| hIgG4 Hinge Region + | ESKYGPPCPSCPAPEFLGGPSVFLFPPKPKDTLMISRTPEVTC | 113 |
| CH2 Region + CH3 | VVVDVSQEDPEVQFNWYVDGVEVHNAKTKPREEQFNSTYRVVS | |
| Region | VLTVLHQDWLNGKEYKCKVSNKGLPSSIEKTISKAKGQPREPQ | |
| With C-terminal Lysine | VYTLPPSQEEMTKNQVSLTCLVKGFYPSDIAVEWESNGQPENN | |
| | YKTTPPVLDSDGSFFLYSRLTVDKSRWQEGNVFSCSVMHEALH | |
| | NHYTQKSLSLSLGK | |
| hIgG4 Hinge Region + | ESKYGPPCPSCPAPEFLGGPSVFLFPPKPKDTLMISRTPEVTC | 114 |
| CH2 Region + CH3 | VVVDVSQEDPEVQFNWYVDGVEVHNAKTKPREEQFNSTYRVVS | |
| Region | VLTVLHQDWLNGKEYKCKVSNKGLPSSIEKTISKAKGQPREPQ | |
| Without C-terminal | VYTLPPSQEEMTKNQVSLTCLVKGFYPSDIAVEWESNGQPENN | |
| Lysine | YKTTPPVLDSDGSFFLYSRLTVDKSRWQEGNVFSCSVMHEALH | |
| | NHYTQKSLSLSLG | |
| hIgG4 Hinge Region + | AESKYGPPCPSCPAPEFLGGPSVFLFPPKPKDTLMISRTPEVT | 115 |
| CH2 Region + CH3 | CVVVDVSQEDPEVQFNWYVDGVEVHNAKTKPREEQFNSTYRVV | |
| Region | SVLTVLHQDWLNGKEYKCKVSNKGLPSSIEKTISKAKGQPREP | |
| (Variant) | QVYTLPPSQEEMTKNQVSLTCLVKGFYPSDIAVEWESNGQPEN | |
| With C-terminal Lysine | NYKTTPPVLDSDGSFFLYSRLTVDKSRWQEGNVFSCSVMHEAL | |
| | HNHYTQKSLSLSLGK | |
| hIgG4 Hinge Region + | AESKYGPPCPSCPAPEFLGGPSVFLFPPKPKDTLMISRTPEVT | 116 |
| CH2 Region + CH3 | CVVVDVSQEDPEVQFNWYVDGVEVHNAKTKPREEQFNSTYRVV | |
| Region | SVLTVLHQDWLNGKEYKCKVSNKGLPSSIEKTISKAKGQPREP | |
| (Variant) | QVYTLPPSQEEMTKNQVSLTCLVKGFYPSDIAVEWESNGQPEN | |
| Without C-terminal | NYKTTPPVLDSDGSFFLYSRLTVDKSRWQEGNVFSCSVMHEAL | |
| Lysine | HNHYTQKSLSLSLG | |
| hIgG4 CH1 + Hinge | ASTKGPSVFPLAPCSRSTSESTAALGCLVKDYFPEPVTVSWNS | 117 |
| Region + CH2 Region + | GALTSGVHTFPAVLQSSGLYSLSSVVTVPSSSLGTKTYTCNVD | |
| CH3 Region | HKPSNTKVDKRVESKYGPPCPSCPAPEFLGGPSVFLFPPKPKD | |
| With C-terminal Lysine | TLMISRTPEVTCVVVDVSQEDPEVQFNWYVDGVEVHNAKTKPR | |
| | EEQFNSTYRVVSVLTVLHQDWLNGKEYKCKVSNKGLPSSIEKT | |
| | ISKAKGQPREPQVYTLPPSQEEMTKNQVSLTCLVKGFYPSDIA | |
| | VEWESNGQPENNYKTTPPVLDSDGSFFLYSRLTVDKSRWQEGN | |
| | VFSCSVMHEALHNHYTQKSLSLSLGK | |
| hIgG4 CH1 + Hinge | ASTKGPSVFPLAPCSRSTSESTAALGCLVKDYFPEPVTVSWNS | 118 |
| Region + CH2 Region + | GALTSGVHTFPAVLQSSGLYSLSSVVTVPSSSLGTKTYTCNVD | |
| CH3 Region | HKPSNTKVDKRVESKYGPPCPSCPAPEFLGGPSVFLFPPKPKD | |
| Without C-terminal | TLMISRTPEVTCVVVDVSQEDPEVQFNWYVDGVEVHNAKTKPR | |
| Lysine | EEQFNSTYRVVSVLTVLHQDWLNGKEYKCKVSNKGLPSSIEKT | |
| | ISKAKGQPREPQVYTLPPSQEEMTKNQVSLTCLVKGFYPSDIA | |
| | VEWESNGQPENNYKTTPPVLDSDGSFFLYSRLTVDKSRWQEGN | |
| | VFSCSVMHEALHNHYTQKSLSLSLG | |
| Ig light chain kappa | RTVAAPSVFIFPPSDEQLKSGTASVVCLLNNFYPREAKVQWKV | 119 |
| constant region (KCL) | DNALQSGNSQESVTEQDSKDSTYSLSSTLTLSKADYEKHKVYA | |
| | CEVTHQGLSSPVTKSFNRGEC | |
| Ig light chain kappa | GQPKANPTVTLFPPSSEELQANKATLVCLISDFYPGAVTVAWK | 120 |
| constant region (ACL) | ADGSPVKAGVETTKPSKQSNNKYAASSYLSLTPEQWKSHRSYS | |
| | CQVTHEGSTVEKTVAPTECS | |

In some embodiments, the amino acid sequence of the heterologous protein comprises an amino acid sequence set forth in Table 4. In some embodiments, the amino acid sequence of the heterologous protein comprises an amino acid sequence set forth in Table 4, and further comprises 1 or more but less than 15% (less than 12%, less than 10%, less than 8%), amino acid variations (e.g., amino acid substitutions, deletions, or additions). In some embodiments, the amino acid sequence of the heterologous protein comprises an amino acid sequence set forth in Table 4, comprising at least about 1, 2, 3, 4, 5, 6, 7, 8, 9, 10, or more amino acid variations (e.g., amino acid substitutions, deletions, or additions). In some embodiments, the amino acid sequence of the heterologous protein comprises an amino acid sequence set forth in Table 4, comprising about 1, 2, 3, 4, 5, 6, 7, 8, 9, 10, or more amino acid variations (e.g., amino acid substitutions, deletions, or additions). In some embodiments, the amino acid sequence of the heterologous protein comprises an amino acid sequence set forth in Table 4, comprising no more than about 1, 2, 3, 4, 5, 6, 7, 8, 9, 10, or more amino acid variations (e.g., amino acid substitutions, deletions, or additions).

In some embodiments, the amino acid sequence of the heterologous protein consists of an amino acid sequence set forth in Table 4. In some embodiments, the amino acid sequence of the heterologous protein consists of an amino acid sequence set forth in Table 4, and further comprises 1 or more but less than 15% (less than 12%, less than 10%, less than 8%), amino acid variations (e.g., amino acid substitutions, deletions, or additions). In some embodiments, the amino acid sequence of the heterologous protein consists of an amino acid sequence set forth in Table 4, comprising at least about 1, 2, 3, 4, 5, 6, 7, 8, 9, 10, or more amino acid variations (e.g., amino acid substitutions, deletions, or additions). In some embodiments, the amino acid sequence of the heterologous protein consists of an amino acid sequence set forth in Table 4, comprising about 1, 2, 3, 4, 5, 6, 7, 8, 9, 10, or more amino acid variations (e.g., amino acid substitutions, deletions, or additions). In some embodiments, the amino acid sequence of the heterologous protein consists of an amino acid sequence set forth in Table 4, comprising no more than about 1, 2, 3, 4, 5, 6, 7, 8, 9, 10, or more amino acid variations (e.g., amino acid substitutions, deletions, or additions).

In some embodiments, the amino acid sequence of the heterologous protein comprises the amino acid sequence set forth in any one of SEQ ID NOS: 90-120 or 197-198. In some embodiments, the amino acid sequence of the heterologous protein comprises the amino acid sequence set forth in any one of SEQ ID NOS: 90-120 or 197-198, and further comprises 1 or more but less than 15% (less than 12%, less than 10%, less than 8%), amino acid variations (e.g., amino acid substitutions, deletions, or additions). In some embodiments, the amino acid sequence of the heterologous protein comprises the amino acid sequence set forth in any one of SEQ ID NOS: 90-120 or 197-198, comprising at least about 1, 2, 3, 4, 5, 6, 7, 8, 9, 10, or more amino acid variations (e.g., amino acid substitutions, deletions, or additions). In some embodiments, the amino acid sequence of the heterologous protein comprises the amino acid sequence set forth in any one of SEQ ID NOS: 90-120 or 197-198, comprising about 1, 2, 3, 4, 5, 6, 7, 8, 9, or 10 amino acid variations (e.g., amino acid substitutions, deletions, or additions). In some embodiments, the amino acid sequence of the heterologous protein comprises the amino acid sequence set forth in any one of SEQ ID NOS: 90-120 or 197-198, comprising no more than about 1, 2, 3, 4, 5, 6, 7, 8, 9, or 10 amino acid variations (e.g., amino acid substitutions, deletions, or additions).

In some embodiments, the amino acid sequence of the heterologous protein consists of the amino acid sequence set forth in any one of SEQ ID NOS: 90-120 or 197-198. In some embodiments, the amino acid sequence of the heterologous protein consists of the amino acid sequence set forth in any one of SEQ ID NOS: 90-120 or 197-198, and further comprising 1 or more but less than 15% (less than 12%, less than 10%, less than 8%), amino acid variations (e.g., amino acid substitutions, deletions, or additions). In some embodiments, the amino acid sequence of the heterologous protein consists of the amino acid sequence set forth in any one of SEQ ID NOS: 90-120 or 197-198, comprising at least about 1, 2, 3, 4, 5, 6, 7, 8, 9, 10, or more amino acid variations (e.g., amino acid substitutions, deletions, or additions). In some embodiments, the amino acid sequence of the heterologous protein consists of the amino acid sequence set forth in any one of SEQ ID NOS: 90-120 or 197-198, comprising about 1, 2, 3, 4, 5, 6, 7, 8, 9, or 10 amino acid variations (e.g., amino acid substitutions, deletions, or additions). In some embodiments, the amino acid sequence of the heterologous protein consists of the amino acid sequence set forth in any one of SEQ ID NOS: 90-120, comprising no more than about 1, 2, 3, 4, 5, 6, 7, 8, 9, or 10 amino acid variations (e.g., amino acid substitutions, deletions, or additions).

In some embodiments, wherein the heterologous protein comprises a CH3 region (e.g., comprises an Fc region; a hinge region, CH2 region, and CH3 region, etc.), the CH3 region lacks the C-terminal lysine (e.g., residue 232 of SEQ ID NO: 99, numbering according to SEQ ID NO: 99; or e.g., residue 229 of SEQ ID NO: 113, numbering according to SEQ ID NO: 113). In some embodiments, the CH3 region further lacks the C-terminal glycine (e.g., residue 231 of SEQ ID NO: 99, numbering according to SEQ ID NO: 99; or e.g., residue 228 of SEQ ID NO: 113, numbering according to SEQ ID NO: 113).

In some embodiments, the heterologous protein comprises one or more mIg heavy chain constant regions (e.g., a CH2 region, a CH3 region, a hinge region, an Fc region). In some embodiments, the mIg is mIgG (mIgG). In some embodiments, the mIgG is mIgG1, mIgG2a, mIgG2c, mIgG2b, or mIgG3. In some embodiments, the mIgG is mIgG1 or mIgG2a. In some embodiments, the mIgG is mIgG1. In some embodiments, the mIgG is mIgG2a.

In some embodiments, the heterologous protein comprises a mIgG CH2 region and a mIgG CH3 region. In some embodiments, the heterologous protein comprises a partial mIgG hinge region, mIgG CH2 region, and mIgG CH3 region. In some embodiments, the heterologous protein comprises a mIgG hinge region, mIgG CH2 region, and mIgG CH3 region. In some embodiments, the heterologous protein comprises a mIgG1 CH2 region and a mIgG1 CH3 region. In some embodiments, the heterologous protein comprises a partial mIgG1 hinge region, mIgG1 CH2 region, and mIgG1 CH3 region. In some embodiments, the heterologous protein comprises a mIgG1 hinge region, mIgG1 CH2 region, and mIgG1 CH3 region. In some embodiments, the heterologous protein comprises a mIgG2a CH2 region and a mIgG2a CH3 region. In some embodiments, the heterologous protein comprises a partial mIgG2a hinge region, mIg2a CH2 region, and mIgG2a CH3 region. In some embodiments, the heterologous protein comprises a mIgG2a hinge region, mIgG2a CH2 region, and mIgG2a CH3 region.

In some embodiments, the heterologous protein comprises a mIg Fc region. In some embodiments, the mIg Fc region comprises at least a portion of a hinge region, a CH2 region, and a CH3 region. In some embodiments, the mIg Fc region comprises a hinge region, a CH2 region, and a CH3 region. In some embodiments, the mIg Fc region comprises at least a portion of a mIgG hinge region, a mIgG CH2 region, and a mIgG CH3 region. In some embodiments, the mIg Fc region comprises a mIgG hinge region, a mIgG CH2 region, and a mIgG CH3 region. In some embodiments, the mIg Fc region comprises at least a portion of a mIgG1 hinge region, a mIgG1 CH2 region, and a mIgG1 CH3 region. In some embodiments, the mIg Fc region comprises a mIgG1 hinge region, a mIgG1 CH2 region, and a mIgG1 CH3 region. In some embodiments, the mIg Fc region comprises at least a portion of a mIgG2a hinge region, a mIgG2a CH2 region, and a mIgG2a CH3 region. In some embodiments, the mIg Fc region comprises a mIgG2a hinge region, a mIgG2a CH2 region, and a mIgG2a CH3 region.

In some embodiments, the heterologous protein consists of a mIgG CH2 region and a mIgG CH3 region. In some embodiments, the heterologous protein consists of a partial mIgG hinge region, mIgG CH2 region, and mIgG CH3 region. In some embodiments, the heterologous protein consists of a mIgG hinge region, mIgG CH2 region, and mIgG CH3 region. In some embodiments, the heterologous protein consists of a mIgG1 CH2 region and a mIgG1 CH3 region. In some embodiments, the heterologous protein consists of a partial mIgG1 hinge region, mIgG1 CH2 region, and mIgG1 CH3 region. In some embodiments, the heterologous protein consists of a mIgG1 hinge region, mIgG1 CH2 region, and mIgG1 CH3 region. In some embodiments, the heterologous protein consists of a mIgG2a CH2 region and a mIgG2a CH3 region. In some embodiments, the heterologous protein consists of a partial mIgG2a hinge region, mIg2a CH2 region, and mIgG2a CH3 region. In some embodiments, the heterologous protein consists of a mIgG2a hinge region, mIgG2a CH2 region, and mIgG2a CH3 region.

In some embodiments, the heterologous protein consists of a mIg Fc region. In some embodiments, the mIg Fc region consists of at least a portion of a hinge region, a CH2 region, and a CH3 region. In some embodiments, the mIg Fc region consists of a hinge region, a CH2 region, and a CH3 region. In some embodiments, the mIg Fc region consists of at least a portion of a mIgG hinge region, a mIgG CH2 region, and a mIgG CH3 region. In some embodiments, the mIg Fc region consists of a mIgG hinge region, a mIgG CH2 region, and a mIgG CH3 region. In some embodiments, the mIg Fc region consists of at least a portion of a mIgG1 hinge region, a mIgG1 CH2 region, and a mIgG1 CH3 region. In some embodiments, the mIg Fc region consists of a mIgG1 hinge region, a mIgG1 CH2 region, and a mIgG1 CH3 region. In some embodiments, the mIg Fc region consists of at least a portion of a mIgG2a hinge region, a mIgG2a CH2 region, and a mIgG2a CH3 region. In some embodiments, the mIg Fc region consists of a mIgG2a hinge region, a mIgG2a CH2 region, and a mIgG2a CH3 region.

The amino acid sequence of exemplary reference mIgG1 and mIgG2a heavy chain constant regions, which can be incorporated in one or more of the embodiments described herein (e.g., fusion proteins and polypeptide), is provided in Table 5.

TABLE 5
The Amino Acid Sequence of Exemplary mIg heavy chain constant region
components.

| | | SEQ |
|---|---|---|
| Description | Amino Acid Sequence | ID NO |
| mIgG1 CH1 Region | AKTTPPSVYPLAPGSAAQTNSMVTLGCLVKGYFPEPVTVTWNSG | 121 |
| | SLSSGVHTFPAVLQSDLYTLSSSVTVPSSPRPSETVTCNVAHPA | |
| | SSTKVDKKI | |
| mIgG1 Hinge Region | VPRDCGCKPCICT | 122 |
| mIgG1 CH2 Region | VPEVSSVFIFPPKPKDVLTITLTPKVTCVVVAISKDDPEVQFSW | 123 |
| | FVDDVEVHTAQTQPREEQFNSTFRSVSELPIMHQDWLNGKEFKC | |
| | RVNSAAFPAPIEKTISKTK | |
| mIgG1 CH3 Region | GRPKAPQVYTIPPPKEQMAKDKVSLTCMITDFFPEDITVEWQWN | 124 |
| With C-terminal Lysine | GQPAENYKNTQPIMNINGSYFVYSKLNVQKSNWEAGNTFTCSVL | |
| | HEGLHNHHTEKSLSHSPGK | |
| mIgG1 CH3 Region | GRPKAPQVYTIPPPKEQMAKDKVSLTCMITDFFPEDITVEWQWN | 125 |
| Without C-terminal | GQPAENYKNTQPIMNINGSYFVYSKLNVQKSNWEAGNTFTCSVL | |
| Lysine | HEGLHNHHTEKSLSHSPG | |
| mIgG1 CH2 Region + | VPEVSSVFIFPPKPKDVLTITLTPKVTCVVVAISKDDPEVQFSW | 126 |
| CH3 Region | FVDDVEVHTAQTQPREEQFNSTFRSVSELPIMHQDWLNGKEFKC | |
| With C-terminal Lysine | RVNSAAFPAPIEKTISKTKGRPKAPQVYTIPPPKEQMAKDKVSL | |
| | TCMITDFFPEDITVEWQWNGQPAENYKNTQPIMNINGSYFVYSK | |
| | LNVQKSNWEAGNTFTCSVLHEGLHNHHTEKSLSHSPGK | |
| mIgG1 CH2 Region + | VPEVSSVFIFPPKPKDVLTITLTPKVTCVVVAISKDDPEVQFSW | 127 |
| CH3 Region | FVDDVEVHTAQTQPREEQFNSTFRSVSELPIMHQDWLNGKEFKC | |
| Without C-terminal | RVNSAAFPAPIEKTISKTKGRPKAPQVYTIPPPKEQMAKDKVSL | |
| Lysine | TCMITDFFPEDITVEWQWNGQPAENYKNTQPIMNINGSYFVYSK | |
| | LNVQKSNWEAGNTFTCSVLHEGLHNHHTEKSLSHSPG | |
| mIgG1 Hinge Region + | VPRDCGCKPCICTVPEVSSVFIFPPKPKDVLTITLTPKVTCVVV | 128 |
| CH2 Region + CH3 | AISKDDPEVQFSWFVDDVEVHTAQTQPREEQFNSTFRSVSELPI | |
| Region | MHQDWLNGKEFKCRVNSAAFPAPIEKTISKTKGRPKAPQVYTIP | |
| With C-terminal Lysine | PPKEQMAKDKVSLTCMITDFFPEDITVEWQWNGQPAENYKNTQP | |
| | IMNTNGSYFVYSKLNVQKSNWEAGNTFTCSVLHEGLHNHHTEKS | |
| | LSHSPGK | |
| mIgG1 Hinge Region + | VPRDCGCKPCICTVPEVSSVFIFPPKPKDVLTITLTPKVTCVVV | 129 |
| CH2 Region + CH3 | AISKDDPEVQFSWFVDDVEVHTAQTQPREEQFNSTERSVSELPI | |
| Region | MHQDWLNGKEFKCRVNSAAFPAPIEKTISKTKGRPKAPQVYTIP | |
| Without C-terminal | PPKEQMAKDKVSLTCMITDFFPEDITVEWQWNGQPAENYKNTQP | |
| Lysine | IMNTNGSYFVYSKLNVQKSNWEAGNTFTCSVLHEGLHNHHTEKS | |
| | LSHSPGK | |
| mIgG2a Hinge Region | EPRGPTIKPCPPCKCP | 130 |
| mIgG2a CH2 Region | APNAAGGPSVFIFLLKIKDVLMISLSPIVTCVVVDVSEDDPDVQ | 131 |
| | ISWFVNNVEVHTAQTQTHREDYNSTLRVVSALPIQHQDWMSGKE | |
| | FKCKVNNKDLPAPIERTISKPKGSVRAPQVYVLPPPEEEMTKKQ | |
| | VT | |
| mIgG2a CH3 Region | LTCMVTDFMPEDIYVEWTNNGKTELNYKNTEPVLDSDGSYFMYS | 132 |
| With C-terminal Lysine | KLRVEKKNWVERNSYSCSVVHEGLHNHHTTKSFSRTPGK | |
| mIgG2a CH3 Region | LTCMVTDFMPEDIYVEWTNNGKTELNYKNTEPVLDSDGSYFMYS | 133 |
| Without C-terminal | KLRVEKKNWVERNSYSCSVVHEGLHNHHTTKSFSRTPG | |
| Lysine | | |
| mIgG2a CH2 Region + | APNAAGGPSVFIFLLKIKDVLMISLSPIVTCVVVDVSEDDPDVQ | 134 |
| CH3 Region | ISWFVNNVEVHTAQTQTHREDYNSTLRVVSALPIQHQDWMSGKE | |
| With C-terminal Lysine | FKCKVNNKDLPAPIERTISKPKGSVRAPQVYVLPPPEEEMTKKQ | |
| | VTLTCMVTDFMPEDIYVEWTNNGKTELNYKNTEPVLDSDGSYFM | |
| | YSKLRVEKKNWVERNSYSCSVVHEGLHNHHTTKSFSRTPGK | |
| mIgG2a CH2 Region + | APNAAGGPSVFIFLLKIKDVLMISLSPIVTCVVVDVSEDDPDVQ | 135 |
| CH3 Region | ISWFVNNVEVHTAQTQTHREDYNSTLRVVSALPIQHQDWMSGKE | |
| Without C-terminal | FKCKVNNKDLPAPIERTISKPKGSVRAPQVYVLPPPEEEMTKKQ | |
| Lysine | VTLTCMVTDEMPEDIYVEWTNNGKTELNYKNTEPVLDSDGSYFM | |
| | YSKLRVEKKNWVERNSYSCSVVHEGLHNHHTTKSFSRTPG | |
| mIgG2a Hinge Region | EPRGPTIKPCPPCKCPAPNAAGGPSVFIFLLKIKDVLMISLSPI | 136 |
| + CH2 Region + CH3 | VTCVVVDVSEDDPDVQISWFVNNVEVHTAQTQTHREDYNSTLRV | |
| Region | VSALPIQHQDWMSGKEFKCKVNNKDLPAPIERTISKPKGSVRAP | |
| With C-terminal Lysine | QVYVLPPPEEEMTKKQVTLTCMVTDFMPEDIYVEWTNNGKTELN | |
| | YKNTEPVLDSDGSYFMYSKLRVEKKNWVERNSYSCSVVHEGLHN | |
| | HHTTKSFSRTPGK | |
| mIgG2a Hinge Region | EPRGPTIKPCPPCKCPAPNAAGGPSVFIFLLKIKDVLMISLSPI | 137 |
| + CH2 Region + CH3 | VTCVVVDVSEDDPDVQISWFVNNVEVHTAQTQTHREDYNSTLRV | |
| Region | VSALPIQHQDWMSGKEFKCKVNNKDLPAPIERTISKPKGSVRAP | |
| Without C-terminal | QVYVLPPPEEEMTKKQVTLTCMVTDFMPEDIYVEWINNGKTELN | |
| Lysine | YKNTEPVLDSDGSYFMYSKLRVEKKNWVERNSYSCSVVHEGLHN | |
| | HHTTKSFSRTPG | |
| mIgG2a CH1 Region + | AKTTPPSVYPLAPGSAAQTNSMVTLGCLVKGYFPEPVTVTWNSG | 138 |
| Hinge Region + CH2 | SLSSGVHTFPAVLQSDLYTLSSSVTVPSSPRPSETVTCNVAHPA | |
| Region + CH3 Region | SSTKVDKKIEPRGPTIKPCPPCKCPAPNAAGGPSVFIFLLKIKD | |
| With C-terminal Lysine | VLMISLSPIVTCVVVDVSEDDPDVQISWFVNNVEVHTAQTQTHR | |
| | EDYNSTLRVVSALPIQHQDWMSGKEFKCKVNNKDLPAPIERTIS | |
| | KPKGSVRAPQVYVLPPPEEEMTKKQVTLTCMVTDFMPEDIYVEW | |
| | TNNGKTELNYKNTEPVLDSDGSYFMYSKLRVEKKNWVERNSYSC | |
| | SVVHEGLHNHHTTKSFSRTPGK | |
| mIgG2a CH1 Region + | AKTTPPSVYPLAPGSAAQTNSMVTLGCLVKGYFPEPVTVTWNSG | 139 |
| Hinge Region + CH2 | SLSSGVHTFPAVLQSDLYTLSSSVTVPSSPRPSETVTCNVAHPA | |
| Region + CH3 Region | SSTKVDKKIEPRGPTIKPCPPCKCPAPNAAGGPSVFIFLLKIKD | |
| Without C-terminal | VLMISLSPIVTCVVVDVSEDDPDVQISWFVNNVEVHTAQTQTHR | |
| Lysine | EDYNSTLRVVSALPIQHQDWMSGKEFKCKVNNKDLPAPIERTIS | |
| | KPKGSVRAPQVYVLPPPEEEMTKKQVTLTCMVTDEMPEDIYVEW | |
| | TNNGKTELNYKNTEPVLDSDGSYFMYSKLRVEKKNWVERNSYSC | |
| | SVVHEGLHNHHTTKSFSRTPG | |
| Ig light chain kappa | RADAAPTVSIFPPSSEQLTSGGASVVCFLNNFYPKDINVKWKID | 140 |
| constant region (KCL) | GSERQNGVLNSWTDQDSKDSTYSMSSTLTLTKDEYERHNSYTCE | |
| | ATHKTSTSPIVKSENRNEC | |
| Ig light chain kappa | QPKSSPSVTLFPPSSEELETNKATLVCTITDFYPGVVTVDWKVD | 141 |
| constant region (ACL) | GTPVTQGMETTQPSKQSNNKYMASSYLTLTARAWERHSSYSCQV | |
| | THEGHTVEKSLSRADCS | |

In some embodiments, the amino acid sequence of the heterologous protein comprises an amino acid sequence set forth in Table 5. In some embodiments, the amino acid sequence of the heterologous protein comprises an amino acid sequence set forth in Table 5, and further comprises 1 or more but less than 15% (less than 12%, less than 10%, less than 8%), amino acid variations (e.g., amino acid substitutions, deletions, or additions). In some embodiments, the amino acid sequence of the heterologous protein comprises an amino acid sequence set forth in Table 5, comprising at least about 1, 2, 3, 4, 5, 6, 7, 8, 9, 10, or more amino acid variations (e.g., amino acid substitutions, deletions, or additions). In some embodiments, the amino acid sequence of the heterologous protein comprises an amino acid sequence set forth in Table 5, comprising about 1, 2, 3, 4, 5, 6, 7, 8, 9, 10, or more amino acid variations (e.g., amino acid substitutions, deletions, or additions). In some embodiments, the amino acid sequence of the heterologous protein comprises an amino acid sequence set forth in Table 5, comprising no more than about 1, 2, 3, 4, 5, 6, 7, 8, 9, 10, or more amino acid variations (e.g., amino acid substitutions, deletions, or additions).

In some embodiments, the amino acid sequence of the heterologous protein consists of an amino acid sequence set forth in Table 5. In some embodiments, the amino acid sequence of the heterologous protein consists of an amino acid sequence set forth in Table 5, and further comprises 1 or more but less than 15% (less than 12%, less than 10%, less than 8%), amino acid variations (e.g., amino acid substitutions, deletions, or additions). In some embodiments, the amino acid sequence of the heterologous protein consists of an amino acid sequence set forth in Table 5, comprising at least about 1, 2, 3, 4, 5, 6, 7, 8, 9, 10, or more amino acid variations (e.g., amino acid substitutions, deletions, or additions). In some embodiments, the amino acid sequence of the heterologous protein consists of an amino acid sequence set forth in Table 5, comprising about 1, 2, 3, 4, 5, 6, 7, 8, 9, 10, or more amino acid variations (e.g., amino acid substitutions, deletions, or additions). In some embodiments, the amino acid sequence of the heterologous protein consists of an amino acid sequence set forth in Table 5, comprising no more than about 1, 2, 3, 4, 5, 6, 7, 8, 9, 10, or more amino acid variations (e.g., amino acid substitutions, deletions, or additions).

In some embodiments, the amino acid sequence of the heterologous protein comprises the amino acid sequence set forth in any one of SEQ ID NOS: 121-141. In some embodiments, the amino acid sequence of the heterologous protein comprises the amino acid sequence set forth in any one of SEQ ID NOS: 121-141, and further comprises 1 or more but less than 15% (less than 12%, less than 10%, less than 8%), amino acid variations (e.g., amino acid substitutions, deletions, or additions). In some embodiments, the amino acid sequence of the heterologous protein comprises the amino acid sequence set forth in any one of SEQ ID NOS: 121-141, comprising at least about 1, 2, 3, 4, 5, 6, 7, 8, 9, 10, or more amino acid variations (e.g., amino acid substitutions, deletions, or additions). In some embodiments, the amino acid sequence of the heterologous protein comprises the amino acid sequence set forth in any one of SEQ ID NOS: 121-141, comprising about 1, 2, 3, 4, 5, 6, 7, 8, 9, or 10 amino acid variations (e.g., amino acid substitutions, deletions, or additions). In some embodiments, the amino acid sequence of the heterologous protein comprises the amino acid sequence set forth in any one of SEQ ID NOS: 121-141, comprising no more than about 1, 2, 3, 4, 5, 6, 7, 8, 9, or 10 amino acid variations (e.g., amino acid substitutions, deletions, or additions).

In some embodiments, the amino acid sequence of the heterologous protein consists of the amino acid sequence set forth in any one of SEQ ID NOS: 121-141. In some embodiments, the amino acid sequence of the heterologous protein consists of the amino acid sequence set forth in any one of SEQ ID NOS: 121-141, and further comprises 1 or more but less than 15% (less than 12%, less than 10%, less than 8%), amino acid variations (e.g., amino acid substitutions, deletions, or additions). In some embodiments, the amino acid sequence of the heterologous protein consists of the amino acid sequence set forth in any one of SEQ ID NOS: 121-141, comprising at least about 1, 2, 3, 4, 5, 6, 7, 8, 9, 10, or more amino acid variations (e.g., amino acid substitutions, deletions, or additions). In some embodiments, the amino acid sequence of the heterologous protein consists of the amino acid sequence set forth in any one of SEQ ID NOS: 121-141, comprising about 1, 2, 3, 4, 5, 6, 7, 8, 9, or 10 amino acid variations (e.g., amino acid substitutions, deletions, or additions). In some embodiments, the amino acid sequence of the heterologous protein consists of the amino acid sequence set forth in any one of SEQ ID NOS: 121-141, comprising no more than about 1, 2, 3, 4, 5, 6, 7, 8, 9, or 10 amino acid variations (e.g., amino acid substitutions, deletions, or additions).

In some embodiments, wherein the heterologous protein comprises a CH3 region (e.g., comprises an Fc region; a hinge region, CH2 region, and CH3 region, etc.), the CH3 region lacks the C-terminal lysine (e.g., residue 227 of SEQ ID NO: 129, numbering according to SEQ ID NO: 129; or e.g., residue 223 of SEQ ID NO: 136, numbering according to SEQ ID NO: 136). In some embodiments, the CH3 region further lacks the C-terminal glycine (e.g., residue 226 of SEQ ID NO: 129, numbering according to SEQ ID NO: 129; or e.g., residue 222 of SEQ ID NO: 136, numbering according to SEQ ID NO: 136).

### 5.4.5.3 Half-Life Extension

In some embodiments, the Ig (e.g., hIg, mIg) Fc region of a fusion protein described herein (an Fc region, an antibody, etc.) exhibits enhanced serum half-life, e.g., relative to a reference Ig (e.g., hIg, mIg) Fc region (e.g., a wild-type Ig (e.g., hIg, mIg) Fc region).

Standard in vitro and/or in vivo assays known in the art can be conducted to evaluate scrum half-life. See, e.g., Ko S, Jo M, Jung S T. Recent Achievements and Challenges in Prolonging the Serum Half-Lives of Therapeutic IgG Antibodies Through Fc Engineering. BioDrugs. 2021; 35 (2): 147-157. doi: 10.1007/s40259-021-00471-0, the entire contents of which are incorporated herein by reference for all purposes.

In some embodiments, the Ig (e.g., hIg, mIg) Fc region of a fusion protein described herein (an Fc region, an antibody, etc.) exhibits enhanced serum half-life (e.g., relative to a reference Ig (e.g., hIg, mIg) Fc region (e.g., a wild-type Ig (e.g., hIg, mIg) Fc region)) through enhanced binding affinity for the FcRn receptor (e.g., the human FcRn receptor) (e.g., relative to a reference Ig (e.g., hIg, mIg) Fc region (e.g., a wild-type Ig (e.g., hIg, mIg) Fc region)).

In some embodiments, the Ig (e.g., hIg, mIg) Fc region of a fusion protein described herein (an Fc region, an antibody, etc.) exhibits enhanced serum half-life (e.g., relative to a reference Ig (e.g., hIg, mIg) Fc region (e.g., a wild-type Ig (e.g., hIg, mIg) Fc region)) through enhanced binding affinity for the FcRn receptor (e.g., the human FcRn receptor) at a pH of from about 5.5-6.5 (e.g., relative to a reference Ig (e.g., hIg, mIg) Fc region (e.g., a wild-type Ig (e.g., hIg, mIg) Fc region)). In some embodiments, the Ig (e.g., hIg, mIg) Fc region of a fusion protein described herein (an Fc region, an antibody, etc.) exhibits enhanced serum half-life (e.g., relative to a reference Ig (e.g., hIg, mIg) Fc region (e.g., a wild-type Ig (e.g., hIg, mIg) Fc region)) through enhanced binding affinity for the FcRn receptor (e.g., the human FcRn receptor) at a pH of from about 5.5-6.5 and no substantial change in binding affinity for the FcRn receptor (e.g., the human FcRn receptor) at a pH of from about 7.0-7.5 (e.g., relative to a reference Ig (e.g., hIg, mIg) Fc region (e.g., a wild-type Ig (e.g., hIg, mIg) Fc region)). In some embodiments, the Ig (e.g., hIg, mIg) Fc region of a fusion protein described herein (an Fc region, an antibody, etc.) exhibits enhanced serum half-life (e.g., relative to a reference Ig (e.g., hIg, mIg) Fc region (e.g., a wild-type Ig (e.g., hIg, mIg) Fc region)) through enhanced binding affinity for the FcRn receptor (e.g., the human FcRn receptor) at a pH of from about 6.0-6.5 (e.g., relative to a reference Ig (e.g., hIg, mIg) Fc region (e.g., a wild-type Ig (e.g., hIg, mIg) Fc region)) and a decrease in binding affinity for the FcRn receptor (e.g., the human FcRn receptor) at a pH of from about 7.0-7.5 (e.g., relative to a reference Ig (e.g., hIg, mIg) Fc region (e.g., a wild-type Ig (e.g., hIg, mIg) Fc region)).

In some embodiments, the Ig (e.g., hIg, mIg) Fc region of a fusion protein described herein (an Fc region, an antibody, etc.) exhibits enhanced serum half-life (e.g., relative to a reference Ig (e.g., hIg, mIg) Fc region (e.g., a wild-type Ig (e.g., hIg, mIg) Fc region)) through enhanced binding affinity for the FcRn receptor (e.g., the human FcRn receptor) at a pH of about 6 (e.g., relative to a reference Ig (e.g., hIg, mIg) Fc region (e.g., a wild-type Ig (e.g., hIg, mIg) Fc region)). In some embodiments, the Ig (e.g., hIg, mIg) Fc region of a fusion protein described herein (an Fc region, an antibody, etc.) exhibits enhanced serum half-life (e.g., relative to a reference Ig (e.g., hIg, mIg) Fc region (e.g., a wild-type Ig (e.g., hIg, mIg) Fc region)) through enhanced binding affinity for the FcRn receptor (e.g., the human FcRn receptor) at a pH of about 6 and no substantial change in binding affinity for the FcRn receptor (e.g., the human FcRn receptor) at a pH of about 7.4 (e.g., relative to a reference Ig (e.g., hIg, mIg) Fc region (e.g., a wild-type Ig (e.g., hIg, mIg) Fc region)). In some embodiments, the Ig (e.g., hIg, mIg) Fc region of a fusion protein described herein (an Fc region, an antibody, etc.) exhibits enhanced serum half-life (e.g., relative to a reference Ig (e.g., hIg, mIg) Fc region (e.g., a wild-type Ig (e.g., hIg, mIg) Fc region)) through enhanced binding affinity for the FcRn receptor (e.g., the human FcRn receptor) at a pH of about 6 (e.g., relative to a reference Ig (e.g., hIg, mIg) Fc region (e.g., a wild-type Ig (e.g., hIg, mIg) Fc region)) and a decrease in binding affinity for the FcRn receptor (e.g., the human FcRn receptor) at a pH of about 7.4 (e.g., relative to a reference Ig (e.g., hIg, mIg) Fc region (e.g., a wild-type Ig (e.g., hIg, mIg) Fc region)).

In some embodiments, the Ig (e.g., hIg, mIg) Fc region of a fusion protein described herein comprises one or more amino acid variation (e.g., relative to a reference Ig (e.g., hIg, mIg) Fc region (e.g., a wild-type Ig (e.g., hIg, mIg) Fc region)) that enhances serum half-life of the fusion protein (e.g., relative to a reference Ig (e.g., hIg, mIg) Fc region (e.g., a wild-type Ig (e.g., hIg, mIg) Fc region)).

In some embodiments, the Ig (e.g., hIg, mIg) Fc region of a fusion protein described herein comprises one or more amino acid variation (e.g., relative to a reference Ig (e.g., hIg, mIg) Fc region (e.g., a wild-type Ig (e.g., hIg, mIg) Fc region)) that enhances serum half-life of the fusion protein (e.g., relative to a reference Ig (e.g., hIg, mIg) Fc region (e.g., a wild-type Ig (e.g., hIg, mIg) Fc region)) through altered binding to the FcRn receptor (e.g., as described herein) (e.g., an FcRn binding profile described herein).

Exemplary amino acid variations of an Ig (e.g., hIg, mIg) Fc region that enhance serum half-life of the Ig Fc region (or a protein comprising the same) are known in the art. See, e.g., Ko 2021 (and references cited therein) (including e.g., Table 1 of Ko 2021); Xinhua Wang, Mary Mathieu, Randall J Brezski, IgG Fc engineering to modulate antibody effector functions, Protein & Cell, Volume 9, Issue 1, January 2018, Pages 63-73, https://doi.org/10.1007/s13238-017-0473-8; U.S. Pat. No. 8,546,543B2; WO2024059652A1; U.S. Pat. No. 11,591,368 (e.g., H433K/N434F); Ko, S., Park, S., Sohn, M. H. et al. An Fc variant with two mutations confers prolonged serum half-life and enhanced effector functions on IgG antibodies. Exp Mol Med 54, 1850-1861 (2022). https://doi.org/10.1038/s12276-022-00870-5; the entire contents of each of which is incorporated herein by reference for all purposes.

Table 12 below, provides exemplary amino acid substitutions (and combinations thereof) and glycoengineering that can be utilized to extend half-life of proteins (e.g., fusion proteins described herein) comprising an Ig Fc region (or fragment thereof). Amino acids in Table 12 are numbered according to the EU numbering scheme. The amino acid substitutions set forth in Table 12 are with reference to an IgG1 Fc region (except where noted). However, a person of ordinary skill in the could identify the corresponding amino acid in a non-IgG1 Fc region, for example in an IgG2 or IgG4 Fc region, should the base amino acid be different between the IgG1 and non-IgG1 Fc region.

TABLE 12
Exemplary hIg Fc Variations to Extend Half-Life.

| | |
|---|---|
| | Exemplary Effects on |
| | Effector Function |
| Variation/Glycoengineering | (Non-Limiting) |

Amino Acid Variations

| | |
|---|---|
| R435H | Extended Half-Life |
| N434A | Extended Half-Life |
| N434W | Extended Half-Life |
| M252Y/S254T/T256E | Extended Half-Life |
| M252Y/T256D | Extended Half-Life |
| M428L/N434S | Extended Half-Life |
| E294Δ/R307P/N434Y | Extended Half-Life |
| T256D/T307Q | Extended Half-Life |
| T256D/T307W | Extended Half-Life |
| T256N/A378V/S383N/N434Y | Extended Half-Life |
| T307Q/Q311V/A378V | Extended Half-Life |
| T256D/H286D/T307R/Q311V/A378V | Extended Half-Life |
| L309D/Q311H/N434S | Extended Half-Life |
| H433K/N434F | Extended Half-Life |
| H433K/N434F (IgG4) | Extended Half-Life |
| E294Δ | Extended Half-Life |

In some embodiments, the Ig Fc region is a hIg Fc region. In some embodiments, the hIg Fc (e.g., IgG1 Fc) region comprises any one or more of the amino acid substitutions set forth in Table 12 (i.e., any one or more amino acid substitution set forth in any set of amino acid substitutions set forth in Table 12). In some embodiments, the hIg Fc (e.g., IgG1 Fc) comprises any one or more of the sets of amino acid substitutions set forth in Table 12. In some embodiments, the hIg Fc (e.g., IgG1 Fc) region comprises any one or more of the glycosylation changes set forth in Table 12.

For example, amino acid variations include, but are not limited to, M428L/N434S, EU numbering according to Kabat; M252Y/S254T/T256E, EU numbering according to Kabat; N434A, EU numbering according to Kabat; N434W, EU numbering according to Kabat; T256D/T307Q, EU numbering according to Kabat; T256D/T307W, EU numbering according to Kabat; M252Y/T256D, EU numbering according to Kabat; T307Q/Q311V/A378V, EU numbering according to Kabat; T256D/H286D/T307R/Q311V/A378V, EU numbering according to Kabat; and L309D/Q311H/N434S, EU numbering according to Kabat. Further amino acid modifications include, H433K/N434F (of IgG1) or H433K/N434F (of IgG4), EU numbering according to Kabat.

In some embodiments, the Ig (e.g., hIg, mIg) Fc region of a fusion protein described herein comprises one or more alteration (including various post-translational modifications e.g., glycosylation, sialylation) that mediates enhanced serum half-life, e.g., relative to a reference (e.g., wild type) Ig (e.g., hIg, mIg) Fc region. In some embodiments, the Ig (e.g., hIg, mIg) Fc region of a fusion protein described herein comprises one or more post-translational modification (e.g., glycosylation, sialylation) that mediates enhanced serum half-life, e.g., relative to a reference (e.g., wild type) Ig (e.g., hIg, mIg) Fc region. In some embodiments, the Ig (e.g., hIg, mIg) Fc region of a fusion protein described herein comprises altered glycosylation that mediates enhanced serum half-life, e.g., relative to a reference (e.g., wild type) Ig (e.g., hIg, mIg) Fc region. In some embodiments, the Ig (e.g., hIg, mIg) Fc region of a fusion protein described herein comprises altered lipidation that mediates enhanced serum half-life, e.g., relative to a reference (e.g., wild type) Ig (e.g., hIg, mIg) Fc region. In some embodiments, the Ig (e.g., hIg, mIg) Fc region of a fusion protein described herein comprises altered sialylation that mediates enhanced serum half-life, e.g., relative to a reference (e.g., wild type) Ig (e.g., hIg, mIg) Fc region. In some embodiments, the Ig (e.g., hIg, mIg) Fc region of a fusion protein described herein is pegylated, which mediates enhanced serum half-life, e.g., relative to a reference (e.g., wild type) Ig (e.g., hIg, mIg) Fc region.

### 5.4.5.4 Ig Effector Function

In some embodiments, the Ig (e.g., hIg, mIg) Fc region of a fusion protein described herein exhibits modulation (e.g., a decrease or increase) of one or more Fc effector function, e.g., relative to a reference (e.g., wild type) Ig (e.g., hIg, mIg) Fc region. Exemplary Ig (e.g., hIg, mIg) Fc effector functions include, but are not limited to, antibody dependent cellular cytotoxicity (ADCC), antibody dependent cellular phagocytosis (ADCP), complement dependent cytotoxicity (CDC), and binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

Standard in vitro and/or in vivo assays known in the art can be conducted to evaluate Fc effector function, including, any one or more of ADCC, CDC, ADCP, Fc receptor (e.g., Fcγ receptor) binding affinity, and C1q binding affinity.

For example, ADCC activity can be assessed utilizing standard (radioactive and non-radioactive) methods known in the art (see, e.g., WO2006/082515, WO2012/130831), the entire contents of each of which is incorporated by reference herein for all purposes). For example, ADCC activity can be assessed using a chromium-5 (⁵¹Cr) assay. Briefly, ⁵¹Cr is pre-loaded into target cells expressing CD20, NK cells are added to the culture, and radioactivity in the cell culture supernatant is assessed (indicative of lysis of the target cells by the NK cells). Similar non-radioactive assays can also be utilized that employ a similar method, but the target cells are pre-loaded with fluorescent dyes, such as calcein-AM, CFSE, BCECF, or lanthanide flurophore (Europium). See, e.g., Parekh, Bhavin S et al. “Development and validation of an antibody-dependent cell-mediated cytotoxicity-reporter gene assay.” mAbs vol. 4, 3 (2012): 310-8. Doi: 10.4161/mabs.19873, the entire contents of which is incorporated by reference herein for all purposes. Exemplary commercially available non-radioactive assays include, for example, ACTI™ non-radioactive cytotoxicity assay for flow cytometry (Cell Technology, Inc. Mountain View, Calif.; and CytoTox 96® non-radioactive cytotoxicity assay (Promega, Madison, Wis.). Additional non-limiting examples of in vitro assays that can be used to assess ADCC activity of a fusion protein described herein include those described in U.S. Pat. Nos. 5,500,362; 5,821,337; Hellstrom, I., et al., Proc. Nat'l Acad. Sci. USA 83 (1986) 7059-7063; Hellstrom, I., et al., Proc. Nat'l Acad. Sci. USA 82 (1985) 1499-1502; and Bruggemann, M., et al., J. Exp. Med. 166 (1987) 1351-1361, the entire contents of each of which is incorporated by reference herein. Alternatively, or additionally, ADCC activity of a fusion protein described herein may be assessed in vivo, e.g., in an animal model such as that disclosed in Clynes, et al., Proc. Nat'l Acad. Sci. USA 95 (1998) 652-656, the entire contents of which is incorporated by reference herein for all purposes.

C1q binding assays can be utilized to assess the ability of a hIg fusion protein described herein to bind C1q (or bind with less affinity than a reference fusion protein) and hence lack (or have decreased) CDC activity. The binding of a hIg fusion protein described herein to C1q can be determined by a variety of in vitro assays (e.g., biochemical or immunological based assays) known in the art for determining Fc-C1q interactions, including e.g., equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4^{th} Ed., Lippincott-Raven, Philadelphia (1999), the entire contents of which is incorporated by reference herein. For example, see, e.g., C1q and C3c binding ELISAs described in WO2006/029879 and WO2005/100402, the entire contents of each of which is incorporated by reference herein for all purposes. Additional CDC activity assays include those described in e.g., Gazzano-Santoro, et al., J. Immunol. Methods 202 (1996) 163; Cragg, M. S., et al., Blood 101 (2003) 1045-1052; and Cragg, M. S., and Glennie, M. J., Blood 103 (2004) 2738-2743), the entire contents of each of which is incorporated by reference herein for all purposes.

ADCP activity can be measured by in vitro or in vivo methods known in the art and also commercially available assays (see, e.g., van de Donk N W, Moreau P, Plesner T, et al. “Clinical efficacy and management of monoclonal antibodies targeting CD38 and SLAMF7 in multiple myeloma,” Blood, 127 (6): 681-695 (2016), the entire contents of each of which is incorporated by reference herein for all purposes). For example, a primary cell based ADCP assay can be used in which fresh human peripheral blood mononuclear cells (PBMCs) are isolated, monocytes isolated and differentiated in culture to macrophages using standard procedures. The macrophages are fluorescently labeled added to cultures containing fluorescently labeled target cells expressing CD20 and a fusion protein described herein. Phagocytosis events can be analyzed using FACS screening and/or microscopy. A modified reporter version of the above described assay can also be used that employs an engineered cell line that stably expresses FcγRIIa (CD32a) as the effector cell line (e.g., an engineered T cell line, e.g., THP-1), removing the requirement for primary cells. Exemplary ADCP assays are described in e.g., Ackerman, M. E. et al. A robust, high-throughput assay to determine the phagocytic activity of clinical antibody samples. J. Immunol. Methods 366, 8-19 (2011); and Mcandrew, E. G. et al. Determining the phagocytic activity of clinical antibody samples. J. Vis. Exp. 3588 (2011). Doi: 10.3791/3588; the entire contents of each of which is incorporated by reference herein.

Binding of a hIg fusion protein described herein to an Ig (e.g., hIg, mIg) Fc receptor can be determined by a variety of in vitro assays (e.g., biochemical or immunological based assays) known in the art for determining Fc-Fc receptor interactions, i.e., specific binding of an Fc region to an Fc receptor. Common assays include equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4″ Ed., Lippincott-Raven, Philadelphia (1999), the entire contents of which is incorporated by reference herein for all purposes.

### (i) Reduced Ig Effector Function

In some embodiments, the Ig Fc region exhibits a decrease in or no detectable activity of one or more Fc effector. As described above, exemplary Ig Fc effector functions include, but are not limited to, ADCC, ADCP, CDC, binding affinity to C1q, and binding affinity to one or more human Fc receptor (e.g., an Fcγ receptor (e.g., FcγRI, FcγRIIa, FcγRIIb, FcγRIIc, FcγRIIIa, and/or FcγRIIIb)).

In some embodiments, the hIg Fc region is modified (e.g., comprises one or more variation (e.g., one or more amino acid substitution, deletion, addition, etc.); altered glycosylation)) (referred to herein as a “modified hIg Fc”). In some embodiments, the modification (e.g., the variation (e.g., one or more amino acid substitution, deletion, addition, etc.); altered glycosylation decreases or abolishes one or more Fc effector function, relative to a reference hIg Fc that does not comprise the modification (e.g., the one or more variation (e.g., the one or more amino acid substitution, deletion, addition, etc.; the altered glycosylation)).

In some embodiments, the modified Ig (e.g., hIg, mIg) Fc fusion protein exhibits no detectable or decreased ADCC compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits no detectable or decreased CDC compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits no detectable or decreased ADCP compared to a reference fusion protein that does not comprise the Ig (e.g., hIg, mIg) Fc modification (e.g., the one or more variation (e.g., one or more amino acid substitution, deletion, or addition)).

In some embodiments, the modified Ig (e.g., hIg, mIg) Fc fusion protein exhibits decreased or no binding affinity to one or more Fc receptor (e.g., human Fc receptor) (e.g., an Fcγ receptor (e.g., an Fcγ receptor (e.g., FcγRI, FcγRIIa, FcγRIIb, FcγRIIc, FcγRIIIa, and/or FcγRIIIb)) compared to a reference fusion protein that does not comprise the Ig (e.g., hIg, mIg) Fc modification (e.g., the one or more variation (e.g., one or more amino acid substitution, deletion, or addition)).

In some embodiments, the modified Ig (e.g., hIg, mIg) Fc fusion protein exhibits decreased or no binding affinity to FcγRI, FcγRIIa, FcγRIIIa, and/or FcγRIIIb compared to a reference fusion protein that does not comprise the hIg Fc modification (e.g., the one or more variation (e.g., one or more amino acid substitution, deletion, or addition)). In some embodiments, the modified Ig (e.g., hIg, mIg) Fc fusion protein exhibits decreased or no binding affinity to FcγRI compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits decreased or no binding affinity to FcγRIIa compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits decreased or no binding affinity to FcγRIIIa compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits decreased or no binding affinity to FcγRIIIb compared to a reference fusion protein that does not comprise the Ig (e.g., hIg, mIg) Fc modification (e.g., the one or more variation (e.g., one or more amino acid substitution, deletion, or addition)).

In some embodiments, the modified Ig (e.g., hIg, mIg) Fc fusion protein exhibits increased binding affinity to one or more Fc receptor (e.g., human Fc receptor) (e.g., an Fcγ receptor (e.g., FcγRIIb)) compared to a reference fusion protein that does not comprise the hIg Fc modification (e.g., the one or more variation (e.g., one or more amino acid substitution, deletion, or addition)). In some embodiments, the modified Ig (e.g., hIg, mIg) Fc fusion protein exhibits increased binding affinity to FcγRIIb compared to a reference fusion protein that does not comprise the Ig (e.g., hIg, mIg) Fc modification (e.g., the one or more variation (e.g., one or more amino acid substitution, deletion, or addition)).

In some embodiments, the modified Ig (e.g., hIg, mIg) Fc fusion protein exhibits decreased or no binding affinity to C1q compared to a reference fusion protein that does not comprise the Ig (e.g., hIg, mIg) Fc modification (e.g., the one or more variation (e.g., one or more amino acid substitution, deletion, or addition)).

Amino acid substitutions that decrease or abolish one or more Ig (e.g., hIg, mIg) Fc effector function are known in the art. See for example, Saunders Kevin, “Conceptual Approaches to Modulating Antibody Effector Functions and Circulation Half-Life,” Frontiers in Immunology, v10 (Jun. 7, 2019) DOI=10.3389/fimmu.2019.01296, the full contents of which is incorporated by reference herein for all purposes, see more particularly for example, e.g., Table 3 of Saunders.

Table 13 below, provides exemplary amino acid substitutions (and combinations thereof) and glycoengineering that can be utilized to decrease one or more hIg Fc effector function. Amino acids in Table 13 are numbered according to the EU numbering scheme. The effects on effector function set forth in Table 13 are exemplary only and not intended to be limiting. The amino acid substitutions set forth in Table 13 are with reference to an IgG1 Fc region (except where noted). However, a person of ordinary skill in the could identify the corresponding amino acid in a non-IgG1 Fc region, for example in an IgG2 or IgG4 Fc region, should the base amino acid be different between the IgG1 and non-IgG1 Fc region.

TABLE 13
Exemplary hIg Fc Variations and Glycoengineering
to Decrease Effector Function.

| | |
|---|---|
| Variation/ | Exemplary Effects on Effector Function |
| Glycoengineering | (Non-Limiting) |

Amino Acid Substitutions

| | |
|---|---|
| L235E | Decreased binding to cell surface FcγRs |
| | Decreased ADCC |
| L234A/L235A | Decreased binding to FcγRI, RII, III |
| | Decreased ADCC, ADCP, CDC |
| S228P/L235E (IgG4) | Decreased binding to FcγRI |
| L234A/L235A/P329G | Eliminates binding to Decreased binding to |
| | FcγRI, RII, III, C1q |
| | Decreased ADCP |
| L234A/L235A/P329A | Eliminates binding to Decreased binding to |
| | FcγRI, RII, III, C1q |
| | Decreased ADCP |
| L235A/G237A/P329G | Reduced ADCC, ADCP, CDC |
| L235A/G237A/P329A | Reduced ADCC, ADCP, CDC |
| P331S/L234E/L235F | Eliminates binding to Decreased binding to |
| | FcγRI, RII, III, C1q |
| | Decreased CDC |
| D235A | Decreased binding to FcγRI, RII, III |
| | Reduced ADCC, ADCP |
| G237A | Decreased binding to FcγRII |
| | Decreased ADCP |
| E318A | Decreased binding to FcγRII |
| | Decreased ADCP |
| E233P | Decreased binding to FcγRI, RII, III |
| G236R/L328R | Decreased binding to all FcγRs |
| | Decreased ADCC |
| A330L | Decreased C1q binding |
| | Decreased CDC |
| D270A | Decreased C1q binding |
| | Decreased CDC |
| K332A | Decreased C1q binding |
| | Decreased CDC |
| P329A | Decreased C1q binding |
| | Decreased CDC |
| P331A | Decreased C1q binding |
| | Decreased CDC |
| V264A | Decreased C1q binding |
| | Decreased CDC |
| F241A | Decreased C1q binding |
| | Decreased CDC |
| N297A | Decreased binding to FcγRI, RIIIa |
| | Decreased C1q binding |
| | Decreased ADCC |
| | Decreased ADCP |
| | Decreased CDC |
| N297G | Decreased binding to FcγRI, RIIIa |
| | Decreased C1q binding |
| | Decreased ADCC |
| | Decreased ADCP |
| | Decreased CDC |
| N297Q | Decreased binding to FcγRI, RIIIa |
| | Decreased C1q binding |
| | Decreased ADCC |
| | Decreased ADCP |
| | Decreased CDC |
| S228P/F234A/L235A | Decreased binding to FcγRI, RIIa, RIIIa |
| (IgG4) | Decreased ADCC |
| | Decreased CDC |
| S228P/F234A/L235E | Decreased binding to FcγRI, RIIa, RIIIa |
| (IgG4) | Decreased ADCC |
| | Decreased CDC |

Glycoengineering

| | |
|---|---|
| High mannose | Decreased C1q binding |
| glycosylation | Decreased CDC |

In some embodiments, the Ig Fc region is a hIg Fc region. In some embodiments, the hIg Fc (e.g., IgG1 Fc) region comprises any one or more of the amino acid substitutions set forth in Table 13 (i.e., any one or more amino acid substitution set forth in any set of amino acid substitutions set forth in Table 13). In some embodiments, the hIg Fc (e.g., IgG1 Fc) comprises any one or more of the sets of amino acid substitutions set forth in Table 13. In some embodiments, the hIg Fc (e.g., IgG1 Fc) region comprises any one or more of the glycosylation changes set forth in Table 13.

In some embodiments, the modified Ig Fc fusion protein comprises a hIg Fc region comprising one or more amino acid variation. In some embodiments, the modified hIg Fc fusion protein comprises a hIg4 Fc region comprising one or more amino acid variation. In some embodiments, the hIgG4 Fc region comprises an amino acid substitution at amino acid positions S228, F234, and/or L235, EU numbering according to Kabat. In some embodiments, the hIgG4 Fc region comprises the following amino acid substitutions S228P, F234A, and/or L235A, EU numbering according to Kabat. In some embodiments, the hIgG4 Fc region comprises the following amino acid substitutions S228P, F234A, and/or L235E, EU numbering according to Kabat. In some embodiments, the hIgG4 Fc comprises the following amino acid substitutions S228P and/or L235E, EU numbering according to Kabat.

In some embodiments, the S228P variation stabilized the hinge region. See, e.g., Silva, John-Paul et al. “The S228P mutation prevents in vivo and in vitro IgG4 Fab-arm exchange as demonstrated using a combination of novel quantitative immunoassays and physiological matrix preparation.” The Journal of biological chemistry vol. 290, 9 (2015): 5462-9. doi: 10.1074/jbc.M114.600973, the entire contents of which is incorporated herein by reference for all purposes.

In some embodiments, the modified hIg Fc fusion protein comprises a hIgG1 Fc region comprising one or more amino acid variations. In some embodiments, the hIgG1 Fc region comprises an amino acid substitution at amino acid positions L234, L235, and/or P329, EU numbering according to Kabat. In some embodiments, the hIgG1 Fc region comprises the following amino acid substitutions L234A and/or L235A, EU numbering according to Kabat. In some embodiments, the hIgG1 Fc region comprises the following amino acid substitutions L234A, L235A, and P329G, EU numbering according to Kabat. In some embodiments, the hIgG1 Fc region comprises the following amino acid substitutions L234A, L235A, and P329A, EU numbering according to Kabat.

In some embodiments, the modified hIg Fc fusion protein comprises a hIgG1 Fc region comprising one or more amino acid variations. In some embodiments, the hIgG1 Fc region comprises an amino acid substitution at amino acid positions L235, G237, and/or P329, EU numbering according to Kabat. In some embodiments, the hIgG1 Fc region comprises the following amino acid substitutions L235A and/or G237A, EU numbering according to Kabat. In some embodiments, the hIgG1 Fc region comprises the following amino acid substitutions L235A, G237A, and P329G, EU numbering according to Kabat. In some embodiments, the hIgG1 Fc region comprises the following amino acid substitutions L235A, G237A, and P329A, EU numbering according to Kabat.

The amino acid sequence of exemplary variant hIg Fc regions that are known in the art to exhibit a decrease in one more effector function is provided in Table 6.

TABLE 6
The amino acid sequence of exemplary variant hlg Fc Regions.

| | | SEQ |
|---|---|---|
| Description | Amino Acid Sequence | ID NO |
| hIgG1 CH2 Region + | PCPAPEAAGGPSVFLFPPKPKDTLMISRTPEVTCVVVDVSHED | 142 |
| CH3 Region | PEVKFNWYVDGVEVHNAKTKPREEQYNSTYRVVSVLTVLHQDW | |
| L234A/L235A | LNGKEYKCKVSNKALPAPIEKTISKAKGQPREPQVYTLPPSRD | |
| With C-terminal Lysine | ELTKNQVSLTCLVKGFYPSDIAVEWESNGQPENNYKTTPPVLD | |
| | SDGSFFLYSKLTVDKSRWQQGNVFSCSVMHEALHNHYTQKSLS | |
| | LSPGK | |
| hIgG1 CH2 Region + | PCPAPEAAGGPSVFLFPPKPKDTLMISRTPEVTCVVVDVSHED | 143 |
| CH3 Region | PEVKFNWYVDGVEVHNAKTKPREEQYNSTYRVVSVLTVLHQDW | |
| L234A/L235A | LNGKEYKCKVSNKALPAPIEKTISKAKGQPREPQVYTLPPSRD | |
| Without C-terminal | ELTKNQVSLTCLVKGFYPSDIAVEWESNGQPENNYKTTPPVLD | |
| Lysine | SDGSFFLYSKLTVDKSRWQQGNVFSCSVMHEALHNHYTOKSLS | |
| | LSPG | |
| hIgG1 Partial Hinge | TCPPCPAPEAAGGPSVFLFPPKPKDTLMISRTPEVTCVVVDVS | 144 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGOPREPQVYTLPP | |
| L234A/L235A | SRDELTKNQVSLTCLVKGFYPSDIAVEWESNGQPENNYKTTPP | |
| With C-terminal Lysine | VLDSDGSFFLYSKLTVDKSRWQQGNVFSCSVMHEALHNHYTQK | |
| | SLSLSPGK | |
| hIgG1 Partial Hinge | TCPPCPAPEAAGGPSVFLFPPKPKDTLMISRTPEVTCVVVDVS | 145 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| L234A/L235A | SRDELTKNQVSLTCLVKGFYPSDIAVEWESNGQPENNYKTTPP | |
| Without C-terminal | VLDSDGSFFLYSKLTVDKSRWQQGNVFSCSVMHEALHNHYTQK | |
| Lysine | SLSLSPG | |
| hIgG1 Hinge Region + | EPKSCDKTHTCPPCPAPEAAGGPSVFLFPPKPKDTLMISRTPE | 146 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| L234A/L235A | EPQVYTLPPSRDELTKNQVSLTCLVKGFYPSDIAVEWESNGOP | |
| With C-terminal Lysine | ENNYKTTPPVLDSDGSFFLYSKLTVDKSRWQQGNVFSCSVMHE | |
| | ALHNHYTQKSLSLSPGK | |
| hIgG1 Hinge Region + | EPKSCDKTHTCPPCPAPEAAGGPSVFLFPPKPKDTLMISRTPE | 147 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| L234A/L235A | EPQVYTLPPSRDELTKNQVSLTCLVKGFYPSDIAVEWESNGQP | |
| Without C-terminal | ENNYKTTPPVLDSDGSFFLYSKLTVDKSRWQQGNVFSCSVMHE | |
| Lysine | ALHNHYTQKSLSLSPG | |
| hIgG4 CH2 Region + | APEAAGGPSVFLFPPKPKDTLMISRTPEVTCVVVDVSQEDPEV | 148 |
| CH3 Region | QFNWYVDGVEVHNAKTKPREEQFNSTYRVVSVLTVLHQDWLNG | |
| S228P/F234A/L235A | KEYKCKVSNKGLPSSIEKTISKAKGQPREPQVYTLPPSQEEMT | |
| With C-terminal Lysine | KNQVSLTCLVKGFYPSDIAVEWESNGQPENNYKTTPPVLDSDG | |
| | SFFLYSRLTVDKSRWQEGNVFSCSVMHEALHNHYTOKSLSLSL | |
| | GK | |
| hIgG4 CH2 Region + | APEAAGGPSVFLFPPKPKDTLMISRTPEVTCVVVDVSQEDPEV | 149 |
| CH3 Region | QFNWYVDGVEVHNAKTKPREEQFNSTYRVVSVLTVLHQDWLNG | |
| S228P/F234A/L235A | KEYKCKVSNKGLPSSIEKTISKAKGQPREPQVYTLPPSQEEMT | |
| Without C-terminal | KNQVSLTCLVKGFYPSDIAVEWESNGOPENNYKTTPPVLDSDG | |
| Lysine | SFFLYSRLTVDKSRWQEGNVFSCSVMHEALHNHYTQKSLSLSL | |
| | G | |
| hIgG4 Partial Hinge | PCPSCPAPEAAGGPSVFLFPPKPKDTLMISRTPEVTCVVVDVS | 150 |
| Region + CH2 Region + | QEDPEVQFNWYVDGVEVHNAKTKPREEQFNSTYRVVSVLTVLH | |
| CH3 Region | QDWLNGKEYKCKVSNKGLPSSIEKTISKAKGQPREPQVYTLPP | |
| S228P/F234A/L235A | SQEEMTKNQVSLTCLVKGFYPSDIAVEWESNGQPENNYKTTPP | |
| With C-terminal Lysine | VLDSDGSFFLYSRLTVDKSRWQEGNVFSCSVMHEALHNHYTQK | |
| | SLSLSLGK | |
| hIgG4 Partial Hinge | PCPPCPAPEAAGGPSVFLFPPKPKDTLMISRTPEVTCVVVDVS | 151 |
| Region + CH2 Region + | QEDPEVQFNWYVDGVEVHNAKTKPREEQFNSTYRVVSVLTVLH | |
| CH3 Region | QDWLNGKEYKCKVSNKGLPSSIEKTISKAKGQPREPQVYTLPP | |
| S228P/F234A/L235A | SQEEMTKNQVSLTCLVKGFYPSDIAVEWESNGQPENNYKTTPP | |
| Without C-terminal | VLDSDGSFFLYSRLTVDKSRWQEGNVFSCSVMHEALHNHYTQK | |
| Lysine | SLSLSLG | |
| hIgG4 Hinge Region + | ESKYGPPCPPCPAPEAAGGPSVFLFPPKPKDTLMISRTPEVTC | 152 |
| CH2 Region + CH3 | VVVDVSQEDPEVQFNWYVDGVEVHNAKTKPREEQFNSTYRVVS | |
| Region | VLTVLHQDWLNGKEYKCKVSNKGLPSSIEKTISKAKGOPREPQ | |
| S228P/F234A/L235A | VYTLPPSQEEMTKNQVSLTCLVKGFYPSDIAVEWESNGQPENN | |
| With C-terminal Lysine | YKTTPPVLDSDGSFFLYSRLTVDKSRWQEGNVFSCSVMHEALH | |
| | NHYTQKSLSLSLGK | |
| hIgG4 Hinge Region + | ESKYGPPCPPCPAPEAAGGPSVFLFPPKPKDTLMISRTPEVTC | 153 |
| CH2 Region + CH3 | VVVDVSQEDPEVQFNWYVDGVEVHNAKTKPREEQFNSTYRVVS | |
| Region | VLTVLHQDWLNGKEYKCKVSNKGLPSSIEKTISKAKGQPREPQ | |
| S228P/F234A/L235A | VYTLPPSQEEMTKNQVSLTCLVKGFYPSDIAVEWESNGQPENN | |
| Without C-terminal | YKTTPPVLDSDGSFFLYSRLTVDKSRWQEGNVFSCSVMHEALH | |
| Lysine | NHYTQKSLSLSLG | |
| hIgG4 Hinge Region + | AESKYGPPCPPCPAPEAAGGPSVFLFPPKPKDTLMISRTPEVT | 154 |
| CH2 Region + CH3 | CVVVDVSQEDPEVQFNWYVDGVEVHNAKTKPREEQFNSTYRVV | |
| Region | SVLTVLHQDWLNGKEYKCKVSNKGLPSSIEKTISKAKGQPREP | |
| (Variant) | QVYTLPPSQEEMTKNQVSLTCLVKGFYPSDIAVEWESNGOPEN | |
| S228P/F234A/L235A | NYKTTPPVLDSDGSFFLYSRLTVDKSRWQEGNVFSCSVMHEAL | |
| With C-terminal Lysine | HNHYTQKSLSLSLGK | |
| hIgG4 Hinge Region + | AESKYGPPCPPCPAPEAAGGPSVFLFPPKPKDTLMISRTPEVT | 155 |
| CH2 Region + CH3 | CVVVDVSQEDPEVQFNWYVDGVEVHNAKTKPREEQFNSTYRVV | |
| Region | SVLTVLHQDWLNGKEYKCKVSNKGLPSSIEKTISKAKGQPREP | |
| (Variant) | QVYTLPPSQEEMTKNQVSLTCLVKGFYPSDIAVEWESNGQPEN | |
| S228P/F234A/L235A | NYKTTPPVLDSDGSFFLYSRLTVDKSRWQEGNVFSCSVMHEAL | |
| Without C-terminal | HNHYTQKSLSLSLG | |
| Lysine | | |

In some embodiments, the variant hIg Fc fusion protein comprises a hIg Fc region comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 6. For example, the variant hIg Fc fusion protein may comprise a hIg Fc region comprising an amino acid sequence at least 85% identical to the amino acid sequence of a polypeptide set forth in Table 6. The variant hIg Fc fusion protein may comprise a hIg Fc region comprising an amino acid sequence at least 90% identical to the amino acid sequence of a polypeptide set forth in Table 6. The variant hIg Fc fusion protein may comprise a hIg Fc region comprising an amino acid sequence at least 95% identical to the amino acid sequence of a polypeptide set forth in Table 6. In some embodiments, the variant hIg Fc fusion protein preferably may comprise a hIg Fc region comprising an amino acid sequence 100% identical to the amino acid sequence of a polypeptide set forth in Table 6.

In some embodiments, the variant hIg Fc fusion protein comprises a hIg Fc region consisting of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 6. For example, the variant hIg Fc fusion protein may comprise a hIg Fc region consisting of an amino acid sequence at least 85% identical to the amino acid sequence of a polypeptide set forth in Table 6. The variant hIg Fc fusion protein may comprise a hIg Fc region consisting of an amino acid sequence at least 90% identical to the amino acid sequence of a polypeptide set forth in Table 6. The variant hIg Fc fusion protein may comprise a hIg Fc region consisting of an amino acid sequence at least 95% identical to the amino acid sequence of a polypeptide set forth in Table 6. In some embodiments, the variant hIg Fc fusion protein preferably may comprise a hIg Fc region consisting of an amino acid sequence 100% identical to the amino acid sequence of a polypeptide set forth in Table 6.

In some embodiments, the amino acid sequence of the variant hIg Fc fusion protein comprises a hIg Fc region that comprises the amino acid sequence of a polypeptide set forth in Table 6, and further comprises 1 or more but less than 15% (less than 12%, less than 10%, less than 8%), amino acid variations (e.g., amino acid substitutions, deletions, or additions). In some embodiments, the amino acid sequence of the variant hIg Fc fusion protein comprises a hIg Fc region that comprises the amino acid sequence of a polypeptide set forth in Table 6, and further comprises at least about 1, 2, 3, 4, 5, 6, 7, 8, 9, or 10 amino acid variations (e.g., substitutions, additions, deletions, etc.). In some embodiments, the amino acid sequence of the variant hIg Fc fusion protein comprises a hIg Fc region that comprises the amino acid sequence of a polypeptide set forth in Table 6, and further comprises about 1, 2, 3, 4, 5, 6, 7, 8, 9, or 10 amino acid variations (e.g., substitutions, additions, deletions, etc.). In some embodiments, the amino acid sequence of the variant hIg Fc fusion protein comprises a hIg Fc region that comprises the amino acid sequence of a polypeptide set forth in Table 6, and further consists of about 1, 2, 3, 4, 5, 6, 7, 8, 9, or 10 amino acid variations (e.g., substitutions, additions, deletions, etc.). In some embodiments, the amino acid sequence of the variant hIg Fc fusion protein comprises a hIg Fc region that comprises the amino acid sequence of a polypeptide set forth in Table 6, and further comprises no more than about 1, 2, 3, 4, 5, 6, 7, 8, 9, or 10 amino acid variations (e.g., substitutions, additions, deletions, etc.).

In some embodiments, the amino acid sequence of the variant hIg Fc fusion protein comprises a hIg Fc region that consists of the amino acid sequence of a polypeptide set forth in Table 6, and further comprises 1 or more but less than 15% (less than 12%, less than 10%, less than 8%), amino acid variations (e.g., amino acid substitutions, deletions, or additions). In some embodiments, the amino acid sequence of the variant hIg Fc fusion protein comprises a hIg Fc region that consists of the amino acid sequence of a polypeptide set forth in Table 6, and further comprises at least about 1, 2, 3, 4, 5, 6, 7, 8, 9, or 10 amino acid variations (e.g., substitutions, additions, deletions, etc.). In some embodiments, the amino acid sequence of the variant hIg Fc fusion protein comprises a hIg Fc region that consists of the amino acid sequence of a polypeptide set forth in Table 6, and further comprises about 1, 2, 3, 4, 5, 6, 7, 8, 9, or 10 amino acid variations (e.g., substitutions, additions, deletions, etc.). In some embodiments, the amino acid sequence of the variant hIg Fc fusion protein comprises a hIg Fc region that consists of the amino acid sequence of a polypeptide set forth in Table 6, and further consists of about 1, 2, 3, 4, 5, 6, 7, 8, 9, or 10 amino acid variations (e.g., substitutions, additions, deletions, etc.). In some embodiments, the amino acid sequence of the variant hIg Fc fusion protein comprises a hIg Fc region that consists of the amino acid sequence of a polypeptide set forth in Table 6, and further comprises no more than about 1, 2, 3, 4, 5, 6, 7, 8, 9, or 10 amino acid variations (e.g., substitutions, additions, deletions, etc.).

In some embodiments, the amino acid sequence of the variant hIg Fc fusion protein comprises a hIg Fc region that comprises an amino acid sequence at least 85%, 86%, 87%, 88%, 89%, 90%, 91%, 92%, 93%, 94%, 95%, 96%, 97%, 98%, or 100% identical to the amino acid sequence set forth in any one of SEQ ID NOS: 142-155. For example, the amino acid sequence of the variant hIg Fc fusion protein may comprise a hIg Fc region that comprises an amino acid sequence at least 85% identical to the amino acid sequence set forth in any one of SEQ ID NOS: 142-155. The amino acid sequence of the variant hIg Fc fusion protein may comprise a hIg Fc region that comprises an amino acid sequence at least 90% identical to the amino acid sequence set forth in any one of SEQ ID NOS: 142-155. The amino acid sequence of the variant hIg Fc fusion protein may comprise a hIg Fc region that comprises an amino acid sequence at least 95% identical to the amino acid sequence set forth in any one of SEQ ID NOS: 142-155. In some embodiments, the amino acid sequence of the variant hIg Fc fusion protein preferably may comprise a hIg Fc region that comprises an amino acid sequence 100% identical to the amino acid sequence set forth in any one of SEQ ID NOS: 142-155.

In some embodiments, the amino acid sequence of the variant hIg Fc fusion protein comprises a hIg Fc region that consists of an amino acid sequence at least 85%, 86%, 87%, 88%, 89%, 90%, 91%, 92%, 93%, 94%, 95%, 96%, 97%, 98%, or 100% identical to the amino acid sequence set forth in any one of SEQ ID NOS: 142-155. For example, the amino acid sequence of the variant hIg Fc fusion protein may comprise a hIg Fc region that consists of an amino acid sequence at least 85% identical to the amino acid sequence set forth in any one of SEQ ID NOS: 142-155. The amino acid sequence of the variant hIg Fc fusion protein may comprise a hIg Fc region that consists of an amino acid sequence at least 90% identical to the amino acid sequence set forth in any one of SEQ ID NOS: 142-155. The amino acid sequence of the variant hIg Fc fusion protein may comprise a hIg Fc region that consists of an amino acid sequence at least 95% identical to the amino acid sequence set forth in any one of SEQ ID NOS: 142-155. In some embodiments, the amino acid sequence of the variant hIg Fc fusion protein preferably may comprise a hIg Fc region that consists of an amino acid sequence 100% identical to the amino acid sequence set forth in any one of SEQ ID NOS: 142-155.

In some embodiments, the amino acid sequence of the variant hIg Fc fusion protein comprises a hIg Fc region that comprises the amino acid sequence set forth in any one of SEQ ID NOS: 142-155, and further comprises 1 or more but less than 15% (less than 12%, less than 10%, less than 8%), amino acid variations (e.g., amino acid substitutions, deletions, or additions). In some embodiments, the amino acid sequence of the variant hIg Fc fusion protein comprises a hIg Fc region that comprises the amino acid sequence set forth in any one of SEQ ID NOS: 142-155, and further comprises or consists of at least about 1, 2, 3, 4, 5, 6, 7, 8, 9, or 10 amino acid variations (e.g., substitutions, additions, deletions, etc.). In some embodiments, the amino acid sequence of the variant hIg Fc fusion protein comprises a hIg Fc region that comprises the amino acid sequence set forth in any one of SEQ ID NOS: 142-155, and further comprises about 1, 2, 3, 4, 5, 6, 7, 8, 9, or 10 amino acid variations (e.g., substitutions, additions, deletions, etc.). In some embodiments, the amino acid sequence of the variant hIg Fc fusion protein comprises a hIg Fc region that comprises the amino acid sequence set forth in any one of SEQ ID NOS: 142-155, and further consists of about 1, 2, 3, 4, 5, 6, 7, 8, 9, or 10 amino acid variations (e.g., substitutions, additions, deletions, etc.). In some embodiments, the amino acid sequence of the variant hIg Fc fusion protein comprises a hIg Fc region that comprises the amino acid sequence set forth in any one of SEQ ID NOS: 142-155, and further comprises no more than about 1, 2, 3, 4, 5, 6, 7, 8, 9, or 10 amino acid variations (e.g., substitutions, additions, deletions, etc.).

In some embodiments, the amino acid sequence of the variant hIg Fc fusion protein comprises a hIg Fc region that consists of the amino acid sequence set forth in any one of SEQ ID NOS: 142-155, and further comprises 1 or more but less than 15% (less than 12%, less than 10%, less than 8%), amino acid variations (e.g., amino acid substitutions, deletions, or additions). In some embodiments, the amino acid sequence of the variant hIg Fc fusion protein comprises a hIg Fc region that consists of the amino acid sequence set forth in any one of SEQ ID NOS: 142-155, and further comprises or consists of at least about 1, 2, 3, 4, 5, 6, 7, 8, 9, or 10 amino acid variations (e.g., substitutions, additions, deletions, etc.). In some embodiments, the amino acid sequence of the variant hIg Fc fusion protein comprises a hIg Fc region that consists of the amino acid sequence set forth in any one of SEQ ID NOS: 142-155, and further comprises about 1, 2, 3, 4, 5, 6, 7, 8, 9, or 10 amino acid variations (e.g., substitutions, additions, deletions, etc.). In some embodiments, the amino acid sequence of the variant hIg Fc fusion protein comprises a hIg Fc region that consists of the amino acid sequence set forth in any one of SEQ ID NOS: 142-155, and further consists of about 1, 2, 3, 4, 5, 6, 7, 8, 9, or 10 amino acid variations (e.g., substitutions, additions, deletions, etc.). In some embodiments, the amino acid sequence of the variant hIg Fc fusion protein comprises a hIg Fc region that consists of the amino acid sequence set forth in any one of SEQ ID NOS: 142-155, and further comprises no more than about 1, 2, 3, 4, 5, 6, 7, 8, 9, or 10 amino acid variations (e.g., substitutions, additions, deletions, etc.).

In some embodiments, the variant mIg Fc fusion protein comprises a mIgG2a Fc region comprising one or more amino acid variations. In some embodiments, the mIgG2a Fc region comprises an amino acid substitution at amino acid positions L234, L235, and/or P329, EU numbering according to Kabat. In some embodiments, the mIgG2a Fc region comprises the following amino acid substitutions L234P and/or L235P, EU numbering according to Kabat. In some embodiments, the mIgG2a Fc region comprises the following amino acid substitutions L234P, L235P, and P329G, EU numbering according to Kabat. In some embodiments, the mIgG2a Fc region comprises the following amino acid substitutions L234P, L235P, and P329A, EU numbering according to Kabat.

In some embodiments, the variant mIg Fc fusion protein comprises a mIgG2a Fc region comprising one or more amino acid variations. In some embodiments, the mIgG2a Fc region comprises an amino acid substitution at amino acid positions L234, L235, and/or P329, EU numbering according to Kabat. In some embodiments, the mIgG2a Fc region comprises the following amino acid substitutions L234A and/or L235A, EU numbering according to Kabat. In some embodiments, the mIgG2a Fc region comprises the following amino acid substitutions L234A, L235A, and P329G, EU numbering according to Kabat. In some embodiments, the mIgG2a Fc region comprises the following amino acid substitutions L234A, L235A, and P329A, EU numbering according to Kabat.

The amino acid sequence of exemplary variant hIg Fc regions that are known in the art to exhibit a decrease in one more effector function is provided in Table 7.

TABLE 7
The amino acid sequence of exemplary variant mlg Fc Regions.

| | | |
|---|---|---|
| | | SEQ |
| Description | Amino Acid Sequence | ID NO |
| mIgG2a CH2 Region + | APNAAGGPSVFIFPPKIKDVLMISLSPIVTCVVVDVSEDDPDV | 156 |
| CH3 Region | QISWFVNNVEVHTAQTQTHREDYNSTLRVVSALPIQHQDWMSG | |
| L234P/L235P | KEFKCKVNNKDLPAPIERTISKPKGSVRAPQVYVLPPPEEEMT | |
| With C-terminal Lysine | KKQVTLTCMVTDEMPEDIYVEWTNNGKTELNYKNTEPVLDSDG | |
| | SYFMYSKLRVEKKNWVERNSYSCSVVHEGLHNHHTTKSFSRTP | |
| | GK | |
| mIgG2a CH2 Region + | APNAAGGPSVFIFPPKIKDVLMISLSPIVTCVVVDVSEDDPDV | 157 |
| CH3 Region | QISWFVNNVEVHTAQTQTHREDYNSTLRVVSALPIQHQDWMSG | |
| L234P/L235P | KEFKCKVNNKDLPAPIERTISKPKGSVRAPQVYVLPPPEEEMT | |
| Without C-terminal | KKQVTLTCMVTDEMPEDIYVEWTNNGKTELNYKNTEPVLDSDG | |
| Lysine | SYFMYSKLRVEKKNWVERNSYSCSVVHEGLHNHHTTKSFSRTP | |
| | G | |
| mIgG2a Hinge Region + | EPRGPTIKPCPPCKCPAPNAAGGPSVFIFPPKIKDVLMISLSP | 158 |
| CH2 Region + CH3 | IVTCVVVDVSEDDPDVQISWFVNNVEVHTAQTQTHREDYNSTL | |
| Region | RVVSALPIQHQDWMSGKEFKCKVNNKDLGAPIERTISKPKGSV | |
| L234P/L235P /P329G | RAPQVYVLPPPEEEMTKKQVTLTCMVTDFMPEDIYVEWINNGK | |
| With C-terminal Lysine | TELNYKNTEPVLDSDGSYFMYSKLRVEKKNWVERNSYSCSVVH | |
| | EGLHNHHTTKSFSRTPGK | |
| mIgG2a Hinge Region + | EPRGPTIKPCPPCKCPAPNAAGGPSVFIFPPKIKDVLMISLSP | 159 |
| CH2 Region + CH3 | IVTCVVVDVSEDDPDVQISWFVNNVEVHTAQTQTHREDYNSTL | |
| Region | RVVSALPIQHQDWMSGKEFKCKVNNKDLGAPIERTISKPKGSV | |
| L234P/L235P /P329G | RAPQVYVLPPPEEEMTKKQVTLTCMVTDFMPEDIYVEWINNGK | |
| Without C-terminal | TELNYKNTEPVLDSDGSYFMYSKLRVEKKNWVERNSYSCSVVH | |
| Lysine | EGLHNHHTTKSFSRTPG | |
| mIgG2a CH2 Region + | APNAAGGPSVFIFAAKIKDVLMISLSPIVTCVVVDVSEDDPDV | 160 |
| CH3 Region | QISWFVNNVEVHTAQTQTHREDYNSTLRVVSALPIQHQDWMSG | |
| L234A/L235A | KEFKCKVNNKDLPAPIERTISKPKGSVRAPQVYVLPPPEEEMT | |
| With C-terminal Lysine | KKQVTLTCMVTDFMPEDIYVEWINNGKTELNYKNTEPVLDSDG | |
| | SYFMYSKLRVEKKNWVERNSYSCSVVHEGLHNHHTTKSFSRTP | |
| | GK | |
| mIgG2a CH2 Region + | APNAAGGPSVFIFAAKIKDVLMISLSPIVTCVVVDVSEDDPDV | 161 |
| CH3 Region | QISWFVNNVEVHTAQTQTHREDYNSTLRVVSALPIQHQDWMSG | |
| L234A/L235A | KEFKCKVNNKDLPAPIERTISKPKGSVRAPQVYVLPPPEEEMT | |
| Without C-terminal | KKQVTLTCMVTDFMPEDIYVEWTNNGKTELNYKNTEPVLDSDG | |
| Lysine | SYFMYSKLRVEKKNWVERNSYSCSVVHEGLHNHHTTKSFSRTP | |
| | G | |
| mIgG2a Hinge Region + | EPRGPTIKPCAACKCPAPNAAGGPSVFIFPPKIKDVLMISLSP | 162 |
| CH2 Region + CH3 | IVTCVVVDVSEDDPDVQISWFVNNVEVHTAQTQTHREDYNSTL | |
| Region | RVVSALPIQHQDWMSGKEFKCKVNNKDLGAPIERTISKPKGSV | |
| L234A/L235A/P329G | RAPQVYVLPPPEEEMTKKQVTLTCMVTDFMPEDIYVEWTNNGK | |
| With C-terminal Lysine | TELNYKNTEPVLDSDGSYFMYSKLRVEKKNWVERNSYSCSVVH | |
| | EGLHNHHTTKSFSRTPGK | |
| mIgG2a Hinge Region + | EPRGPTIKPCAACKCPAPNAAGGPSVFIFPPKIKDVLMISLSP | 163 |
| CH2 Region + CH3 | IVTCVVVDVSEDDPDVQISWFVNNVEVHTAQTQTHREDYNSTL | |
| Region | RVVSALPIQHQDWMSGKEFKCKVNNKDLGAPIERTISKPKGSV | |
| L234A/L235A/P329G | RAPQVYVLPPPEEEMTKKQVTLTCMVTDFMPEDIYVEWTNNGK | |
| Without C-terminal | TELNYKNTEPVLDSDGSYFMYSKLRVEKKNWVERNSYSCSVVH | |
| Lysine | EGLHNHHTTKSFSRTPG | |

In some embodiments, the variant mIg Fc fusion protein comprises a mIg Fc region comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 7. For example, the variant mIg Fc fusion protein may comprise a mIg Fc region comprising an amino acid sequence at least 85% identical to the amino acid sequence of a polypeptide set forth in Table 7. The variant mIg Fc fusion protein may comprise a mIg Fc region comprising an amino acid sequence at least 90% identical to the amino acid sequence of a polypeptide set forth in Table 7. The variant mIg Fc fusion protein may comprise a mIg Fc region comprising an amino acid sequence at least 95% identical to the amino acid sequence of a polypeptide set forth in Table 7. In some embodiments, the variant mIg Fc fusion protein preferably may comprise a mIg Fc region comprising an amino acid sequence 100% identical to the amino acid sequence of a polypeptide set forth in Table 7.

In some embodiments, the amino acid sequence of the variant mIg Fc fusion protein comprises a mIg Fc region that comprises or consists of the amino acid sequence of a polypeptide set forth in Table 7, and further comprises 1 or more but less than 15% (less than 12%, less than 10%, less than 8%), amino acid variations (e.g., amino acid substitutions, deletions, or additions). In some embodiments, the amino acid sequence of the variant mIg Fc fusion protein comprises a mIg Fc region that comprises or consists of the amino acid sequence of a polypeptide set forth in Table 7, and further comprises or consists of at least about 1, 2, 3, 4, 5, 6, 7, 8, 9, or 10 amino acid variations (e.g., substitutions, additions, deletions, etc.). In some embodiments, the amino acid sequence of the variant mIg Fc fusion protein comprises a mIg Fc region that comprises or consists of the amino acid sequence of a polypeptide set forth in Table 7, and further comprises or consists of about 1, 2, 3, 4, 5, 6, 7, 8, 9, or 10 amino acid variations (e.g., substitutions, additions, deletions, etc.). In some embodiments, the amino acid sequence of the variant mIg Fc fusion protein comprises a mIg Fc region that comprises or consists of the amino acid sequence of a polypeptide set forth in Table 7, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the variant mIg Fc fusion protein comprises a mIg Fc region that comprises or consists of the amino acid sequence of a polypeptide set forth in Table 7, and further comprises 1 or more but less than 15% (less than 12%, less than 10%, less than 8%), amino acid substitutions. In some embodiments, the amino acid sequence of the variant mIg Fc fusion protein comprises a mIg Fc region that comprises or consists of the amino acid sequence of a polypeptide set forth in Table 7, and further comprises or consists of at least about 1, 2, 3, 4, 5, 6, 7, 8, 9, or 10 amino acid substitutions. In some embodiments, the amino acid sequence of the variant mIg Fc fusion protein comprises a mIg Fc region that comprises or consists of the amino acid sequence of a polypeptide set forth in Table 7, and further comprises or consists of about 1, 2, 3, 4, 5, 6, 7, 8, 9, or 10 amino acid substitutions. In some embodiments, the amino acid sequence of the variant mIg Fc fusion protein comprises a mIg Fc region that comprises or consists of the amino acid sequence of a polypeptide set forth in Table 7, and further comprises or consists of no more than about 1, 2, 3, 4, 5, 6, 7, 8, 9, or 10 amino acid substitutions.

In some embodiments, the amino acid sequence of the variant mIg Fc fusion protein comprises a mIg Fc region that comprises an amino acid sequence at least 85%, 86%, 87%, 88%, 89%, 90%, 91%, 92%, 93%, 94%, 95%, 96%, 97%, 98%, or 100% identical to the amino acid sequence set forth in any one of SEQ ID NOS: 156-163. For example, the amino acid sequence of the variant mIg Fc fusion protein may comprise a mIg Fc region that comprises an amino acid sequence at least 85% identical to the amino acid sequence set forth in any one of SEQ ID NOS: 156-163. The amino acid sequence of the variant mIg Fc fusion protein may comprise a mIg Fc region that comprises an amino acid sequence at least 90% identical to the amino acid sequence set forth in any one of SEQ ID NOS: 156-163. The amino acid sequence of the variant mIg Fc fusion protein may comprise a mIg Fc region that comprises an amino acid sequence at least 95% identical to the amino acid sequence set forth in any one of SEQ ID NOS: 156-163. In some embodiments, the amino acid sequence of the variant mIg Fc fusion protein preferably may comprise a mIg Fc region that comprises an amino acid sequence 100% identical to the amino acid sequence set forth in any one of SEQ ID NOS: 156-163. In some embodiments, the amino acid sequence of the variant mIg Fc fusion protein comprises a mIg Fc region that consists of an amino acid sequence at least 85%, 86%, 87%, 88%, 89%, 90%, 91%, 92%, 93%, 94%, 95%, 96%, 97%, 98%, or 100% identical to the amino acid sequence set forth in any one of SEQ ID NOS: 156-163. For example, the amino acid sequence of the variant mIg Fc fusion protein may comprise a mIg Fc region that consists of an amino acid sequence at least 85% identical to the amino acid sequence set forth in any one of SEQ ID NOS: 156-163. The amino acid sequence of the variant mIg Fc fusion protein may comprise a hIg Fc region that consists of an amino acid sequence at least 90% identical to the amino acid sequence set forth in any one of SEQ ID NOS: 156-163. The amino acid sequence of the variant mIg Fc fusion protein may comprise a mIg Fc region that consists of an amino acid sequence at least 95% identical to the amino acid sequence set forth in any one of SEQ ID NOS: 156-163. In some embodiments, the amino acid sequence of the variant mIg Fc fusion protein preferably may comprise a mIg Fc region that consists of an amino acid sequence 100% identical to the amino acid sequence set forth in any one of SEQ ID NOS: 156-163.

In some embodiments, the amino acid sequence of the variant mIg Fc fusion protein comprises a mIg Fc region that comprises or consists of the amino acid sequence set forth in any one of SEQ ID NOS: 156-163, and further comprises 1 or more but less than 15% (less than 12%, less than 10%, less than 8%), amino acid variations (e.g., amino acid substitutions, deletions, or additions). In some embodiments, the amino acid sequence of the variant mIg Fc fusion protein comprises a mIg Fc region that comprises or consists of the amino acid sequence set forth in any one of SEQ ID NOS: 156-163, and further comprises or consists of at least about 1, 2, 3, 4, 5, 6, 7, 8, 9, or 10 amino acid variations (e.g., substitutions, additions, deletions, etc.). In some embodiments, the amino acid sequence of the variant mIg Fc fusion protein comprises a mIg Fc region that comprises or consists of the amino acid sequence set forth in any one of SEQ ID NOS: 156-163, and further comprises or consists of about 1, 2, 3, 4, 5, 6, 7, 8, 9, or 10 amino acid variations (e.g., substitutions, additions, deletions, etc.). In some embodiments, the amino acid sequence of the variant mIg Fc fusion protein comprises a mIg Fc region that comprises or consists of the amino acid sequence set forth in any one of SEQ ID NOS: 156-163, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the variant mIg Fc fusion protein comprises a mIg Fc region that comprises or consists of the amino acid sequence set forth in any one of SEQ ID NOS: 156-163, and further comprises 1 or more but less than 15% (less than 12%, less than 10%, less than 8%), amino acid substitutions. In some embodiments, the amino acid sequence of the variant mIg Fc fusion protein comprises a mIg Fc region that comprises or consists of the amino acid sequence set forth in any one of SEQ ID NOS: 156-163, and further comprises or consists of at least about 1, 2, 3, 4, 5, 6, 7, 8, 9, or 10 amino acid substitutions. In some embodiments, the amino acid sequence of the variant mIg Fc fusion protein comprises a mIg Fc region that comprises or consists of the amino acid sequence set forth in any one of SEQ ID NOS: 156-163, and further comprises or consists of about 1, 2, 3, 4, 5, 6, 7, 8, 9, or 10 amino acid substitutions. In some embodiments, the amino acid sequence of the variant mIg Fc fusion protein comprises a mIg Fc region that comprises or consists of the amino acid sequence set forth in any one of SEQ ID NOS: 156-163, and further comprises or consists of no more than about 1, 2, 3, 4, 5, 6, 7, 8, 9, or 10 amino acid substitutions.

### (ii) Enhanced Ig Effector Function

In some embodiments, the Ig Fc region exhibits an enhancement (e.g., an increase) in one or more Fc effector function relative to a reference (e.g., wild type) Ig Fc region. Exemplary Ig Fc effector functions include, but are not limited to, ADCC, ADCP, CDC, binding affinity to C1q, and binding affinity to one or more human Fc receptor (e.g., an Fcγ receptor (e.g., (e.g., FcγRI, FcγRIIa, FcγRIIIa, and/or FcγRIIIb). In some embodiments, the Fc region exhibits one or more enhanced Fc effector function, relative to a reference Ig (e.g., hIg, mIg).

In some embodiments, the hIg Fc region is modified (e.g., comprises one or more variation (e.g., one or more amino acid substitution, deletion, addition, etc.); altered glycosylation (e.g., afucosylation))) (referred to herein as a “modified hIg Fc”). In some embodiments, the modification (e.g., the variation (e.g., one or more amino acid substitution, deletion, addition, etc.); altered glycosylation (e.g., afucosylation))) enhances (e.g., increases) one or more Fc effector function, relative to a reference hIg Fc that does not comprise the modification (e.g., the one or more variation (e.g., the one or more amino acid substitution, deletion, addition, etc.; the altered glycosylation (e.g., afucosylation))).

In some embodiments, the modified Ig (e.g., hIg, mIg) Fc fusion protein exhibits enhanced ADCC compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits enhanced CDC compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits enhanced ADCP compared to a reference fusion protein that does not comprise the Ig (e.g., hIg, mIg) Fc modification (e.g., the one or more variation (e.g., one or more amino acid substitution, deletion, or addition)).

In some embodiments, the modified Ig (e.g., hIg, mIg) Fc fusion protein exhibits enhanced binding affinity to one or more Fc receptor (e.g., human Fc receptor) (e.g., an Fcγ receptor (e.g., FcγRI, FcγRIIa, FcγRIIIa, and/or FcγRIIIb) compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits enhanced binding affinity to FcγRI, FcγRIIa, FcγRIIIa, and/or FcγRIIIb compared to a reference fusion protein that does not comprise the hIg Fc modification (e.g., the one or more variation (e.g., one or more amino acid substitution, deletion, or addition)). In some embodiments, the modified Ig (e.g., hIg, mIg) Fc fusion protein exhibits enhanced binding affinity to FcγRI compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits enhanced binding affinity to FcγRIIa compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits enhanced binding affinity to FcγRIIIa compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits enhanced binding affinity to FcγRIIIb compared to a reference fusion protein that does not comprise the Ig (e.g., hIg, mIg) Fc modification (e.g., the one or more variation (e.g., one or more amino acid substitution, deletion, or addition)).

In some embodiments, the modified Ig (e.g., hIg, mIg) Fc fusion protein exhibits reduced binding affinity to one or more Fc receptor (e.g., human Fc receptor) ((e.g., an Fcγ receptor (e.g., FcγRIIb)) compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits reduced binding affinity to FcγRIIb compared to a reference fusion protein that does not comprise the Ig (e.g., hIg, mIg) Fc modification (e.g., the one or more variation (e.g., one or more amino acid substitution, deletion, or addition)).

In some embodiments, the modified Ig (e.g., hIg, mIg) Fc fusion protein exhibits enhanced binding affinity to C1q compared to a reference fusion protein that does not comprise the Ig (e.g., hIg, mIg) Fc modification (e.g., the one or more variation (e.g., one or more amino acid substitution, deletion, or addition)).

Amino acid substitutions and glycoengineering that enhance (e.g., increase) one or more hIg Fc effector function are known in the art. See for example, Liu R, Oldham R J, Teal E, Beers S A, Cragg M S. Fc-Engineering for Modulated Effector Functions-Improving Antibodies for Cancer Treatment. Antibodies (Basel). 2020; 9 (4): 64. Published 2020 Nov. 17. doi: 10.3390/antib9040064; van der Horst H J, Nijhof I S, Mutis T, Chamuleau M E D. Fc-Engineered Antibodies with Enhanced Fc-Effector Function for the Treatment of B-Cell Malignancies. Cancers (Basel). 2020; 12 (10): 3041. Published 2020 Oct. 19. Doi:10.3390/cancers12103041; and Saunders Kevin, “Conceptual Approaches to Modulating Antibody Effector Functions and Circulation Half-Life,” Frontiers in Immunology, v10 (Jun. 7, 2019) DOI=10.3389/fimmu.2019.01296, the full contents of each of which is incorporated by reference herein for all purposes.

Table 14 below, provides exemplary amino acid substitutions (and combinations thereof) and glycoengineering that can be utilized to increase one or more hIg Fc effector function. Amino acids in Table 14 are numbered according to the EU numbering scheme. The effects on effector function set forth in Table 14 are exemplary only and not intended to be limiting. The amino acid substitutions set forth in Table 14 are with reference to an IgG1 Fc region (except where noted). However, a person of ordinary skill in the could identify the corresponding amino acid in a non-IgG1 Fc region, for example in an IgG2 or IgG4 Fc region, should the base amino acid be different between the IgG1 and non-IgG1 Fc region.

TABLE 14
Exemplary hIg Fc Variations and Glycoengineering to Increase Effector Function.

| | Exemplary Effects on Effector Function |
|---|---|
| Variation/Glycoengineering | (Non-Limiting) |

Amino Acid Substitutions

| S298A/E333A/K334A | | Increases ADCC |
|---|---|---|
| S239D/I332E | | Increases ADCC |
| P247I/A339Q | | Increases ADCC |
| S239D/A330L/I332E | | Increases ADCC |
| G236A/S239D/I332E | | Increases ADCC |
| F243L/R292P/Y300L/V305I/P396L | | Increases ADCC |
| L235V/F243L/R292P/Y300L/P396L | | Increases ADCC |
| One Heavy Chain: | Opposing Heavy | Increases ADCC |
| L234Y/L235Q/G236W/ | Chain: | |
| S239M/H268D/D270E/ | D270E/K326D/ | |
| S298A | A330M/K334E | |

| | |
|---|---|
| F243L/R292P/Y300L/V305I/P396L | Increases ADCP |
| S239D/I332E/A330L | Increases ADCP |
| S239D/I332E/A330L/G236A | Increases ADCP |
| S239D/I332E/G326A | Increases ADCP |
| G236A | Increases ADCP |
| G236A/S239D/I332E | Increases ADCP |
| S239D/I332E | Increases ADCP |
| K326W/E333S | Increases C1q Binding and CDC |
| S267E/H268E/S324T | Increases C1q Binding and CDC |
| S298A/E333A/K334A | Enhances FcγRIIIa binding |
| S239D/I332E | Enhances FcγRIIIa binding |
| P247I/A339Q | Enhances FcγRIIIa binding |
| F243L/R292P/Y300L/V305I/P396L | Enhances FcγRIIa binding; decreases FcβRIIb binding |
| G236A | Enhances FcγRIIa binding |

Glycoengineering

| | |
|---|---|
| Afucosylation | Increases ADCC, Increases ADCP |
| Galactosylation | Increases CDC |

In some embodiments, the Ig Fc region is a hIg Fc region. In some embodiments, the hIg Fc (e.g., IgG1 Fc) region comprises any one or more of the amino acid substitutions set forth in Table 14 (i.e., any one or more amino acid substitution set forth in any set of amino acid substitutions set forth in Table 14). In some embodiments, the hIg Fc (e.g., IgG1 Fc) comprises any one or more of the sets of amino acid substitutions set forth in Table 14. In some embodiments, the hIg Fc (e.g., IgG1 Fc) region comprises any one or more of the glycosylation changes set forth in Table 14.

In some embodiments, the hIg Fc (e.g., IgG1 Fc) region comprises an amino acid substitution at any one or more (e.g., 1, 2, 3, 4, 5, 6, 7, 8, 9, or 10, or more) of amino acid positions S298, E333, K334, S239, 1332, P247, A339, A330, G236, F243, R292, Y300, V305, P396, L235, F243, R292, Y300, P396, F243, R292, Y300, V305, P396, K326, E333, S267E, H268, S324, S298, E333, K334, L234, L235, G236, S239, H268, D270, S298 D270, K326, A330, and/or K334. In some embodiments, the hIg Fc (e.g., IgG1 Fc) region comprises an amino acid substitution at from about 1-10 (e.g., 1-9, 1-8, 1-7, 1-6, 1-5, 1-4, 1-3, or 1-2) of the following amino acid positions S298, E333, K334, S239, 1332, P247, A339, A330, G236, F243, R292, Y300, V305, P396, L235, F243, R292, Y300, P396, F243, R292, Y300, V305, P396, K326, E333, S267E, H268, S324, S298, E333, K334, L234, L235, G236, S239, H268, D270, S298 D270, K326, A330, and/or K334.

In some embodiments, the hIg Fc (e.g., IgG1 Fc) region comprises one or more (e.g., 1, 2, 3, 4, 5, 6, 7, 8, 9, or 10, or more) of the following amino acid substitutions S298A, E333A, K334A, S239D, 1332E, P2471, A339Q, A330L, G236A, F243L, R292P, Y300L, V305I, P396L, L235V, F243L, R292P, Y300L, P396L, F243L, R292P, Y300L, V305I, P396L, K326W, E333S, S267E, H268E, S324T, S298A, E333A, K334A, L234Y, L235Q, G236W, S239M, H268D, D270E, S298A D270E, K326D, A330M, and/or K334E.

In some embodiments, the hIg Fc (e.g., IgG1 Fc) region comprises from about 1-10 (e.g., 1-9, 1-8, 1-7, 1-6, 1-5, 1-4, 1-3, or 1-2) of the following amino acid substitutions S298A, E333A, K334A, S239D, 1332E, P2471, A339Q, A330L, G236A, F243L, R292P, Y300L, V305I, P396L, L235V, F243L, R292P, Y300L, P396L, F243L, R292P, Y300L, V305I, P396L, K326W, E333S, S267E, H268E, S324T, S298A, E333A, K334A, L234Y, L235Q, G236W, S239M, H268D, D270E, S298A D270E, K326D, A330M, and/or K334E.

In some embodiments, the hIg Fc region comprises a hIgG1 Fc region comprising one or more amino acid variation relative to a reference hIgG1 Fc region.

In some embodiments, the hIgG1 Fc region comprises an amino acid substitution at one or more (e.g., 1, 2, or 3) of amino acid positions S298, E333, K334, EU numbering according to Kabat. In some embodiments, the hIgG1 Fc region comprises one or more (e.g., 1, 2, or 3) of the following amino acid substitutions S298A, E333A, and/or K334A, EU numbering according to Kabat.

In some embodiments, the hIgG1 Fc region comprises an amino acid substitution at one or more (e.g., 1 or 2) of amino acid positions S239 and/or 1332, EU numbering according to Kabat. In some embodiments, the hIgG1 Fc region comprises one or more (e.g., 1 or 2) of the following amino acid substitutions S239D and/or 1332E, EU numbering according to Kabat.

In some embodiments, the hIgG1 Fc region comprises an amino acid substitution at one or more (e.g., 1 or 2) of amino acid positions P247 and/or A339, EU numbering according to Kabat. In some embodiments, the hIgG1 Fc region comprises one or more (e.g., 1 or 2) of the following amino acid substitutions P2471 and/or A339Q, EU numbering according to Kabat.

In some embodiments, the hIgG1 Fc region comprises an amino acid substitution at one or more (e.g., 1, 2, or 3) of amino acid positions S239, A330, and/or 1332, EU numbering according to Kabat. In some embodiments, the hIgG1 Fc region comprises one or more (e.g., 1, 2, or 3) of the following amino acid substitutions S239D, A330L, and/or 1332E, EU numbering according to Kabat.

In some embodiments, the hIgG1 Fc region comprises an amino acid substitution at one or more (e.g., 1, 2, or 3) of amino acid positions G236, S239, and/or 1332, EU numbering according to Kabat. In some embodiments, the hIgG1 Fc region comprises one or more (e.g., 1, 2, or 3) of the following amino acid substitutions G236A, S239D, and/or 1332E, EU numbering according to Kabat.

In some embodiments, the hIgG1 Fc region comprises an amino acid substitution at one or more (e.g., 1, 2, 3, 4, or 5) of amino acid positions F243, R292, Y300, V305, and/or P396, EU numbering according to Kabat. In some embodiments, the hIgG1 Fc region comprises one or more (e.g., 1, 2, 3, 4, or 5) of the following amino acid substitutions F243L, R292P, Y300L, V305I, and/or P396L, EU numbering according to Kabat.

In some embodiments, the hIgG1 Fc region comprises an amino acid substitution at one or more (e.g., 1, 2, 3, 4, or 5) of amino acid positions L235, F243, R292, Y300, and P396, EU numbering according to Kabat. In some embodiments, the hIgG1 Fc region comprises one or more (e.g., 1, 2, 3, 4, or 5) of the following amino acid substitutions L235V, F243L, R292P, Y300L, and/or P396L, EU numbering according to Kabat.

In some embodiments, the hIgG1 Fc region comprises an amino acid substitution at one or more (e.g., 1, 2, 3, 4, 5, 6, or 7) of amino acid positions L234, L235, G236, S239, H268, D270, and/or S298, EU numbering according to Kabat. In some embodiments, the hIgG1 Fc region comprises one or more (e.g., 1, 2, 3, 4, 5, 6, or 7) of the following amino acid substitutions L234Y, L235Q, G236W, S239M, H268D, D270E, and/or S298A, EU numbering according to Kabat.

In some embodiments, the hIgG1 Fc region comprises an amino acid substitution at one or more (e.g., 1, 2, 3, or 4) of amino acid positions D270, K326, A330, and/or K334, EU numbering according to Kabat. In some embodiments, the hIgG1 Fc region comprises one or more (e.g., 1, 2, 3, or 4) of the following amino acid substitutions D270E, K326D, A330M, and/or K334E, EU numbering according to Kabat.

In some embodiments, the hIgG1 Fc region comprises an amino acid substitution at one or more (e.g., 1, 2, 3, 4, or 5) of amino acid positions F243, R292, Y300, V305, and/or P396, EU numbering according to Kabat. In some embodiments, the hIgG1 Fc region comprises one or more (e.g., 1, 2, 3, 4, or 5) of the following amino acid substitutions F243L, R292P, Y300L, V305I, and/or P396L, EU numbering according to Kabat.

In some embodiments, the hIgG1 Fc region comprises an amino acid substitution at one or more (e.g., 1, 2, or 3) of amino acid positions S239, 1332, and/or A330, EU numbering according to Kabat. In some embodiments, the hIgG1 Fc region comprises one or more (e.g., 1, 2, or 3) of the following amino acid substitutions S239D, 1332E, and/or A330L, EU numbering according to Kabat.

In some embodiments, the hIgG1 Fc region comprises an amino acid substitution at one or more (e.g., 1, 2, 3, or 4) of amino acid positions S239, 1332, A330, and/or G236, EU numbering according to Kabat. In some embodiments, the hIgG1 Fc region comprises one or more (e.g., 1, 2, 3, or 4) of the following amino acid substitutions S239D, 1332E, A330L and/or G236A, EU numbering according to Kabat.

In some embodiments, the hIgG1 Fc region comprises an amino acid substitution at one or more (e.g., 1, 2, or 3) of amino acid positions S239, 1332, and/or G326, EU numbering according to Kabat. In some embodiments, the hIgG1 Fc region comprises one or more (e.g., 1, 2, or 3) of the following amino acid substitutions S239D, 1332E, and/or G326A, EU numbering according to Kabat.

In some embodiments, the hIgG1 Fc region comprises an amino acid substitution at amino acid position G326, EU numbering according to Kabat. In some embodiments, the hIgG1 Fc region comprises a G326A amino acid substitution, EU numbering according to Kabat.

In some embodiments, the hIgG1 Fc region comprises an amino acid substitution at one or more (e.g., 1, 2, or 3) of amino acid positions G236, S239, and/or 1332, EU numbering according to Kabat. In some embodiments, the hIgG1 Fc region comprises one or more (e.g., 1, 2, or 3) of the following amino acid substitutions G236A, S239D, and/or 1332E, EU numbering according to Kabat.

In some embodiments, the hIgG1 Fc region comprises an amino acid substitution at one or more (e.g., 1 or 2) of amino acid positions S239 and/or 1332, EU numbering according to Kabat. In some embodiments, the hIgG1 Fc region comprises one or more (e.g., 1 or 2) of the following amino acid substitutions S239D and/or 1332E, EU numbering according to Kabat.

In some embodiments, the hIgG1 Fc region comprises an amino acid substitution at one or more (e.g., 1 or 2) of amino acid positions K326 and/or E333, EU numbering according to Kabat. In some embodiments, the hIgG1 Fc region comprises one or more (e.g., 1 or 2) of the following amino acid substitutions K326W and/or E333S, EU numbering according to Kabat.

In some embodiments, the hIgG1 Fc region comprises an amino acid substitution at one or more (e.g., 1, 2, or 3) of amino acid positions S267, H268, and/or S324, EU numbering according to Kabat. In some embodiments, the hIgG1 Fc region comprises one or more (e.g., 1, 2, or 3) of the following amino acid substitutions S267E, H268E, and/or S324T, EU numbering according to Kabat.

In some embodiments, the hIgG1 Fc region comprises an amino acid substitution at one or more (e.g., 1, 2, or 3) of amino acid positions S298, E333, and/or K334, EU numbering according to Kabat. In some embodiments, the hIgG1 Fc region comprises one or more (e.g., 1, 2, or 3) of the following amino acid substitutions S298A, E333A, and/or K334A, EU numbering according to Kabat.

In some embodiments, the hIgG1 Fc region comprises an amino acid substitution at one or more (e.g., 1 or 2) of amino acid positions S239 and/or 1332, EU numbering according to Kabat. In some embodiments, the hIgG1 Fc region comprises one or more (e.g., 1 or 2) of the following amino acid substitutions S239D and/or 1332E, EU numbering according to Kabat.

In some embodiments, the hIgG1 Fc region comprises an amino acid substitution at one or more (e.g., 1 or 2) of amino acid positions P247 and/or A339, EU numbering according to Kabat. In some embodiments, the hIgG1 Fc region comprises one or more (e.g., 1 or 2) of the following amino acid substitutions P2471 and/or A339Q, EU numbering according to Kabat.

In some embodiments, the hIg Fc region comprises one or more changes to the glycosylation. In some embodiments, the hIg Fc region is afucosylated. In some embodiments, the hIg Fc region is afucosylated and exhibits enhanced (e.g., increased) ADCC compared to a reference hIg Fc region that is not afucosylated. Afucosylated antibodies can be made, e.g., through modifying the amino acid sequence, utilizing an engineered cell line (e.g., CHOK1-FUT8), and utilizing specific cell culture media. See, e.g., Pereira, Natasha A et al. “The “less-is-more” in therapeutic antibodies: Afucosylated anti-cancer antibodies with enhanced antibody-dependent cellular cytotoxicity.” mAbs vol. 10, 5 (2018): 693-711. doi: 10.1080/19420862.2018.1466767, the entire contents of which are incorporated herein by reference for all purposes.

### 5.4.6 Linkers

As described herein, the heterologous moiety (e.g., heterologous protein) can be directly operably connected or indirectly operably connected to the immunoreceptor inhibitory protein (e.g., described herein). In some embodiments, the heterologous protein is directly operably connected to the immunoreceptor inhibitory protein (e.g., described herein) via a peptide bond. In some embodiments, the heterologous protein is indirectly operably connected to the immunoreceptor inhibitory protein (e.g., described herein) via a linker. In some embodiments, the heterologous protein is indirectly operably connected to the immunoreceptor inhibitory protein (e.g., described herein) via a linker (e.g., a peptide linker). In some embodiments, the heterologous protein is indirectly operably connected to the immunoreceptor inhibitory protein (e.g., described herein) via a peptide linker.

In some embodiments, the linker (e.g., peptide linker) is one or any combination of a cleavable linker, a non-cleavable linker, a flexible linker, a rigid linker, a helical linker, and/or a non-helical linker. In some embodiments, the peptide linker is one or any combination of a cleavable linker, a non-cleavable linker, a flexible linker, a rigid linker, a helical linker, and/or a non-helical linker.

In some embodiments, the linker is a peptide linker, e.g., a peptide linker having an amino acid sequence comprising glycine, serine, or both glycine and serine amino acid residues. In some embodiments, the amino acid sequence of the peptide linker comprises glycine, serine, and proline amino acid residues. In some embodiments, the amino acid sequence of the peptide linker consists of glycine, serine, or both glycine and serine amino acid residues. In some embodiments, the amino acid sequence of the peptide linker consists of glycine, serine, and proline amino acid residues.

In some embodiments, the amino acid sequence of the peptide linker comprises from or from about 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, or 5-10 amino acid residues. In some embodiments, the amino acid sequence of the peptide linker comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the amino acid sequence of the peptide linker comprises about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the amino acid sequence of the peptide linker consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the amino acid sequence of the peptide linker comprises no more than about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the amino acid sequence of the peptide linker consists of no more than about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In certain embodiments, the amino acid sequence of the peptide linker comprises between 3-25 amino acid residues, each of the 3-25 amino acid residues being glycine or serine. A linker as described herein may also be referred to as a “spacer”.

The amino acid sequence of exemplary peptide linkers, which can be incorporated in one or more of the embodiments described herein (e.g., fusion proteins), is set provided in Table 8. The exemplary glycine-serine linkers provided in Table 8 are exemplary only and not intended to be limiting. As such, additional peptide linkers (e.g., described herein, otherwise known in the art, etc.) will be applicable in the same way to the fusion proteins described herein, independent of the specific species (e.g., specific amino acid sequence) of the peptide linker.

TABLE 8
The Amino Acid Sequence of Exemplary
Peptide Linkers.

| Descrip- | | SEQ ID |
|---|---|---|
| tion | Amino Acid Sequence | NO |
| A | GGGGGGGS | 164 |
| B | GGGGGGGSGGGGGGGS | 165 |
| C | GGGGGGGSGGGGGGGSGGGGGGGS | 166 |
| D | GGGGS | 167 |
| E | GGGGSGGGGS | 168 |
| F | GGGGGGGGSGGGGS | 169 |
| G | GGGS | 170 |
| H | GGGSGGGS | 171 |
| I | GGGSGGGSGGGS | 172 |
| J | GGGGGGGSGGGGSGGGGS | 173 |

In some embodiments, the amino acid sequence of the peptide linker comprises the amino acid sequence of any one of the linkers set forth in Table 8. In some embodiments, the amino acid sequence of the peptide linker consists of the amino acid sequence of any one of the linkers set forth in Table 8. In some embodiments, the amino acid sequence of the peptide linker comprises the amino acid sequence of any one of the linkers set forth in Table 8, and further comprises 1 or more but less than 15% (less than 12%, less than 10%, less than 8%), amino acid variations (e.g., amino acid substitutions, deletions, or additions). In some embodiments, the amino acid sequence of the peptide linker consists of the amino acid sequence of any one of the linkers set forth in Table 8, and further comprises 1 or more but less than 15% (less than 12%, less than 10%, less than 8%), amino acid variations (e.g., amino acid substitutions, deletions, or additions). In some embodiments, the amino acid sequence of the peptide linker comprises the amino acid sequence of any one of the linkers set forth in Table 8, comprising 1, 2, or 3 amino acid variations (e.g., substitutions, deletions, additions). In some embodiments, the amino acid sequence of the peptide linker consists of the amino acid sequence of any one of the linkers set forth in Table 8, comprising 1, 2, or 3 amino acid variations (e.g., substitutions, deletions, additions). In some embodiments, the amino acid sequence of the peptide linker comprises the amino acid sequence of any one of the linkers set forth in Table 8, and further comprises 1 or more but less than 15% (less than 12%, less than 10%, less than 8%), amino acid substitutions. In some embodiments, the amino acid sequence of the peptide linker consists of the amino acid sequence of any one of the linkers set forth in Table 8, and further comprises 1 or more but less than 15% (less than 12%, less than 10%, less than 8%), amino acid substitutions. In some embodiments, the amino acid sequence of the peptide linker comprises the amino acid sequence of any one of the linkers set forth in Table 8, comprising 1, 2, or 3 amino acid substitutions. In some embodiments, the amino acid sequence of the peptide linker consists of the amino acid sequence of any one of the linkers set forth in Table 8, comprising 1, 2, or 3 amino acid substitutions.

In some embodiments, the amino acid sequence of the peptide linker comprises the amino acid sequence set forth in any one of SEQ ID NOS: 164-173. In some embodiments, the amino acid sequence of the peptide linker consists of the amino acid sequence set forth in any one of SEQ ID NOS: 164-173. In some embodiments, the amino acid sequence of the peptide linker comprises the amino acid sequence set forth in any one of SEQ ID NOS: 164-173, and further comprises 1 or more but less than 15% (less than 12%, less than 10%, less than 8%), amino acid variations (e.g., amino acid substitutions, deletions, or additions). In some embodiments, the amino acid sequence of the peptide linker consists of the amino acid sequence set forth in any one of SEQ ID NOS: 164-173, and further comprises 1 or more but less than 15% (less than 12%, less than 10%, less than 8%), amino acid variations (e.g., amino acid substitutions, deletions, or additions). In some embodiments, the amino acid sequence of the peptide linker comprises the amino acid sequence set forth in any one of SEQ ID NOS: 164-173, comprising 1, 2, or 3 amino acid variations (e.g., substitutions, deletions, additions). In some embodiments, the amino acid sequence of the peptide linker consists of the amino acid sequence set forth in any one of SEQ ID NOS: 164-173, comprising 1, 2, or 3 amino acid variations (e.g., substitutions, deletions, additions). In some embodiments, the amino acid sequence of the peptide linker comprises the amino acid sequence set forth in any one of SEQ ID NOS: 164-173, and further comprises 1 or more but less than 15% (less than 12%, less than 10%, less than 8%), amino acid substitutions. In some embodiments, the amino acid sequence of the peptide linker consists of the amino acid sequence set forth in any one of SEQ ID NOS: 164-173, and further comprises 1 or more but less than 15% (less than 12%, less than 10%, less than 8%), amino acid substitutions. In some embodiments, the amino acid sequence of the peptide linker comprises the amino acid sequence set forth in any one of SEQ ID NOS: 164-173, comprising 1, 2, or 3 amino acid substitutions. In some embodiments, the amino acid sequence of the peptide linker consists of the amino acid sequence set forth in any one of SEQ ID NOS: 164-173, comprising 1, 2, or 3 amino acid substitutions.

In some embodiments, the amino acid sequence of the peptide linker comprises the amino acid sequence set forth in SEQ ID NO: 173. In some embodiments, the amino acid sequence of the peptide linker consists of the amino acid sequence set forth in SEQ ID NO: 173. In some embodiments, the amino acid sequence of the peptide linker comprises the amino acid sequence set forth in SEQ ID NO: 173, and further comprises 1 or more but less than 15% (less than 12%, less than 10%, less than 8%), amino acid variations (e.g., amino acid substitutions, deletions, or additions). In some embodiments, the amino acid sequence of the peptide linker consists of the amino acid sequence set forth in SEQ ID NO: 173, and further comprises 1 or more but less than 15% (less than 12%, less than 10%, less than 8%), amino acid variations (e.g., amino acid substitutions, deletions, or additions). In some embodiments, the amino acid sequence of the peptide linker comprises the amino acid sequence set forth in SEQ ID NO: 173, comprising 1, 2, or 3 amino acid variations (e.g., substitutions, deletions, additions). In some embodiments, the amino acid sequence of the peptide linker consists of the amino acid sequence set forth in SEQ ID NO: 173, comprising 1, 2, or 3 amino acid variations (e.g., substitutions, deletions, additions). In some embodiments, the amino acid sequence of the peptide linker comprises the amino acid sequence set forth in SEQ ID NO: 173, and further comprises 1 or more but less than 15% (less than 12%, less than 10%, less than 8%), amino acid substitutions. In some embodiments, the amino acid sequence of the peptide linker consists of the amino acid sequence set forth in SEQ ID NO: 173, and further comprises 1 or more but less than 15% (less than 12%, less than 10%, less than 8%), amino acid substitutions. In some embodiments, the amino acid sequence of the peptide linker comprises the amino acid sequence set forth in SEQ ID NO: 173, comprising 1, 2, or 3 amino acid substitutions. In some embodiments, the amino acid sequence of the peptide linker consists of the amino acid sequence set forth in SEQ ID NO: 173, comprising 1, 2, or 3 amino acid substitutions.

In some embodiments, the linker or spacer is a non-peptide linker or spacer. Such non-peptide linkers or spacers, such as synthetic polymers that provide flexibility and biocompatibility, are known in the art and can include, e.g., polyethylene glycol (PEG), poly(ethylene oxide) (PEO), or poly(propylene glycol) (PPG).

### 5.4.7 Orientation

The heterologous moiety (e.g., heterologous protein) and the immunoreceptor inhibitory protein (e.g., described herein) can be arranged in any configuration or order as long as the immunoreceptor inhibitory protein (e.g., described herein) maintains the ability to mediate its function (e.g., bind to its cognate partner) and in the embodiments wherein the heterologous moiety (e.g., heterologous protein) has a specific function, the heterologous moiety (e.g., heterologous protein) can mediate its function.

In some embodiments, the heterologous moiety is a heterologous protein (e.g., an Ig (e.g., hIg, mIg) Fc region (e.g., an Ig (e.g., hIg, mIg) Fc region described herein)) forming a fusion protein. In some embodiments, the fusion protein comprises from N- to C-terminus: an immunoreceptor inhibitory protein (e.g., described herein) and a heterologous protein (e.g., an Ig (e.g., hIg, mIg) Fc region (e.g., an Ig (e.g., hIg, mIg) Fc region described herein)). In some embodiments, the fusion protein comprises from N- to C-terminus: an immunoreceptor inhibitory protein (e.g., described herein), a peptide linker (e.g., described herein), and a heterologous protein (e.g., an Ig (e.g., hIg, mIg) Fc region (e.g., an Ig (e.g., hIg, mIg) Fc region described herein)). In this specific orientation, the N-terminus of the immunoreceptor inhibitory protein (e.g., described herein) is operably connected to the C-terminus of the heterologous protein (e.g., an Ig (e.g., hIg, mIg) Fc region (e.g., an Ig (e.g., hIg, mIg) Fc region described herein)) either directly or indirectly through the peptide linker (e.g., described herein).

In some embodiments, the fusion protein comprises from N- to C-terminus: a heterologous protein (e.g., an Ig (e.g., hIg, mIg) Fc region (e.g., an Ig (e.g., hIg, mIg) Fc region described herein)) and an immunoreceptor inhibitory protein (e.g., described herein). In some embodiments, the fusion protein comprises from N- to C-terminus: a heterologous protein (e.g., an Ig (e.g., hIg, mIg) Fc region (e.g., an Ig (e.g., hIg, mIg) Fc region described herein)), a peptide linker (e.g., described herein), and an immunoreceptor inhibitory protein (e.g., described herein). In this specific orientation, the C-terminus of the immunoreceptor inhibitory protein (e.g., described herein) is operably connected to the N-terminus of the heterologous protein (e.g., an Ig (e.g., hIg, mIg) Fc region (e.g., an Ig (e.g., hIg, mIg) Fc region described herein)) either directly or indirectly through the peptide linker (e.g., described herein).

### 5.4.8 Multimeric Fusion Proteins

Provided herein are multimeric (e.g., dimeric) proteins comprising at least two fusion proteins or conjugates described herein (e.g., Ig (e.g., hIg, mIg) Fc fusion proteins described herein). In some embodiments, the protein is dimeric. In some embodiments, the protein is homodimeric. In some embodiments, the protein is heterodimeric. In some embodiments, the at least two fusion proteins described herein (e.g., Ig (e.g., hIg, mIg) Fc fusion proteins described herein) or conjugates associate via covalent or non-covalent interactions. In some embodiments, the at least two fusion proteins described herein (e.g., Ig (e.g., hIg, mIg) Fc fusion proteins described herein) or conjugates associate via at least one covalent interaction. In some embodiments, the at least two fusion proteins (e.g., Ig (e.g., hIg, mIg) Fc fusion proteins) or conjugates associate via one or more disulfide bond. In some embodiments, the at least two fusion proteins (e.g., Ig (e.g., hIg, mIg) Fc fusion proteins) or conjugates associate via 1, 2, 3, 4, or more disulfide bonds.

In some embodiments, the protein is dimeric comprising a first fusion protein (e.g., a hIg Fc fusion protein) or conjugate described herein and a second fusion protein (e.g., an Ig (e.g., hIg, mIg) Fc fusion protein) or conjugate described herein, wherein the amino acid sequence of the first protein comprises an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the second protein. For example, the amino acid sequence of the first protein may comprise an amino acid sequence at least about 85% identical to the amino acid sequence of the second protein. For example, the amino acid sequence of the first protein may comprise an amino acid sequence at least about 90% identical to the amino acid sequence of the second protein. For example, the amino acid sequence of the first protein may comprise an amino acid sequence at least about 95% identical to the amino acid sequence of the second protein. In some embodiments, the amino acid sequence of the first protein may preferably comprise an amino acid sequence 100% identical to the amino acid sequence of the second protein.

In some embodiments, the protein is dimeric comprising a first Ig (e.g., hIg, mIg) Fc fusion protein and a second Ig (e.g., hIg, mIg) Fc fusion protein. In some embodiments, the dimeric protein is homodimeric. In some embodiments, the dimeric protein is heterodimeric. In some embodiments, the amino acid sequence of the first Ig (e.g., hIg, mIg) Fc fusion protein comprises an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the second Ig (e.g., hIg, mIg) Fc fusion protein.

An exemplary dimeric Ig (e.g., hIg, mIg) Fc fusion protein includes, for example, a protein comprising (i) a first Ig (e.g., hIg, mIg) Fc fusion protein comprising from N- to C-terminus: a first Ig (e.g., hIg, mIg) Fc region (e.g., described herein), a first peptide linker (e.g., described herein), and a first immunoreceptor inhibitory protein (e.g., described herein); and (ii) a second Ig (e.g., hIg, mIg) Fc fusion protein comprising from N- to C-terminus: a second Ig (e.g., hIg, mIg) Fc region (e.g., described herein), a second peptide linker (e.g., described herein), and a second immunoreceptor inhibitory protein (e.g., described herein). In some embodiments, the amino acid sequence of the first Ig (e.g., hIg, mIg) Fc fusion protein is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the second Ig (e.g., hIg, mIg) Fc fusion protein. In this specific embodiment, the N-terminus of the immunoreceptor inhibitory protein (e.g., described herein) is operably connected to the C-terminus of the Ig (e.g., hIg, mIg) Fc region through the peptide linker (e.g., described herein).

Another exemplary dimeric Ig (e.g., hIg, mIg) Fc fusion protein includes, for example, a protein comprising (i) a first Ig (e.g., hIg, mIg) Fc fusion protein comprising from N- to C-terminus: a first immunoreceptor inhibitory protein (e.g., described herein), a first peptide linker (e.g., described herein), and a first Ig (e.g., hIg, mIg) Fc region (e.g., described herein); and (ii) a second immunoreceptor inhibitory protein (e.g., described herein), a second peptide linker (e.g., described herein), and a second Ig (e.g., hIg, mIg) Fc region (e.g., described herein). In some embodiments, the amino acid sequence of the first Ig (e.g., hIg, mIg) Fc fusion protein is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the second Ig (e.g., hIg, mIg) Fc fusion protein. In this specific embodiment, the C-terminus of the immunoreceptor inhibitory protein (e.g., described herein) is operably connected to the N-terminus of the Ig (e.g., hIg, mIg) Fc region either directly or indirectly through the peptide linker (e.g., described herein).

### 5.4.9 Exemplary Ig Fusion Proteins

The amino acid sequence of exemplary immunoreceptor inhibitory fusion proteins (IIFPs) described herein is provided in Table 9. Each of the IIFPs 1-4 and 32-33 comprising the amnio acid sequence set forth in any one of SEQ ID NOS: 174, 177, 180, 183, 191, and 194 comprises from N- to C-terminus the hIL-2 signal sequence (hIL-2ss), an effector function reduced hIgG4 Fc region, a peptide linker, and an immunoreceptor inhibitory protein identified herein (IIPs 1-4 and 32-33) (e.g., see Table 2, SEQ ID NOS: 23-26 and 187-188). Each of the IIFPs 1˜4 and 32-33 comprising the amnio acid sequence set forth in any one of SEQ ID NOS: 175-176, 178-179, 181-182, 184-185, 192-193, and 195-196 comprises from N- to C-terminus an effector function reduced hIgG4 Fc region, a peptide linker, and an immunoreceptor inhibitory protein identified herein (IIPs 1-4 and 32-33) (e.g., see Table 2, SEQ ID NOS: 23-26 and 187-188).

The fusion proteins provided in Table 9 are exemplary only, and not intended to be limiting. Similar fusion proteins can be made utilizing the additional IIPs listed in Table 2, e.g., any one of IIPs 1-34. Likewise, the individual components of the fusion proteins provided in Table 9 are not intended to be limiting. For example, other peptide linkers (e.g., described herein, known in the art, etc.) also allowing for operable connection of the IIP to the heterologous moiety may be utilized. Additionally, other heterologous moieties (e.g., half-life extension moieties) that e.g., share common properties (e.g., half-life extension) with the IgG4 Fc region may be utilized regardless of the specific species (e.g., IgG1 Fc region).

TABLE 9
The Amino Acid Sequence of Exemplary Ig Fusion Proteins.

| | | SEQ |
|---|---|---|
| Description | Amino Acid Sequence | ID NO |
| IIFP-1 | MYRMQLLSCIALSLALVINSAESKYGPPCPPCPAPEAAGGPSVFLFPPKP | 174 |
| with hIL-2 | KDTLMISRTPEVTCVVVDVSQEDPEVQFNWYVDGVEVHNAKTKPREEQFN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSTVYVVKSGVNKGKICDTCPPGTYKKRDCDRSLP | |
| | TVCEPCGDGEYTSMNNSLPECLSCNHCYDPTEIEITPCNATTNTVCSCKE | |
| | GYTFDSSIQGCI | |
| IIFP-1 | AESKYGPPCPPCPAPEAAGGPSVFLFPPKPKDTLMISRTPEVTCVVVDVS | 175 |
| without hIL-2 | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSTVY | |
| | VVKSGVNKGKICDTCPPGTYKKRDCDRSLPTVCEPCGDGEYTSMNNSLPE | |
| | CLSCNHCYDPTEIEITPCNATTNTVCSCKEGYTFDSSIQGCI | |
| IIFP-1 | ESKYGPPCPPCPAPEAAGGPSVFLFPPKPKDTLMISRTPEVTCVVVDVSQ | 176 |
| without hIL-2 | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSTVYV | |
| | VKSGVNKGKICDTCPPGTYKKRDCDRSLPTVCEPCGDGEYTSMNNSLPEC | |
| | LSCNHCYDPTEIEITPCNATTNTVCSCKEGYTEDSSIQGCI | |
| IIFP-2 | MYRMQLLSCIALSLALVINSAESKYGPPCPPCPAPEAAGGPSVFLFPPKP | 177 |
| with hIL-2 | KDTLMISRTPEVTCVVVDVSQEDPEVQFNWYVDGVEVHNAKTKPREEQFN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSSTYRVRSSGLTCSTCPPGTHKERDCSLNTETIC | |
| | KACGEGEYTAHNNSLPKCLACKSCFNATEIETKSCDPTSDTICACREGYS | |
| | INNLGECN | |
| IIFP-2 | AESKYGPPCPPCPAPEAAGGPSVFLFPPKPKDTLMISRTPEVTCVVVDVS | 178 |
| without hIL-2 | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSSTY | |
| | RVRSSGLTCSTCPPGTHKERDCSLNTETICKACGEGEYTAHNNSLPKCLA | |
| | CKSCFNATEIETKSCDPTSDTICACREGYSINNLGECN | |
| IIFP-2 | ESKYGPPCPPCPAPEAAGGPSVFLFPPKPKDTLMISRTPEVTCVVVDVSQ | 179 |
| without hIL-2 | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSSTYR | |
| | VRSSGLTCSTCPPGTHKERDCSLNTETICKACGEGEYTAHNNSLPKCLAC | |
| | KSCFNATEIETKSCDPTSDTICACREGYSINNLGECN | |
| IIFP-3 | MYRMQLLSCIALSLALVINSAESKYGPPCPPCPAPEAAGGPSVFLFPPKP | 180 |
| with hIL-2 | KDTLMISRTPEVTCVVVDVSQEDPEVQFNWYVDGVEVHNAKTKPREEQFN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSSTYRVRSSGLTCSMCPPGTYKERDCSLNTETLC | |
| | KACGEGEYTAHKNSLPKCLACKSCFNATEIETKSCDPISDTICTCREGYS | |
| | INNLGECN | |
| IIFP-3 | AESKYGPPCPPCPAPEAAGGPSVFLFPPKPKDTLMISRTPEVTCVVVDVS | 181 |
| without hIL-2 | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSSTY | |
| | RVRSSGLTCSMCPPGTYKERDCSLNTETLCKACGEGEYTAHKNSLPKCLA | |
| | CKSCFNATEIETKSCDPISDTICTCREGYSINNLGECN | |
| IIFP-3 | ESKYGPPCPPCPAPEAAGGPSVFLFPPKPKDTLMISRTPEVTCVVVDVSQ | 182 |
| without hIL-2 | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSSTYR | |
| | VRSSGLTCSMCPPGTYKERDCSLNTETLCKACGEGEYTAHKNSLPKCLAC | |
| | KSCFNATEIETKSCDPISDTICTCREGYSINNLGECN | |
| IIFP-4 | MYRMQLLSCIALSLALVINSAESKYGPPCPPCPAPEAAGGPSVFLFPPKP | 183 |
| with hIL-2 | KDTLMISRTPEVTCVVVDVSQEDPEVQFNWYVDGVEVHNAKTKPREEQFN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSRSKINSSLICDMCPPGYYKNKDCTSTSTTICLP | |
| | CGEGEYTAYNNSLTKCIRCKDCYEENEKIFKPCNSTSDTICTCIDGYTKD | |
| | ETTDSCI | |
| IIFP-4 | AESKYGPPCPPCPAPEAAGGPSVFLFPPKPKDTLMISRTPEVTCVVVDVS | 184 |
| without hIL-2 | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSRSK | |
| | INSSLICDMCPPGYYKNKDCTSTSTTICLPCGEGEYTAYNNSLIKCIRCK | |
| | DCYEENEKIFKPCNSTSDTICTCIDGYTKDETTDSCI | |
| IIFP-4 | ESKYGPPCPPCPAPEAAGGPSVFLFPPKPKDTLMISRTPEVTCVVVDVSQ | 185 |
| without hIL-2 | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSRSKI | |
| | NSSLICDMCPPGYYKNKDCTSTSTTICLPCGEGEYTAYNNSLIKCIRCKD | |
| | CYEENEKIFKPCNSTSDTICTCIDGYTKDETTDSCI | |
| IIFP-32 | MYRMQLLSCIALSLALVTNSAESKYGPPCPPCPAPEAAGGPSVFLFPPKP | 191 |
| with hIL-2 | KDTLMISRTPEVTCVVVDVSQEDPEVQFNWYVDGVEVHNAKTKPREEQFN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSMSTYNTKTSPSLKCDMCPPGYYKHEDCTSNTKT | |
| | VCSPCGEGEYTAYNNSLTKCLRCSDCYGENEITTKQCTNTSNTVCECMDG | |
| | YTKDETIDACIK | |
| IIFP-32 | AESKYGPPCPPCPAPEAAGGPSVFLFPPKPKDTLMISRTPEVTCVVVDVS | 192 |
| without hIL-2 | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSMST | |
| | YNTKTSPSLKCDMCPPGYYKHEDCTSNTKTVCSPCGEGEYTAYNNSLTKC | |
| | LRCSDCYGENEITTKQCTNTSNTVCECMDGYTKDETIDACIK | |
| IIFP-32 | ESKYGPPCPPCPAPEAAGGPSVFLFPPKPKDTLMISRTPEVTCVVVDVSQ | 193 |
| without hIL-2 | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSMSTY | |
| | NTKTSPSLKCDMCPPGYYKHEDCTSNTKTVCSPCGEGEYTAYNNSLIKCL | |
| | RCSDCYGENEITTKQCTNTSNTVCECMDGYTKDETIDACIK | |
| IIFP-33 | MYRMQLLSCIALSLALVTNSAESKYGPPCPPCPAPEAAGGPSVFLFPPKP | 194 |
| with hIL-2 | KDTLMISRTPEVTCVVVDVSQEDPEVQFNWYVDGVEVHNAKTKPREEQFN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSASTYTSKIDASLICDMCPPGSYKYKDCTYDSKT | |
| | VCLPCGDGEYTSYNNSLAKCLRCDDCYDENEVTAKPCDSTSNTICKCMDG | |
| | YTKDNTISACVKTSHIT | |
| IIFP-33 | AESKYGPPCPPCPAPEAAGGPSVFLFPPKPKDTLMISRTPEVTCVVVDVS | 195 |
| without hIL-2 | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSAST | |
| | YTSKIDASLICDMCPPGSYKYKDCTYDSKTVCLPCGDGEYTSYNNSLAKC | |
| | LRCDDCYDENEVTAKPCDSTSNTICKCMDGYTKDNTISACVKTSHIT | |
| IIFP-33 | ESKYGPPCPPCPAPEAAGGPSVFLFPPKPKDTLMISRTPEVTCVVVDVSQ | 196 |
| without hIL-2 | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSASTY | |
| | TSKIDASLICDMCPPGSYKYKDCTYDSKTVCLPCGDGEYTSYNNSLAKCL | |
| | RCDDCYDENEVTAKPCDSTSNTICKCMDGYTKDNTISACVKTSHIT | |
| IIFP-34 | MYRMQLLSCIALSLALVINSAESKYGPPCPPCPAPEAAGGPSVFLFPPKP | 201 |
| with hIL-2 | KDTLMISRTPEVTCVVVDVSQEDPEVQFNWYVDGVEVHNAKTKPREEQFN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSKVPTWTDPVTGLTCDRCPAGTHVVKQCTATTPT | |
| | ECGACPANHYTEFWNYLNRCLYCGVRCTEQQVEKSTCSATHNRVCECKPG | |
| | YHIYADDFCLRHSTCPPGQGLLVAGTPTSNTRCGPCQAGYFSAEDSTEAC | |
| | KPHTPCKDTERSVPGTATQDTFCTSCQARKCDITAPPAPDKAVCDDAFID | |
| | FVERYPLNAKKAKNLMRKLRKMGKGKTIRESFQAIMAKRNDQPLACEVLS | |
| | TLNDVDSDMGCRIKTFFLGWNEDEC | |
| IIFP-34 | AESKYGPPCPPCPAPEAAGGPSVFLFPPKPKDTLMISRTPEVTCVVVDVS | 202 |
| without hIL-2 | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSKVP | |
| | TWTDPVTGLTCDRCPAGTHVVKQCTATTPTECGACPANHYTEFWNYLNRC | |
| | LYCGVRCTEQQVEKSTCSATHNRVCECKPGYHIYADDFCLRHSTCPPGQG | |
| | LLVAGTPTSNTRCGPCQAGYFSAEDSTEACKPHTPCKDTERSVPGTATQD | |
| | TFCTSCQARKCDITAPPAPDKAVCDDAFIDFVERYPLNAKKAKNLMRKLR | |
| | KMGKGKTIRESFQAIMAKRNDQPLACEVLSTLNDVDSDMGCRIKTFFLGW | |
| | NEDEC | |
| IIFP-34 | ESKYGPPCPPCPAPEAAGGPSVFLFPPKPKDTLMISRTPEVTCVVVDVSQ | 203 |
| without hIL-2 | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSKVPT | |
| | WTDPVTGLTCDRCPAGTHVVKQCTATTPTECGACPANHYTEFWNYLNRCL | |
| | YCGVRCTEQQVEKSTCSATHNRVCECKPGYHIYADDFCLRHSTCPPGQGL | |
| | LVAGTPTSNTRCGPCQAGYFSAEDSTEACKPHTPCKDTERSVPGTATQDT | |
| | FCTSCQARKCDITAPPAPDKAVCDDAFIDFVERYPLNAKKAKNLMRKLRK | |
| | MGKGKTIRESFQAIMAKRNDQPLACEVLSTLNDVDSDMGCRIKTFFLGWN | |
| | EDEC | |

In some embodiments, the amino acid sequence of the immunoreceptor inhibitory fusion protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 9. In some embodiments, the amino acid sequence of the immunoreceptor inhibitory fusion protein comprises an amino acid sequence at least 85% identical to the amino acid sequence of a polypeptide set forth in Table 9. In some embodiments, the amino acid sequence of the immunoreceptor inhibitory fusion protein comprises an amino acid sequence at least 90% identical to the amino acid sequence of a polypeptide set forth in Table 9. In some embodiments, the amino acid sequence of the immunoreceptor inhibitory fusion protein comprises an amino acid sequence at least 95% identical to the amino acid sequence of a polypeptide set forth in Table 9. In some embodiments, the amino acid sequence of the immunoreceptor inhibitory fusion protein comprises an amino acid sequence at least 100% identical to the amino acid sequence of a polypeptide set forth in Table 9.

In some embodiments, the amino acid sequence of the immunoreceptor inhibitory fusion protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 9. In some embodiments, the amino acid sequence of the immunoreceptor inhibitory fusion protein consists of an amino acid sequence at least 85% identical to the amino acid sequence of a polypeptide set forth in Table 9. In some embodiments, the amino acid sequence of the immunoreceptor inhibitory fusion protein consists of an amino acid sequence at least 90% identical to the amino acid sequence of a polypeptide set forth in Table 9. In some embodiments, the amino acid sequence of the immunoreceptor inhibitory fusion protein consists of an amino acid sequence at least 95% identical to the amino acid sequence of a polypeptide set forth in Table 9. In some embodiments, the amino acid sequence of the immunoreceptor inhibitory fusion protein consists of an amino acid sequence at least 100% identical to the amino acid sequence of a polypeptide set forth in Table 9.

In some embodiments, the amino acid sequence of the immunoreceptor inhibitory fusion protein comprises the amino acid sequence of a polypeptide set forth in Table 9, and further comprises 1 or more but less than 15% (less than 12%, less than 10%, less than 8%), amino acid variations (e.g., substitutions, additions, deletions, etc.). In some embodiments, the amino acid sequence of the immunoreceptor inhibitory fusion protein comprises the amino acid sequence of a polypeptide set forth in Table 9, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunoreceptor inhibitory fusion protein comprises the amino acid sequence of a polypeptide set forth in Table 9, and further comprises about 1, 2, 3, 4, 5, 6, 7, 8, 9, or 10 amino acid variations (e.g., substitutions, additions, deletions, etc.). In some embodiments, the amino acid sequence of the immunoreceptor inhibitory fusion protein comprises the amino acid sequence of a polypeptide set forth in Table 9, and further consists of about 1, 2, 3, 4, 5, 6, 7, 8, 9, or 10 amino acid variations (e.g., substitutions, additions, deletions, etc.). In some embodiments, the amino acid sequence of the immunoreceptor inhibitory fusion protein comprises the amino acid sequence of a polypeptide set forth in Table 9, and further comprises no more than about 1, 2, 3, 4, 5, 6, 7, 8, 9, or 10 amino acid variations (e.g., substitutions, additions, deletions, etc.).

In some embodiments, the amino acid sequence of the immunoreceptor inhibitory fusion protein consists of the amino acid sequence of a polypeptide set forth in Table 9, and further comprises 1 or more but less than 15% (less than 12%, less than 10%, less than 8%), amino acid variations (e.g., substitutions, additions, deletions, etc.). In some embodiments, the amino acid sequence of the immunoreceptor inhibitory fusion protein consists of the amino acid sequence of a polypeptide set forth in Table 9, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunoreceptor inhibitory fusion protein consists of the amino acid sequence of a polypeptide set forth in Table 9, and further comprises about 1, 2, 3, 4, 5, 6, 7, 8, 9, or 10 amino acid variations (e.g., substitutions, additions, deletions, etc.). In some embodiments, the amino acid sequence of the immunoreceptor inhibitory fusion protein consists of the amino acid sequence of a polypeptide set forth in Table 9, and further consists of about 1, 2, 3, 4, 5, 6, 7, 8, 9, or 10 amino acid variations (e.g., substitutions, additions, deletions, etc.). In some embodiments, the amino acid sequence of the immunoreceptor inhibitory fusion protein consists of the amino acid sequence of a polypeptide set forth in Table 9, and further comprises no more than about 1, 2, 3, 4, 5, 6, 7, 8, 9, or 10 amino acid variations (e.g., substitutions, additions, deletions, etc.).

In some embodiments, the amino acid sequence of the immunoreceptor inhibitory fusion protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 174-185 or 191-196. In some embodiments, the amino acid sequence of the immunoreceptor inhibitory fusion protein comprises an amino acid sequence at least 85% identical to the amino acid sequence set forth in any one of SEQ ID NOS: 174-185 or 191-196. In some embodiments, the amino acid sequence of the immunoreceptor inhibitory fusion protein comprises an amino acid sequence at least 90% identical to the amino acid sequence set forth in any one of SEQ ID NOS: 174-185 or 191-196. In some embodiments, the amino acid sequence of the immunoreceptor inhibitory fusion protein comprises an amino acid sequence at least 95% identical to the amino acid sequence set forth in any one of SEQ ID NOS: 174-185 or 191-196. In some embodiments, the amino acid sequence of the immunoreceptor inhibitory fusion protein comprises an amino acid sequence at least 100% identical to the amino acid sequence set forth in any one of SEQ ID NOS: 174-185 or 191-196.

In some embodiments, the amino acid sequence of the immunoreceptor inhibitory fusion protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 174-185 or 191-196. In some embodiments, the amino acid sequence of the immunoreceptor inhibitory fusion protein consists of an amino acid sequence at least 85% identical to the amino acid sequence set forth in any one of SEQ ID NOS: 174-185 or 191-196. In some embodiments, the amino acid sequence of the immunoreceptor inhibitory fusion protein consists of an amino acid sequence at least 90% identical to the amino acid sequence set forth in any one of SEQ ID NOS: 174-185 or 191-196. In some embodiments, the amino acid sequence of the immunoreceptor inhibitory fusion protein consists of an amino acid sequence at least 95% identical to the amino acid sequence set forth in any one of SEQ ID NOS: 174-185 or 191-196. In some embodiments, the amino acid sequence of the immunoreceptor inhibitory fusion protein consists of an amino acid sequence at least 100% identical to the amino acid sequence set forth in any one of SEQ ID NOS: 174-185 or 191-196.

In some embodiments, the amino acid sequence of the immunoreceptor inhibitory fusion protein comprises the amino acid sequence set forth in any one of SEQ ID NOS: 174-185 or 191-196, and further comprises 1 or more but less than 15% (less than 12%, less than 10%, less than 8%), amino acid variations (e.g., substitutions, additions, deletions, etc.). In some embodiments, the amino acid sequence of the immunoreceptor inhibitory fusion protein comprises the amino acid sequence set forth in any one of SEQ ID NOS: 174-185 or 191-196, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunoreceptor inhibitory fusion protein comprises the amino acid sequence set forth in any one of SEQ ID NOS: 174-185 or 191-196, and further comprises about 1, 2, 3, 4, 5, 6, 7, 8, 9, or 10 amino acid variations (e.g., substitutions, additions, deletions, etc.). In some embodiments, the amino acid sequence of the immunoreceptor inhibitory fusion protein comprises the amino acid sequence set forth in any one of SEQ ID NOS: 174-185 or 191-196, and further consists of about 1, 2, 3, 4, 5, 6, 7, 8, 9, or 10 amino acid variations (e.g., substitutions, additions, deletions, etc.). In some embodiments, the amino acid sequence of the immunoreceptor inhibitory fusion protein comprises the amino acid sequence set forth in any one of SEQ ID NOS: 174-185 or 191-196, and further comprises no more than about 1, 2, 3, 4, 5, 6, 7, 8, 9, or 10 amino acid variations (e.g., substitutions, additions, deletions, etc.).

In some embodiments, the amino acid sequence of the immunoreceptor inhibitory fusion protein consists of the amino acid sequence set forth in any one of SEQ ID NOS: 174-185 or 191-196, and further comprises 1 or more but less than 15% (less than 12%, less than 10%, less than 8%), amino acid variations (e.g., substitutions, additions, deletions, etc.). In some embodiments, the amino acid sequence of the immunoreceptor inhibitory fusion protein consists of the amino acid sequence set forth in any one of SEQ ID NOS: 174-185 or 191-196, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunoreceptor inhibitory fusion protein consists of the amino acid sequence set forth in any one of SEQ ID NOS: 174-185 or 191-196, and further comprises about 1, 2, 3, 4, 5, 6, 7, 8, 9, or 10 amino acid variations (e.g., substitutions, additions, deletions, etc.). In some embodiments, the amino acid sequence of the immunoreceptor inhibitory fusion protein consists of the amino acid sequence set forth in any one of SEQ ID NOS: 174-185 or 191-196, and further consists of about 1, 2, 3, 4, 5, 6, 7, 8, 9, or 10 amino acid variations (e.g., substitutions, additions, deletions, etc.). In some embodiments, the amino acid sequence of the immunoreceptor inhibitory fusion protein consists of the amino acid sequence set forth in any one of SEQ ID NOS: 174-185 or 191-196, and further comprises no more than about 1, 2, 3, 4, 5, 6, 7, 8, 9, or 10 amino acid variations (e.g., substitutions, additions, deletions, etc.).

In specific embodiments, the amino acid sequence of the immunoreceptor inhibitory fusion protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 177-179, 194-196, or 201-203. In specific embodiments, the amino acid sequence of the immunoreceptor inhibitory fusion protein comprises an amino acid sequence at least 85% identical to the amino acid sequence set forth in any one of SEQ ID NOS: 177-179, 194-196, or 201-203. In specific embodiments, the amino acid sequence of the immunoreceptor inhibitory fusion protein comprises an amino acid sequence at least 90% identical to the amino acid sequence set forth in any one of SEQ ID NOS: 177-179, 194-196, or 201-203. In specific embodiments, the amino acid sequence of the immunoreceptor inhibitory fusion protein comprises an amino acid sequence at least 95% identical to the amino acid sequence set forth in any one of SEQ ID NOS: 177-179, 194-196, or 201-203. In specific embodiments, the amino acid sequence of the immunoreceptor inhibitory fusion protein comprises an amino acid sequence at least 100% identical to the amino acid sequence set forth in any one of SEQ ID NOS: 177-179, 194-196, or 201-203.

In specific embodiments, the amino acid sequence of the immunoreceptor inhibitory fusion protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 177-179, 194-196, or 201-203. In specific embodiments, the amino acid sequence of the immunoreceptor inhibitory fusion protein consists of an amino acid sequence at least 85% identical to the amino acid sequence set forth in any one of SEQ ID NOS: 177-179, 194-196, or 201-203. In specific embodiments, the amino acid sequence of the immunoreceptor inhibitory fusion protein consists of an amino acid sequence at least 90% identical to the amino acid sequence set forth in any one of SEQ ID NOS: 177-179, 194-196, or 201-203. In specific embodiments, the amino acid sequence of the immunoreceptor inhibitory fusion protein consists of an amino acid sequence at least 95% identical to the amino acid sequence set forth in any one of SEQ ID NOS: 177-179, 194-196, or 201-203. In specific embodiments, the amino acid sequence of the immunoreceptor inhibitory fusion protein consists of an amino acid sequence at least 100% identical to the amino acid sequence set forth in any one of SEQ ID NOS: 177-179, 194-196, or 201-203.

In specific embodiments, the amino acid sequence of the immunoreceptor inhibitory fusion protein comprises the amino acid sequence set forth in any one of SEQ ID NOS: 177-179, 194-196, or 201-203, and further comprises 1 or more but less than 15% (less than 12%, less than 10%, less than 8%), amino acid variations (e.g., substitutions, additions, deletions, etc.). In specific embodiments, the amino acid sequence of the immunoreceptor inhibitory fusion protein comprises the amino acid sequence set forth in any one of SEQ ID NOS: 177-179, 194-196, or 201-203, and further comprises or consists of at least about 1, 2, 3, 4, 5, 6, 7, 8, 9, or 10 amino acid variations (e.g., substitutions, additions, deletions, etc.). In specific embodiments, the amino acid sequence of the immunoreceptor inhibitory fusion protein comprises the amino acid sequence set forth in any one of SEQ ID NOS: 177-179, 194-196, or 201-203, and further comprises about 1, 2, 3, 4, 5, 6, 7, 8, 9, or 10 amino acid variations (e.g., substitutions, additions, deletions, etc.). In specific embodiments, the amino acid sequence of the immunoreceptor inhibitory fusion protein comprises the amino acid sequence set forth in any one of SEQ ID NOS: 177-179, 194-196, or 201-203, and further consists of about 1, 2, 3, 4, 5, 6, 7, 8, 9, or 10 amino acid variations (e.g., substitutions, additions, deletions, etc.). In specific embodiments, the amino acid sequence of the immunoreceptor inhibitory fusion protein comprises the amino acid sequence set forth in any one of SEQ ID NOS: 177-179, 194-196, or 201-203, and further comprises no more than about 1, 2, 3, 4, 5, 6, 7, 8, 9, or 10 amino acid variations (e.g., substitutions, additions, deletions, etc.).

In specific embodiments, the amino acid sequence of the immunoreceptor inhibitory fusion protein consists of the amino acid sequence set forth in any one of SEQ ID NOS: 177-179, 194-196, or 201-203, and further comprises 1 or more but less than 15% (less than 12%, less than 10%, less than 8%), amino acid variations (e.g., substitutions, additions, deletions, etc.). In specific embodiments, the amino acid sequence of the immunoreceptor inhibitory fusion protein consists of the amino acid sequence set forth in any one of SEQ ID NOS: 177-179, 194-196, or 201-203, and further comprises or consists of at least about 1, 2, 3, 4, 5, 6, 7, 8, 9, or 10 amino acid variations (e.g., substitutions, additions, deletions, etc.). In specific embodiments, the amino acid sequence of the immunoreceptor inhibitory fusion protein consists of the amino acid sequence set forth in any one of SEQ ID NOS: 177-179, 194-196, or 201-203, and further comprises about 1, 2, 3, 4, 5, 6, 7, 8, 9, or 10 amino acid variations (e.g., substitutions, additions, deletions, etc.). In specific embodiments, the amino acid sequence of the immunoreceptor inhibitory fusion protein consists of the amino acid sequence set forth in any one of SEQ ID NOS: 177-179, 194-196, or 201-203, and further consists of about 1, 2, 3, 4, 5, 6, 7, 8, 9, or 10 amino acid variations (e.g., substitutions, additions, deletions, etc.). In specific embodiments, the amino acid sequence of the immunoreceptor inhibitory fusion protein consists of the amino acid sequence set forth in any one of SEQ ID NOS: 177-179, 194-196, or 201-203, and further comprises no more than about 1, 2, 3, 4, 5, 6, 7, 8, 9, or 10 amino acid variations (e.g., substitutions, additions, deletions, etc.).

### 5.5 Immunogenic Peptides & Proteins

In one aspect, provided herein are immunogenic peptides or proteins comprising at least an immunogenic fragment of an immunoreceptor inhibitory protein described herein.

In some embodiments, the immunogenic peptide or protein does not specifically bind a TNFSF ligand (e.g., hTL1A) or binds a TNFSF ligand (e.g., hTL1A) with lower affinity relative to a reference immunoreceptor inhibitory protein described herein. Binding to and/or affinity for the TNF superfamily member (e.g., described herein, e.g., TL1A) can be assessed using standard methods known in the art and described herein, see, e.g., § 5.3.

In some embodiments, the immunogenic peptide or protein further comprises one or more heterologous peptide or protein element, or a nucleic acid molecule (e.g., described herein) that encodes at least one heterologous peptide or protein element. In some embodiments, the at least one heterologous peptide or protein element may impart an additional function to the immunogenic peptide or protein, e.g., to promote or improve secretion of the encoded immunogenic peptide or protein (e.g., a signal peptide (e.g., described herein), promote or improve anchoring of the encoded immunogenic peptide or protein described herein in the plasma membrane (e.g., via transmembrane elements), promote or improve formation of immunogen complexes (e.g., via multimerization domains or immunogen clustering elements), or promote or improve virus-like particle formation (VLP forming sequence).

In some embodiments, the immunogenic peptide or protein is formulated with an adjuvant.

### 5.5.1 Fragments of Immunoreceptor Inhibitory Proteins

In some embodiments, the immunogenic peptide or protein comprises an immunogenic fragment of an immunoreceptor inhibitory protein described herein. In some embodiments, the immunogenic peptide or protein consists of an immunogenic fragment of an immunoreceptor inhibitory protein described herein. In some embodiments, the immunogenic peptide or protein comprises an immunoreceptor inhibitory protein described herein. In some embodiments, the immunogenic peptide or protein consists of an immunoreceptor inhibitory protein described herein. In some embodiments, the immunogenic peptide or protein comprises a full-length immunoreceptor inhibitory protein described herein. In some embodiments, the immunogenic peptide or protein consists of a full-length immunoreceptor inhibitory protein described herein.

In some embodiments, the immunogenic peptide or protein comprises at least about 10, 15, 20, 30, 40, 50, 60, 70, 80, 90, 100, 110, 120, or 130 amino acids. In some embodiments, the immunogenic peptide or protein comprises from about 10-130, 10-120, 10-110, 10-100, 10-90, 10-80, 10-70, 10-60, 10-50, 10-40, 10-30, or 10-20 amino acids. In some embodiments, the immunogenic peptide or protein comprises at least about 10, 15, 20, 30, 40, 50, 60, 70, 80, 90, 100, 110, 120, or 130 amino acids. In some embodiments, the immunogenic peptide or protein comprises about 10, 15, 20, 30, 40, 50, 60, 70, 80, 90, 100, 110, 120, or 130 amino acids. In some embodiments, the immunogenic peptide or protein consists of about 10, 15, 20, 30, 40, 50, 60, 70, 80, 90, 100, 110, 120, or 130 amino acids. In some embodiments, the immunogenic peptide or protein comprises or consists of no more than about 10, 15, 20, 30, 40, 50, 60, 70, 80, 90, 100, 110, 120, or 130 amino acids.

In some embodiments, the immunogenic peptide or protein comprises at least a portion of an immunoreceptor inhibitory protein described herein. In some embodiments, the immunogenic peptide or protein comprises or consists of at least about 10, 15, 20, 30, 40, 50, 60, 70, 80, 90, 100, 110, 120, or 130 contiguous amino acids of an immunoreceptor inhibitory protein described herein. In some embodiments, the immunogenic peptide or protein comprises or consists of about 10, 15, 20, 30, 40, 50, 60, 70, 80, 90, 100, 110, 120, or 130 contiguous amino acids of an immunoreceptor inhibitory protein described herein. In some embodiments, the immunogenic peptide or protein comprises or consists of no more than about 10, 15, 20, 30, 40, 50, 60, 70, 80, 90, 100, 110, 120, or 130 contiguous amino acids of an immunoreceptor inhibitory protein described herein. In some embodiments, the immunogenic peptide or protein comprises or consists of from about 10-130, 10-120, 10-110, 10-100, 10-90, 10-80, 10-70, 10-60, 10-50, 10-40, 10-30, or 10-20 contiguous amino acids of an immunoreceptor inhibitory protein described herein.

In some embodiments, immunogenic peptide or protein comprises an amino acid sequence that is at least 80%, 85%, 90%, 91%, 92%, 93%, 94%, 95%, 96%, 97%, 98%, 99% or 100% identical to a portion of the amino acid sequence of an immunoreceptor inhibitory protein described herein. In some embodiments, the immunogenic peptide or protein comprises an amino acid sequence that is at least about 80%, 85%, 90%, 91%, 92%, 93%, 94%, 95%, 96%, 97%, 98%, 99% or 100% identical to a contiguous stretch of at least about 10, 15, 20, 30, 40, 50, 60, 70, 80, 90, 100, 110, 120, or 130 amino acids of an immunoreceptor inhibitory protein described herein. In some embodiments, the immunogenic peptide or protein comprises an amino acid sequence that is at least 80%, 85%, 90%, 91%, 92%, 93%, 94%, 95%, 96%, 97%, 98%, 99% or 100% identical to a contiguous stretch of at least about 10, 15, 20, 30, 40, 50, 60, 70, 80, 90, 100, 110, 120, or 130 amino acids of an immunoreceptor inhibitory protein described herein. In some embodiments, the immunogenic peptide or protein comprises an amino acid sequence that is at least 80%, 85%, 90%, 91%, 92%, 93%, 94%, 95%, 96%, 97%, 98%, 99% or 100% identical to a contiguous stretch of about 10, 15, 20, 30, 40, 50, 60, 70, 80, 90, 100, 110, 120, or 130 amino acids of an immunoreceptor inhibitory protein described herein. In some embodiments, the immunogenic peptide or protein comprises an amino acid sequence that is at least 80%, 85%, 90%, 91%, 92%, 93%, 94%, 95%, 96%, 97%, 98%, 99% or 100% identical to a contiguous stretch of no more than about 10, 15, 20, 30, 40, 50, 60, 70, 80, 90, 100, 110, 120, or 130 amino acids of an immunoreceptor inhibitory protein described herein.

In some embodiments, the amino acid sequence of the immunogenic peptide or protein comprises 1 or more but less than 15% (less than 12%, less than 10%, less than 8%), amino acid variations (e.g., substitution, addition, deletion) relative to the amino acid sequence of an immunoreceptor inhibitory protein described herein. In some embodiments, the amino acid sequence of an immunogenic peptide or protein comprises at least one amino acid variation (e.g., substitution, addition, deletion) relative to the amino acid sequence of an immunoreceptor inhibitory protein described herein. In some embodiments, the amino acid sequence of the immunogenic peptide or protein comprises at least about 1, 2, 3, 4, 5, 6, 7, 8, 9, 10 or more amino acid variations (e.g., substitution, addition, deletion) relative to the amino acid sequence of an immunoreceptor inhibitory protein described herein. In some embodiments, the amino acid sequence of the immunogenic peptide or protein comprises or consists of about 1, 2, 3, 4, 5, 6, 7, 8, 9, 10 or more amino acid variations (e.g., substitution, addition, deletion) relative to the amino acid sequence of an immunoreceptor inhibitory protein described herein. In some embodiments, the amino acid sequence of the immunogenic peptide or protein comprises or consists of no more than about 1, 2, 3, 4, 5, 6, 7, 8, 9, 10 amino acid variations (e.g., substitution, addition, deletion) relative to the amino acid sequence of an immunoreceptor inhibitory protein described herein.

In some embodiments, the immunogenic peptide or protein comprises at least a portion of an immunoreceptor inhibitory protein described herein. In some embodiments, the immunogenic peptide or protein comprises or consists of at least about 10, 15, 20, 30, 40, 50, 60, 70, 80, 90, 100, 110, 120, or 130 contiguous amino acids of an immunoreceptor inhibitory protein set forth in Table 2. In some embodiments, the immunogenic peptide or protein comprises or consists of about 10, 15, 20, 30, 40, 50, 60, 70, 80, 90, 100, 110, 120 or 130 contiguous amino acids of an immunoreceptor inhibitory protein set forth in Table 2. In some embodiments, the immunogenic peptide or protein comprises or consists of no more than about 10, 15, 20, 30, 40, 50, 60, 70, 80, 90, 100, 110, 120, 130 contiguous amino acids of an immunoreceptor inhibitory protein set forth in Table 2. In some embodiments, the immunogenic peptide or protein comprises or consists of from about 10-130, 10-120, 10-110, 10-100, 10-90, 10-80, 10-70, 10-60, 10-50, 10-40, 10-30, or 10-20 contiguous amino acids of an immunoreceptor inhibitory protein set forth in Table 2.

In some embodiments, immunogenic peptide or protein comprises an amino acid sequence that is at least 80%, 85%, 90%, 91%, 92%, 93%, 94%, 95%, 96%, 97%, 98%, 99% or 100% identical to a portion of the amino acid sequence of an immunoreceptor inhibitory protein set forth in Table 2. In some embodiments, the immunogenic peptide or protein comprises an amino acid sequence that is at least about 80%, 85%, 90%, 91%, 92%, 93%, 94%, 95%, 96%, 97%, 98%, 99% or 100% identical to a contiguous stretch of at least about 10, 15, 20, 30, 40, 50, 60, 70, 80, 90, 100, 110, 120, or 130 amino acids of an immunoreceptor inhibitory protein set forth in Table 2. In some embodiments, the immunogenic peptide or protein comprises an amino acid sequence that is at least 80%, 85%, 90%, 91%, 92%, 93%, 94%, 95%, 96%, 97%, 98%, 99% or 100% identical to a contiguous stretch of at least about 10, 15, 20, 30, 40, 50, 60, 70, 80, 90, 100, 110, 120, or 130 amino acids of an immunoreceptor inhibitory protein set forth in Table 2. In some embodiments, the immunogenic peptide or protein comprises an amino acid sequence that is at least 80%, 85%, 90%, 91%, 92%, 93%, 94%, 95%, 96%, 97%, 98%, 99% or 100% identical to a contiguous stretch of about 10, 15, 20, 30, 40, 50, 60, 70, 80, 90, 100, 110, 120, or 130 amino acids of an immunoreceptor inhibitory protein set forth in Table 2. In some embodiments, the immunogenic peptide or protein comprises an amino acid sequence that is at least 80%, 85%, 90%, 91%, 92%, 93%, 94%, 95%, 96%, 97%, 98%, 99% or 100% identical to a contiguous stretch of no more than about 10, 15, 20, 30, 40, 50, 60, 70, 80, 90, 100, 110, 120, or 130 amino acids of an immunoreceptor inhibitory protein set forth in Table 2.

In some embodiments, the amino acid sequence of the immunogenic peptide or protein comprises 1 or more but less than 15% (less than 12%, less than 10%, less than 8%), amino acid variations (e.g., substitution, addition, deletion) relative to the amino acid sequence of an immunoreceptor inhibitory protein set forth in Table 2. In some embodiments, the amino acid sequence of the immunogenic peptide or protein comprises at least one amino acid variation (e.g., substitution, addition, deletion) relative to the amino acid sequence of an immunoreceptor inhibitory protein set forth in Table 2. In some embodiments, the amino acid sequence of the immunogenic peptide or protein comprises at least about 1, 2, 3, 4, 5, 6, 7, 8, 9, 10 or more amino acid variations (e.g., substitution, addition, deletion) relative to the amino acid sequence of an immunoreceptor inhibitory protein set forth in Table 2. In some embodiments, the amino acid sequence of the immunogenic peptide or protein comprises or consists of about 1, 2, 3, 4, 5, 6, 7, 8, 9, 10 or more amino acid variations (e.g., substitution, addition, deletion) relative to the amino acid sequence of an immunoreceptor inhibitory protein set forth in Table 2. In some embodiments, the amino acid sequence of the immunogenic peptide or protein comprises or consists of no more than about 1, 2, 3, 4, 5, 6, 7, 8, 9, 10 amino acid variations (e.g., substitution, addition, deletion) relative to the amino acid sequence of an immunoreceptor inhibitory protein set forth in Table 2.

In some embodiments, the immunogenic peptide or protein comprises at least a portion of an immunoreceptor inhibitory protein described herein. In some embodiments, the immunogenic peptide or protein comprises or consists of at least about 10, 15, 20, 30, 40, 50, 60, 70, 80, 90, 100, 110, 120, or 130 contiguous amino acids of an immunoreceptor inhibitory protein set forth the in any one of SEQ ID NOS: 23-84, 187-190, or 199-200. In some embodiments, the immunogenic peptide or protein comprises or consists of about 10, 15, 20, 30, 40, 50, 60, 70, 80, 90, 100, 150, or 200 contiguous amino acids of an immunoreceptor inhibitory protein set forth the in any one of SEQ ID NOS: 23-84, 187-190, or 199-200. In some embodiments, the immunogenic peptide or protein comprises or consists of no more than about 10, 15, 20, 30, 40, 50, 60, 70, 80, 90, 100, 110, 120, or 130 contiguous amino acids of an immunoreceptor inhibitory protein set forth the in any one of SEQ ID NOS: 23-84, 187-190, or 199-200. In some embodiments, the immunogenic peptide or protein comprises or consists of from about 10-130, 10-120, 10-110, 10-100, 10-90, 10-80, 10-70, 10-60, 10-50, 10-40, 10-30, or 10-20 contiguous amino acids of an immunoreceptor inhibitory protein set forth the in any one of SEQ ID NOS: 23-84, 187-190, or 199-200.

In some embodiments, immunogenic peptide or protein comprises an amino acid sequence that is at least 80%, 85%, 90%, 91%, 92%, 93%, 94%, 95%, 96%, 97%, 98%, 99% or 100% identical to a portion of the amino acid sequence of an immunoreceptor inhibitory protein set forth the in any one of SEQ ID NOS: 23-84, 187-190, or 199-200. In some embodiments, the immunogenic peptide or protein comprises an amino acid sequence that is at least about 80%, 85%, 90%, 91%, 92%, 93%, 94%, 95%, 96%, 97%, 98%, 99% or 100% identical to a contiguous stretch of at least about 10, 15, 20, 30, 40, 50, 60, 70, 80, 90, 100, 110, 120, or 130 amino acids of an immunoreceptor inhibitory protein set forth in any one of SEQ ID NOS: 23-84, 187-190, or 199-200. In some embodiments, the immunogenic peptide or protein comprises an amino acid sequence that is at least 80%, 85%, 90%, 91%, 92%, 93%, 94%, 95%, 96%, 97%, 98%, 99% or 100% identical to a contiguous stretch of at least about 10, 15, 20, 30, 40, 50, 60, 70, 80, 90, 100, 110, 120, or 130 amino acids of an immunoreceptor inhibitory protein set forth the in any one of SEQ ID NOS: 23-84, 187-190, or 199-200. In some embodiments, the immunogenic peptide or protein comprises an amino acid sequence that is at least 80%, 85%, 90%, 91%, 92%, 93%, 94%, 95%, 96%, 97%, 98%, 99% or 100% identical to a contiguous stretch of about 10, 15, 20, 30, 40, 50, 60, 70, 80, 90, 100, 150, or 200 amino acids of an immunoreceptor inhibitory protein set forth the in any one of SEQ ID NOS: 23-84, 187-190, or 199-200. In some embodiments, the immunogenic peptide or protein comprises an amino acid sequence that is at least 80%, 85%, 90%, 91%, 92%, 93%, 94%, 95%, 96%, 97%, 98%, 99% or 100% identical to a contiguous stretch of no more than about 10, 15, 20, 30, 40, 50, 60, 70, 80, 90, 100, 110, 120, or 130 amino acids of an immunoreceptor inhibitory protein set forth the in any one of SEQ ID NOS: 23-84, 187-190, or 199-200.

In some embodiments, the amino acid sequence of the immunogenic peptide or protein comprises 1 or more but less than 15% (less than 12%, less than 10%, less than 8%), amino acid variations (e.g., substitution, addition, deletion) relative to the amino acid sequence of an immunoreceptor inhibitory protein set forth the in any one of SEQ ID NOS: 23-84, 187-190, or 199-200. In some embodiments, the amino acid sequence of the immunogenic peptide or protein comprises at least one amino acid variation (e.g., substitution, addition, deletion) relative to the amino acid sequence of an immunoreceptor inhibitory protein set forth the in any one of SEQ ID NOS: 23-84, 187-190, or 199-200. In some embodiments, the amino acid sequence of the immunogenic peptide or protein comprises at least about 1, 2, 3, 4, 5, 6, 7, 8, 9, 10 or more amino acid variations (e.g., substitution, addition, deletion) relative to the amino acid sequence of an immunoreceptor inhibitory protein set forth the in any one of SEQ ID NOS: 23-84, 187-190, or 199-200. In some embodiments, the amino acid sequence of the immunogenic peptide or protein comprises or consists of about 1, 2, 3, 4, 5, 6, 7, 8, 9, 10 or more amino acid variations (e.g., substitution, addition, deletion) relative to the amino acid sequence of an immunoreceptor inhibitory protein set forth the in any one of SEQ ID NOS: 23-84, 187-190, or 199-200. In some embodiments, the amino acid sequence of the immunogenic peptide or protein comprises or consists of no more than about 1, 2, 3, 4, 5, 6, 7, 8, 9, 10 amino acid variations (e.g., substitution, addition, deletion) relative to the amino acid sequence of an immunoreceptor inhibitory protein set forth the in any one of SEQ ID NOS: 23-84, 187-190, or 199-200.

### 5.5.2 Variants of Immunoreceptor Inhibitory Proteins

In some embodiments, the immunogenic peptide or protein comprises at least one amino acid variation (e.g., substitution, addition, deletion) relative to the amino acid sequence of an immunoreceptor inhibitory protein described herein. In some embodiments, the amino acid sequence of the immunogenic peptide or protein comprises at least 2, 3, 4, 5, 6, 7, 8, 9, 10, or more amino acid variations (e.g., substitutions, additions, deletions) relative to the amino acid sequence of an immunoreceptor inhibitory protein described herein. In some embodiments, the amino acid sequence of the immunogenic peptide or protein comprises 1 or more but less than 15% (less than 12%, less than 10%, less than 8%), amino acid variations (e.g., substitution, addition, deletion) relative to the amino acid sequence of an immunoreceptor inhibitory protein described herein.

In some embodiments, the amino acid sequence of the immunogenic peptide or protein comprises or consists of the amino acid sequence of an immunoreceptor inhibitory protein described herein comprising 1 or more but less than 15% (less than 12%, less than 10%, less than 8%), amino acid variations (e.g., substitution, addition, deletion) relative to the amino acid sequence of the immunoreceptor inhibitory protein described herein.

In some embodiments, the amino acid sequence of the immunogenic peptide or protein comprises or consists of the amino acid sequence of an immunoreceptor inhibitory protein described herein comprising at least one amino acid variation (e.g., substitution, addition, deletion). In some embodiments, the amnio acid sequence of the immunogenic peptide or protein comprises or consists of the amino acid sequence of an immunoreceptor inhibitory protein described herein comprising at least about 1, 2, 3, 4, 5, 6, 7, 8, 9, 10 or more amino acid variations (e.g., substitution, addition, deletion). In some embodiments, the amino acid sequence of the immunogenic peptide or protein comprises or consists of the amino acid sequence of an immunoreceptor inhibitory protein described herein comprising no more than about 1, 2, 3, 4, 5, 6, 7, 8, 9, 10 amino acid variations (e.g., substitution, addition, deletion).

In some embodiments, the amino acid sequence of the immunogenic peptide or protein comprises or consists of the amino acid sequence of an immunoreceptor inhibitory protein described herein comprising one or more amino acid variation (e.g., substitution, addition, deletion). In some embodiments, the immunogenic peptide or protein comprises an amino acid sequence that, other than the one or more amino acid variation (e.g., substitution, addition, deletion), is at least 80%, 85%, 90%, 91%, 92%, 93%, 94%, 95%, 96%, 97%, 98%, 99% or 100% identical to a contiguous stretch of at least 10, 15, 20, 30, 40, 50, 60, 70, 80, 90, 100, 110, 120, or 130 amino acids of an immunoreceptor inhibitory protein described herein. In some embodiments, immunogenic peptide or protein comprises an amino acid sequence that, other than the one or more amino acid substitution, is at least 80%, 85%, 90%, 91%, 92%, 93%, 94%, 95%, 96%, 97%, 98%, 99% or 100% identical to the amino acid sequence of an immunoreceptor inhibitory protein described herein.

In some embodiments, the immunogenic peptide or protein comprises at least one amino acid variation (e.g., substitution, addition, deletion) relative to the amino acid sequence of an immunoreceptor inhibitory protein set forth in Table 2. In some embodiments, the amino acid sequence of the immunogenic peptide or protein comprises at least 2, 3, 4, 5, 6, 7, 8, 9, 10, or more amino acid variations (e.g., substitutions, additions, deletions) relative to the amino acid sequence of a reference immunoreceptor inhibitory protein set forth in Table 2. In some embodiments, the amino acid sequence of the immunogenic peptide or protein comprises 1 or more but less than 15% (less than 12%, less than 10%, less than 8%), amino acid variations (e.g., substitution, addition, deletion) relative to the amino acid sequence of an immunoreceptor inhibitory protein set forth in Table 2.

In some embodiments, the amino acid sequence of the immunogenic peptide or protein comprises or consists of the amino acid sequence of an immunoreceptor inhibitory protein set forth in Table 2 comprising 1 or more but less than 15% (less than 12%, less than 10%, less than 8%), amino acid variations (e.g., substitution, addition, deletion) relative to the amino acid sequence of the immunoreceptor inhibitory protein set forth in Table 2.

In some embodiments, the amino acid sequence of the immunogenic peptide or protein comprises or consists of the amino acid sequence of an immunoreceptor inhibitory protein set forth in Table 2 comprising at least one amino acid variation (e.g., substitution, addition, deletion). In some embodiments, the amnio acid sequence of the immunogenic peptide or protein comprises or consists of the amino acid sequence of an immunoreceptor inhibitory protein set forth in Table 2 comprising at least about 1, 2, 3, 4, 5, 6, 7, 8, 9, 10 or more amino acid variations (e.g., substitution, addition, deletion). In some embodiments, the amino acid sequence of the immunogenic peptide or protein comprises or consists of the amino acid sequence of an immunoreceptor inhibitory protein set forth in Table 2 comprising no more than about 1, 2, 3, 4, 5, 6, 7, 8, 9, 10 amino acid variations (e.g., substitution, addition, deletion).

In some embodiments, the amino acid sequence of the immunogenic peptide or protein comprises or consists of the amino acid sequence of an immunoreceptor inhibitory protein set forth in Table 2 comprising one or more amino acid variation (e.g., substitution, addition, deletion). In some embodiments, the immunogenic peptide or protein comprises an amino acid sequence that, other than the one or more amino acid variation (e.g., substitution, addition, deletion), is at least 80%, 85%, 90%, 91%, 92%, 93%, 94%, 95%, 96%, 97%, 98%, 99% or 100% identical to a contiguous stretch of at least 10, 15, 20, 30, 40, 50, 60, 70, 80, 90, 100, 110, 120, or 130 amino acids of an immunoreceptor inhibitory protein set forth in Table 2. In some embodiments, immunogenic peptide or protein comprises an amino acid sequence that, other than the one or more amino acid substitution, is at least 80%, 85%, 90%, 91%, 92%, 93%, 94%, 95%, 96%, 97%, 98%, 99% or 100% identical to the amino acid sequence of an immunoreceptor inhibitory protein described herein.

In some embodiments, the immunogenic peptide or protein comprises at least one amino acid variation (e.g., substitution, addition, deletion) relative to the amino acid sequence of an immunoreceptor inhibitory protein set forth in any one of SEQ ID NOS: 23-84, 187-190, or 199-200. In some embodiments, the amino acid sequence of the immunogenic peptide or protein comprises at least 2, 3, 4, 5, 6, 7, 8, 9, 10, or more amino acid variations (e.g., substitutions, additions, deletions) relative to a reference immunogenic protein set forth in any one of SEQ ID NOS: 23-84, 187-190, or 199-200. In some embodiments, the amino acid sequence of the immunogenic peptide or protein comprises 1 or more but less than 15% (less than 12%, less than 10%, less than 8%), amino acid variations (e.g., substitution, addition, deletion) relative to the amino acid sequence of the immunoreceptor inhibitory protein set forth in any one of SEQ ID NOS: 23-84, 187-190, or 199-200.

In some embodiments, the amino acid sequence of the immunogenic peptide or protein comprises or consists of the amino acid sequence of an immunoreceptor inhibitory protein set forth in any one of SEQ ID NOS: 23-84, 187-190, or 199-200 comprising 1 or more but less than 15% (less than 12%, less than 10%, less than 8%), amino acid variations (e.g., substitution, addition, deletion) relative to the amino acid sequence of the immunoreceptor inhibitory protein set forth in any one of SEQ ID NOS: 23-84, 187-190, or 199-200.

In some embodiments, the amino acid sequence of the immunogenic peptide or protein comprises or consists of the amino acid sequence of an immunoreceptor inhibitory protein set forth in any one of SEQ ID NOS: 23-84, 187-190, or 199-200 comprising at least one amino acid variation (e.g., substitution, addition, deletion). In some embodiments, the amnio acid sequence of the immunogenic peptide or protein comprises or consists of the amino acid sequence of an immunoreceptor inhibitory protein set forth in any one of SEQ ID NOS: 23-84, 187-190, or 199-200 comprising at least about 1, 2, 3, 4, 5, 6, 7, 8, 9, 10 or more amino acid variations (e.g., substitution, addition, deletion). In some embodiments, the amino acid sequence of the immunogenic peptide or protein comprises or consists of the amino acid sequence of an immunoreceptor inhibitory protein set forth in any one of SEQ ID NOS: 23-84, 187-190, or 199-200 comprising no more than about 1, 2, 3, 4, 5, 6, 7, 8, 9, 10 amino acid variations (e.g., substitution, addition, deletion).

In some embodiments, the amino acid sequence of the immunogenic peptide or protein comprises or consists of the amino acid sequence of an immunoreceptor inhibitory protein set forth in any one of SEQ ID NOS: 23-84, 187-190, or 199-200 comprising one or more amino acid variation (e.g., substitution, addition, deletion). In some embodiments, the immunogenic peptide or protein comprises an amino acid sequence that, other than the one or more amino acid variation (e.g., substitution, addition, deletion), is at least 80%, 85%, 90%, 91%, 92%, 93%, 94%, 95%, 96%, 97%, 98%, 99% or 100% identical to a contiguous stretch of at least 10, 15, 20, 30, 40, 50, 60, 70, 80, 90, 100, 110, 120, or 130 amino acids of an immunoreceptor inhibitory protein set forth in any one of SEQ ID NOS: 23-84, 187-190, or 199-200. In some embodiments, immunogenic peptide or protein comprises an amino acid sequence that, other than the one or more amino acid substitution, is at least 80%, 85%, 90%, 91%, 92%, 93%, 94%, 95%, 96%, 97%, 98%, 99% or 100% identical to the amino acid sequence of an immunoreceptor inhibitory protein set forth in any one of SEQ ID NOS: 23-84, 187-190, or 199-200.

### 5.5.3 Peptide and Protein-Based Vaccines

In some embodiments, an immunogenic peptide or protein described herein forms the basis for a pharmaceutical composition (e.g., a peptide or protein-based vaccine composition). Therefore, provided herein are pharmaceutical compositions (e.g., vaccine compositions) comprising an immunogenic peptide or protein described herein.

In some embodiments, the vaccine composition comprises a plurality of the immunogenic peptides or proteins (e.g., described herein). In some embodiments, the pharmaceutical composition (e.g., vaccine composition) comprises a plurality of substantially the same immunogenic peptide or protein (e.g., described herein). In some embodiments, the pharmaceutical composition (e.g., vaccine composition) comprises a plurality of different immunogenic peptide or protein (e.g., described herein). In some embodiments, the pharmaceutical composition (e.g., vaccine composition) comprises at least 1, 2, 3, 4, 5, 6, 7, 8, 9, or 10 different immunogenic peptides or proteins (e.g., described herein). In some embodiments, the pharmaceutical composition (e.g., vaccine composition) comprises or consists of 1, 2, 3, 4, 5, 6, 7, 8, 9, or 10 different immunogenic peptides or proteins (e.g., described herein). In some embodiments, the pharmaceutical composition (e.g., vaccine composition) comprises at least one (e.g., 2, 3, 4, 5, 6) immunogenic peptide or protein described herein and at least one immunogenic peptide or protein from a different virus.

### 5.5.4 Nucleic Acid-Based Vaccines

In some embodiments, a nucleic acid molecule encoding an immunogenic peptide or protein described herein (also referred to herein as an “immunogenic nucleic acid molecule”) forms the basis for a pharmaceutical composition (e.g., a vaccine composition (e.g., a nucleic acid-based vaccine)). In some embodiments, the nucleic acid molecule is RNA (e.g., mRNA or circular RNA) or DNA. In some embodiments, the nucleic acid molecule is mRNA. In some embodiments, the nucleic acid molecule is circular RNA (see, e.g., WO2019118919, the full contents of which are incorporated by reference herein for all purposes).

In some embodiments, the segment of the nucleic acid molecule encoding the immunogenic peptide or protein (e.g., described herein) comprises from about 30 to about 20000 nucleotides, about 50 to about 20000 nucleotides, about 500 to about 10000 nucleotides, about 1000 to about 10000 nucleotides, about 1000 to about 5000 nucleotides, or about 2000 to about 5000 nucleotides. In some embodiments, the segment of the nucleic acid molecule encoding the immunogenic peptide or protein (e.g., described herein) comprises at least 30 nucleotides, 50 nucleotides, 100 nucleotides, 200 nucleotides, 300 nucleotides, 400 nucleotides, 500 nucleotides, 1000 nucleotides, 2000 nucleotides, 3000 nucleotides, or 5000 nucleotides.

In some embodiments, the nucleic acid molecule is modified or varied (compared to a reference nucleic acid sequence), e.g., to impart one or more of (a) improved resistance to in vivo degradation, (b) improved stability in vivo, (c) reduced secondary structures, and/or (d) improved translatability in vivo, compared to the reference nucleic acid sequence. Alterations include, without limitation, e.g., codon optimization, nucleotide modification (see, e.g., description below), etc.

In some embodiments, the nucleic acid sequence is codon optimized, e.g., for expression in humans. Codon optimization, in some embodiments, may be used to match codon frequencies in target and host organisms to ensure proper folding; bias guanosine (G) and/or cytosine (C) content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g., glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the nucleic acid molecule. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified or varied to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

In some embodiments, the pharmaceutical composition comprises a plurality of substantially the same nucleic acid molecules encoding a plurality of immunogenic peptides or proteins (e.g., described herein). In some embodiments, the pharmaceutical composition comprises a plurality of different nucleic acid molecules encoding a plurality of different immunogenic peptides or proteins (e.g., described herein). In some embodiments, the pharmaceutical composition comprises at least 1, 2, 3, 4, 5, 6, 7, 8, 9, or 10 different nucleic acid molecules encoding at least 1, 2, 3, 4, 5, 6, 7, 8, 9, or 10 different immunogenic peptides or proteins (e.g., described herein). In some embodiments, the pharmaceutical composition comprises or consists of 1, 2, 3, 4, 5, 6, 7, 8, 9, or 10 different nucleic acid molecules encoding 1, 2, 3, 4, 5, 6, 7, 8, 9, or 10 different immunogenic peptides or proteins (e.g., described herein).

### 5.5.4.1 DNA Molecules

In some embodiments, the nucleic acid molecule is a DNA molecule. In some embodiments, the DNA molecule is a linear coding DNA construct, contained within a plasmid, or contained within a viral vector. In some embodiments, the DNA molecule is a linear coding DNA construct. In some embodiments, the DNA molecule is contained within a plasmid. In some embodiments, the DNA molecule is contained with a viral vector. A more detailed description of viral vectors for both RNA and DNA molecules is provided in § 5.8.2.

The coding DNA may also comprise one or more heterologous nucleic acid elements to mediate expression of the coding region. These include, e.g., promoter(s), enhancer(s), polyadenylation signal(s), synthetic introns, transcriptional termination signals, polyadenylation sequences, and other transcription regulatory elements. A person of ordinary skill in the art is familiar with the transcriptional regulatory elements needed for expression of the coding DNA and can optimize the expression construct (e.g., linear DNA or a plasmid) accordingly.

In some embodiments, a promoter is operably linked to the respective coding nucleic acid sequence encoding the immunogenic peptide or protein. The person of ordinary skill in the art is aware of various promoters that can be employed, for example, a promoter from simian virus 40 (SV40), a mouse mammary tumor virus (MMTV) promoter, a human immunodeficiency virus (HIV) promoter, bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene, for example, from human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727, the entire contents of which is incorporated by reference herein for all purposes. Exemplary polyadenylation signals include, but are not limited to, the bovine growth hormone (BGH) polyadenylation site, SV40 polyadenylation signals, and LTR polyadenylation signals.

In some embodiments, the DNA is contained within a plasmid. A person of ordinary skill in the art is aware of suitable plasmids for expression of the DNA of interest. For example, Suitable plasmid DNA may be generated to allow efficient production of the encoded immunogens in cell lines, e.g., in insect cell lines, for example using vectors as described in WO2009150222A2 and as defined in PCT claims 1 to 33, the disclosure relating to claims 1 to 33 of WO2009150222A2 the entire contents of which is incorporated by reference herein for all purposes.

### 5.5.4.2 RNA Molecules

In some embodiments, the nucleic acid molecule is an RNA molecule. In some embodiments, the RNA molecule is a translatable RNA molecule. In some embodiments, the RNA is selected from an mRNA, a self-replicating RNA, a circular RNA (e.g., a covalently closed RNA), a viral RNA, or a replicon RNA.

In some embodiments, the RNA molecule a circular RNA. Exemplary circular RNAs are described in e.g., U.S. Pat. No. 11,458,156, US20220143062, US20230212629, US20230072532, U.S. Pat. Nos. 11,203,767, 11,352,641, US20210371494, U.S. Pat. No. 11,766,449, US20230226096, WO2021189059, US20190345503, US20220288176, U.S. Pat. No. 11,560,567, WO2022271965, WO2022037692, WO2023024500, WO2023115732, WO2023133684, WO2023143541, WO2023134611, and WO2022247943, the entire contents of each of which are incorporated herein by reference for all purposes.

In some embodiments, the RNA is a mRNA. The basic components of an mRNA molecule typically include at least one coding region (herein a coding region encoding at least one immunogenic peptide or protein described herein), a 5′-untranslated region (UTR), a 3′-UTR, a 5′ cap, and a poly-A tail.

In some embodiments, the RNA (e.g., mRNA) comprises at least one heterologous UTR. The UTRs may harbor regulatory sequence elements that determine the RNA (e.g., mRNA) turnover, stability, localization, and/or expression of operably linked coding sequence(s). The heterologous UTRs may be derived from a naturally occurring gene or may be synthetically engineered. In some embodiments, the 5′-UTR comprises elements for controlling gene expression, e.g., ribosomal binding sites, miRNA binding sites. The 5′-UTR may be post-transcriptionally modified or varied, e.g., by enzymatic or post-transcriptional addition of a 5′cap structure. In some embodiments, the 3′-UTR comprises a polyadenylation signal. In some embodiments, the RNA (e.g., mRNA) comprises at least one coding region encoding the immunogenic peptide or protein described herein and 5′-UTR and/or a 3′-UTR. In some embodiments, the RNA (e.g., mRNA) comprises at least one coding sequence encoding an immunogenic peptide or protein described herein operably connected to at least one heterologous 5′-UTR and at least one 3′-UTR.

In some embodiments, the RNA (e.g., mRNA, circular RNA) comprises a poly(A) sequence. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides. In some embodiments, poly(A) sequence comprises at least 10, 20, 30, 40, 50, 60, 70, 80, 90, 100, 200, 300, 400, or 500 adenosine nucleotides. In some embodiments, the RNA (e.g., mRNA) comprises a poly(A) sequence. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides, wherein the 3′ terminal nucleotide of said nucleic acid molecule is an adenosine. In some embodiments, poly(A) sequence comprises at least 10, 20, 30, 40, 50, 60, 70, 80, 90, 100, 200, 300, 400, or 500 adenosine nucleotides, wherein the 3′ terminal nucleotide of said nucleic acid molecule is an adenosine.

In some embodiments, the RNA (e.g., mRNA, circular RNA) comprises a poly(A)(U) sequence. In some embodiments, the RNA (e.g., mRNA, circular RNA) comprises a poly(A)(C) sequence.

In some embodiments, the RNA (e.g., mRNA) comprises a 5′-cap structure. In some embodiments, the 5′-cap structure stabilizes the RNA (e.g., mRNA), enhances expression of the encoded immunogen, and/or reduces the stimulation of the innate immune system (e.g., after administration to a subject).

Exemplary 5′-cap structures include, but are not limited to, cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g., phosphorothioate modified ARCA), inosine, N1-methyi-guanosine, 2′-fluoro-guanosine, 7-deaza-guanosine, 8-oxo-guanosine, 2-amino-guanosine, LNA-guanosine, and 2-azido-guanosine. In some embodiments, the 5′ cap structure comprises m7G, cap0, cap1, cap2, a modified capO, or a modified cap1 structure.

In some embodiments, the RNA (e.g., mRNA) comprises nucleotide analogues/modifications, e.g., backbone modifications, sugar modifications, and/or base modifications. A backbone modification in the context of the present disclosure is a modification, in which phosphates of the backbone of the nucleotides of the RNA (e.g., mRNA) are chemically modified. A sugar modification in the context of the present disclosure is a chemical modification of the sugar of the nucleotides of the RNA (e.g., mRNA). A base modification in the context of the present disclosure is a chemical modification of the base moiety of the nucleotides of the RNA (e.g., mRNA).

In some embodiments, the RNA (e.g., mRNA) comprises at least one modified nucleotide. Exemplary nucleotide analogues/modifications include, but are not limited to, 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thiocytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphatej-uridine, 5′-O-(1-thiophosphate)-pseudouridine, 6-aza-cytidine, 2-thio-cytidine, alpha-thio-cytidine, Pseudoiso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thioguanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thioadenosine, 8-azido-adenosine, and 7-deaza-adenosine.

In some embodiments, the RNA (e.g., mRNA) comprises pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and/or 2′-O-methyl uridine.

In some embodiments, the RNA (e.g., mRNA) comprises one or more pseudouridine (ψ), N1-methylpseudouridine (m1ψ), 5-methylcytosine, and 5-methoxyuridine. In some embodiments, essentially all, e.g., essentially 100% of the uracil in the coding sequence of the RNA (e.g., mRNA) have a chemical modification, preferably a chemical modification is in the 5-position of the uracil. Incorporating modified nucleotides such as e.g., pseudouridine (ψ), N1-methylpseudouridine (m1ψ), 5-methylcytosine, and/or 5-methoxyuridine into the coding sequence may be advantageous as unwanted innate immune responses (upon administration of the coding RNA or the vaccine) may be adjusted or reduced (if required).

In one embodiment, the mRNA encoding an immunogen described herein comprises: (i) a 5′-cap structure; (ii) a 5′-UTR; (iii) N1-methyl-pseudouridine, cytosine, adenine, and guanine; (iv) a 3′-UTR; and (v) a poly-A region.

RNA (e.g., mRNA) described herein can be generated by e.g., in vitro transcription. In vitro transcription is a method well known to those of ordinary skill in the art for the production of RNA (e.g., mRNA). Generally, the RNA is obtained by DNA-dependent in vitro transcription of an appropriate DNA template, e.g., a linearized plasmid DNA template or a PCR-amplified DNA template. The promoter for controlling RNA in vitro transcription can be any promoter for any DNA-dependent RNA polymerase. Examples of DNA-dependent RNA polymerases include the 17, T3, SP6, or Syn5 RNA polymerases. In some instances, the DNA template is linearized with a suitable restriction enzyme before it is subjected to RNA in vitro transcription. Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); a DNA-dependent RNA polymerase capable of binding to the promoter sequence within the DNA template (e.g., T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCh, which supplies Mg2+ ions as a co-factor for the polymerase; a buffer (TRIS or HEPES) to maintain a suitable pH value, which can also contain antioxidants (e.g., DTT), and/or polyamines such as spermidine at optimal concentrations, e.g., a buffer system comprising TRIS-Citrate as disclosed in WO2017109161. The obtained RNA (e.g., mRNA) products can be purified according to methods known in art. For example, using PureMessenger® (Cure Vac, Tubingen, Germany; RP-HPLC according to WO2008077592) and/or tangential flow filtration (as described in WO2016193206) and/or oligo d(T) purification (see WO2016180430); or using RP-HPLC, e.g., using Reversed-Phase High pressure liquid chromatography (RP-HPLC), the entire contents of each reference is incorporated by reference herein for all purposes.

### 5.6 Methods of Making Proteins

Any protein described herein, e.g., including an immunoreceptor inhibitory protein described herein (see, e.g., § 5.2), a fusion protein described herein (see, e.g., § 5.4), a conjugate described herein (see, e.g., § 5.4), an immunogenic peptide or protein described herein (see, e.g., § 5.5), or an antibody described herein (see, e.g., § 5.10), may be produced using standard methods known in the art. For example, each of the above may be produced by recombinant technology in host cells (e.g., yeast cells, insect cells, mammalian cells, bacteria) that have been transfected or transduced with a nucleic acid expression vector (e.g., plasmid, viral vector (e.g., a baculoviral expression vector)) encoding the subject polypeptide (e.g., immunoreceptor inhibitory protein, fusion protein, immunogenic peptide or protein, or antibody). Such general methods are common knowledge in the art. The expression vector typically contains an expression cassette that includes nucleic acid sequences capable of bringing about expression of the nucleic acid molecule encoding the protein of interest, such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the protein of interest are cultured under conditions conducive to expression of the nucleic acid molecule encoding the immunogenic peptide or protein. Culture media is available from various vendors, and a suitable medium can be routinely chosen for a host cell to express a protein of interest. Host cells can be adherent or suspension cultures, and a person of ordinary skill in the art can optimize culture methods for specific host cells selected. For example, suspension cells can be cultured in, for example, bioreactors in e.g., a batch process or a fed-batch process. The produced protein may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The protein may be concentrated, buffer exchanged by ultrafiltration, and the retentate from the ultrafiltration may be filtered through an appropriate filter, e.g., a 0.22 μm filter. See, e.g., Hacker, David (Ed.), Recombinant Protein Expression in Mammalian Cells: Methods and Protocols (Methods in Molecular Biology), Humana Press (2018); and McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. No. 5,762,939, the entire contents of each of which is incorporated by reference herein for all purposes.

The proteins described herein (e.g., including immunoreceptor inhibitory proteins, fusion proteins and polypeptides, immunogenic peptides and proteins, and antibodies described herein) may be produced synthetically. The proteins described herein (e.g., including the immunoreceptor inhibitory proteins, fusion proteins, immunogenic peptides and proteins, and antibodies described herein) and particularly the immunogenic peptides and proteins described herein, may be produced by using an egg-based manufacturing method. In some embodiments, the proteins described herein are produced in yeast.

In some aspects and embodiments, the disclosure features methods of making a protein described herein (e.g., an immunoreceptor inhibitory protein described herein, a fusion protein described herein, a conjugate described herein, an immunogenic peptide or protein described herein, or an antibody described herein). The method includes (a) recombinantly expressing the protein (e.g., the immunoreceptor inhibitory protein described herein, fusion protein described herein, immunogenic peptide or protein described herein, or antibody described herein); (b) enriching, e.g., purifying, the protein (e.g., the immunoreceptor inhibitory protein described herein, fusion protein described herein, immunogenic peptide or protein described herein, or antibody described herein); (c) evaluating the protein (e.g., the immunoreceptor inhibitory protein described herein, fusion protein described herein, immunogenic peptide or protein described herein, or antibody described herein) for the presence of a process impurity or contaminant, and (d) formulating the protein (e.g., the immunoreceptor inhibitory protein described herein, fusion protein described herein, immunogenic peptide or protein described herein, or antibody described herein) as a pharmaceutical composition if the protein (e.g., the immunoreceptor inhibitory protein described herein, fusion protein described herein, immunogenic peptide or protein described herein, or antibody described herein) meets a threshold specification for the process impurity or contaminant. The process impurity or contaminant evaluated may be one or more of, e.g., a process-related impurity such as host cell proteins, host cell DNA, or a cell culture component (e.g., inducers, antibiotics, or media components); a product-related impurity (e.g., precursors, fragments, aggregates, degradation products); or contaminants, e.g., endotoxin, bacteria, viral contaminants.

### 5.7 Nucleic Acid Molecules

In one aspect, provided herein are nucleic acid molecules (e.g., DNA molecules, RNA molecules, hybrid RNA/DNA molecules) encoding any protein described herein (including, e.g., an immunoreceptor inhibitory protein described herein (see, e.g., § 5.2), a fusion protein described herein (see, e.g., § 5.4), a conjugate described herein (see, e.g., § 5.4), an immunogenic peptide or protein described herein (see, e.g., § 5.5), an antibody described herein (see, e.g., § 5.10)). In some embodiments, the nucleic acid molecule is a DNA molecule or an RNA molecule.

In some embodiments, the nucleic acid molecule is codon optimized. Codon optimization may be used to match codon frequencies in target and host organisms to ensure proper folding; bias guanosine (G) and/or cytosine content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g., glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the nucleic acid molecule. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

### 5.7.1 DNA Molecules

In some embodiments, the nucleic acid molecule is a DNA molecule. In some embodiments, the DNA molecule is a linear coding DNA construct, contained within a plasmid, or contained within a viral vector. In some embodiments, the DNA molecule is a linear coding DNA construct. In some embodiments, the DNA molecule is contained within a plasmid. In some embodiments, the DNA molecule is contained with a viral vector. A more detailed description of viral vectors for both RNA and DNA molecules is provided in § 5.8.2.

The coding DNA may also comprise one or more heterologous nucleic acid elements to mediate expression of the coding region. These include, e.g., promoter(s), enhancer(s), polyadenylation signal(s), synthetic introns, transcriptional termination signals, polyadenylation sequences, and other transcription regulatory elements. A person of ordinary skill in the art is familiar with the transcriptional regulatory elements needed for expression of the coding DNA and can optimize the expression construct (e.g., linear DNA or a plasmid) accordingly.

In some embodiments, a promoter is operably linked to the respective coding nucleic acid sequence encoding the immunogenic peptide or protein. The person of ordinary skill in the art is aware of various promoters that can be employed, for example, a promoter from simian virus 40 (SV40), a mouse mammary tumor virus (MMTV) promoter, a human immunodeficiency virus (HIV) promoter, bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene, for example, from human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727, the entire contents of which is incorporated by reference herein for all purposes. Exemplary polyadenylation signals include, but are not limited to, the bovine growth hormone (BGH) polyadenylation site, SV40 polyadenylation signals, and LTR polyadenylation signals.

In some embodiments, the DNA is contained within a plasmid. A person of ordinary skill in the art is aware of suitable plasmids for expression of the DNA of interest. For example, Suitable plasmid DNA may be generated to allow efficient production of the encoded immunogens in cell lines, e.g., in insect cell lines, for example using vectors as described in WO2009150222A2 and as defined in PCT claims 1 to 33, the disclosure relating to claims 1 to 33 of WO2009150222A2 the entire contents of which is incorporated by reference herein for all purposes.

### 5.7.2 RNA Molecules

In some embodiments, the nucleic acid molecule is an RNA molecule. In some embodiments, the RNA molecule is a translatable RNA molecule. In some embodiments, the RNA is selected from an mRNA, a self-replicating RNA, a circular RNA (e.g., a covalently closed RNA), a viral RNA, or a replicon RNA.

In some embodiments, the RNA molecule a circular RNA. Exemplary circular RNAs are described in e.g., U.S. Pat. No. 11,458,156, US20220143062, US20230212629, US20230072532, U.S. Pat. Nos. 11,203,767, 11,352,641, US20210371494, U.S. Pat. No. 11,766,449, US20230226096, WO2021189059, US20190345503, US20220288176, U.S. Pat. No. 11,560,567, WO2022271965, WO2022037692, WO2023024500, WO2023115732, WO2023133684, WO2023143541, WO2023134611, and WO2022247943, the entire contents of each of which are incorporated herein by reference for all purposes.

In some embodiments, the RNA is a mRNA. The basic components of an mRNA molecule typically include at least one coding region (herein a coding region encoding at least one immunogenic peptide or protein described herein), a 5′-untranslated region (UTR), a 3′-UTR, a 5′ cap, and a poly-A tail.

In some embodiments, the RNA (e.g., mRNA, circular RNA) comprises at least one heterologous UTR. The UTRs may harbor regulatory sequence elements that determine the RNA (e.g., mRNA) turnover, stability, localization, and/or expression of operably linked coding sequence(s). The heterologous UTRs may be derived from a naturally occurring gene or may be synthetically engineered. In some embodiments, the 5′-UTR comprises elements for controlling gene expression, e.g., ribosomal binding sites, miRNA binding sites. The 5′-UTR may be post-transcriptionally modified or varied, e.g., by enzymatic or post-transcriptional addition of a 5′cap structure. In some embodiments, the 3′-UTR comprises a polyadenylation signal. In some embodiments, the RNA (e.g., mRNA, circular RNA) comprises at least one coding region encoding the immunogenic peptide or protein described herein and 5′-UTR and/or a 3′-UTR. In some embodiments, the RNA (e.g., mRNA, circular RNA) comprises at least one coding sequence encoding an immunogenic peptide or protein described herein operably connected to at least one heterologous 5′-UTR and at least one 3′-UTR.

In some embodiments, the RNA (e.g., mRNA, circular RNA) comprises a poly(A) sequence. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides. In some embodiments, poly(A) sequence comprises at least 10, 20, 30, 40, 50, 60, 70, 80, 90, 100, 200, 300, 400, or 500 adenosine nucleotides. In some embodiments, the RNA (e.g., mRNA) comprises a poly(A) sequence. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides, wherein the 3′ terminal nucleotide of said nucleic acid molecule is an adenosine. In some embodiments, poly(A) sequence comprises at least 10, 20, 30, 40, 50, 60, 70, 80, 90, 100, 200, 300, 400, or 500 adenosine nucleotides, wherein the 3′ terminal nucleotide of said nucleic acid molecule is an adenosine.

In some embodiments, the RNA (e.g., mRNA, circular RNA) comprises a poly(A)(U) sequence. In some embodiments, the RNA (e.g., mRNA, circular RNA) comprises a poly(A)(C) sequence.

In some embodiments, the RNA (e.g., mRNA) comprises a 5′-cap structure. In some embodiments, the 5′-cap structure stabilizes the RNA (e.g., mRNA), enhances expression of the encoded immunogen, and/or reduces the stimulation of the innate immune system (e.g., after administration to a subject).

Exemplary 5′-cap structures include, but are not limited to, cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g., phosphorothioate modified ARCA), inosine, N1-methyi-guanosine, 2′-fluoro-guanosine, 7-deaza-guanosine, 8-oxo-guanosine, 2-amino-guanosine, LNA-guanosine, and 2-azido-guanosine. In some embodiments, the 5′ cap structure comprises m7G, cap0, cap1, cap2, a modified capO, or a modified cap1 structure.

In some embodiments, the RNA (e.g., mRNA, circular RNA) comprises nucleotide analogues/modifications, e.g., backbone modifications, sugar modifications, and/or base modifications. A backbone modification in the context of the present disclosure is a modification, in which phosphates of the backbone of the nucleotides of the RNA (e.g., mRNA) are chemically modified. A sugar modification in the context of the present disclosure is a chemical modification of the sugar of the nucleotides of the RNA (e.g., mRNA). A base modification in the context of the present disclosure is a chemical modification of the base moiety of the nucleotides of the RNA (e.g., mRNA).

In some embodiments, the RNA (e.g., mRNA, circular RNA) comprises at least one modified nucleotide. Exemplary nucleotide analogues/modifications include, but are not limited to, 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thiocytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphatej-uridine, 5′-O-(1-thiophosphate)-pseudouridine, 6-aza-cytidine, 2-thio-cytidine, alpha-thio-cytidine, Pseudoiso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thioguanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thioadenosine, 8-azido-adenosine, and 7-deaza-adenosine.

In some embodiments, the RNA (e.g., mRNA) comprises pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and/or 2′-O-methyl uridine.

RNA (e.g., mRNA) described herein can be generated by e.g., in vitro transcription. In vitro transcription is a method well known to those of ordinary skill in the art for the production of RNA (e.g., mRNA). Generally, the RNA is obtained by DNA-dependent in vitro transcription of an appropriate DNA template, e.g., a linearized plasmid DNA template or a PCR-amplified DNA template. The promoter for controlling RNA in vitro transcription can be any promoter for any DNA-dependent RNA polymerase. Examples of DNA-dependent RNA polymerases include the 17, T3, SP6, or Syn5 RNA polymerases. In some instances, the DNA template is linearized with a suitable restriction enzyme before it is subjected to RNA in vitro transcription. Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); a DNA-dependent RNA polymerase capable of binding to the promoter sequence within the DNA template (e.g., T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCh, which supplies Mg2+ ions as a co-factor for the polymerase; a buffer (TRIS or HEPES) to maintain a suitable pH value, which can also contain antioxidants (e.g., DTT), and/or polyamines such as spermidine at optimal concentrations, e.g., a buffer system comprising TRIS-Citrate as disclosed in WO2017109161. The obtained RNA (e.g., mRNA) products can be purified according to methods known in the art. For example, using PureMessenger® (Cure Vac, Tubingen, Germany; RP-HPLC according to WO2008077592) and/or tangential flow filtration (as described in WO2016193206) and/or oligo d(T) purification (see WO2016180430); or using RP-HPLC, e.g., using Reversed-Phase High pressure liquid chromatography (RP-HPLC), the entire contents of each reference is incorporated by reference herein for all purposes.

### 5.8 Vectors

In one aspect, provided herein are vectors comprising a nucleic acid molecule (e.g., DNA molecule, RNA molecule) described herein (e.g., a nucleic acid molecule described in § 5.7) (e.g., an immunoreceptor inhibitory protein described herein (see, e.g., § 5.2), a fusion protein described herein (see, e.g., § 5.4), a conjugate described herein (see, e.g., § 5.4), an immunogenic peptide or protein described herein (see, e.g., § 5.5), or an antibody described herein (see, e.g., § 5.10)). In some embodiments, the vector is linear. In some embodiments, the vector is a viral vector. In some embodiments, the vector is a non-viral vector (e.g., a plasmid, a minicircle).

### 5.8.1 Non-Viral Vectors

In some embodiments, the vector is a non-viral vector. In some embodiments, the vector is a minicircle. In some embodiments, the vector is a plasmid. A person of ordinary skill in the art is aware of suitable plasmids for expression of the DNA of interest. For example, plasmid DNA may be generated to allow efficient production of the encoded endonucleases in cell lines, e.g., in insect cell lines, for example using vectors as described in WO2009150222A2 and as defined in PCT claims 1 to 33, the disclosure relating to claims 1 to 33 of WO2009150222A2 the entire contents of which is incorporated by reference herein for all purposes.

### 5.8.2 Viral Vectors

In some embodiments, the nucleic acid molecules (e.g., DNA or RNA) encoding an immunogenic peptide or protein described herein are contained in a viral vector. Thus, also provided herein are viral vectors comprising the nucleic acid molecules encoding an immunogenic peptide or protein described herein. Such vectors can be easily manipulated by methods well known to the ordinary person of skill in the art. The vector used can be any vector that is suitable for cloning nucleic acids that can be used for transcription of the nucleic acid molecule of interest.

Viral vectors include both RNA and DNA based vectors. The vectors can be designed to meet a variety of specifications. For example, viral vectors can be engineered to be capable or incapable of replication in prokaryotic and/or eukaryotic cells. In some embodiments, the vector is replication deficient. In some embodiments, the vector is replication competent. Vectors can be engineered or selected that either will (or will not) integrate in whole or in part into the genome of host cells, resulting (or not (e.g., episomal expression)) in stable host cells comprising the desired nucleic acid in their genome.

Exemplary viral vectors include, but are not limited to, adenovirus vectors, adeno-associated virus vectors, lentivirus vectors, retrovirus vectors, poxvirus vectors, parapoxivirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, or lymphocytic choriomeningitis virus vectors. In some embodiments, the viral vector is an adenovirus vector, adeno-associated virus vector, lentivirus vector, anellovector (as described, for example, in U.S. Pat. No. 11,446,344, the entire contents of which is incorporated by reference herein for all purposes).

In some embodiments, the vector is an adenoviral vector (e.g., human adenoviral vector, e.g., HAdV or AdHu). In some embodiments, the adenovirus vector has the E1 region deleted, rendering it replication-deficient in human cells. Other regions of the adenovirus such as E3 and E4 may also be deleted. Exemplary adenovirus vectors include, but are not limited to, those described in e.g., WO2005071093 or WQ2006048215, the entire contents of each of which is incorporated by reference herein for all purposes. In some embodiments, the adenovirus-based vector used is a simian adenovirus, thereby avoiding dampening of the immune response after vaccination by pre-existing antibodies to common human entities such as AdHu5. Exemplary, simian adenovirus vectors include AdCh63 (see, e.g., WO2005071093, the entire contents of which is incorporated by reference herein for all purposes) or AdCh68.

Viral vectors can be generated through the use of a packaging/producer cell line (e.g., a mammalian cell line) using standard methods known to the person of ordinary skill in the art. Generally, a nucleic acid construct (e.g., a plasmid) encoding the transgene (e.g., an immunogenic peptide or protein described herein) (along with additional elements e.g., a promoter, inverted terminal repeats (ITRs) flanking the transgene, a plasmid encoding e.g., viral replication and structural proteins, along with one or more helper plasmids a host cell (e.g., a host cell line) are transfected into a host cell line (i.e., the packing/producer cell line). In some instances, depending on the viral vector, a helper plasmid may also be needed that includes helper genes from another virus (e.g., in the instance of adeno-associated viral vectors). Eukaryotic expression plasmids are commercially available from a variety of suppliers, for example the plasmid series: pcDNA™, pCR3.1™, pCMV™, pFRT™, pVAX1™, pCI™, Nanoplasmid™, and Pcaggs. The person of ordinary skill in the art is aware of numerous transfection methods and any suitable method of transfection may be employed (e.g., using a biochemical substance as carrier (e.g., lipofectamine), by mechanical means, or by electroporation,). The cells are cultured under conditions suitable and for a sufficient time for plasmid expression. The viral particles may be purified from the cell culture medium using standard methods known to the person of ordinary skill in the art. For example, by centrifugation followed by e.g., chromatography or ultrafiltration.

In some embodiments, an immunoreceptor inhibitory protein described herein (see, e.g., § 5.2), a fusion protein described herein (see, e.g., § 5.4), or a conjugate described herein (see, e.g., § 5.4), can be incorporated into a viral particle for e.g., targeting of a viral particle encoding a gene therapy cassette to a specific location within a subject (e.g., a specific cell, tissue, or organ). As such, further provided herein are, inter alia, viral particles displaying on their surface an immunoreceptor inhibitory protein described herein, a fusion protein described herein, or a or conjugate described herein. Suitable methods of incorporating targeting proteins into viral particles are known in the art, including e.g., genetic fusion to viral capsid or envelope proteins, complexing with bispecific adapters, and chemical linkage. See, e.g., Nettelbeck, Dirk M. “Bispecific Antibodies and Gene Therapy.” Bispecific Antibodies 327-347. 1 Jul. 2011, doi: 10.1007/978-3-642-20910-9_18, the entire contents of which are incorporated herein by reference for all purposes.

### 5.9 Cells

In one aspect, provided herein are cells (e.g., host cells) (or populations of cells) (e.g., populations of host cels) comprising any one or more of an immunoreceptor inhibitory protein described herein (see, e.g., § 5.2), a fusion protein described herein (see, e.g., § 5.4), a conjugate described herein (see, e.g., § 5.4), an immunogenic peptide or protein described herein (see, e.g., § 5.5), an antibody described herein (see, e.g., § 5.10), a nucleic acid molecule described herein (see, e.g., § 5.7), a vector described herein (see, e.g., § 5.8), or a carrier described herein (see, e.g., § 5.11).

In some embodiments, the cell (or population of cells) is a eukaryotic cell. In some embodiments, the cell (or population of cells) is mammalian cell. In some embodiments, the cell (or population of cells) is an animal cell. In some embodiments, the cell (or population of cells) is a human cell. In some embodiments, the cell (or population of cells) is in vitro. In some embodiments, the cell (or population of cells) is in vivo. In some embodiments, the cell (or population of cells) is ex vivo.

Standard methods known in the art can be utilized to deliver any one of the foregoing (e.g., immunoreceptor inhibitory protein, fusion protein, vector, nucleic acid molecule, carrier, etc.) into a cell (e.g., a host cell). Standard methods known in the art can be utilized to culture cells (e.g., host cells) in vitro or ex vivo.

In some embodiments, the cell (or population of cells) expresses a protein comprising an immunoreceptor inhibitory protein described herein or a fusion protein described herein. In some embodiments, the cell (or population of cells) has been genetically engineered to comprise (e.g., within the cell's genome) a nucleic acid molecule (e.g., described herein) that encodes an immunoreceptor inhibitory protein described herein or a fusion protein described herein. In some embodiments, the cell (or population of cells) expresses a protein comprising an immunoreceptor inhibitory protein described herein or a fusion protein described herein on the surface of the cell.

In some embodiments, the cell (or population of cells) is a therapeutic cell. In some embodiments, the therapeutic cell (or population of cells) has been genetically engineered to comprise (e.g., within the cell's genome) a nucleic acid molecule (e.g., described herein) that encodes an immunoreceptor inhibitory protein described herein or a fusion protein described herein. In some embodiments, the therapeutic cell (or population of cells) expresses a protein comprising an immunoreceptor inhibitory protein described herein or a fusion protein described herein on the surface of the cell. In some embodiments, an immunoreceptor inhibitory protein described herein or a fusion protein described herein on the surface of the cell acts as a targeting moiety. In some embodiments, the therapeutic cell is an immune cell. In some embodiments, the therapeutic cell is a T cell (e.g., a CD8+ T cell, a CD4+ T cell). In some embodiments, the therapeutic cell is a natural killer cell.

In some embodiments, the cell expresses and/or encodes a chimeric antigen receptor comprising an immunoreceptor inhibitory protein (e.g., described herein) (also referred to herein as a CAR cell). As such, in one aspect, provided herein are cells expressing and/or genetically encoding a chimeric antigen receptor comprising an immunoreceptor inhibitory protein (e.g., utilized as the antigen binding domain of the extracellular domain of the chimeric antigen receptor). Such chimeric antigen receptors are described herein (see, e.g., § 5.4.2). In some embodiments, the cell is a T cell (e.g., a CD8+ T cell, a CD4+ T cell). In some embodiments, the cell is a natural killer cell.

### 5.10 Antibodies

In one aspect, provided herein are antibodies (and functional fragments and variants thereof) that specifically bind an immunoreceptor inhibitory protein described herein. In some embodiments, the antibody inhibits reduces (e.g., prevents) binding of the immunoreceptor inhibitory protein to one or more TNF superfamily member (e.g., described herein, e.g., TL1A). The antibody can be for example, a full-length antibody, a Fab, a scFv, or a single domain antibody. In some embodiments, the antibody (e.g., an antibody that specifically binds an immunoreceptor inhibitory protein described herein) is labeled with a tag (e.g., a fluorescent tag) to aid in detection. In some embodiments, the antibody is utilized in a therapeutic method, e.g., a method of preventing or treating a viral infection (see, e.g., § 5.14.11). In some embodiments, the antibody is utilized in a diagnostic method, e.g., a method of diagnosing a subject with a viral infection (see, e.g., § 5.14.12). In some embodiments, the antibody is contained in a kit described herein (see, e.g., § 5.15).

### 5.11 Carriers

The immunoreceptor inhibitory protein described herein (see, e.g., § 5.2), a fusion protein described herein (see, e.g., § 5.4), a conjugate described herein (see, e.g., § 5.4), an immunogenic peptide or protein described herein (see, e.g., § 5.5), an antibody described herein (see, e.g., § 5.10), a nucleic acid molecule described herein (see, e.g., § 5.7), or a vector described herein (see, e.g., § 5.8), can be formulated within a carrier (see, e.g., § 5.11.1) and/or conjugated to a carrier (e.g., as a targeting moiety) (see, e.g., § 5.11.2).

Exemplary carriers include, but are not limited to, lipid-based carriers (e.g., lipid nanoparticles (LNPs), liposomes, lipoplexes, and nanoliposomes). In some embodiments, the carrier is a lipid-based carrier. In some embodiments, the carrier is an LNP. In some embodiments, the LNP comprises a cationic lipid, a neutral lipid, a cholesterol, and/or a PEG lipid. Lipid based carriers are further described below in § 5.11.3.

### 5.11.1 Carriers of IIPs

In some embodiments, an immunoreceptor inhibitory protein described herein (see, e.g., § 5.2), a fusion protein described herein (see, e.g., § 5.4), a conjugate described herein (see, e.g., § 5.4), an immunogenic peptide or protein described herein (see, e.g., § 5.5), an antibody described herein (see, e.g., § 5.10), a nucleic acid molecule described herein (see, e.g., § 5.7), or a vector described herein (see, e.g., § 5.8), is formulated within a carrier.

As, such, the disclosure provides, inter alia, carriers comprising any one of more of an immunoreceptor inhibitory protein described herein, a fusion protein described herein, a conjugate described herein, an immunogenic peptide or protein described herein, an antibody described herein, a nucleic acid molecule described herein (e.g., a nucleic acid molecule encoding an immunoreceptor inhibitory protein described herein, a fusion protein described herein, an immunogenic peptide or protein described herein, an antibody described herein, etc.), or a vector described herein (e.g., a vector comprising a nucleic acid molecule described herein).

Any of the foregoing agents described herein (e.g., proteins, nucleic acid molecules, vectors, etc.) described herein can be encapsulated within a carrier, chemically conjugated to a carrier, associated with the carrier. In this context, the term “associated” refers to the essentially stable combination of an agent described herein (e.g., a protein, nucleic acid molecule, vector, etc.) with one or more molecules of a carrier (e.g., one or more lipids of a lipid-based carrier, e.g., an LNP, liposome, lipoplex, and/or nanoliposome) into larger complexes or assemblies without covalent binding. In this context, the term “encapsulation” refers to the incorporation of an agent described herein (e.g., a protein, nucleic acid molecule, vector, etc.,) is entirely contained within the interior space of the carrier (e.g., the lipid-based carrier, e.g., the LNP, liposome, lipoplex, and/or nanoliposome).

In some embodiments, the carrier is a viral particle. As such, further provided herein are viral particles comprising any one of more of an immunoreceptor inhibitory protein described herein, a fusion protein described herein, a conjugate described herein, an immunogenic peptide or protein described herein, an antibody described herein, a nucleic acid molecule described herein (e.g., a nucleic acid molecule encoding an immunoreceptor inhibitory protein described herein, a fusion protein described herein, an immunogenic peptide or protein described herein, an antibody described herein, etc.), or a vector described herein (e.g., a vector comprising a nucleic acid molecule described herein).

Exemplary carriers are further described herein (see, e.g., § 5.11.3).

### 5.11.2 Carriers Conjugated to IIPs

In some embodiments, an immunoreceptor inhibitory protein described herein (see, e.g., § 5.2), a fusion protein described herein (see, e.g., § 5.4), a conjugate described herein (see, e.g., § 5.4), an immunogenic peptide or protein described herein (see, e.g., § 5.5), an antibody described herein (see, e.g., § 5.10), a nucleic acid molecule described herein (see, e.g., § 5.7), or a vector described herein (see, e.g., § 5.8), is conjugated to a carrier described herein (e.g., to target the carrier (e.g., within a human subject)).

As, such, the disclosure provides, inter alia, carriers conjugated to any one of more of an immunoreceptor inhibitory protein described herein, a fusion protein described herein, a conjugate described herein, an immunogenic peptide or protein described herein, an antibody described herein, a nucleic acid molecule described herein (e.g., a nucleic acid molecule encoding an immunoreceptor inhibitory protein described herein, a fusion protein described herein, an immunogenic peptide or protein described herein, an antibody described herein, etc.), or a vector described herein (e.g., a vector comprising a nucleic acid molecule described herein). In one aspect, provided herein are carriers conjugated to an immunoreceptor inhibitory protein described herein. As described above, the conjugation of an immunoreceptor inhibitory protein described herein to a carrier can be utilized to generate a targeted carrier (e.g., for targeting the carrier and the associated/encapsulated payload to a specific part of the body, e.g., specific tissue, cell, organ, etc.).

In some embodiments, the carrier is a viral particle. As such, further provided herein are viral particles conjugated to any one of more of an immunoreceptor inhibitory protein described herein, a fusion protein described herein, a conjugate described herein, an immunogenic peptide or protein described herein, an antibody described herein, a nucleic acid molecule described herein (e.g., a nucleic acid molecule encoding an immunoreceptor inhibitory protein described herein, a fusion protein described herein, an immunogenic peptide or protein described herein, an antibody described herein, etc.), or a vector described herein (e.g., a vector comprising a nucleic acid molecule described herein).

Exemplary carriers are further described herein (see, e.g., § 5.11.3).

### 5.11.3 Lipid Based Carriers/Lipid Nanoformulations

In some embodiments, an agent described herein (e.g., a protein, nucleic acid molecule, vector, etc.) is encapsulated or associated with one or more lipids (e.g., cationic lipids and/or neutral lipids), thereby forming lipid-based carriers such as lipid nanoparticles (LNPs), liposomes, lipoplexes, or nanoliposomes.

In some embodiments, an agent described herein (e.g., a protein, nucleic acid molecule, vector, etc.) is encapsulated in one or more lipids (e.g., cationic lipids and/or neutral lipids), thereby forming lipid-based carriers such as lipid nanoparticles (LNPs), liposomes, lipoplexes, or nanoliposomes. In some embodiments, an agent described herein (e.g., a protein, nucleic acid molecule, vector, etc.) is associated with one or more lipids (e.g., cationic lipids and/or neutral lipids), thereby forming lipid-based carriers such as lipid nanoparticles (LNPs), liposomes, lipoplexes, or nanoliposomes. In some embodiments, an agent described herein (e.g., a protein, nucleic acid molecule, vector, etc.) is encapsulated in LNPs (e.g., as described herein).

The agent (e.g., the protein, nucleic acid molecule, vector, etc.) may be completely or partially located in the interior space of the LNPs, liposomes, lipoplexes, and/or nanoliposomes, within the lipid layer/membrane, or associated with the exterior surface of the lipid layer/membrane. One purpose of incorporating an agent (e.g., a protein, nucleic acid molecule, vector, etc.) into LNPs, liposomes, lipoplexes, and/or nanoliposomes is to protect the agent from an environment which may contain enzymes or chemicals or conditions that degrade the agent from molecules or conditions that cause the rapid excretion of the agent. Moreover, incorporating an agent (e.g., a protein, nucleic acid molecule, vector, etc.) into LNPs, liposomes, lipoplexes, and/or nanoliposomes may promote the uptake of the agent, and hence, may enhance the therapeutic effect of the agent. Accordingly, incorporating an agent described herein (e.g., a protein, nucleic acid molecule, vector, etc.) into LNPs, liposomes, lipoplexes, and/or nanoliposomes may be particularly suitable for a pharmaceutical composition described herein, e.g., for intramuscular and/or intradermal administration.

In some embodiments, an agent described herein (e.g., a protein, nucleic acid molecule, vector, etc.) is formulated into a lipid-based carrier (or lipid nanoformulation). In some embodiments, the lipid-based carrier (or lipid nanoformulation) is a liposome or a lipid nanoparticle (LNP). In one embodiment, the lipid-based carrier is an LNP.

In some embodiments, an agent described herein (e.g., a protein, nucleic acid molecule, vector, etc.) is conjugated to a lipid-based carrier (e.g., described herein) (e.g., an LNP) (e.g., forming targeted lipid-based carriers (targeted LNPs)).

In some embodiments, the lipid-based carrier (or lipid nanoformulation) comprises a cationic lipid (e.g., an ionizable lipid), a non-cationic lipid (e.g., phospholipid), a structural lipid (e.g., cholesterol), and a PEG-modified lipid. In some embodiments, the lipid-based carrier (or lipid nanoformulation) contains one or more an agent described herein (e.g., a protein, nucleic acid molecule, vector, etc.), or a pharmaceutically acceptable salt thereof.

As described herein, suitable compounds to be used in the lipid-based carrier (or lipid nanoformulation) include all the isomers and isotopes of the compounds described above, as well as all the pharmaceutically acceptable salts, solvates, or hydrates thereof, and all crystal forms, crystal form mixtures, and anhydrides or hydrates.

In addition to the one or more agent described herein (e.g., a protein, nucleic acid molecule, vector, etc.), the lipid-based carrier (or lipid nanoformulation) may further include a second lipid. In some embodiments, the second lipid is a cationic lipid, a non-cationic (e.g., neutral, anionic, or zwitterionic) lipid, or an ionizable lipid.

One or more naturally occurring and/or synthetic lipid compounds may be used in the preparation of the lipid-based carrier (or lipid nanoformulation).

The lipid-based carrier (or lipid nanoformulation) may contain positively charged (cationic) lipids, neutral lipids, negatively charged (anionic) lipids, or a combination thereof.

### 5.11.3.1 Cationic Lipids (Positively Charged) and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 3β-[N-(N′,N′-dimethylaminocthyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleyloxypropyl-3-dimethyl-hydroxy ethyl ammonium chloride (DORI), N,N-dioleyl-N,N-dimethylammonium chloride (DODAC), N,N-dimethyl-2,3-dioleyloxy)propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLin DMA), N,N-Dimethyl-3,4-dioleyloxybenzylamine (DMOBA), and the cationic lipids described in e.g. Martin et al., Current Pharmaceutical Design, pages 1-394, the entire contents of which are incorporated by reference herein for all purposes. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, the entire contents of which are incorporated by reference herein for all purposes.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

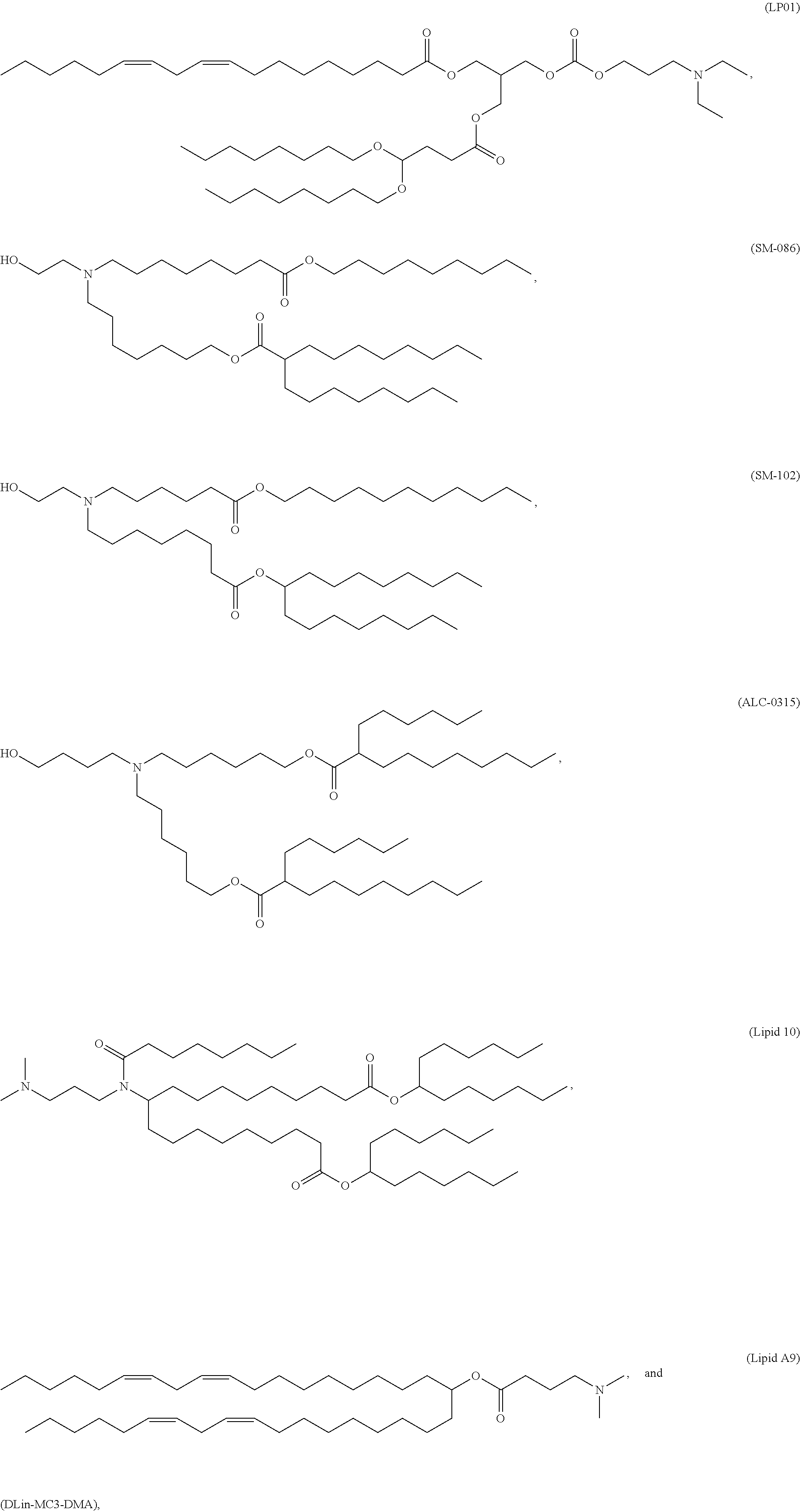

(see WO2017004143A1, the entire contents of which is incorporated herein by reference for all purposes).

In some embodiments, the lipid-based carrier (or lipid nanoformulation) further comprises one or more compounds described by WO 2021/113777 (e.g., a lipid of Formula (3) such as a lipid of Table 3 of WO 2021/113777), the entire contents of which are incorporated by reference herein for all purposes.

In one embodiment, the ionizable lipid is a lipid disclosed in Hou, X., et al. Nat Rev Mater 6, 1078-1094 (2021). https://doi.org/10.1038/s41578-021-00358-0 (e.g., L319, C12-200, and DLin-MC3-DMA), (the entire contents of which are incorporated by reference herein for all purposes).

Examples of other ionizable lipids that can be used in lipid-based carrier (or lipid nanoformulation) include, without limitation, one or more of the following formulas: X of US 2016/0311759; I of US20150376115 or in US 2016/0376224; Compound 5 or Compound 6 in US 2016/0376224; I, IA, or II of U.S. Pat. No. 9,867,888; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y-Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; III-3 of WO 2018/081480; I-5 or I-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-013 or 503-013 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, the entire contents of each of which are incorporated by reference herein for all purposes.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, the entire contents of each of which are incorporated by reference herein for all purposes.

### 5.11.3.2 Non-Cationic Lipids (e.g., Phospholipids)

In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, the entire contents of which are incorporated by reference herein for all purposes. Such lipids include, in some embodiments, plant lipids found to improve liver transfection with mRNA (e.g., DGTS).

In some embodiments, the lipid-based carrier (or lipid nanoformulation) may comprise a combination of distearoylphosphatidylcholine/cholesterol, dipalmitoylphosphatidylcholine/cholesterol, dimyrystoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US2018/0028664, the entire contents of each of which are incorporated by reference herein for all purposes.

In one embodiment, the lipid-based carrier (or lipid nanoformulation) further comprises one or more non-cationic lipid that is oleic acid or a compound of Formula I, II, or IV of US2018/0028664, the entire contents of which are incorporated by reference herein for all purposes.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

### 5.11.3.3 Structural Lipids

The lipid-based carrier (or lipid nanoformulation) described herein may further comprise one or more structural lipids. As used herein, the term “structural lipid” refers to sterols (e.g., cholesterol) and also to lipids containing sterol moieties.

Incorporation of structural lipids in the lipid nanoparticle may help mitigate aggregation of other lipid in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO2009/127060 or US2010/0130588, the entire contents of each of which are incorporated by reference herein for all purposes. Additional exemplary sterols include phytosterols, including those described in Eygeris et al. (2020), Nano Lett. 2020; 20 (6): 4543-4549, the entire contents of which are incorporated by reference herein for all purposes.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in WO 2009/127060 and US 2010/0130588, the entire contents of each of which are incorporated by reference herein for all purposes.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

### 5.11.3.4 Polymers and Polyethylene Glycol (PEG)—Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid. Examples of useful PEG-lipids include, but are not limited to, 1,2-Diacyl-sn-Glycero-3-Phosphoethanolamine-N-[Methoxy(Polyethylene glycol)-350] (mPEG 350 PE); 1,2-Diacyl-sn-Glycero-3-Phosphoethanolamine-N-[Methoxy(Polyethylene glycol)-550] (mPEG 550 PE); 1,2-Diacyl-sn-Glycero-3-Phosphoethanolamine-N-[Methoxy(Polyethylene glycol)-750] (mPEG 750 PE); 1,2-Diacyl-sn-Glycero-3-Phosphoethanolamine-N-[Methoxy(Polyethylene glycol)-1000] (mPEG 1000 PE); 1,2-Diacyl-sn-Glycero-3-Phosphoethanolamine-N-[Methoxy(Polyethylene glycol)-2000] (mPEG 2000 PE); 1,2-Diacyl-sn-Glycero-3-Phosphoethanolamine-N-[Methoxy(Polyethylene glycol)-3000] (mPEG 3000 PE); 1,2-Diacyl-sn-Glycero-3-Phosphoethanolamine-N-[Methoxy(Polyethylene glycol)-5000] (mPEG 5000 PE); N-Acyl-Sphingosine-1-[Succinyl(Methoxy Polyethylene Glycol) 750] (mPEG 750 Ceramide); N-Acyl-Sphingosine-1-[Succinyl(Methoxy Polyethylene Glycol) 2000] (mPEG 2000 Ceramide); and N-Acyl-Sphingosine-1-[Succinyl(Methoxy Polyethylene Glycol) 5000] (mPEG 5000 Ceramide). In some embodiments, the PEG lipid is a polyethyleneglycol-diacylglycerol (i.e., polyethyleneglycol diacylglycerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 12 of WO2019/217941, the entire contents of which are incorporated by reference herein for all purposes). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristoylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (the entire contents of which are incorporated by reference herein for all purposes), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, the entire contents of each of which are incorporated by reference herein for all purposes.

In some embodiments, the PEG-lipid is a compound of Formula III, III-a-I, III-a-2, III-b-1, III-b-2, or V of US 2018/0028664, which is incorporated herein by reference in its entirety. In some embodiments, the PEG-lipid is of Formula II of US 2015/0376115 or US 2016/0376224, the entire contents of each of which are incorporated by reference herein for all purposes. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

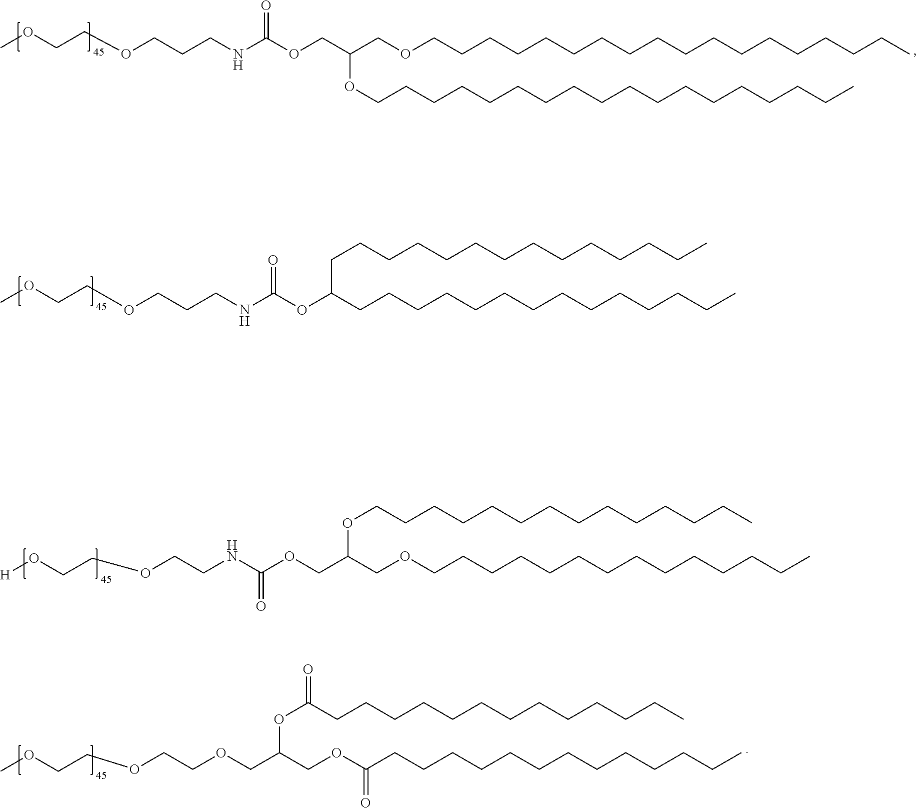

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, the entire contents of which are incorporated by reference herein for all purposes.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289:1128-1133 (1999); Gabizon et al., J Controlled Release 53:275-279 (1998); Adlakha Hutcheon et al., Nat Biotechnol 17:775-779 (1999); and Koning et al., Biochim Biophys Acta 1420:153-167 (1999), the entire contents of each of which are incorporated by reference herein for all purposes.

### 5.11.3.5 Percentages of Lipid Nanoformulation Components

In some embodiments, the lipid-based carrier (or lipid nanoformulation) comprises a non-cationic lipid (e.g., a phospholipid), a sterol, a neutral lipid, and optionally conjugated lipid (e.g., a PEGylated lipid) that inhibits aggregation of particles. The amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid components, a conjugated lipid (e.g., a PEGylated lipid) in an amount from about 0.5 mol % to about 20 mol % (e.g., 1-10 mol % or 5-10%) of the total lipid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid components, provided that the total mol % of the lipid component does not exceed 100%.

In some embodiments, the lipid-based carrier (or lipid nanoformulation) comprises about 25-100 mol % of the ionizable lipid including the lipid compounds described herein, about 0-50 mol % phospholipid, about 0-50 mol % sterol, and about 0-10 mol % PEGylated lipid.

In some embodiments, the lipid-based carrier comprises a lipid nanoparticle, wherein the lipid nanoparticle comprises about 25-100 mol % of the ionizable lipid including the lipid compounds described herein, about 0-50 mol % phospholipid, about 0-50 mol % sterol, and about 0-10 mol % PEGylated lipid. In some embodiments, the encapsulation efficiency of the agent may be at least 70%.

In one embodiment, the lipid-based carrier (or lipid nanoformulation) comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier comprises a lipid nanoparticle, wherein the lipid nanoparticle comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid. In some embodiments, the encapsulation efficiency of an agent described herein may be at least 70%.

In some embodiments, the lipid-based carrier (or lipid nanoformulation) comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, the lipid-based carrier comprises a lipid nanoparticle, wherein the lipid nanoparticle comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid. In some embodiments, the encapsulation efficiency of an agent described herein may be at least 70%.

In some embodiments, molar ratios of ionizable lipid/sterol/phospholipid (or another structural lipid)/PEG-lipid/additional components is varied in the following ranges: ionizable lipid (25-100%); phospholipid (DSPC) (0-40%); sterol (0-50%); and PEG lipid (0-5%).

In some embodiments, the lipid-based carrier comprises a lipid nanoparticle, wherein the lipid nanoparticle comprises molar ratios of ionizable lipid/sterol/phospholipid (or another structural lipid)/PEG-lipid/additional components in the following ranges: ionizable lipid (25-100%); phospholipid (DSPC) (0-40%); sterol (0-50%); and PEG lipid (0-5%). In some embodiments, the encapsulation efficiency of an agent described herein may be at least 70%.

In some embodiments, the lipid-based carrier (or lipid nanoformulation) comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid).

In some embodiments, the lipid-based carrier comprises a lipid nanoparticle, wherein the lipid nanoparticle comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid). In some embodiments, the encapsulation efficiency of an agent described herein may be at least 70%.

In some embodiments, the lipid-based carrier (or lipid nanoformulation) comprises (i) a cationic lipid comprising from 50 mol % to 65 mol % of the total lipid present in the lipid-based carrier; (ii) a non-cationic lipid comprising a mixture of a phospholipid and a cholesterol derivative thereof, wherein the phospholipid comprises from 3 mol % to 15 mol % of the total lipid present in the lipid-based carrier and the cholesterol or derivative thereof comprises from 30 mol % to 40 mol % of the total lipid present in the lipid-based carrier; and (iii) a conjugated lipid comprising 0.5 mol % to 2 mol % of the total lipid present in the particle.

In some embodiments, the lipid-based carrier (or lipid nanoformulation) comprises (i) an agent described herein (e.g., a protein, nucleic acid molecule, vector, etc.); (ii) a cationic lipid comprising from 50 mol % to 65 mol % of the total lipid present in the lipid-based carrier; (iii) a non-cationic lipid comprising a mixture of a phospholipid and a cholesterol derivative thereof, wherein the phospholipid comprises from 3 mol % to 15 mol % of the total lipid present in the lipid-based carrier and the cholesterol or derivative thereof comprises from 30 mol % to 40 mol % of the total lipid present in the lipid-based carrier; and (iv) a conjugated lipid comprising 0.5 mol % to 2 mol % of the total lipid present in the particle.

In some embodiments, the lipid-based carrier (or lipid nanoformulation) comprises (i) a cationic lipid comprising from 50 mol % to 85 mol % of the total lipid present in the lipid-based carrier; (ii) a non-cationic lipid comprising from 13 mol % to 49.5 mol % of the total lipid present in the lipid-based carrier; and (iii) a conjugated lipid comprising from 0.5 mol % to 2 mol % of the total lipid present in the lipid-based carrier.

In some embodiments, the lipid-based carrier (or lipid nanoformulation) comprises (i) an agent described herein (e.g., a protein, nucleic acid molecule, vector, etc.); (ii) a cationic lipid comprising from 50 mol % to 85 mol % of the total lipid present in the lipid-based carrier; (iii) a non-cationic lipid comprising from 13 mol % to 49.5 mol % of the total lipid present in the lipid-based carrier; and (iv) a conjugated lipid comprising from 0.5 mol % to 2 mol % of the total lipid present in the lipid-based carrier.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, (or any fraction of these ranges) of the total lipid components. In some embodiments, the lipid-based carrier (or lipid nanoformulation) is phospholipid-free.

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from 30 mol % to 35 mol %, from 35 mol % to 40 mol %, from 27 mol % to 37 mol %, or from 27 mol % to 35 mol % (or any fraction of these ranges) of the total lipid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid components.

The ratio of total lipid components to the agent can be varied as desired. For example, the total lipid components to the agent (mass or weight) ratio can be from about 10:1 to about 30:1. In some embodiments, the total lipid components to the agent ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the agent can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation's) overall lipid content can range from about 5 mg/ml to about 30 mg/mL. Nitrogen:phosphate ratios (N:P ratio) is evaluated at values between 0.1 and 100.

The efficiency of encapsulation of an agent described herein (e.g., a protein, nucleic acid molecule, vector, etc.), describes the amount of the agent that is encapsulated or otherwise associated with a lipid nanoformulation (e.g., liposome or LNP) after preparation, relative to the initial amount provided. The encapsulation efficiency is desirably high (e.g., at least 70%. 80%. 90%. 95%, close to 100%). The encapsulation efficiency may be measured, for example, by comparing the amount of the agent in a solution containing the liposome or LNP before and after breaking up the liposome or LNP with one or more organic solvents or detergents. An anion exchange resin may be used to measure the amount of free the agent in a solution. Fluorescence may be used to measure the amount of free the agent in a solution. For the lipid-based carrier (or lipid nanoformulation) described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 70%. In some embodiments, the encapsulation efficiency may be at least 80%. In some embodiments, the encapsulation efficiency may be at least 90%. In some embodiments, the encapsulation efficiency may be at least 95%.

### 5.12 Adjuvants

The pharmaceutical compositions described herein (e.g., including vaccine compositions) (e.g., pharmaceutical compositions and vaccine compositions comprising an immunogenic peptide or protein (e.g., described herein) or a nucleic acid molecule (e.g., DNA, RNA (e.g., mRNA)) encoding the immunogenic peptide or protein (e.g., described herein)) may comprise one or more adjuvants or be co-administered with one or more adjuvants. Adjuvants are known in the art to further increase the immune response to an immunogen. General categories of adjuvants include, but are not limited to, inorganic adjuvants, small molecule adjuvants, oil in water emulsions, lipids or polymers, peptides or peptidoglycans, carbohydrates or polysaccharides, RNA-based adjuvants, DNA-based adjuvants, viral particles, bacterial adjuvants, inorganic nanoparticles, and multi-component adjuvants. Examples of adjuvants include, but are not limited to, aluminum salts such as aluminum hydroxide and/or aluminum phosphate; oil-emulsion compositions (or oil-in-water compositions), including squalene-water emulsions, such as MF59 (see, e.g., WO90/14837), MF59, AS03, and Montanide; saponin formulations, such as for example QS21 and Immunostimulating Complexes (ISCOMS) (see, e.g., U.S. Pat. No. 5,057,540; WO90/03184, WO96/11711, WO2004/004762, WO2005/002620, the entire contents of each of which is incorporated by reference herein for all purposes); protamine or a protamine salt (e.g., protamine sulfate); calcium salt; bacterial or microbial derivatives, examples of which include monophosphoryl lipid A (MPL), 3-O-deacylated MPL (3dMPL), CpG-motif containing nucleic acid molecules, ADP-ribosylating bacterial toxins or mutants thereof, such as E. coli heat labile enterotoxin LT, cholera toxin CT, and the like; eukaryotic proteins (e.g., antibodies or fragments thereof (e.g., directed against the immunogen itself or CD1a, CD3, CD7, CD80) and ligands to receptors (e.g., CD40L, GMCSF, GCSF, etc.).

Exemplary RNA-based adjuvants include, but are not limited to, Poly IC, Poly IC:LC, hairpin RNAs, e.g., with a 5′PPP containing sequence, viral sequences, polyU containing sequences, dsRNA, natural or synthetic immunostimulatory RNA sequences, nucleic acids analogs, optionally cyclic GMP-AMP or a cyclic dinucleotide such as cyclic di-GMP, and immunostimulatory base analogs, e.g., C8-substituted or an N7,C8-disubstituted guanine ribonucleotide. Exemplary DNA-based adjuvants include, but are not limited to, CpGs, dsDNA, or natural or synthetic immunostimulatory DNA sequences. Exemplary bacteria-based adjuvants include, but are not limited to, bacterial adjuvant is flagellin, LPS, or a bacterial toxin, e.g., enterotoxins, heat-labile toxins, and Cholera toxins. Exemplary carbohydrate or polysaccharide adjuvants include, but are not limited to, dextran (branched microbial polysaccharide), dextran-sulfate, Lentinan, zymosan, Betaglucan, Deltin, Mannan, and Chitin. Exemplary small molecule adjuvants, include, but are not limited to, imiquimod, resiquimod, and gardiquimod. Exemplary lipid or polymer adjuvants, include, but are not limited to, polymeric nanoparticles (e.g., PLGA, PLG, PLA, PGA, or PHB), liposomes (e.g., Virosomes and CAF01), LNPs or a component thereof, lipopolysaccharide (LPS) (e.g., monophosphoryl lipid A (MPLA) or glucopyranosyl Lipid A (GLA)), lipopeptides (e.g., Pam2 (Pam2CSK4) or Pam3 (Pam3CSK4)), and glycolipid (e.g., trehalose dimycolate). Exemplary peptides or peptidoglycan include, but are not limited to, N-acetyl-muramyl-L-alanyl-D-isoglutamine (MDP), flagellin-fusion protein, mannose-binding lectin (MBL), cytokines, and chemokine. Exemplary inorganic nanoparticle adjuvants, include, but are not limited to, gold nanorods, silica-based nanoparticles (e.g., mesoporous silica nanoparticles (MSN)). Exemplary multicomponent adjuvants include, but are not limited to, AS01, AS03, AS04, Complete Freunds Adjuvant, and CAF01.

### 5.13 Pharmaceutical Compositions

In one aspect, provided herein are pharmaceutical compositions comprising any one or more of an immunoreceptor inhibitory protein described herein (see, e.g., § 5.2), a fusion protein described herein (see, e.g., § 5.4), a conjugate described herein (see, e.g., § 5.4), an immunogenic peptide or protein described herein (see, e.g., § 5.5), an antibody described herein (see, e.g., § 5.10), a nucleic acid molecule described herein (see, e.g., § 5.7), a vector described herein (see, e.g., § 5.8), a cell described herein (see, e.g., § 5.9), or a carrier described herein (see, e.g., § 5.11), and a pharmaceutically acceptable excipient (see, e.g., Remington's Pharmaceutical Sciences (1990) Mack Publishing Co., Easton, PA, the entire contents of which is incorporated by reference herein for all purposes).

Also provided herein are pharmaceutical compositions comprising an immunoreceptor inhibitory protein described herein, a fusion protein described herein, a conjugate described herein, an immunogenic peptide or protein described herein, an antibody described herein, a nucleic acid molecule described herein, a vector described herein, a host cell described herein, or a carrier described herein, wherein the pharmaceutical composition lacks a predetermined threshold amount or a detectable amount of a process impurity or contaminant, e.g., lacks a predetermined threshold amount or a detectable amount of a process-related impurity such as host cell proteins, host cell DNA, or a cell culture component (e.g., inducers, antibiotics, or media components); a product-related impurity (e.g., precursors, fragments, aggregates, degradation products); or a contaminant, e.g., endotoxin, bacteria, viral contaminant.

In one aspect, also provided herein are methods of making pharmaceutical compositions described herein comprising providing an immunoreceptor inhibitory protein described herein, a fusion protein described herein, a conjugate described herein, an immunogenic peptide or protein described herein, an antibody described herein, a nucleic acid molecule described herein, a vector described herein, a host cell described herein, or a carrier described herein, and formulating it into a pharmaceutically acceptable composition by the addition of one or more pharmaceutically acceptable excipient.

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

A pharmaceutical composition may be formulated for any route of administration to a subject. The skilled person knows the various possibilities to administer a pharmaceutical composition described herein a in order to induce an immune response to the immunogen(s) in the pharmaceutical composition. Non-limiting embodiments include parenteral administration, such as intramuscular, intradermal, subcutaneous, transcutaneous, or mucosal administration, e.g., inhalation, intranasal, oral, and the like. In one embodiment, the pharmaceutical composition is formulated for administration by intramuscular, intradermal, or subcutaneous injection. In one embodiment, the pharmaceutical composition is formulated for administration by intramuscular injection. In one embodiment, the pharmaceutical composition is formulated for administration by intradermal injection. In one embodiment, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; or sodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 5.14 Methods of Use

Provided herein are various methods of utilizing any one or more agent described herein, including e.g., an immunoreceptor inhibitory protein described herein (see, e.g., § 5.2), a fusion protein described herein (see, e.g., § 5.4), a conjugate described herein (see, e.g., § 5.4), an immunogenic peptide or protein described herein (see, e.g., § 5.5), an antibody described herein (see, e.g., § 5.10), a nucleic acid molecule described herein (see, e.g., § 5.7), a vector described herein (see, e.g., § 5.8), a cell described herein (see, e.g., § 5.9), a carrier described herein (see, e.g., § 5.11), a vaccine composition described herein (see, e.g., § 5.5), or a pharmaceutical composition described herein (see, e.g., § 5.13). Exemplary subjects include mammals, e.g., humans, non-human mammals, e.g., non-human primates. In some embodiments, the subject is a human.

The dosage of an agent described herein (e.g., an immunoreceptor inhibitory protein described herein, a fusion protein described herein, a conjugate described herein, an immunogenic peptide or protein described herein, an antibody described herein, a nucleic acid molecule described herein, a vector described herein, a host cell described herein, a carrier described herein, a vaccine composition, or a pharmaceutical composition described herein) to be administered to a subject in accordance with any of the methods described herein can be determined in accordance with standard techniques known to those of ordinary skill in the art, including the route of administration, the age and weight of the subject, and the type (if any) adjuvant is used.

Various methods described herein comprise the administration of an immunoreceptor inhibitory protein described herein (or a fusion protein or conjugate thereof), a nucleic acid molecule encoding the same (or a vector comprising the same) (or a host cell, carrier, or a pharmaceutical composition comprising any of the foregoing) to a subject. In some embodiments, the immunoreceptor inhibitory protein exhibits tolerable immunogenicity after administration to the subject. In some embodiments, the immunoreceptor inhibitory protein exhibits reduced immunogenicity after administration to the subject relative to a protein that specifically binds the same target but is larger in size (e.g., at least 2×, 3×, 4×, or 5× larger in size). In some embodiments, the immunoreceptor inhibitory protein does not elicit an intolerable anti-immunoreceptor inhibitory protein response after administration to the subject. In some embodiments, the immunoreceptor inhibitory protein elicits a reduced anti-immunoreceptor inhibitory protein immune response relative to a protein that specifically binds the same target but is larger in size (e.g., at least 2×, 3×, 4×, or 5× larger in size).

In some embodiments, an agent described herein (e.g., an immunoreceptor inhibitory protein described herein, a fusion protein described herein, a conjugate described herein, an immunogenic peptide or protein described herein, an antibody described herein, a nucleic acid molecule described herein, a vector described herein, a host cell described herein, a carrier described herein, a vaccine composition, or a pharmaceutical composition described herein) is administered in combination with another agent (e.g., therapeutic agent). In some embodiments, the other agent (e.g., therapeutic agent) comprises an antibody. In specific embodiments, the antibody specifically binds a cytokine (e.g., an interleukin). In specific embodiments, the antibody specifically binds an interleukin (e.g., a human interleukin). In specific embodiments, the antibody specifically binds interleukin 23 (IL-23). In specific embodiments, the antibody specifically binds human IL-23.

### 5.14.1 Methods of Delivery

Provided herein are methods of delivering (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.6); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) to a subject, the method comprising administering the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition to the subject, to thereby deliver the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition to the subject.

In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition is administered to the subject in an amount and for a time sufficient to deliver the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition to the subject.

### 5.14.2 Methods of Inhibiting or Reducing Binding of a TNFSF Ligand to One or More Cognate TNFSF Receptor

Provided herein are methods of inhibiting or reducing (e.g., preventing) binding of one or more TNFSF ligand to one or more of the TNFSF ligand's cognate TNFSF receptors in a subject in need thereof, the method comprising administering to the subject (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.6); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13); to thereby inhibit or reduce (e.g., prevent) binding of one or more TNFSF ligand to one or more of the TNFSF ligand's cognate TNFSF receptors in the subject. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition is administered to the subject the in an amount and for a time sufficient to inhibit or reduce (e.g., prevent) binding of one or more TNFSF ligand to one or more of the TNFSF ligand's cognate TNFSF receptors in the subject.

In some embodiments, the method comprises inhibiting or reducing (e.g., preventing) binding of any one or more (e.g., 1, 2, 3, 4, 5 and/or 6) of the following: (a) binding of TLA1 to DR3, (b) binding of TNFα to TNFR2, (c) binding of TNFα to TNFR1, (d) binding of LIGHT to HVEM, (e) binding of LIGHT to LTβR, and/or (f) binding of FASL to FAS. In some embodiments, the method comprises inhibiting or reducing (e.g., preventing) binding of any 1, 2, 3, 4, 5, and/or 6 of the following: (a) binding of TLA1 to DR3, (b) binding of TNFα to TNFR2, (c) binding of TNFα to TNFR1, (d) binding of LIGHT to HVEM, (e) binding of LIGHT to LTβR, and/or (f) binding of FASL to FAS. In some embodiments, the method comprises inhibiting or reducing (e.g., preventing) binding of a plurality of the following: (a) binding of TLA1 to DR3, (b) binding of TNFα to TNFR2, (c) binding of TNFα to TNFR1, (d) binding of LIGHT to HVEM, (e) binding of LIGHT to LTβR, and/or (f) binding of FASL to FAS. In some embodiments, the method comprises inhibiting or reducing (e.g., preventing) binding of the following: (a) binding of TLA1 to DR3, (b) binding of TNFα to TNFR2, (c) binding of TNFα to TNFR1, (d) binding of LIGHT to HVEM, (e) binding of LIGHT to LTβR, and (f) binding of FASL to FAS.

Provided herein is (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.6); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) for use in a method of inhibiting or reducing (e.g., preventing) binding of one or more TNFSF ligand to one or more of the TNFSF ligand's cognate TNFSF receptors in a subject in need thereof, the method comprising administering to the subject the protein, the fusion protein, the conjugate, the immunogenic peptide or protein, the antibody, the nucleic acid molecule, the mRNA molecule, the vector, the cell, the carrier, the lipid nanoparticle, the vaccine composition, or the pharmaceutical composition, to thereby inhibit or reduce (e.g., prevent) binding of one or more TNFSF ligand to one or more of the TNFSF ligand's cognate TNFSF receptors in the subject.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.6); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for inhibiting or reducing (e.g., preventing) binding of one or more TNFSF ligand to one or more of the TNFSF ligand's cognate TNFSF receptors in a subject in need thereof.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.6); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for inhibiting or reducing (e.g., preventing) binding of one or more TNFSF ligand to one or more of the TNFSF ligand's cognate TNFSF receptors in a subject in need thereof, comprising administering to the subject the protein, the fusion protein, the conjugate, the immunogenic peptide or protein, the antibody, the nucleic acid molecule, the mRNA molecule, the expression vector, the cell, the carrier, the lipid nanoparticle, the vaccine composition, or the pharmaceutical composition, to thereby inhibit or reduce (e.g., prevent) binding of one or more TNFSF ligand to one or more of the TNFSF ligand's cognate TNFSF receptors in the subject.

5.14.3 Methods of Inhibiting or Reducing Signaling Mediated by TNFSF Ligand binding to One or More Cognate TNFSF Receptor

Provided herein are methods of inhibiting or reducing (e.g., preventing) signaling mediated by the binding of one or more TNFSF ligand to one or more of the TNFSF ligand's cognate TNFSF receptors in a subject in need thereof, the method comprising administering to the subject (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.6); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13); to thereby inhibit or reduce (e.g., prevent) signaling mediated by the binding of one or more TNFSF ligand to one or more of the TNFSF ligand's cognate TNFSF receptors in the subject. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition is administered to the subject the in an amount and for a time sufficient to inhibit or reduce (e.g., prevent) signaling mediated by the binding of one or more TNFSF ligand to one or more of the TNFSF ligand's cognate TNFSF receptors in the subject.

In some embodiments, the method comprises inhibiting or reducing (e.g., preventing) signaling mediated by binding of any one or more (e.g., 1, 2, 3, 4, 5 and/or 6) of the following: (a) binding of TLA1 to DR3, (b) binding of TNFα to TNFR2, (c) binding of TNFα to TNFR1, (d) binding of LIGHT to HVEM, (e) binding of LIGHT to LTβR, and/or (f) binding of FASL to FAS. In some embodiments, the method comprises inhibiting or reducing (e.g., preventing) signaling mediated by binding of 1, 2, 3, 4, 5, and/or 6 of the following: (a) binding of TLA1 to DR3, (b) binding of TNFα to TNFR2, (c) binding of TNFα to TNFR1, (d) binding of LIGHT to HVEM, (e) binding of LIGHT to LTβR, and/or (f) binding of FASL to FAS. In some embodiments, the method comprises inhibiting or reducing (e.g., preventing) signaling mediated by binding of a plurality of the following: (a) binding of TLA1 to DR3, (b) binding of TNFα to TNFR2, (c) binding of TNFα to TNFR1, (d) binding of LIGHT to HVEM, (e) binding of LIGHT to LTβR, and/or (f) binding of FASL to FAS. In some embodiments, the method comprises inhibiting or reducing (e.g., preventing) signaling mediated by binding of the following: (a) binding of TLA1 to DR3, (b) binding of TNFα to TNFR2, (c) binding of TNFα to TNFR1, (d) binding of LIGHT to HVEM, (e) binding of LIGHT to LTβR, and (f) binding of FASL to FAS.

Provided herein is (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.6); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) for use in a method of inhibiting or reducing (e.g., preventing) signaling mediated by the binding of one or more TNFSF ligand to one or more of the TNFSF ligand's cognate TNFSF receptors in a subject in need thereof, the method comprising administering to the subject the protein, the fusion protein, the conjugate, the immunogenic peptide or protein, the antibody, the nucleic acid molecule, the mRNA molecule, the vector, the cell, the carrier, the lipid nanoparticle, the vaccine composition, or the pharmaceutical composition, to thereby inhibit or reduce (e.g., prevent) signaling mediated by the binding of one or more TNFSF ligand to one or more of the TNFSF ligand's cognate TNFSF receptors in the subject.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.6); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for inhibiting or reducing (e.g., preventing) signaling mediated by the binding of one or more TNFSF ligand to one or more of the TNFSF ligand's cognate TNFSF receptors in a subject in need thereof.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.6); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for inhibiting or reducing (e.g., preventing) signaling mediated by the binding of one or more TNFSF ligand to one or more of the TNFSF ligand's cognate TNFSF receptors in a subject in need thereof, comprising administering to the subject the protein, the fusion protein, the conjugate, the immunogenic peptide or protein, the antibody, the nucleic acid molecule, the mRNA molecule, the expression vector, the cell, the carrier, the lipid nanoparticle, the vaccine composition, or the pharmaceutical composition, to thereby inhibit or reduce (e.g., prevent) signaling mediated by the binding of one or more TNFSF ligand to one or more of the TNFSF ligand's cognate TNFSF receptors in the subject.

### 5.14.4 Methods of Inhibiting or Reducing Binding of TL1A to DR3

Provided herein are methods of inhibiting or reducing (e.g., preventing) binding of TL1A to DR3 in a subject in need thereof, the method comprising administering to the subject (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13); to thereby inhibit or reduce (e.g., prevent) binding of TL1A to DR3 in the subject. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition is administered to the subject the in an amount and for a time sufficient to inhibit or reduce (e.g., prevent) binding of TL1A to DR3 in the subject.

In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition selectively inhibits or reduces (e.g., prevents) of TL1A to DR3 (e.g., relative to inhibiting or reducing (e.g., preventing) binding of one or more other TNFSF ligands to one or more of their cognate receptors). In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) binding of any one or more (e.g., 1, 2, 3, 4, and/or 5) of the following: (a) binding of TNFα to TNFR2, (b) binding of TNFα to TNFR1, (c) binding of LIGHT to HVEM, (d) binding of LIGHT to LTβR, and/or (e) binding of FASL to FAS. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) binding of any 1, 2, 3, 4, and/or 5 of the following: a) binding of TNFα to TNFR2, (b) binding of TNFα to TNFR1, (c) binding of LIGHT to HVEM, (d) binding of LIGHT to LTβR, and/or (e) binding of FASL to FAS. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) binding of a plurality of the following: a) binding of TNFα to TNFR2, (b) binding of TNFα to TNFR1, (c) binding of LIGHT to HVEM, (d) binding of LIGHT to LTβR, and/or (e) binding of FASL to FAS. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) binding of the following: (a) binding of TNFα to TNFR2, (b) binding of TNFα to TNFR1, (c) binding of LIGHT to HVEM, (d) binding of LIGHT to LTβR, and (e) binding of FASL to FAS.

Provided herein is (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) for use in a method of inhibiting or reducing (e.g., preventing) binding of TL1A to DR3 in a subject in a subject in need thereof, the method comprising administering to the subject the protein, the fusion protein, the conjugate, the immunogenic peptide or protein, the antibody, the nucleic acid molecule, the mRNA molecule, the vector, the cell, the carrier, the lipid nanoparticle, the vaccine composition, or the pharmaceutical composition, to thereby inhibit or reduce (e.g., prevent) binding of TL1A to DR3 in a subject in the subject.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for inhibiting or reducing (e.g., preventing) binding TL1A to DR3 in a subject in a subject in need thereof.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for inhibiting or reducing (e.g., preventing) binding TL1A to DR3 in a subject in a subject in need thereof, comprising administering to the subject the protein, the fusion protein, the conjugate, the immunogenic peptide or protein, the antibody, the nucleic acid molecule, the mRNA molecule, the expression vector, the cell, the carrier, the lipid nanoparticle, the vaccine composition, or the pharmaceutical composition, to thereby inhibit or reduce (e.g., prevent) binding of TL1A to DR3 in the subject.

### 5.14.5 Methods of Inhibiting of Reducing Signaling Mediated by TL1A Binding to DR3

Provided herein are methods of inhibiting or reducing (e.g., preventing) signaling mediated by the binding of TL1A to DR3 in a subject in need thereof, the method comprising administering to the subject (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13); to thereby inhibit or reduce (e.g., prevent) signaling mediated by the binding of TL1A to DR3 in the subject. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition is administered to the subject the in an amount and for a time sufficient to inhibit or reduce (e.g., prevent) signaling mediated by the binding of TL1A to DR3 in the subject.

In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) signaling mediated by binding of any one or more (e.g., 1, 2, 3, 4, and/or 5) of the following: (a) binding of TNFα to TNFR2, (b) binding of TNFα to TNFR1, (c) binding of LIGHT to HVEM, (d) binding of LIGHT to LTβR, and/or (e) binding of FASL to FAS. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) signaling mediated by binding of 1, 2, 3, 4, and/or 5 of the following: (a) binding of TNFα to TNFR2, (b) binding of TNFα to TNFR1, (c) binding of LIGHT to HVEM, (d) binding of LIGHT to LTβR, and/or (e) binding of FASL to FAS. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) signaling mediated by binding of a plurality of the following: (a) binding of TNFα to TNFR2, (b) binding of TNFα to TNFR1, (c) binding of LIGHT to HVEM, (d) binding of LIGHT to LTβR, and/or (e) binding of FASL to FAS. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) signaling mediated by binding of the following: (a) binding of TNFα to TNFR2, (b) binding of TNFα to TNFR1, (c) binding of LIGHT to HVEM, (d) binding of LIGHT to LTβR, and/or (e) binding of FASL to FAS.

Provided herein is (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) for use in a method of inhibiting or reducing (e.g., preventing) signaling mediated by the binding of TL1A to DR3 in a subject in need thereof, the method comprising administering to the subject the protein, the fusion protein, the conjugate, the immunogenic peptide or protein, the antibody, the nucleic acid molecule, the mRNA molecule, the vector, the cell, the carrier, the lipid nanoparticle, the vaccine composition, or the pharmaceutical composition, to thereby inhibit or reduce (e.g., prevent) signaling mediated by the binding of TL1A to DR3 in the subject.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for inhibiting or reducing (e.g., preventing) signaling mediated by the binding of TL1A to DR3 in a subject in need thereof.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for inhibiting or reducing (e.g., preventing) signaling mediated by the binding of TL1A to DR3 in a subject in need thereof, comprising administering to the subject the protein, the fusion protein, the conjugate, the immunogenic peptide or protein, the antibody, the nucleic acid molecule, the mRNA molecule, the expression vector, the cell, the carrier, the lipid nanoparticle, the vaccine composition, or the pharmaceutical composition, to thereby inhibit or reduce (e.g., prevent) signaling mediated by the binding of TL1A to DR3 in the subject.

### 5.14.6 Methods of Inhibiting or Reducing Binding of TL1A to DcR3

Provided herein are methods of inhibiting or reducing (e.g., preventing) binding of TL1A to DcR3 in a subject in need thereof, the method comprising administering to the subject (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13); to thereby or reduce (e.g., prevent) binding of TL1A to DcR3 in the subject. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition is administered to the subject the in an amount and for a time sufficient to inhibit or reduce (e.g., prevent) binding of TL1A to DcR3 in the subject.

In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition selectively inhibits or reduces (e.g., prevents) of TL1A to DcR3 (e.g., relative to inhibiting or reducing (e.g., preventing) binding of one or more other TNFSF ligands to one or more of their cognate receptors). In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) binding of any one or more (e.g., 1, 2, 3, 4, and/or 5) of the following: (a) binding of TNFα to TNFR2, (b) binding of TNFα to TNFR1, (c) binding of LIGHT to HVEM, (d) binding of LIGHT to LTβR, and/or (e) binding of FASL to FAS. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) binding of any 1, 2, 3, 4, and/or 5 of the following: a) binding of TNFα to TNFR2, (b) binding of TNFα to TNFR1, (c) binding of LIGHT to HVEM, (d) binding of LIGHT to LTβR, and/or (e) binding of FASL to FAS. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) binding of a plurality of the following: a) binding of TNFα to TNFR2, (b) binding of TNFα to TNFR1, (c) binding of LIGHT to HVEM, (d) binding of LIGHT to LTβR, and/or (e) binding of FASL to FAS. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) binding of the following: (a) binding of TNFα to TNFR2, (b) binding of TNFα to TNFR1, (c) binding of LIGHT to HVEM, (d) binding of LIGHT to LTβR, and (e) binding of FASL to FAS.

Provided herein is (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) for use in a method of inhibiting or reducing (e.g., preventing) binding of TL1A to DcR3 in a subject in a subject in need thereof, the method comprising administering to the subject the protein, the fusion protein, the conjugate, the immunogenic peptide or protein, the antibody, the nucleic acid molecule, the mRNA molecule, the vector, the cell, the carrier, the lipid nanoparticle, the vaccine composition, or the pharmaceutical composition, to thereby inhibit or reduce (e.g., prevent) binding of TL1A to DcR3 in a subject in the subject.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for inhibiting or reducing (e.g., preventing) binding of TL1A to DcR3 in a subject in a subject in need thereof.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for inhibiting or reducing (e.g., preventing) binding of TL1A to DcR3 in a subject in a subject in need thereof, comprising administering to the subject the protein, the fusion protein, the conjugate, the immunogenic peptide or protein, the antibody, the nucleic acid molecule, the mRNA molecule, the expression vector, the cell, the carrier, the lipid nanoparticle, the vaccine composition, or the pharmaceutical composition, to thereby inhibit or reduce (e.g., prevent) binding of TL1A to DcR3 in the subject.

### 5.14.7 Methods of Inhibiting or Reducing Signaling Mediated by TL1A Binding to DcR3

Provided herein are methods of inhibiting or reducing (e.g., preventing) signaling mediated by the binding of TL1A to DcR3 in a subject in need thereof, the method comprising administering to the subject (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13); to thereby inhibit or reduce (e.g., prevent) signaling mediated by the binding of TL1A to DcR3 in the subject. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition is administered to the subject the in an amount and for a time sufficient to inhibit or reduce (e.g., prevent) signaling mediated by the binding of TL1A to DcR3 in the subject.

In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) signaling mediated by binding of any one or more (e.g., 1, 2, 3, 4, and/or 5) of the following: (a) binding of TNFα to TNFR2, (b) binding of TNFα to TNFR1, (c) binding of LIGHT to HVEM, (d) binding of LIGHT to LTβR, and/or (e) binding of FASL to FAS. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) signaling mediated by binding of 1, 2, 3, 4, and/or 5 of the following: (a) binding of TNFα to TNFR2, (b) binding of TNFα to TNFR1, (c) binding of LIGHT to HVEM, (d) binding of LIGHT to LTβR, and/or (e) binding of FASL to FAS. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) signaling mediated by binding of a plurality of the following: (a) binding of TNFα to TNFR2, (b) binding of TNFα to TNFR1, (c) binding of LIGHT to HVEM, (d) binding of LIGHT to LTβR, and/or (e) binding of FASL to FAS. In some embodiments, the immunoreceptor inhibitory protein, the fusion protein, the conjugate, the immunogenic peptide or protein, the antibody, the nucleic acid molecule, the vector, the host cell, the carrier, the vaccine composition, or the pharmaceutical composition does not inhibit or reduce (e.g., prevent) signaling mediated by binding of the following: (a) binding of TNFα to TNFR2, (b) binding of TNFα to TNFR1, (c) binding of LIGHT to HVEM, (d) binding of LIGHT to LTβR, and/or (e) binding of FASL to FAS.

Provided herein is (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) for use in a method of inhibiting or reducing (e.g., preventing) signaling mediated by the binding of TL1A to DcR3 in a subject in need thereof, the method comprising administering to the subject the protein, the fusion protein, the conjugate, the immunogenic peptide or protein, the antibody, the nucleic acid molecule, the mRNA molecule, the vector, the cell, the carrier, the lipid nanoparticle, the vaccine composition, or the pharmaceutical composition, to thereby inhibit or reduce (e.g., prevent) signaling mediated by the binding of TL1A to DcR3 in the subject.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for inhibiting or reducing (e.g., preventing) signaling mediated by the binding of TL1A to DcR3 in a subject in need thereof.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for inhibiting or reducing (e.g., preventing) signaling mediated by the binding of TL1A to DcR3 in a subject in need thereof, comprising administering to the subject the protein, the fusion protein, the conjugate, the immunogenic peptide or protein, the antibody, the nucleic acid molecule, the mRNA molecule, the expression vector, the cell, the carrier, the lipid nanoparticle, the vaccine composition, or the pharmaceutical composition, to thereby inhibit or reduce (e.g., prevent) signaling mediated by the binding of TL1A to DcR3 in the subject.

### 5.14.8 Methods of Suppressing or Preventing a Pro-Inflammatory Immune Response

Provided herein are methods of suppressing or preventing a pro-inflammatory immune response in a subject in need thereof, the method comprising administering to the subject (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13), to thereby suppress or prevent a pro-inflammatory immune response in the subject. In some embodiments, the immunoreceptor inhibitory protein, the fusion protein, the conjugate, the immunogenic peptide or protein, the antibody, the nucleic acid molecule, the vector, the host cell, the carrier, or the pharmaceutical composition is administered to the subject the in an amount and for a time sufficient to suppress or prevent a pro-inflammatory immune response in the subject.

Provided herein is (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) for use in a method of suppressing or preventing a pro-inflammatory immune response in a subject in need thereof, the method comprising administering to the subject the protein, the fusion protein, the conjugate, the immunogenic peptide or protein, the antibody, the nucleic acid molecule, the mRNA molecule, the vector, the cell, the carrier, the lipid nanoparticle, the vaccine composition, or the pharmaceutical composition, to thereby suppress or prevent a pro-inflammatory immune response in the subject.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for suppressing or preventing a pro-inflammatory immune response in a subject in need thereof.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for suppressing or preventing a pro-inflammatory immune response in a subject in need thereof, comprising administering to the subject the protein, the fusion protein, the conjugate, the immunogenic peptide or protein, the antibody, the nucleic acid molecule, the mRNA molecule, the expression vector, the cell, the carrier, the lipid nanoparticle, the vaccine composition, or the pharmaceutical composition, to thereby suppress or prevent a pro-inflammatory immune response in the subject.

### 5.14.9 Methods of Preventing, Treating, or Ameliorating a Disease in a Subject in Need Thereof

Provided herein are methods of preventing, treating, or ameliorating a disease in a subject in a subject in need thereof, the method comprising administering to the subject (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13), to thereby prevent, treat, or ameliorate the disease in the subject. In some embodiments, the immunoreceptor inhibitory protein, the fusion protein, the conjugate, the immunogenic peptide or protein, the antibody, the nucleic acid molecule, the vector, the host cell, the carrier, or the pharmaceutical composition is administered to the subject the in an amount and for a time sufficient to prevent, treat, or ameliorate the disease in the subject.

In some embodiments, the disease is a pro-inflammatory disease, an autoimmune disease, or a metabolic inflammatory disease. In some embodiments, the disease is inflammatory bowel disease, Crohn's disease, ulcerative colitis, rheumatoid arthritis, psoriatic arthritis, ankylosing spondylitis, asthma, systemic lupus erythematosus, or fibrosis. In some embodiments, the disease is inflammatory bowel disease, Crohn's disease, ulcerative colitis, rheumatoid arthritis, psoriatic arthritis, ankylosing spondylitis, asthma, systemic lupus erythematosus. In some embodiments, the disease is fibrosis.

Provided herein is (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) for use in a method of preventing, treating, or ameliorating a disease in a subject in need thereof, the method comprising administering to the subject the protein, the fusion protein, the conjugate, the immunogenic peptide or protein, the antibody, the nucleic acid molecule, the mRNA molecule, the vector, the cell, the carrier, the lipid nanoparticle, the vaccine composition, or the pharmaceutical composition, to thereby prevent, treat, or ameliorate the disease in the subject.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for preventing, treating, or ameliorating a disease in a subject in need thereof.

Provided herein are uses of (i) an immunoreceptor inhibitory protein described herein (e.g., described in § 5.2); (ii) a fusion protein described herein (e.g., described in § 5.4); (iii) a conjugate described herein (e.g., described in § 5.4); (iv) an immunogenic peptide or protein described herein (e.g., described in § 5.5; (v) an antibody described herein (e.g., described in § 5.10); (vi) a nucleic acid molecule described herein (e.g., described in § 5.7); (vii) a vector described herein (e.g., described in § 5.8); (viii) a cell described herein (e.g., described in § 5.9); (ix) a carrier described herein (e.g., described in § 5.11); (x) a vaccine composition described herein (e.g., described in § 5.5); or (xi) a pharmaceutical composition described herein (e.g., described in § 5.13) in the manufacture of a medicament for preventing, treating, or ameliorating a disease in a subject in need thereof, comprising administering to the subject the protein, the fusion protein, the conjugate, the immunogenic peptide or protein, the antibody, the nucleic acid molecule, the mRNA molecule, the expression vector, the cell, the carrier, the lipid nanoparticle, the vaccine composition, or the pharmaceutical composition, to thereby prevent, treat, or ameliorate the disease in the subject.

### 5.14.10 Methods of Inducing or Enhancing an Immune Response

Provided herein are vaccines and methods of inducing or enhancing an immune response in a subject in need thereof, comprising administering to the subject (i) an immunogenic peptide or protein described herein (e.g., described in § 5.5) (or a conjugate or fusion protein thereof); (ii) a nucleic acid molecule encoding (i); (iii) a vector comprising (ii); (iv) a carrier (e.g., described herein) comprising (i), (ii), or (iii); (v) a vaccine composition (e.g., described herein) comprising (i), (ii), (iii), or (iv); or (vi) a pharmaceutical composition (e.g., described herein) comprising (i), (ii), (iii), (iv), or (v); to thereby induce or enhance an immune response (e.g., to the immunogenic peptide or protein described herein) an in the subject. In some embodiments, the immunogenic peptide or protein (or the conjugate or fusion protein thereof), the nucleic acid molecule, the vector, the carrier, the vaccine composition, or the pharmaceutical composition is administered to the subject the in an amount and for a time sufficient to induce or enhance an immune response (e.g., to the immunogenic peptide or protein described herein) in the subject.

Provided herein is (i) an immunogenic peptide or protein described herein (e.g., described in § 5.5) (or a conjugate or fusion protein thereof); (ii) a nucleic acid molecule encoding (i); (iii) a vector comprising (ii); (iv) a carrier (e.g., described herein) comprising (i), (ii), or (iii); (v) a vaccine composition (e.g., described herein) comprising (i), (ii), (iii), or (iv); or (vi) a pharmaceutical composition (e.g., described herein) comprising (i), (ii), (iii), (iv), or (v) for use in a method of inducing or enhancing an immune response in a subject in need thereof, the method comprising administering to the subject the immunogenic peptide or protein (or the conjugate or fusion protein thereof), the nucleic acid molecule, the vector, the carrier, the vaccine composition, or the pharmaceutical composition, to thereby induce or enhance an immune response in the subject.

Provided herein are uses of (i) an immunogenic peptide or protein described herein (e.g., described in § 5.5) (or a conjugate or fusion protein thereof); (ii) a nucleic acid molecule encoding (i); (iii) a vector comprising (ii); (iv) a carrier (e.g., described herein) comprising (i), (ii), or (iii); (v) a vaccine composition (e.g., described herein) comprising (i), (ii), (iii), or (iv); or (vi) a pharmaceutical composition (e.g., described herein) comprising (i), (ii), (iii), (iv), or (v) in the manufacture of a medicament for inducing or enhancing an immune response in a subject in need thereof.

Provided herein are uses of (i) an immunogenic peptide or protein described herein (e.g., described in § 5.5) (or a conjugate or fusion protein thereof); (ii) a nucleic acid molecule encoding (i); (iii) a vector comprising (ii); (iv) a carrier (e.g., described herein) comprising (i), (ii), or (iii); (v) a vaccine composition (e.g., described herein) comprising (i), (ii), (iii), or (iv); or (vi) a pharmaceutical composition (e.g., described herein) comprising (i), (ii), (iii), (iv), or (v) in the manufacture of a medicament for inducing or enhancing an immune response in a subject in need thereof, comprising administering to the subject the immunogenic peptide or protein (or the conjugate or fusion protein thereof), the nucleic acid molecule, the vector, the carrier, the vaccine composition, or the pharmaceutical composition, to thereby induce or enhance an immune response in the subject.

In some embodiments, the immune response is immunogen specific. In some embodiments, the immune response is specific for the immunogenic peptide or protein described herein.

Enhancing an immune response (e.g., an immunogen specific immune response) includes e.g., increasing the duration of an immune response, increasing the magnitude of an immune response, and/or changing the nature of the immune response.

An immune response in a subject can be measured by common methods known to those of skill in the art. For example, serological assays can be employed to detect a humoral response by measuring titers of anti-immunogen (e.g., anti-immunoreceptor inhibitory protein) IgG antibodies post administration. For example, an enzyme-linked immunosorbent assay (ELISA) is a standard laboratory test for detecting and quantifying antibodies well known to the person of skill in the art. Generally, blood is collected from a consenting subject, centrifuged, and the serum isolated according to standard techniques. The recombinant target immunogen (e.g., immunoreceptor inhibitory protein) is immobilized in microplate wells. The microplate is blocked by through the incubation with an irrelevant immunogen (e.g., bovine serum albumin). The serum sample from the subject is prepared and added to the blocked wells to allow for binding of an immunogen specific antibodies to the immobilized immunogen. The bound antibodies are detected using a secondary tagged antibody that binds to the previously bound antibodies (e.g., anti-human IgG antibodies). See, e.g., Yannick G. et al. Humoral Responses and Serological Assays in SARS-CoV-2 Infections, Frontiers in Immunology, Vol 11 (2020) 10.3389/fimmu.2020.610688; Forgacs David et al., SARS-COV-2 mRNA Vaccines Elicit Different Responses in Immunologically Naïve and Pre-Immune Humans; Front. Immunol., Vol 12 (27 Sep. 2021) https://doi.org/10.3389/fimmu.2021.728021, the entire contents of each of which is incorporated by reference herein for all purposes.

Cell based assays can also be utilized to detect a cell based immune response (e.g., T cell immune response). For example, immunogen specific T cells (e.g., CD4+ or CD8+ T cells) can be measured using an enzyme-linked immunospot (ELISpot), an intracellular cytokine staining (ICS) assay, or an activation induced marker assay (AIM). Each of these assays is commonly used to detect cell based (e.g., T cell) immune responses to vaccines and well known to the person of ordinary skill in the art. See, e.g., Bowyer, Georgina et al. “Activation-induced Markers Detect Vaccine-Specific CD4⁺ T Cell Responses Not Measured by Assays Conventionally Used in Clinical Trials.” Vaccines vol. 6, 3 50. 31 Jul. 2018, doi: 10.3390/vaccines6030050, the entire contents of which is incorporated by reference herein for all purposes.

### 5.14.11 Methods of Vaccinating a Subject

Provided herein are methods of vaccinating a subject (e.g., against a viral infection), comprising administering to the subject administering to the subject (i) an immunogenic peptide or protein described herein (e.g., described in § 5.5) (or a conjugate or fusion protein thereof); (ii) a nucleic acid molecule encoding (i); (iii) a vector comprising (ii); (iv) a carrier (e.g., described herein) comprising (i), (ii), or (iii); (v) a vaccine composition (e.g., described herein) comprising (i), (ii), (iii), or (iv); or (vi) a pharmaceutical composition (e.g., described herein) comprising (i), (ii), (iii), (iv), or (v); to thereby; to thereby vaccinate the subject (e.g., against a viral infection). In some embodiments, the immunogenic peptide or protein (or the conjugate or fusion protein thereof), the nucleic acid molecule, the vector, the carrier, the vaccine composition, or the pharmaceutical composition is administered to the subject the in an amount and for a time sufficient to vaccinate the subject (e.g., against a viral infection).

Provided herein is (i) an immunogenic peptide or protein described herein (e.g., described in § 5.5) (or a conjugate or fusion protein thereof); (ii) a nucleic acid molecule encoding (i); (iii) a vector comprising (ii); (iv) a carrier (e.g., described herein) comprising (i), (ii), or (iii); (v) a vaccine composition (e.g., described herein) comprising (i), (ii), (iii), or (iv); or (vi) a pharmaceutical composition (e.g., described herein) comprising (i), (ii), (iii), (iv), or (v) for use in a method of vaccinating a subject (e.g., against a viral infection), the method comprising administering to the subject the protein, the fusion protein, the conjugate, the immunogenic peptide or protein, the antibody, the nucleic acid molecule, the mRNA molecule, the vector, the cell, the carrier, the lipid nanoparticle, the vaccine composition, or the pharmaceutical composition, to thereby vaccinate the subject (e.g., against a viral infection).

Provided herein are uses of (i) an immunogenic peptide or protein described herein (e.g., described in § 5.5) (or a conjugate or fusion protein thereof); (ii) a nucleic acid molecule encoding (i); (iii) a vector comprising (ii); (iv) a carrier (e.g., described herein) comprising (i), (ii), or (iii); (v) a vaccine composition (e.g., described herein) comprising (i), (ii), (iii), or (iv); or (vi) a pharmaceutical composition (e.g., described herein) comprising (i), (ii), (iii), (iv), or (v) in the manufacture of a medicament for vaccinating a subject (e.g., against a viral infection).

Provided herein are uses of (i) an immunogenic peptide or protein described herein (e.g., described in § 5.5) (or a conjugate or fusion protein thereof); (ii) a nucleic acid molecule encoding (i); (iii) a vector comprising (ii); (iv) a carrier (e.g., described herein) comprising (i), (ii), or (iii); (v) a vaccine composition (e.g., described herein) comprising (i), (ii), (iii), or (iv); or (vi) a pharmaceutical composition (e.g., described herein) comprising (i), (ii), (iii), (iv), or (v) in the manufacture of a medicament for vaccinating a subject (e.g., against a viral infection), comprising administering to the subject the protein, the fusion protein, the conjugate, the immunogenic peptide or protein, the antibody, the nucleic acid molecule, the mRNA molecule, the expression vector, the cell, the carrier, the lipid nanoparticle, the vaccine composition, or the pharmaceutical composition, to vaccinate the subject (e.g., against a viral infection).

In some embodiments, the pharmaceutical composition is administered to the subject as a prophylactic treatment. In some embodiments, the pharmaceutical composition is administered as a treatment after the onset of at least one symptom of an infection or disease. The pharmaceutical compositions described herein may be administered as a prime and/or a boost in a homologous or heterologous prime-boost regimen.

In some embodiments, the pharmaceutical composition prevents infection with the virus, reduces the likelihood or severity of infection with the virus, reduces the likelihood of developing an established infection after challenge with the virus, prevents or delays onset one or more symptoms of a disease associated with the viral infection, reduces in frequency and/or severity one or more symptoms of the disease, and/or reduces the risk of hospitalization or death associated with the disease.

Provided herein are methods of treating a viral infection in a subject, the method comprising (a) receiving testing results that determined the presence of an immunoreceptor inhibitory protein described herein (or a fragment or variant thereof) or a nucleic acid molecule encoding the immunoreceptor inhibitory protein described herein (or a fragment or variant thereof) in a sample from the subject, (b) diagnosing the subject as having the viral infection, and (c) administering a therapeutic agent to treat the viral infection.

An appropriate therapeutic agent for treatment of the viral infection can be selected by a person of ordinary skill in art according to standard practices. For example, the antiviral agent may be an attachment inhibitor, post-attachment inhibitor, fusion inhibitor, entry inhibitor, uncoating inhibitor, protease inhibitor, polymerase inhibitor, nucleotide reverse transcriptase inhibitor, nucleoside reverse transcriptase inhibitor, non-nucleoside reverse transcriptase inhibitor, and/or integrase inhibitor. In some embodiments, the antiviral agent is a capsid inhibitor, a secretion inhibitor, a microRNA, an antisense RNA agent, an RNAi agent, or other agent designed to inhibit viral RNA. In some embodiments, the antiviral agent is a small molecule, a lipid, a nucleic acid molecule, a peptide, or an antibody. In some embodiments, the antiviral agent is a small molecule antiviral agent. In some embodiments, the antiviral agent is a nucleoside analog, a peptide, or a nonribosomal peptide.

In some embodiments, the antiviral agent targets a DNA virus. In some embodiments, the antiviral agent targets an RNA virus. In some embodiments, the antiviral agent has broad spectrum activity against numerous types of viruses, e.g., and is capable of targeting both a DNA virus and an RNA virus.

The antiviral agent may function by targeting a specific viral function, such as inhibiting a viral nucleic acid polymerase, viral protease, viral integrase, or viral neuraminidase. In another embodiment, the antiviral agent functions by targeting a host cell function required for successful viral replication, such as viral entry into a host cell, nucleic acid synthesis, protein synthesis, viral capsid assembly, or viral exit from the host cell.

Exemplary antiviral agents include, but are not limited to, abacavir, acyclovir, amantadine, ampligen, amprenavir, umifenovir, atripia, alazanavir, biktarvy, baloxavir marboxil, bulevirtide, boceprevir, chloroquine, cidofovir, cobicistat, combivir, daclatasvir, darunavir, delavirdine, descovy, didanosine, docosanol, dolutegravir, doravirine, edoxudine, efavirenz, elvitegravir, emtricitabine, enfuvirtide, entecavir, etravirine, ensitrelvir, famciclovir, favipirvir, fomivirsen, fosamprenavir, foscamet, ganciclovir, hydroxychloroquine, ibacitabine, ibalizumab, idoxuridine, imiquimod, imunovir, ivermectin, indinavir, lamivudine, letermovir, lopinavir, loviride, maraviroc, methisazone, moroxydine, nelfinavir, nexavir, nitazoxanide, norvir, oseltamivir, penciclovir, peramvir, pleconaril, pieconaril, raltegravir, rilpivirine, ribavirin, remdesivir, ritonavir, saquinavir, sofosbuvir, taribavirin, telaprevir, tenofovir, telbivudine, trizivir, tipranavir, truvada, tromantadine, trifluridine, vidarabine, umifenovir, umifenovir, valaciclovir, vicriviroc, vidarabine, zalcitabine, zanamivir, and zicovidinr.

### 5.14.12 Diagnostic Methods

Further provided herein are diagnostic methods. Some of the methods described herein (e.g., diagnostic methods (see, e.g., § 5.14.12) and therapeutic methods (see, e.g., § 5.14.11)) utilize a sample from a subject. A suitable sample source, size, etc. can be determined by a person of ordinary skill in the art in accordance with use in the selected method. Exemplary subject samples include, but are not limited to, blood, plasma, cell, tissue, saliva sample, and nasal swab. Other samples include, but are not limited to, semen, sputum, mucous, sweat, urine, and feces. A diagnostic method herein may be an in vitro method.

Provided herein are methods of determining the presence of a virus in a subject, the method comprising (a) obtaining a sample from a subject, and (b) determining the presence or absence of an immunoreceptor inhibitory protein described herein (or a fragment or variant thereof) or a nucleic acid molecule encoding the immunoreceptor inhibitory protein described herein (or a fragment or variant thereof) in the sample.

Provided herein are methods of diagnosing a viral infection in a subject, the method comprising (a) obtaining a sample from a subject, (b) determining the presence or absence of an immunoreceptor inhibitory protein described herein (or a fragment or variant thereof) or a nucleic acid molecule encoding the immunoreceptor inhibitory protein described herein (or a fragment or variant thereof) in the sample, and (c) diagnosing the subject as having the viral infection if the immunoreceptor inhibitory protein described herein (or a fragment or variant thereof) or the nucleic acid molecule encoding the immunoreceptor inhibitory protein described herein (or a fragment or variant thereof), is determined to be present in the sample in step (b).

Provided herein are therapeutic agents to treat the viral infection for use in a method of treating a viral infection in a subject, the method comprising (a) receiving testing results that determined the presence of an immunoreceptor inhibitory protein described herein (or a fragment or variant thereof) or a nucleic acid molecule encoding the immunoreceptor inhibitory protein described herein (or the fragment or variant thereof) in a sample from the subject, (b) diagnosing the subject as having the viral infection, and (c) administering the therapeutic agent to treat the viral infection.

Provided herein are uses of a therapeutic agent to treat the viral infection in the manufacture of a medicament for treating a viral infection in a subject, comprising (a) receiving testing results that determined the presence of an immunoreceptor inhibitory protein described herein (or a fragment or variant thereof) or a nucleic acid molecule encoding the immunoreceptor inhibitory protein described herein (or the fragment or variant thereof) in a sample from the subject, (b) diagnosing the subject as having the viral infection, and (c) administering the therapeutic agent to treat the viral infection.

In some embodiments, an antibody described herein (e.g., an antibody that specifically binds an immunoreceptor inhibitory protein described herein) is utilized in any one of the diagnostic methods in order to determine the presence or absence of an immunoreceptor inhibitory protein described herein (or a fragment or variant thereof). In some embodiments, the antibody (e.g., an antibody that specifically binds an immunoreceptor inhibitory protein described herein) is labeled with a tag (e.g., a fluorescent tag) to aid in detection.

Exemplary viral infections include, but are not limited to, infections with a virus of family Herpesviridae (a herpes virus) (e.g., a betaherpesvirus (e.g., a cytomegalovirus, a muromegalovirus, a proboscivirus, a quwivirus, a roseolovirus); a gammaherpesvirus (e.g., a bossavirus, a lymphocryptovirus, a macavirus, a manticavirus, a patagivirus, a percavirus, a rhadinovirus); an alphaherpesvirus (e.g., an iltovirus a mardivirus, a scutavirus, a simplexvirus, a varicellovirus). In some embodiments, the virus is a human herpesviridae virus. in some embodiments, the virus is a cytomegalovirus (e.g., a human cytomegalovirus). In some embodiments, the virus is an Epstein-Barr virus (e.g., a human Epstein-Barr virus).

In some embodiments, the virus is a virus of the family Poxviridae (a poxvirus) (e.g., chordopoxvirus (e.g., a parapoxvirus (e.g., an ORF virus), a pseudocowpox virus)). In some embodiments, the virus is a chordopoxvirus (e.g., a human chordopoxvirus). In some embodiments, the virus is a parapoxvirus (e.g., a human parapoxvirus) (e.g., an ORF virus (e.g., a human ORF virus)). In some embodiments, the virus is an ORF virus (e.g., a human ORF virus). In some embodiments, the virus is a pseudocowpox virus (e.g., a human pseudocowpox virus).

### 5.15 Kits

Provided herein are kits comprising an agent described herein, e.g., an immunoreceptor inhibitory protein described herein (see, e.g., § 5.2), a fusion protein described herein (see, e.g., § 5.4), a conjugate described herein (see, e.g., § 5.4), an immunogenic peptide or protein described herein (see, e.g., § 5.5), an antibody described herein (see, e.g., § 5.10), a nucleic acid molecule described herein (see, e.g., § 5.7), a vector described herein (see, e.g., § 5.8), a cell described herein (see, e.g., § 5.9), a carrier described herein (see, e.g., § 5.11), a vaccine composition described herein (see, e.g., § 5.5), or a pharmaceutical composition described herein (see, e.g., § 5.13). In addition, the kit may comprise a liquid vehicle for solubilizing or diluting, and/or technical instructions. The technical instructions of the kit may contain information about administration and dosage and subject groups.

In some embodiments, the agent described herein, e.g., the immunoreceptor inhibitory protein described herein, the fusion protein described herein, the conjugate described herein, the immunogenic peptide or protein described herein, the antibody described herein, the nucleic acid molecule described herein, the vector described herein, the host cell described herein, the carrier described herein, the vaccine composition described herein, or the pharmaceutical composition described herein is provided in a separate part of the kit, wherein the agent, e.g., the immunoreceptor inhibitory protein described herein, the fusion protein described herein, the conjugate, the immunogenic peptide or protein described herein, the antibody described herein, the nucleic acid molecule described herein, the vector described herein, the host cell described herein, the carrier described herein, the vaccine composition described herein, or the pharmaceutical composition described herein is optionally lyophilized, spray-dried, or spray-freeze dried. The kit may further contain as a part a vehicle (e.g., buffer solution) for solubilizing the dried or lyophilized agent, e.g., immunoreceptor inhibitory protein described herein, fusion protein described herein, the conjugate, immunogenic peptide or protein described herein, antibody described herein, nucleic acid molecule described herein, vector described herein, host cell described herein, carrier described herein, the vaccine composition described herein, or pharmaceutical composition described herein.

In some embodiments, the kit comprises a single dose container. In some embodiments, the kit comprises a multi-dose container. In some embodiments, the kit comprises an administration device (e.g., an injector for intradermal injection or a syringe for intramuscular injection). In some embodiments, the kit comprises adjuvant in a separate container. The kit may further contain technical instructions for mixing the adjuvant prior to administration or for co-administration.

In some embodiments, the kit comprises an antibody described herein that specifically binds an immunoreceptor inhibitory protein described herein. In some embodiments, the antibody (e.g., an antibody that specifically binds an immunoreceptor inhibitory protein described herein) is labeled with a tag (e.g., a fluorescent tag) to aid in detection. In some embodiments, the kit comprises one or more reagent (e.g., a buffer) for a sample described herein. In some embodiments, the kit is for use in a method of determining the presence of a virus in a subject or a method of diagnosing a subject with a viral infection.

Any of the kits described herein may be used in any of the methods described herein (see, e.g., § 5.14).

### 6. EXAMPLES

TABLE OF CONTENTS

| | |
|---|---|
| 6.1 | Example 1. Immunoreceptor Inhibitory Protein Identification and Expression. |
| 6.2 | Example 2. Immunoreceptor Inhibitory Fusion Protein Binding to TNF |
| | Superfamily Ligands. |
| 6.3 | Example 3. Immunoreceptor Inhibitory Fusion Protein Binding to Human TL1A. |
| 6.4 | Example 4. Immunoreceptor Inhibitory Fusion Protein Binding to Membrane |
| | Bound Human TL1A. |
| 6.5 | Example 5. Immunoreceptor Inhibitory Fusion Protein Mediated Inhibition of |
| | TL1A Induced NFκB Activation. |
| 6.6 | Example 6. Immunoreceptor Inhibitory Fusion Protein Mediated Inhibition of DR3 |
| | Binding to TL1A. |
| 6.7 | Example 7. Immunoreceptor Inhibitory Fusion Protein Mediated Inhibition of |
| | DcR3 Binding to TL1A. |
| 6.8 | Example 8. Immunoreceptor Inhibitory Fusion Protein Mediated Inhibition of |
| | TL1A Induced Apoptosis. |
| 6.9 | Example 9. Summary of Exemplary Functional Attributes of Immunoreceptor |
| | Inhibitory Fusion Proteins. |
| 6.10 | Example 10. Immunoreceptor Inhibitory Fusion Protein Preferential Binding to |
| | Trimeric versus Monomeric TL1A. |
| 6.11 | Example 11. In Vivo IFNγ Expression in Mice Treated with Immunoreceptor |
| | Inhibitory Fusion Proteins. |
| 6.12 | Example 12. Immunoreceptor Inhibitory Fusion Protein Mediated In Vivo |
| | Reduction of Joint Inflammation and Local/Systemic Proinflammatory Cytokine |
| | Signals in Rheumatoid Arthritis Mouse Model. |
| 6.1 | Example 1. Immunoreceptor Inhibitory Protein Identification and Expression. |

Immunoreceptor Inhibitory Proteins 1-34 (IIPs 1-34) (SEQ ID NOS: 23-53, 187-188, and 199 respectively) were identified by the inventors through a process of searching, screening, and analysis of over 750 proteins for select proteins having the ability to bind hTL1A.

Subsequently, an exemplary subset of the identified IIPs were selected for use in the generation of exemplary immunoreceptor inhibitory fusion proteins (IIFPs), namely IIPs 1-4 and 32-34 (SEQ ID NOS: 23-26, 187, or 188, and 199 respectively). Each IIFP comprised from N- to C-terminus the hIL-2 signal sequence, an effector function reduced hIgG4 Fc region, a peptide linker, and one of IIPs 1-4 and 32-34 (SEQ ID NOS: 23-26, 187-188, and 199).

The IIFPs were generated using standard methods known in the art. Briefly, a DNA nucleic acid molecule encoding the fusion protein was synthesized and inserted into an expression plasmid. Expi293 cells (Thermo Fisher #A14527) were transfected using the Expi293 expression kit (Thermo Fisher #A14635) according to the manufacturer's protocol. Briefly, Expi293 cells were grown in suspension at 37° C., 8% CO₂ in Expi293 growth medium (Thermo Fisher #A1435101). The cells were counted using a hemocytometer to ensure a density of 2.5-3 million cells per mL, and a viability above 95%, prior to transfection. Transfections were performed in 2.5 ml of cell containing medium (7.5-9 million cells per reaction). 1 μg/ml of plasmid DNA was pre-incubated with Opti-MEM for 5 minutes at room temperature (RT) and ExpiFectamine was pre-incubated with Opti-MEM for 5 minutes at RT. The plasmid mixture was subsequently mixed with the ExpiFectamine mixture and incubated for 10-20 minutes at RT. After incubation, the mixture was added to the Expi293 cells and incubated overnight. On day 1 post-transfection, ExpiFectamine Enhancer 1 and ExpiFectamine Enhancer 2 were added to the cell culture. On day 3 post-transfection, the supernatant was removed and maintained at −20° C., and the cells were discarded. The amino acid sequence of generated mature IIFPs-1-4 and 32-34, is set forth in SEQ ID NOS: 175, 178, 181 184, 191, 194, and 202 respectively (see Table 9 herein). IIFPs were also produced in CHO cells using standard protein expression methods known in the art.

The concentration of each IIFP in the supernatant was determined using Octet Protein A Biosensors. Briefly, a standard curve was created using IgG4-Fc (externally sourced, in purified form, that binds Protein A) at 400 ug/mL and a 2-fold serial dilution, which established the linear range in biolayer interferometry reading. The standard curve served as a reference for quantifying each IIFP. The Octet system was then calibrated according to the manufacturer's instructions. Before introducing IIFP samples, the Protein A biosensors were hydrated in 190 μL of the same buffer the IIFPs were in to strip away the protective sugar coating as well as establish a baseline reading. The biosensors were then exposed to 80 μL of sample supernatant, allowing each IIFP to bind to the immobilized Protein A surface on the fiber optic tip. The binding process induced a change in the optical thickness at the biosensor surface, which the Octet system detects and measures via changes in light refraction. This optical change is directly proportional to the amount of protein bound to the biosensor. After an incubation period, the biosensors were removed from the IIFP samples, and the system measured the final optical readings. These readings were then compared against the standard curve to quantify the IIFP concentration in each of the samples. Samples were typically run both neat (undiluted) and diluted 5× or 10× in buffer to ensure that the readings fell well within the standard curve range. IIFPs-1-4 and 32-34 were consistently expressed in the supernatant across for separate lots. Purified IIFPs-1-4 and 32-34 were further quantified using a standard bicinchoninic acid assay.

### 6.2 Example 2. Immunoreceptor Inhibitory Fusion Protein Binding to TNF Superfamily Ligands

The ability of IIFPs to bind various members of the TNFSF (including hTL1A, hTNFα, hLIGHT, and hFASL) was assessed utilizing an exemplary subset of the IIFPs produced in Example 1, namely IIFPs-1-4 and 34.

In addition to IIFPs-1-4 and 34 produced in Example 1, a series of reference proteins were expressed according to the general methods detailed in Example 1. The amino acid sequence of the reference proteins is provided in Table 10.

TABLE 10
The Amino Acid Sequence of Reference Proteins

| | | SEQ ID |
|---|---|---|
| Description | Amino Acid Sequence | NO |
| TNFR2 ECD-hFc | LPAQVAFTPYAPEPGSTCRLREYYDQTAQMCCSKCSPGQHAKVFCT | 186 |
| | KTSDTVCDSCEDSTYTQLWNWVPECLSCGSRCSSDQVETQACTREQ | |
| | NRICTCRPGWYCALSKQEGCRLCAPLRKCRPGFGVARPGTETSDVV | |
| | CKPCAPGTFSNTTSSTDICRPHQICNVVAIPGNASMDAVCTSTSPT | |
| | RSMAPGAVHLPQPVSTRSQHTQPTPEPSTAPSTSFLLPMGPSPPAE | |
| | GSTGD | |
| hFc | AESKYGPPCPPCPAPEAAGGPSVFLFPPKPKDTLMISRTPEVTCVV | 155 |
| | VDVSQEDPEVQFNWYVDGVEVHNAKTKPREEQFNSTYRVVSVLIVL | |
| | HQDWLNGKEYKCKVSNKGLPSSIEKTISKAKGQPREPQVYTLPPSQ | |
| | EEMTKNQVSLTCLVKGFYPSDIAVEWESNGQPENNYKTTPPVLDSD | |
| | GSFFLYSRLTVDKSRWQEGNVFSCSVMHEALHNHYTQKSLSLSLG | |

Briefly, Maxisorp 384 well plates (Thermo Nunc Cat #P6366-1CS) were coated with 25 μL of 2 μg/mL of recombinant human TNFSF ligands: TNFα (Acro Bio TNA-H5228), TL1A (Acro Bio TLA-H5244), FASL (Acro Bio HAL-H5241), or LIGHT (Acro Bio LIT-H5242) in PBS or were coated with PBS alone overnight at 4° C. The plates were washed 3 times with 0.1% PBS-T and incubated with SuperBlock blocking buffer (Thermo Fisher 37515) for 1 hour at room temperature with shaking at 400 RPM. The plates were then washed 3 times with 0.1% PBS-T. The plates were subsequently incubated with a titration of one of the following proteins: IIFP-1 (SEQ ID NO: 175), IIFP-2 (SEQ ID NO: 178), IIFP-3 (SEQ ID NO: 181), IIFP-4 (SEQ ID NO: 184), IIFP-34 (SEQ ID NO: 202), DcR3-hFc (Acro Bio TNB-H5255 (DcR3 protein fused to hIgG1 Fc at the C-terminus)), TNFR2 ECD-hFc (Ichor Bio ICH4022 (two TNFR2 receptor extracellular domains fused to hIgG1 Fc at the C-terminus)) (SEQ ID NO: 186), or a human IgG4 Fc (referred to in the corresponding figures as “hFc”) (SEQ ID NO: 155), starting at 10 nM in an 11 point 3-fold serial dilution. Plates were incubated for 1 hour at room temperature with shaking at 400 RPM. ELISA plates were washed 3 times with 0.1% PBS-T and incubated with an anti-human Fc HRP conjugated secondary antibody (ThermoFisher Scientific Cat #H10307) 1:2000 in 0.1% PBS-T for 1 hour at room temperature with shaking at 400 RPM. The plates were washed 3 times with 0.1% PBS-T and incubated with 20 μL of TMB (ThermoFisher Scientific Cat #34028) per well. The reaction was stopped at 5 minutes by adding 20 μL of Stop Solution (BioFX LSTP-1000-01). The plates were read immediately on a plate reader, measuring absorbance (OD) at 450 nM. Blank subtracted ODs were calculated by subtracting the ODs from the blank plate from the readings from each of the ligand plates and data was plotted using PRISM (version 10.0.2).

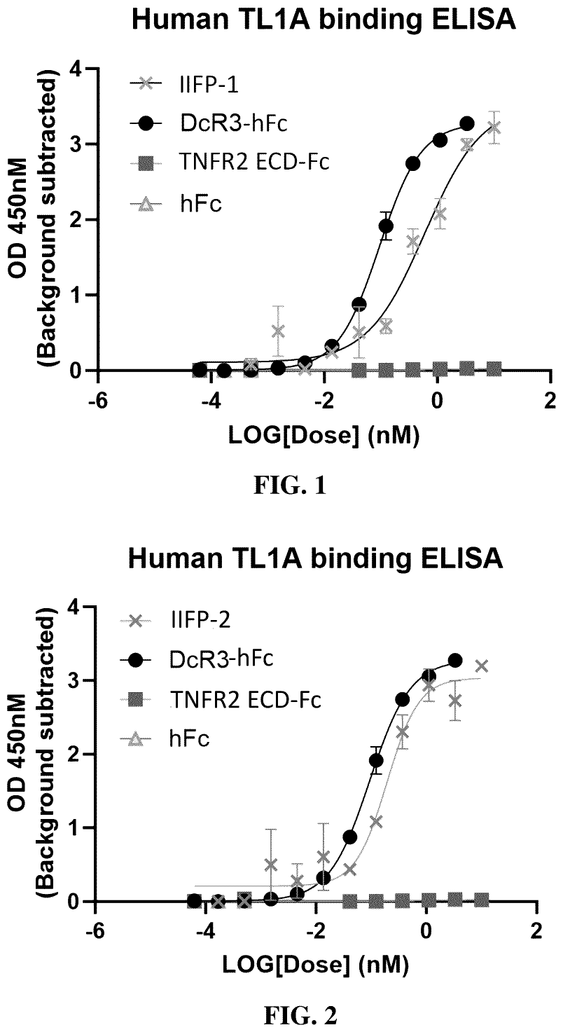

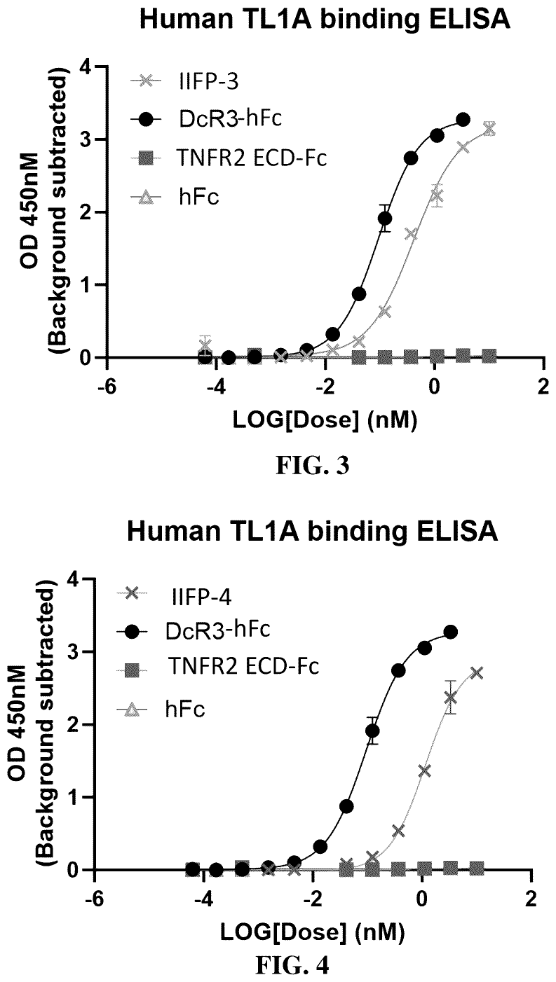

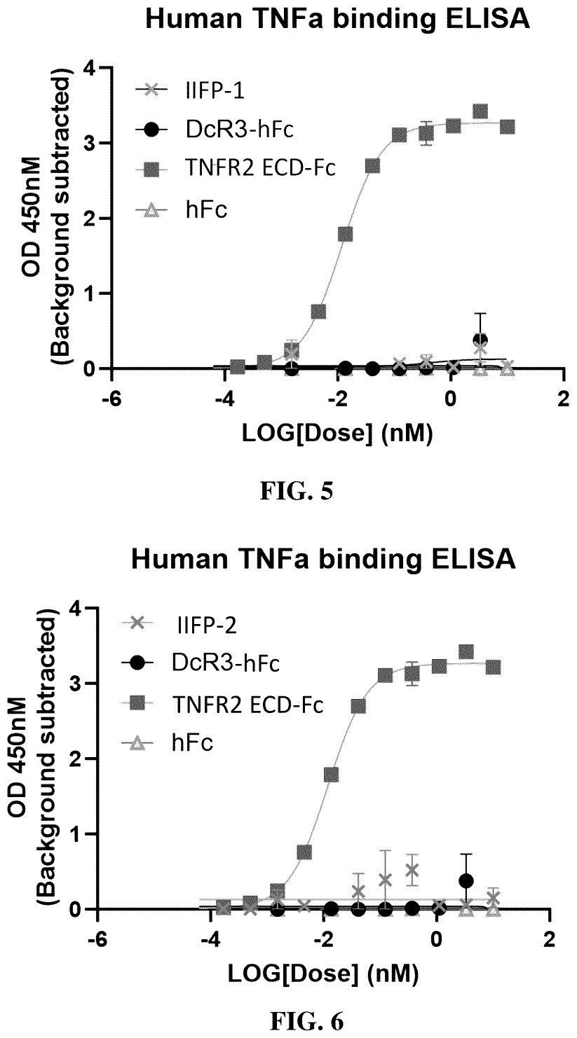

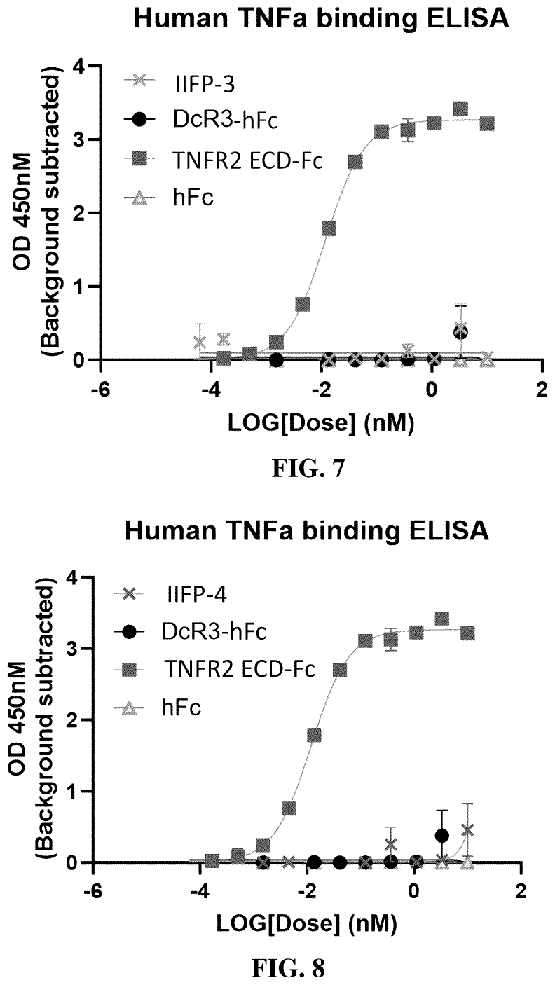

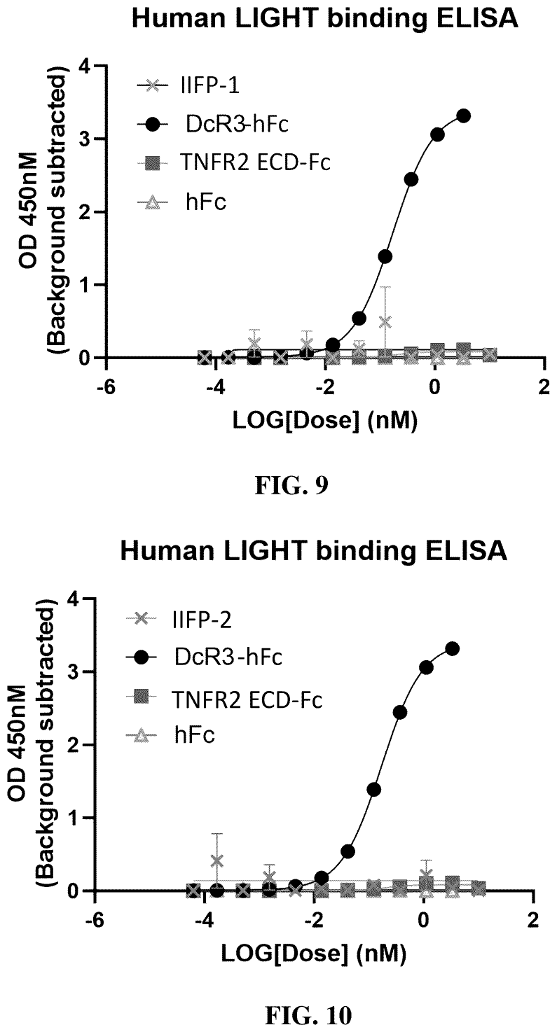

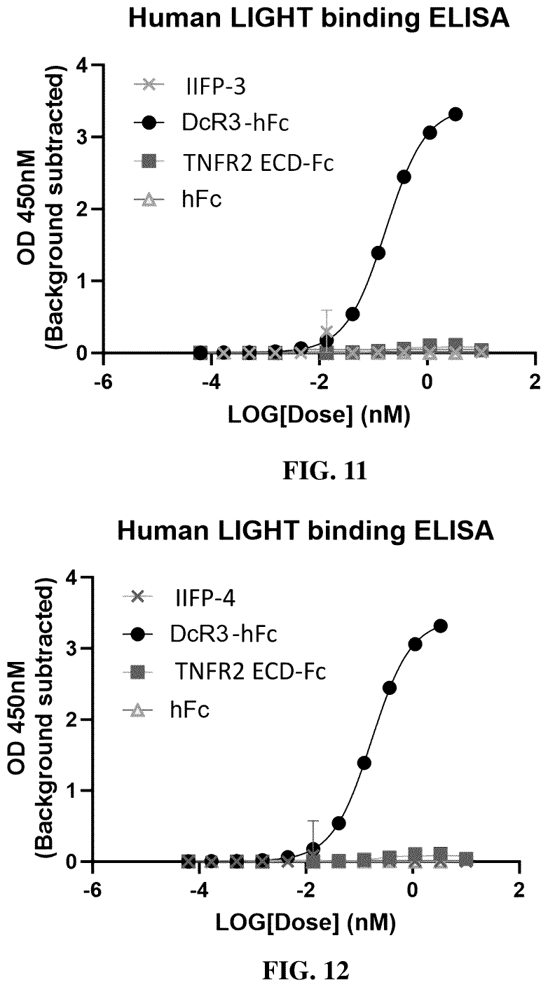

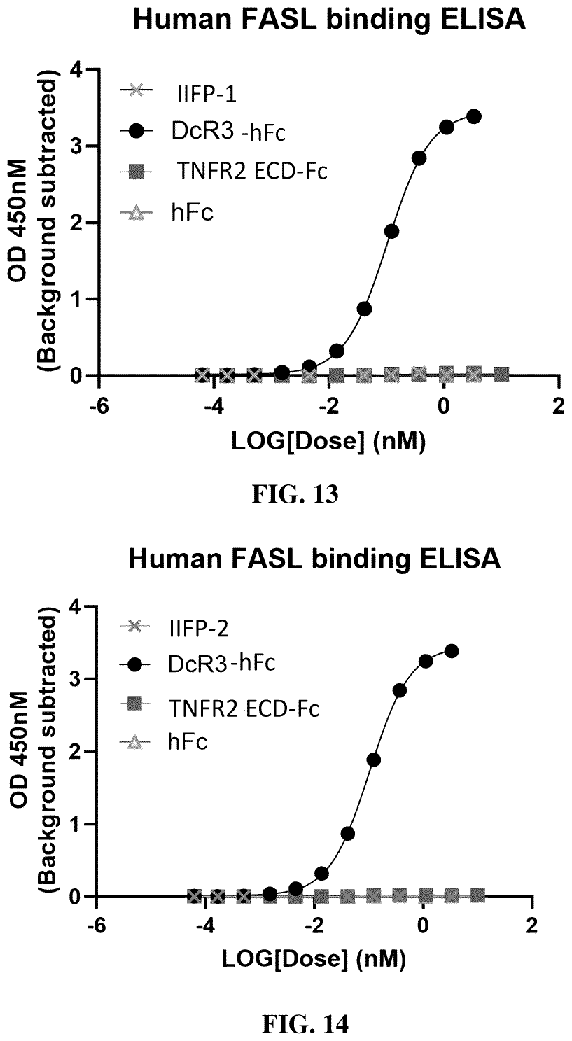

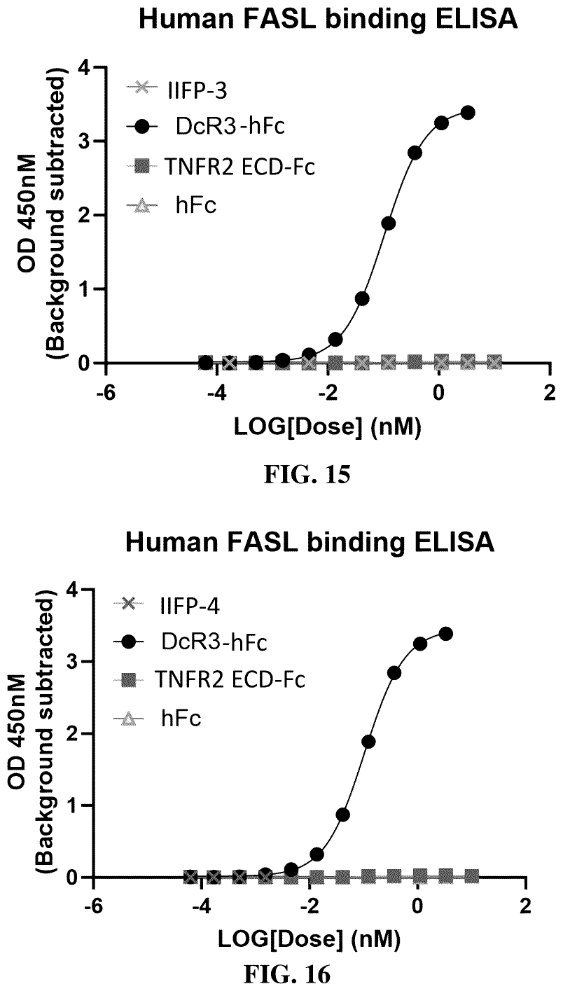

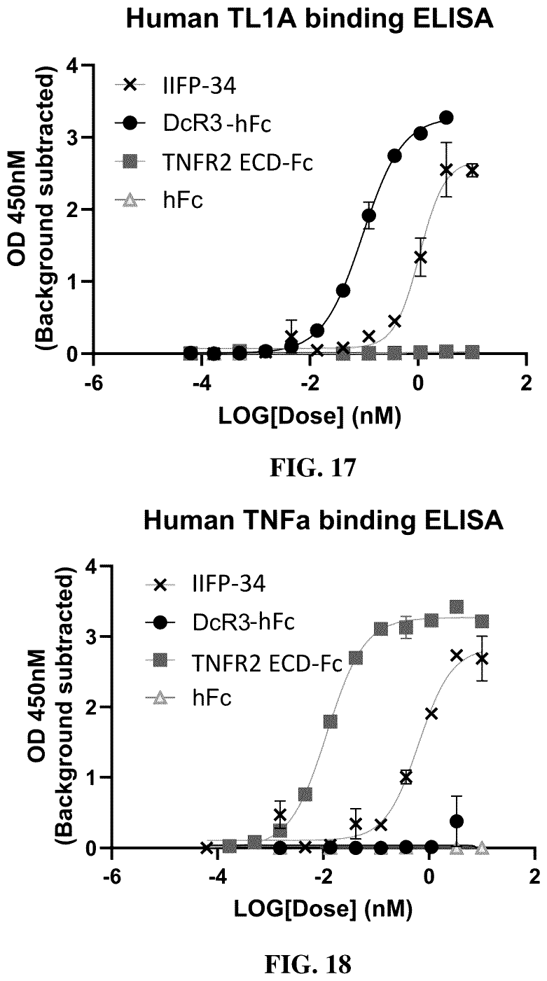

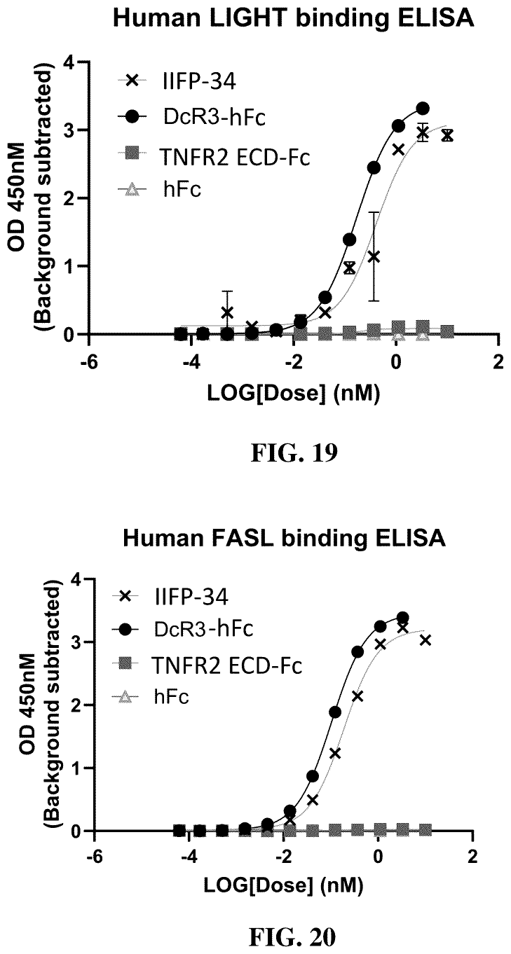

Each of IIFPs-1-4 were shown to specifically bind hTL1A (FIGS. 1-4). In addition, each of IIFPs-1-4 were shown not to bind hTNFα (FIGS. 5-8), hLIGHT (FIGS. 9-12), and hFASL (FIGS. 13-16). IIFP-34 was shown to bind hTL1A (FIG. 17), hTNFα (FIG. 18), hLIGHT (FIG. 19), and hFASL (FIG. 20).

### 6.3 Example 3. Immunoreceptor Inhibitory Fusion Protein Binding to Human TL1A

The ability of the fusion proteins comprising an IIP (IIFPs) to specifically binding hTL1A was assessed utilizing an exemplary subset of IIFPs made in Example 1, namely IIFPs-2, 32, and 33.

Briefly, Maxisorp 384 well plates (Thermo Nunc Cat #P6366-1CS) were coated with 25 μL of 2 μg/mL of recombinant human TL1A (Acro Bio TLA-H5244) in PBS O/N at 4° C. Plates were washed 3 times with 0.05% PBS-T and incubated with SuperBlock blocking buffer (Scytek Laboratories Super Block, AAA999) for 1 hour at room temperature with shaking at 400 RPM. Plates were then washed 3 times with 0.05% PBS-T. Plates were incubated with a titration of Fc tagged proteins of interest: hFc-IgG4, IIFP-1, IIFP-32 and IIFP-33 starting at 20 nM in a 12 point 2-fold serial dilution. Plates were incubated for 1 hour at room temperature with shaking at 400 RPM. ELISA plates were washed 3 times with 0.05% PBS-T and incubated with an anti-human Fc HRP conjugated secondary antibody (ThermoFisher Scientific Cat #H10307) 1:2000 in Scytek Laboratories Super Block, AAA999 for 1 hour at room temperature shaking at 400 RPM. Data analysis was conducted using GraphPad Prism software, employing an [Agonist] vs. response curve with a variable slope (four parameters).

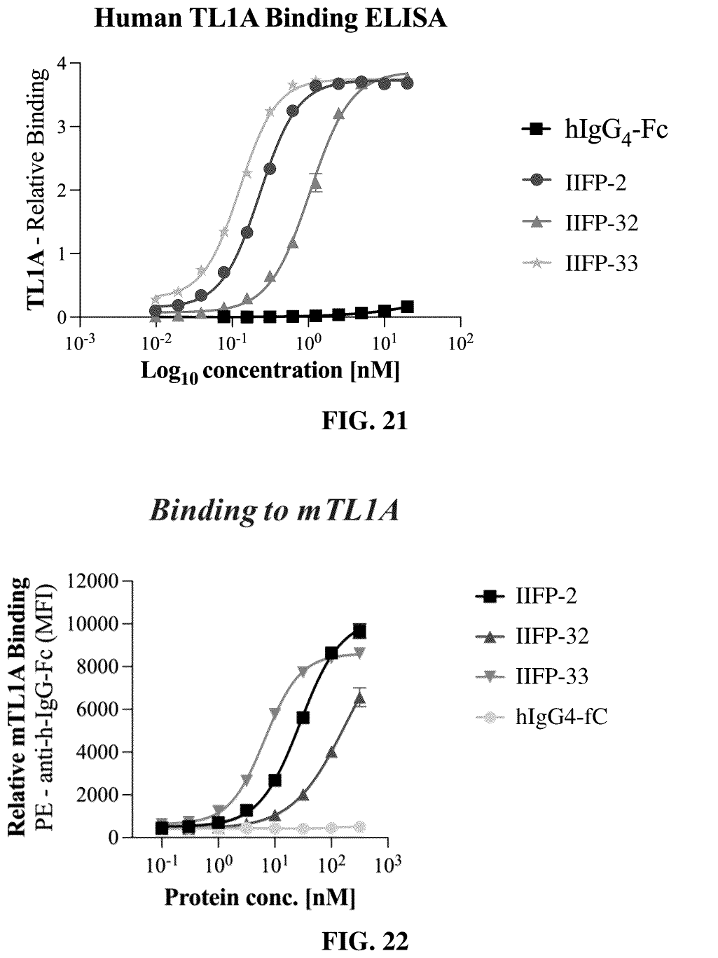

As shown in FIG. 21, IIFP-2, IIFP-32, and IIFP-33 each specifically bound plate bound human TL1A.

### 6.4 Example 4. Immunoreceptor Inhibitory Fusion Protein Binding to Membrane Bound Human TL1A

The ability of IIFPs to bind membrane bound hTL1A was assessed, further utilizing the exemplary subset of IIFPs-2, 32, and 33.

Briefly, HEK293/Human TL1A Stable Cell Line (Acro Biosystems, Catalog #CHEK-ATP142-2Vials) engineered to express mTL1A were harvested by centrifugation at 300×g for 5 minutes and aliquoted at 0.5 million cells per well into a 96-well U-bottom plate. The cells were washed twice with FACS buffer (PBS supplemented with 2% FBS) to remove residual media or debris. Viral TL1A inhibitors were then added in a dose titration, and the cells were incubated at 4° C. for 1 hour. Following incubation, the cells were washed twice with FACS buffer to remove unbound protein. Subsequently, Goat anti-human IgG (H+L) cross-absorbed Alexa Fluor 555-conjugated antibody (Invitrogen, Catalog #A21433) was added at a final concentration of 10 μg/mL, and the cells were incubated at 4° C. for 1 hour, protected from light to maintain fluorescence integrity. The cells were then washed twice with FACS buffer, resuspended between 150 μL-300 μL PBS per sample, and analyzed using a CytoFlex flow cytometer. Flow cytometric data were processed and analyzed with FlowJo software.

As shown in FIG. 22, IIFPs-2, 32, and 33 each bound membrane bound hTL1A expressed by HEK293 cells.

### 6.5 Example 5. Immunoreceptor Inhibitory Fusion Protein Mediated Inhibition of TL1A Induced NFκB Activation

The ability of IIFPs to inhibit TL1A induced NFκB activation was assessed, further utilizing the exemplary subset of IIFPs-2, 32, and 33.

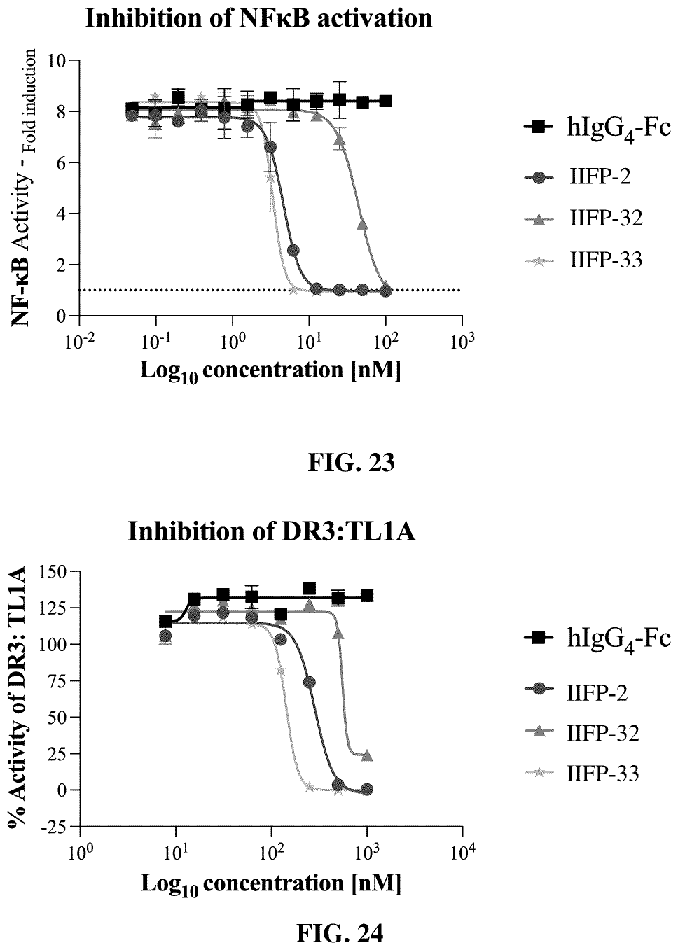

A commercially available TL1A responsive luciferase reporter Jurkat cell line was utilized. The cell line expresses luciferase under the control of NFκB response element and stably expresses human DR3. Activation of the NFκB signaling pathway by TL1A binding to DR3 induces luciferase expression. Briefly, a serial dilution of hIgG4-Fc (control), IIFP-2, IIFP-32, and IIFP-33 was prepared and pre-incubated with 10 nM of human TL1A for one hour. Subsequently, each mixture was added to approximately 32,000 TL1A responsive luciferase reporter Jurkat cells per well in a white, clear-bottomed 96-well plate. The plate was then incubated at 37° C. and 5% CO₂ for about five hours. Afterward, 100 μL of OneStep™ Luciferase Detection Reagent (BPS Bioscience #60690) was added to each well. The plate was shaken at 400 RPM at room temperature for approximately 15 minutes before the luminescence was measured using a Varioskan. The results are shown in FIG. 23 as fold induction of luciferase reporter expression, determined by comparing luciferase activity of stimulated cells against the activity of the control wells without treatment.

As shown in FIG. 23, each of IIFP-2, IIFP-32, and IIFP-33 mediated inhibition of TL1A induced NFκB activation.

### 6.6 Example 6. Immunoreceptor Inhibitory Fusion Protein Mediated Inhibition of DR3 Binding to TL1A

The ability of IIFPs to inhibit the binding of human DR3 to human TL1A was assessed, further utilizing the exemplary subset of IIFPs-2, 32, and 33.

A commercially available kit (DR3:TL1A Inhibitor Screening Assay Kit (BPS Biosciences, cat #82241-1)) was utilized and the assay conducted according to the manufacturer's instructions. Briefly, DR3 was first coated onto a 96-well or 384-well plate. Subsequently, a serial dilution of each of the following agents: hIgG4-Fc (control), IIFP-2, IIFP-32, and IIFP-33 was prepared and added to the plate. After 30 minutes, biotinylated TL1A was incubated with the coated DR3/agent. Streptavidin-HRP and an HRP substrate were subsequently added to the plate, producing chemiluminescence. The intensity of the chemiluminescence signal is directly proportional to the binding interaction between DR3 and TL1A. Results are expressed in FIG. 24 as percent of % activity of binding to DR3 upon inhibition, in which the binding measured in the absence of inhibitor is set to 100%.

As shown in FIG. 24, each of IIFP-2, IIFP-32, and IIFP-33 inhibited the binding of human DR3 to human TL1A.

### 6.7 Example 7. Immunoreceptor Inhibitory Fusion Protein Mediated Inhibition of DcR3 Binding to TL1A

The ability of IIFPs to inhibit the binding of human DcR3 to human TL1A was assessed, further utilizing the exemplary subset of IIFPs-2, 32, and 33.

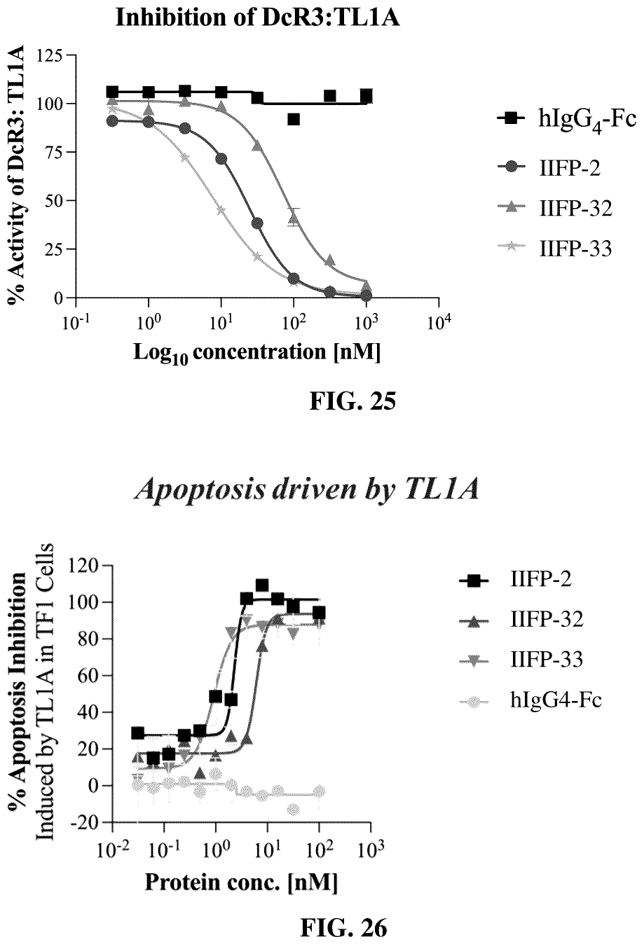

A commercially available kit (DcR3:TL1A Inhibitor Screening Assay Kit (BPS Biosciences, cat #82160)) was utilized and the assay conducted according to the manufacturer's instructions. Briefly, DcR3 is first coated onto a 96-well or 384-well plate. Subsequently, a serial dilution of each of the following agents: hIgG4-Fc (control), IIFP-2, IIFP-32, and IIFP-33 was prepared and added to plate. After 30 minutes, biotinylated TL1A was incubated with the coated DcR3/agent. Streptavidin-HRP and an HRP substrate were subsequently added to the plate, producing chemiluminescence. The intensity of the chemiluminescence signal is directly proportional to the binding interaction between DcR3 and TL1A. Results are expressed in FIG. 25 as percent of % activity of binding to DcR3 upon inhibition, in which the binding measured in the absence of inhibitor is set to 100%.

As shown in FIG. 25, each of IIFP-2, IIFP-32, and IIFP-33 inhibited the binding of human DcR3 to human TL1A.

### 6.8 Example 8. Immunoreceptor Inhibitory Fusion Protein Mediated Inhibition of TL1A Induced Apoptosis

The ability of IIFPs inhibit TL1A induced cellular apoptosis was assessed, further utilizing the exemplary subset of IIFPs-2, 32, and 33.

Briefly, TF-1 cells (ATCC, Catalog #CRL-2003) were cultured and split the day prior to the experiment at a density of 0.5 million cells/mL. A serial dilution of viral TL1A inhibitors was prepared, starting at a concentration of 100 nM and diluted 22 times in half-log increments. The viral TL1A inhibitors were preincubated with a fixed final dose of 400 pM TL1A trimer (Acro Biosystem, catalog #TLA-H5243-100 ug) for 30 minutes at 4° C. After preincubation, 10 μL of the mixture was added to wells of a Falcon® 384-well Optilux White/Clear Flat Bottom TC-treated (Catalog #353963) with 10,000 TF-1 cells in 20 μL of RPMI-1640 (ATCC, Catalog #30-2001) medium containing 2 ng/ml recombinant human GM-CSF and fetal bovine serum to a final concentration of 10%. Freshly prepared Cycloheximide (MP Biomedicals, Catalog #194527) at 10 μg/mL was then added in 10 μL, bringing the total well volume to 40 μL. The plate was incubated at 37° C. in 5% CO₂ for 6 hours. Subsequently, 40 μL of Caspase-Glo® 3/7 Reagent (Promega, Catalog #G8090) was added to each well, and the plate was shaken at room temperature in the dark for 1 hour. Luminescence was measured using the Varioskan plate reader. Results are expressed as a percentage of apoptosis inhibition.

As shown in FIG. 26, IIFPs-2, 32, and 33 each inhibited TL1A induced apoptosis of TF-1 cells.

### 6.9 Example 9. Summary of Exemplary Functional Attributes of Immunoreceptor Inhibitory Fusion Proteins

Table 11 below provides a summary of the EC₅₀/IC₅₀ (nM) of each of the functional attributes assessed in Examples 3-8 above, providing a quantitative measure of each proteins potency and inhibitory effectiveness in the assays described in Examples 3-8.

TABLE 11
EC₅₀/IC₅₀ (nM) for Exemplary Functional Attributes
of Immunoreceptor Inhibitory Fusion Proteins.

| | EC₅₀ /IC₅₀ [nM] | | |
|---|---|---|---|
| Activity | IIFP-2 | IIFP-32 | IIFP-33 |
| Binding to human | >0.1 ≤ 0.5 | ≥ 0.5 ≤ 1.0 | >0.1 ≤ 0.5 |
| TL1A | | | |
| Binding to membrane | >10 ≤ 100 | >31.6 ≤ 316 | ≥1 ≤ 10 |
| bound human TL1A | | | |
| Inhibition of | >250 ≤ 300 | >500 ≤ 550 | >100 ≤ 150 |
| DR3:TL1A binding | | | |
| Inhibition of | >20 ≤ 30 | >70 ≤ 80 | >1 ≤ 10 |
| DcR3:TL1A binding | | | |
| Inhibition of NFKB | >1 ≤ 10 | >40 ≤ 50 | >1 ≤ 10 |
| signaling | | | |
| Mediated Inhibition | ≥1 ≤ 10 | ≥1 ≤ 10 | ≥0.1 ≤ 1 |
| of TL1A Induced | | | |
| Apoptosis | | | |

### 6.10 Example 10. Immunoreceptor Inhibitory Fusion Protein Preferential Binding to Trimeric Versus Monomeric TL1A

TL1A both trimeric and monomeric forms with the trimeric forms being the predominantly active form (data known shown). As such, the binding kinetics of IIFPs for trimeric and monomeric forms of TL1A were assessed, further utilizing the exemplary subset of IIFPs-2, 32, and 33.

Binding kinetics were performed on a Biacore T200. Briefly, anti-human Fc (Cytiva Cat #BR100839) was coupled to a Series S sensor chip (Cytiva Cat #29104988) using an amine coupling kit (Cytiva Cat #29234600). Samples were diluted and run in running buffer (Cytiva Cat #BR100669) and run at a flow rate of 50 μL/min at 25° C. Samples were stored at 10° C. during the run. CHO produced viral variants were loaded onto the chip and the TL1A monomer (Catalog: TLA-H5246, Acro biosystems, top concentration 125 nM) or trimer (Catalog: TLA-H5243, Acro biosystems, top concentration (62.5 nM) were run at various dilutions with an association time of 5 minutes and a dissociation time of 10 minutes. Multi-cycle kinetics were performed with data fit to a kinetic 1:1 binding model. In between the cycles, the chip was regenerated with 3M MgCl2 for 40 seconds at 30 L/min flow rate. After regeneration, the ligand was captured fresh for the next cycle.

Table 15 shows the binding kinetics of each of IIFP-2, 32, and 33 (including kₐ (1/Ms), k_{d} (1/s), and K_{D} (pM)) for monomeric or trimeric human TL1A.

TABLE 15
Binding Kinetics of IIFPs for Monomeric and Trimeric Forms of TL1A.

| IIFP | Analyte | kₐ (1/Ms) | k_{d} (1/s) | K_{D} (pM) |
|---|---|---|---|---|
| IIFP-32 | Monomer TL1A | >7.3e05 < 8.1e05 | >5.8e−05 < 2.1e−05 | >29 < 71 |
| IIFP-32 | Trimer TL1A | >3.1e06 < 3.4e06 | >2.5e−05 < 1.6e−05 | >5 < 8 |
| IIFP-33 | Monomer TL1A | >5.2e05 < 5.7e05 | >4.6e−05 < 2.1e−05 | >39 < 80 |
| IIFP-33 | Trimer TL1A | >1.9e06 < 2.1e06 | >2.5e−05 < 2.1e−05 | >11 < 12 |
| IIFP-2 | Monomer TL1A | >5.2e05 < 5.7e05 | >4.2e−05 < 1.7e−05 | >34 < 73 |
| IIFP-2 | Trimer TL1A | >1.9e06 < 2.1e06 | >2.9e−05 < 2.2e−05 | >11 < 15 |

As shown above in Table 15, IIFP-2, 32, and 33 each exhibited preferential binding to the trimeric form of TL1A (relative to the monomeric form).

### 6.11 Example 11. In Vivo IFNγ Expression in Mice Treated with Immunoreceptor Inhibitory Fusion Proteins

The in vivo effect of IIFP treatment on the concentration of serum IFNγ in a mouse model was assessed. IIFP-2 was selected as an exemplary IIFP for the assessment.

Briefly, 8-week-old male C57BL/6 mice (Jax Lab, #000664) were dosed with one of the following agents: PBS (control), human Fc (control), anti-mTL1A antibody (5G4.6) (20 mg/kg, 10 mg/kg, or 5 mg/kg), or IIFP-2 (20 mg/kg, 10 mg/kg, or 5 mg/kg) intravenously (IV) via the tail vein. 1 hour later, mice were dosed with 20 mg/kg ConA (Sigma C5275) IV via the tail vein. 18 hours after ConA dosing, blood was collected via terminal cardiac puncture into serum separator tubes (BD 365967), and serum was collected by centrifugation. The IFNγ concentration in the serum was quantified by MSD using a V-PLEX Proinflammatory Panel 1 Mouse Kit (Meso Scale Discovery K15048D) following manufacturer's instructions. Data was collected using a MESO QuickPlex SQ 120MM. Statistical significance was calculated by one-way ANOVA with a Sidak correction for multiple comparisons.

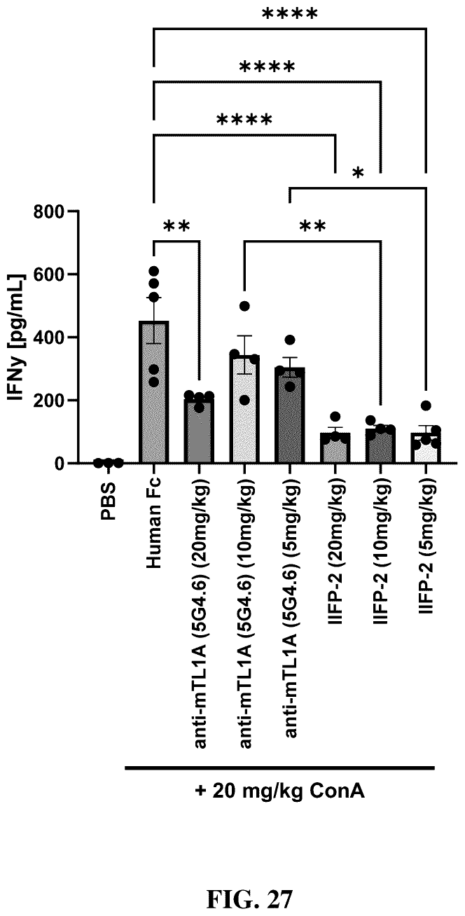

As shown in FIG. 27, IIFP-2 treatment resulted in a significant decrease in the concentration of serum IFNγ, relative to both the human Fc control and the reference anti-mTL1A antibody treatments.

### 6.12 Example 12. Immunoreceptor Inhibitory Fusion Protein Mediated In Vivo Reduction of Joint Inflammation and Local/Systemic Proinflammatory Cytokine Signals in Rheumatoid Arthritis Mouse Model

The in vivo anti-inflammatory efficacy of IFPs was assessed utilizing a collagen antibody induced arthritis (CAIA) mouse model of rheumatoid arthritis, further utilizing IIFP-2 as an exemplary IIFP.

Briefly, 8-week-old, male, DBA/1 mice (Jackson Laboratory, Strain #: 000670) were injected with 1.5 mg of anti-collagen antibodies (Chondrex 5-clone Cocktail Kit, #53100) on day 0 via IV injection. On day 3 mice were injected IP with LPS (LPS E. coli 0111: B4, Chondrex 5-clone Cocktail Kit, #53100). Mice were injected with IFP-2 or hIgG4 Fc control, IP, 10 mg/kg, on days 4, 7, and 11 (n=4 or 5 mice per group). Disease was scored daily following methods previously reported in Khachigian L M. Collagen antibody-induced arthritis. Nat Protoc. 2006; 1 (5): 2512-6. doi: 10.1038/nprot.2006.393. PMID: 17406499, the entire contents of which are incorporated herein by reference for all purposes. Briefly, each limb was scored 0-4, with 0 being normal and 4 being maximally inflamed. The average score for each mouse was calculated and plotted against time. Significance was calculated by two-way ANOVA with Šídák's multiple comparisons test using GraphPad Prism version 10.2.3. Area under the disease scoring curves for hIgG4 Fc and IFP-2 were calculated in GraphPad Prism version 10.2.3. using the trapezoid rule. Groups were compared for statistical significance using an un-paired Student's T-test.

On study day 14, mice were sacrificed, and serum was collected. Left side paws were isolated, stripped of skin, and snap frozen. Snap frozen paws were manually crushed in lysis buffer (Cell Lysis Buffer II, Invitrogen #FNN0021) containing 1× Protease and Phosphatase Inhibitor Cocktail (Abcam #ab201119), centrifuged, and the supernatant collected for analysis. Total protein from crushed joint samples was calculated using a Pierce BCA Protein Assay Kit (Thermo Scientific #A55864) following manufacturer's instructions. Pro-inflammatory cytokines were measured in serum and crushed joint supernatant using a V-PLEX Proinflammatory Panel 1 Mouse MSD Kit (Meso Scale Discovery #K15048D-2) following manufacturer's instructions. Samples were quantified using a MESO QuickPlex SQ 120MM and analyzed using DISCOVERY WORKBENCH software version 4.0. Cytokine levels were expressed as either pg/mL for serum samples or normalized to mg of total protein for crushed joint samples.

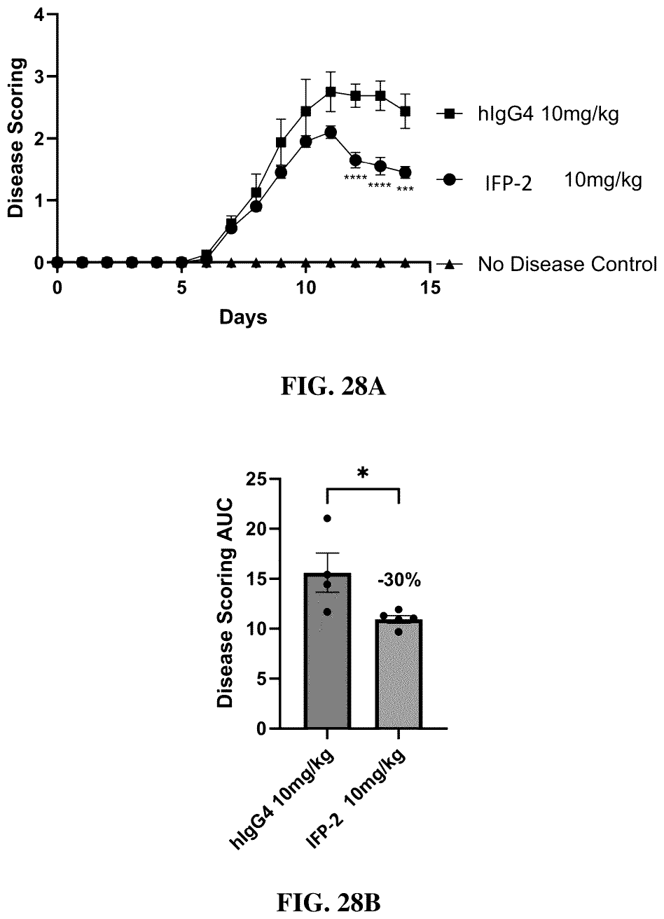

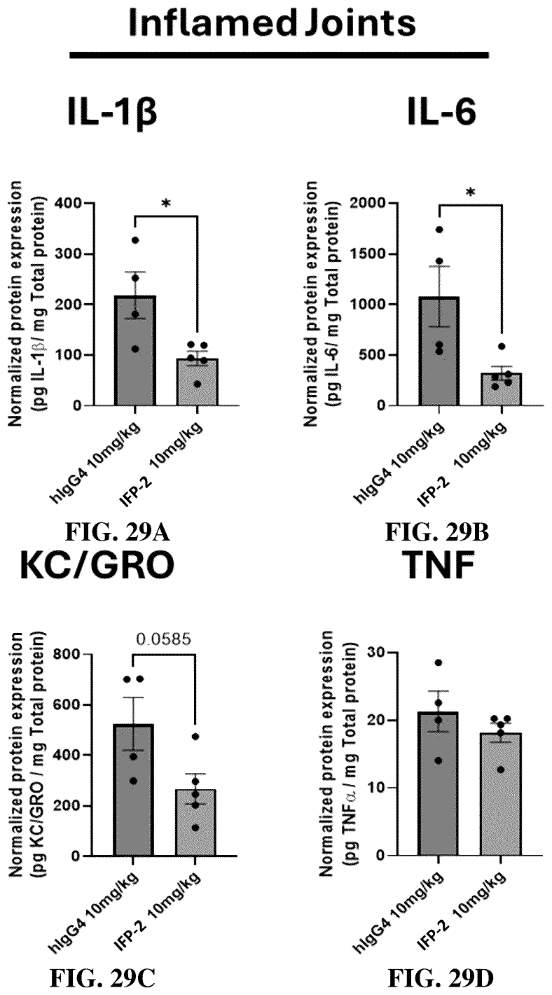

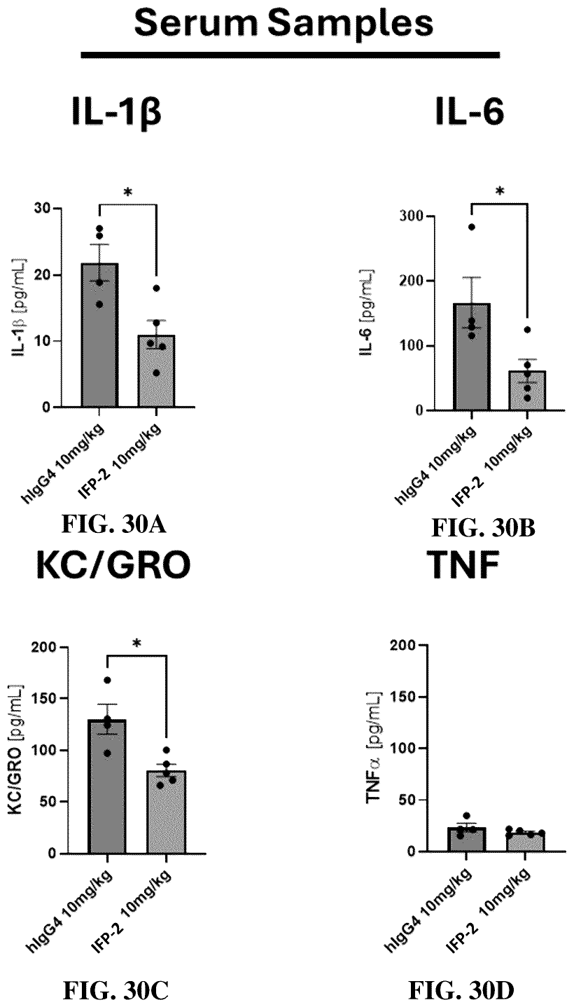

Mice treated with IFP-2 showed abrogated clinical disease score (FIG. 28A and FIG. 28B) in the CAIA mouse model of rheumatoid arthritis, as well as a significant reduction in pro-inflammatory cytokines, including IL-1β, IL-6, and KC/GRO, both locally within the joints (FIGS. 29A-29D) and systematically within the serum (FIGS. 30A-30D).

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1. An isolated protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence of any protein set forth in Table 2 or set forth in any one of SEQ ID NOS: 23-84, 187-190, or 199-200.

2.-25. (canceled)

26. A conjugate comprising the protein of claim 1 operably connected to a heterologous moiety.

27. A radioligand comprising the protein of claim 1 operably connected to a radionuclide.

28. A fusion protein comprising the protein of claim 1 operably connected to a heterologous protein.

29.-90. (canceled)

91. An immunogenic peptide or protein comprising at least an immunogenic fragment of the protein of claim 1.

92.-100. (canceled)

101. An isolated antibody that specifically binds to a protein of claim 1.

102. A nucleic acid molecule encoding the protein of claim 1.

103.-109. (canceled)

110. A vector comprising the nucleic acid molecule of claim 102.

111. (canceled)

112. A viral particle conjugated to the protein of claim 1.

113. A cell comprising the protein of claim 1.

114. A cell expressing and/or genetically encoding the protein of claim 1.

115. A carrier comprising the protein of any one of claim 1.

116. A carrier conjugated to the protein of claim 1.

117.-119. (canceled)

120. A vaccine composition comprising the immunogenic peptide or protein of claim 91.

121. A pharmaceutical composition comprising the protein of claim 1; and a pharmaceutically acceptable excipient.

122. A kit comprising the protein of claim 1; and optionally instructions for using any one or more of the foregoing.

123. A method of delivering a protein to a subject, the method comprising administering to the subject the protein of claim 1, to thereby deliver the protein to a subject.

124. A method of inhibiting or reducing binding of one or more TNFSF ligand to one or more of the TNFSF ligand's cognate TNFSF receptors in a subject in need thereof, the method comprising administering to the subject the protein of claim 1, to thereby inhibit or binding of one or more TNFSF ligand to one or more of the TNFSF ligand's cognate TNFSF receptors in the subject.

125. (canceled)

126. A method of inhibiting or reducing signaling mediated by the binding of one or more TNFSF ligand to one or more of the TNFSF ligand's cognate TNFSF receptors in a subject in need thereof, the method comprising administering to the subject the protein of claim 1, to thereby inhibit or reduce signaling mediated by the binding of one or more TNFSF ligand to one or more of the TNFSF ligand's cognate TNFSF receptors in the subject.

127.-132. (canceled)

133. A method of suppressing or preventing a pro-inflammatory immune response in a subject in need thereof, the method comprising administering to the subject the protein of claim 1, to thereby suppress or prevent a pro-inflammatory immune response in the subject.

134. A method of preventing, treating, or ameliorating a disease in a subject in need thereof, the method comprising administering to the subject the protein of claim 1, to thereby prevent, treat, or ameliorate the disease in the subject.

135.-137. (canceled)

138. A method of inducing or enhancing an immune response in a subject in need thereof, the method comprising administering to the subject (i) the immunogenic peptide or protein of claim 91 (or a conjugate or a fusion protein thereof); (ii) a nucleic acid molecule encoding (i); (iii) a vector comprising (ii); (iv) a carrier comprising (i), (ii), or (iii); a vaccine composition comprising (i), (ii), (iii), or (iv); or a pharmaceutical composition comprising (i), (ii), (iii), (iv), or (v), to thereby induce or enhance an immune response in the subject.

139. A method of vaccinating a subject in need thereof (e.g., against a viral infection), the method comprising administering to the subject (i) the immunogenic peptide or protein of claim 1 (or a conjugate or a fusion protein thereof); (ii) a nucleic acid molecule encoding (i); (iii) a vector comprising (ii); (iv) a carrier comprising (i), (ii), or (iii); a vaccine composition comprising (i), (ii), (iii), or (iv); or a pharmaceutical composition comprising (i), (ii), (iii), (iv), or (v), to thereby vaccinate the subject in need thereof (e.g., against a virus).

140. A method of determining the presence of a virus in a subject, the method comprising
(a) obtaining the sample from a subject or providing a sample that has been obtained from a subject, and
(b) determining the presence or absence of the protein of claim 1 or a nucleic acid molecule encoding the protein (or the fragment or variant thereof) in the sample.

141. A method of diagnosing a viral infection in a subject, the method comprising
(a) obtaining a sample from a subject or providing a sample that has been obtained from a subject,
(b) determining the presence or absence of the protein of claim 1 (or a fragment or variant thereof) or a nucleic acid molecule encoding the protein (or a fragment or variant thereof), and
(c) diagnosing the subject as having the viral infection if the protein or a nucleic acid molecule encoding the protein (or the fragment or variant thereof) is determined to be present in the sample in step (b).

142. (canceled)

143. A method of treating a viral infection in a subject, the method comprising
(a) receiving testing results that determined the presence of the protein of any one of claim 1 (or a fragment or variant thereof) or a nucleic acid molecule encoding the protein (or the fragment or variant thereof) in a sample from the subject,
(b) diagnosing the subject as having the viral infection, and
(c) administering a therapeutic agent to treat the viral infection.

144.-146. (canceled)
